(19) United States
(12) Patent Application Publication
(10) Pub. No.: US 2023/0257393 A1
(43) Pub. Date: Aug. 17, 2023

(54) MACROMOLECULE-SUPPORTED THIENOAZEPINE COMPOUNDS, AND USES THEREOF

(71) Applicant: BOLT BIOTHERAPEUTICS, INC., Rewood City, CA (US)

(72) Inventors: Romas KUDIRKA, Redwood City, CA (US); Brian SAFINA, Redwood City, CA (US)

(73) Assignee: BOLT BIOTHERAPEUTICS, INC., Redwood City, CA (US)

(21) Appl. No.: 17/768,165

(22) PCT Filed: Oct. 23, 2020
(86) PCT No.: PCT/US2020/057162

§ 371 Date: Apr. 11, 2022

(51) Int. Cl. C07D 495/04; C07D 519/00

(52) U.S. Cl. CPC C07D 495/04; C07D 519/00; C07B 2200/05; C07B 2200/11

(57) ABSTRACT

The invention provides macromolecule-supported compounds of Formula I comprising a macromolecular support linked by conjugation to one or more thienoazepine derivatives. The invention also provides thienoazepine derivative intermediate compositions comprising a reactive functional group. Such intermediate compositions are suitable substrates for formation of the macromolecule-supported compounds through a linker or linking moiety. The invention further provides methods of treating cancer with the macromolecule-supported compounds.

### CROSS REFERENCE TO RELATED APPLICATIONS

This non-provisional application claims the benefit of priority to U.S. Provisional Application No. 62/926,324, filed 25 Oct. 2019, which is incorporated by reference in its entirety.

### FIELD OF THE INVENTION

The invention relates generally to a macromolecule-supported compound comprising a macromolecular support conjugated to one or more thienoazepine molecules.

### BACKGROUND OF THE INVENTION

New compositions and methods for the delivery of dendritic cell adjuvants are needed in order to reach inaccessible tumors and/or to expand treatment options for cancer patients and other subjects. The invention provides such compositions and methods.

### BRIEF DESCRIPTION OF THE INVENTION

The invention is generally directed to macromolecule-supported compounds comprising a macromolecular support linked by conjugation to one or more thienoazepine derivatives. The invention is further directed to thienoazepine derivative intermediate compositions comprising a reactive functional group. Such intermediate compositions are suitable substrates for formation of macromolecule-supported compounds wherein a macromolecular support may be covalently bound by a linker L to an thienoazepine (TAZ) moiety having the formula:

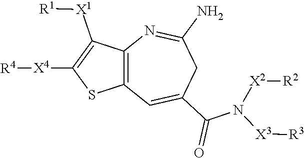

where one of R¹, R², R³ and R⁴ is attached to L. The R¹, R², R³ and R⁴ substituents are defined herein.

An aspect of the invention is a macromolecule-supported compound comprising a macromolecular support covalently attached to a linker which is covalently attached to one or more thienoazepine moieties.

Another aspect of the invention is an thienoazepine-linker compound.

Another aspect of the invention is a method for treating cancer comprising administering a therapeutically effective amount of a macromolecule-supported compound comprising a macromolecular support linked by conjugation to one or more thienoazepine moieties.

Another aspect of the invention is a use of a macromolecule-supported compound comprising a macromolecular support linked by conjugation to one or more thienoazepine moieties for treating cancer.

Another aspect of the invention is a method of preparing a macromolecule-supported compound by conjugation of one or more thienoazepine moieties with a macromolecular support.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

“Adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the macromolecular support or after cleavage (e.g., enzymatic cleavage) from the macromolecular support following administration of a macromolecule-supported compound to the subject.

The terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

As used herein, the phrase “macromolecule-supported compound” refers to a macromolecular support that is covalently bonded to a TLR agonist via a linking moiety.

As used herein, the terms “macromolecule support,” “macromolecular support,” or “macromolecule” can be used interchangeably to refer to an organic or inorganic structure having a chemical moiety on a surface of the structure that can be modified. In some embodiments, the macromolecular support is a resin, bead, probe, tag, well, plate, or any other surface that can be used for therapeutics, diagnostics, or chemical assays. The resin, bead, probe, tag, well, plate, or any other surface can be made of any suitable material so long as the material can be surface modified. In some embodiments, the macromolecular support is a chemical structure (e.g., a biological structure or an inorganic framework) having a molecular weight of at least about 200 Da (e.g., at least about 500 Da, at least about 1,000 Da, at least about 2,000 Da, at least about 5,000 Da, or at least about 10,000 Da). As a singular entity, the macromolecular support can be biologically active or biologically inactive relative to the TLR agonist described herein. However, when used in combination with the TLR agonist, the biological activity of the TLR agonist desirably is enhanced, for example, by providing a targeted effect (i.e., TLR activity), beneficial off-target effects (i.e., biological activity other than TLR activity), improved pharmacokinetic properties (e.g., half-life extension), enhanced biological delivery (e.g., tumor penetration), or additional biological stimulation, differentiation, up-regulation, and/or down-regulation. In certain embodiments, the biological effect of the macromolecular support and the TLR agonist is synergistic, i.e., greater than the sum of the biological activity of each of the macromolecular support and TLR agonist as singular entities. For example, the macromolecular support can be a biopolymer (e.g., a glycopolymer, a cellulosic polymer, etc.), a nanoparticle (e.g., a carbon nanotube, a quantum dot, a metal nanoparticle (e.g., silver, gold, titanium dioxide, silicon dioxide, zirconium dioxide, aluminum oxide, or ytterbium trifluoride), etc.), a lipid (e.g., lipid vesicles, micelles, liposomes, etc.), a carbohydrate (e.g., sugar, starch, cellulose, glycogen, etc.), a peptide (e.g., a polypeptide, a protein, a peptide mimetic, a glycopeptide, etc.), an antibody construct (e.g., antibody, an antibody-derivative (including Fc fusions, Fab fragments and scFvs), etc.), a nucleotide (e.g., RNA, DNA, antisense, siRNA, an aptamer, etc.), or any combination thereof. In some embodiments, the macromolecular support is a peptide, a nucleotide, a sugar, a lipid, or an antibody. In certain embodiments, the macromolecular support is an immune checkpoint inhibitor.

As used herein, the term “biopolymer” refers to any polymer produced by a living organism. For example, biopolymer can include peptides, polypeptides, proteins, oligonucleotides, nucleic acids (e.g., RNA and DNA) antibodies, polysaccharides, carbohydrates, sugars, peptide hormones, glycoproteins, glycogen, etc. Alternatively, a subunit of a biopolymer, such as a fatty acid, glucose, an amino acid, a succinate, a ribonucleotide, a ribonucleoside, a deoxyribonucleotide, and a deoxyribonucleoside can be used. Illustrative examples include antibodies or fragments thereof; extracellular matrix proteins such as laminin, fibronectin, growth factors, peptide hormones, and other polypeptides. In some embodiments, the biopolymer comprises suberin, melanin, lignin, or cellulose, or the biopolymer is glycosidic.

As used herein, the term “nanoparticle” refers to a support structure having a diameter of about 1 nm to about 100 nm. Exemplary structure types include nanopowders, nanoparticles, nanoclusters, nanorods, nanotubes, nanocrystals, nanospheres, nanochains, nanoreefs, nanoboxes, and quantum dots. The nanoparticles can contain an inorganic material (e.g., silver, gold, hydroxyapatite, clay, titanium dioxide, silicon dioxide, zirconium dioxide, carbon (graphite), diamond, aluminum oxide, ytterbium trifluoride, etc.) or an organic material (e.g., micelles, dendrimers, vesicles, liposomes, etc.). Alternatively, the nanoparticle can have a mixture of organic and inorganic material.

As used herein the term “lipid” refers to a hydrophobic or amphiphilic biomolecule. Exemplary lipids include fatty acids, waxes, sterols, fat-soluble vitamins, monoglycerides, diglycerides, triglycerides, phospholipids, sphingolipids, saccharolipids, polyketides, sterol lipids, glycerophospholipids, prenol lipids, etc. The lipid can exist in any suitable macromolecular structure, for example, a vesicle, a micelle, a liposome, etc.

As used herein, the term “carbohydrate” refers to any chemical entity comprising a monosaccharide, disaccharide, oligosaccharide, or polysaccharide. For example, the chemical entity can comprise a sugar (e.g., fructose, glucose, sucrose, lactose, galactose, etc.), starch, glycogen, or cellulose.

The terms “polypeptide,” “peptide,” and “protein” are used interchangeably herein to refer to a polymer of amino acid residues. The terms also apply to amino acid polymers in which one or more amino acid residues is an artificial chemical mimetic of a corresponding naturally occurring amino acid, as well as to naturally occurring amino acid polymers and non-naturally occurring amino acid polymers. The peptide can have any suitable posttranslational modification (e.g., phosphorylation, hydroxylation, sulfonation, palmitoylation, glycosylation, disulfide formation, galactosylation, fucosylation, etc.).

As used herein, the phrase “alternative protein scaffold” refers to a non-immunoglobulin derived protein or peptide. Such proteins and peptides are generally amenable to engineering and can be designed to confer monospecificity against a given antigen, bispecificity, or multispecificity. Engineering of an alternative protein scaffold can be conducted using several approaches. A loop grafting approach can be used where sequences of known specificity are grafted onto a variable loop of a scaffold. Sequence randomization and mutagenesis can be used to develop a library of mutants, which can be screened using various display platforms (e.g., phage display) to identify a novel binder. Site-specific mutagenesis can also be used as part of a similar approach. Alternative protein scaffolds exist in a variety of sizes, ranging from small peptides with minimal secondary structure to large proteins of similar size to a full-sized antibody. Examples of scaffolds include, but are not limited to, cystine knotted miniproteins (also known as knottins), cyclic cystine knotted miniproteins (also known as cyclotides), avimers, affibodies, the tenth type III domain of human fibronectin, DARPins (designed ankyrin repeats), and anticalins (also known as lipocalins). Naturally occurring ligands with known specificity can also be engineered to confer novel specificity against a given target. Examples of naturally occurring ligands that may be engineered include the EGF ligand and VEGF ligand. Engineered proteins can either be produced as monomeric proteins or as multimers, depending on the desired binding strategy and specificities. Protein engineering strategies can be used to fuse alternative protein scaffolds to Fc domains.

As used herein, the term “nucleotide” refers to any chemical entity comprising deoxyribonucleic acid (“DNA”), ribonucleic acid (“RNA”), a deoxyribonucleic acid derivative, or a ribonucleic acid derivative. Exemplary nucleotide-based structures include RNA, DNA, antisense oligonucleotides, siRNA, aptamers, etc. As used herein, the terms “deoxyribonucleic acid derivative” and “ribonucleic acid derivative” refer to DNA and RNA, respectively, that have been modified, such as, for example, by removing the phosphate backbone, methylating a hydroxyl group, or replacing a hydroxyl group with a thiol group.

As used herein, the phrase “antibody construct” refers to polypeptide comprising an antigen binding domain and an Fc domain. An antibody construct can comprise or be an antibody.

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Antibody construct” refers to an antibody or a fusion protein comprising (i) an antigen binding domain and (ii) an Fe domain.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

“Biosimilar” refers to an approved antibody construct that has active properties similar to, for example, a PD-L1-targeting antibody construct previously approved such as atezolizumab (TECENTRIQ™, Genentech, Inc.), durvalumab (IMFINZI™, AstraZeneca), and avelumab (BAVENCIO™, EMD Serono, Pfizer); a HER2-targeting antibody construct previously approved such as trastuzumab (HERCEPTIN™, Genentech, Inc.), and pertuzumab (PERJETA™, Genentech, Inc.); or a CEA-targeting antibody such as labetuzumab (CEA-CIDE™, MN-14, hMN14, Immunomedics) CAS Reg. No. 219649-07-7).

“Biobetter” refers to an approved antibody construct that is an improvement of a previously approved antibody construct, such as atezolizumab, durvalumab, avelumab, trastuzumab, pertuzumab, and labetuzumab. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in a compound or material. For example, the linking moiety can serve to covalently bond an adjuvant moiety to an antibody construct in a macromolecule-supported compound.

“Linking moiety” refers to a functional group that covalently bonds two or more moieties in a compound or material. For example, the linking moiety can serve to covalently bond an adjuvant moiety to a macromolecular support in a macromolecule-supported compound. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line (“ [symbol] ”) represents a point of attachment of the specified chemical moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡CH), propynyl (propargyl, —CH₂C≡CH), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system.. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

“PD-L1 expression” refers to a cell that has a PD-L1 receptor on the cell's surface. As used herein “PD-L1 overexpression” refers to a cell that has more PD-L1 receptors as compared to corresponding non-cancer cell.

“HER2” refers to the protein human epidermal growth factor receptor 2.

“HER2 expression” refers to a cell that has a HER2 receptor on the cell's surface. For example, a cell may have from about 20,000 to about 50,000 HER2 receptors on the cell's surface. As used herein “HER2 overexpression” refers to a cell that has more than about 50,000 HER2 receptors. For example, a cell 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed in about 25% to about 30% of breast cancers.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as a macromolecule-supported compound that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the macromolecule-supported compound may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the macromolecule-supported compound may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the macromolecule-supported compounds disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the macromolecule-supported compound may be administered (as measured by the total number of antibody constructs or the total number of adjuvants administered as part of the macromolecule-supported compound) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### Thienoazepine Adjuvant Compounds

The macromolecule-supported compound of the invention comprises a thienoazepine adjuvant moiety. The adjuvant moiety described herein is a compound that elicits an immune response (i.e., an immunostimulatory agent). Generally, the adjuvant moiety described herein is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-κB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several TRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, TL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

Exemplary thienoazepine compounds (TAZ) of the invention are shown in Tables 1a-c. Each compound was synthesized, purified, and characterized by mass spectrometry and shown to have the mass indicated. Additional experimental procedures are found in the Examples. Activity against HEK293 NFKB reporter cells expressing human TLR7 or human TLR8 was measured according to Example 202. The thienoazepine compounds of Tables 1a-c demonstrate the surprising and unexpected property of TLR8 agonist selectivity which may predict useful therapeutic activity to treat cancer and other disorders.

TABLE 1a
Thienoazepine compounds (TAZ)

| | | | HEK293 | HEK293 |
|---|---|---|---|---|
| TAZ | | | hTLR7 | hTLR8 |
| No. | Structure | MW | EC50 (nM) | EC50 (nM) |
| TAZ-1 | 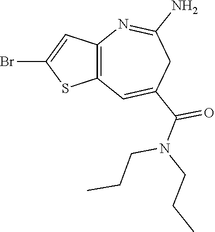 [SMILES: CCCN(CCC)C(=O)C1=Cc2sc(Br)cc2N=C(N)C1] | 370.3 | >9000 | >9000 |
| TAZ-2 | 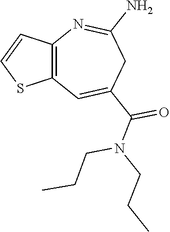 [SMILES: CCCN(CCC)C(=O)C1=Cc2sccc2N=C(N)C1] | 291.4 | >9000 | 2390 |
| TAZ-3 | 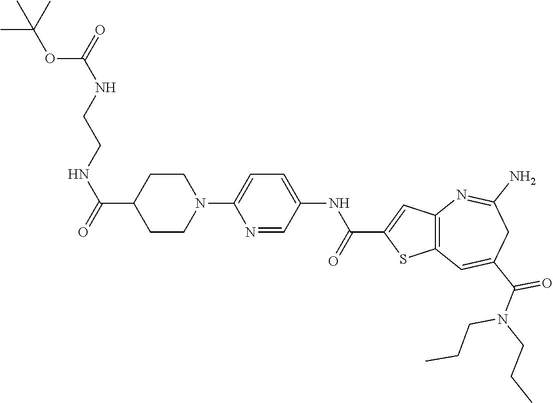 [SMILES: CCCN(CCC)C(=O)C1=Cc2sc(C(=O)Nc3ccc(N4CCC(C(=O)NCCNC(=O)OC(C)(C)C)CC4)nc3)cc2N=C(N)C1] | 680.9 | >9000 | >9000 |
| TAZ-4 | 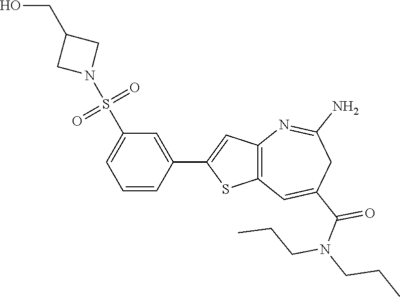 [SMILES: CCCN(CCC)C(=O)C1=Cc2sc(-c3cccc(S(=O)(=O)N4CC(CO)C4)c3)cc2N=C(N)C1] | 516.7 | >9000 | >9000 |
| TAZ-5 | 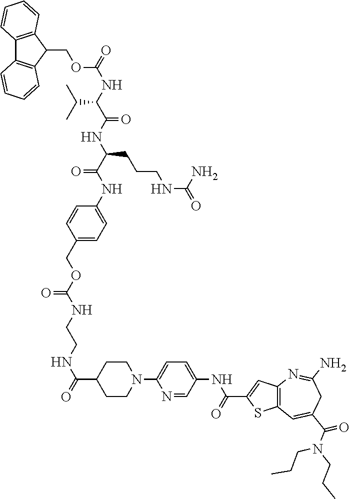 [SMILES: CCCN(CCC)C(=O)C1=Cc2sc(C(=O)Nc3ccc(N4CCC(C(=O)NCCNC(=O)OCc5ccc(NC(=O)[C@H](CCCNC(N)=O)NC(=O)[C@@H](NC(=O)OCC6c7ccccc7-c7ccccc76)C(C)C)cc5)CC4)nc3)cc2N=C(N)C1] | 1208.4 | Not Determined | Not Determined |
| TAZ-6 | 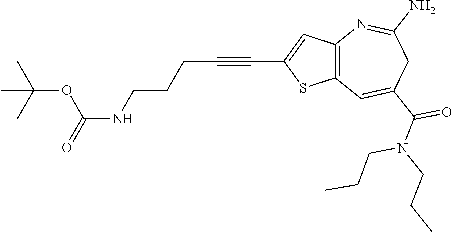 [SMILES: CCCN(CCC)C(=O)C1=Cc2sc(C#CCCCNC(=O)OC(C)(C)C)cc2N=C(N)C1] | 472.7 | >9000 | >9000 |
| TAZ-7 | 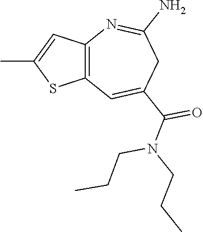 [SMILES: CCCN(CCC)C(=O)C1=Cc2sc(C)cc2N=C(N)C1] | 305.4 | >9000 | >9000 |
| TAZ-8 | 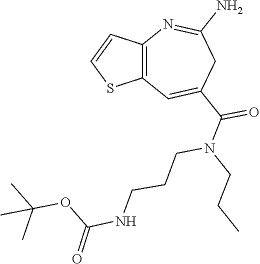 [SMILES: CCCN(CCCNC(=O)OC(C)(C)C)C(=O)C1=Cc2sccc2N=C(N)C1] | 406.5 | 589 | 1533 |
| TAZ-9 | 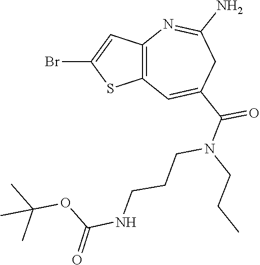 [SMILES: CCCN(CCCNC(=O)OC(C)(C)C)C(=O)C1=Cc2sc(Br)cc2N=C(N)C1] | 485.4 | >9000 | >9000 |
| TAZ-10 | 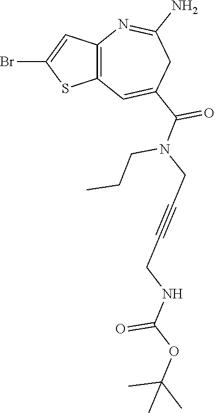 [SMILES: CCCN(CC#CCNC(=O)OC(C)(C)C)C(=O)C1=Cc2sc(Br)cc2N=C(N)C1] | 495.4 | 7767 | >9000 |
| TAZ-11 | 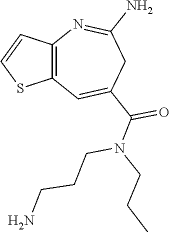 [SMILES: CCCN(CCCN)C(=O)C1=Cc2sccc2N=C(N)C1] | 306.4 | >9000 | >9000 |
| TAZ-12 | 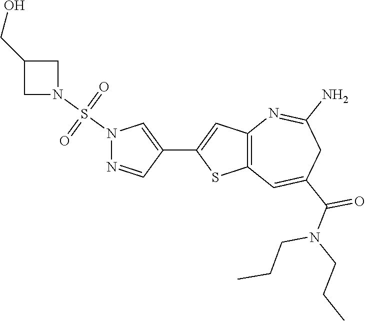 [SMILES: CCCN(CCC)C(=O)C1=Cc2sc(-c3cnn(S(=O)(=O)N4CC(CO)C4)c3)cc2N=C(N)C1] | 506.6 | >9000 | >9000 |
| TAZ-13 | 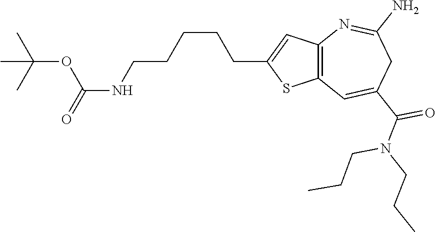 [SMILES: CCCN(CCC)C(=O)C1=Cc2sc(CCCCCNC(=O)OC(C)(C)C)cc2N=C(N)C1] | 476.7 | >9000 | >9000 |
| TAZ-14 | 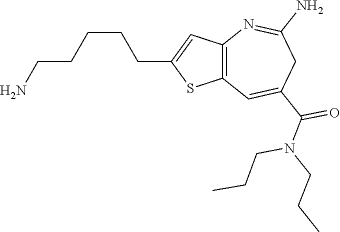 [SMILES: CCCN(CCC)C(=O)C1=Cc2sc(CCCCCN)cc2N=C(N)C1] | 376.6 | >9000 | 4940 |
| TAZ-15 | 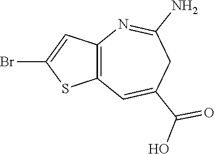 [SMILES: NC1=Nc2cc(Br)sc2C=C(C(=O)O)C1] | 287.1 | Not Determined | Not Determined |
| TAZ-16 | 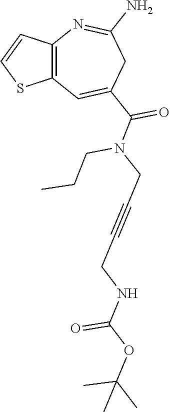 [SMILES: CCCN(CC#CCNC(=O)OC(C)(C)C)C(=O)C1=Cc2sccc2N=C(N)C1] | 416.5 | >9000 | 6111 |
| TAZ-17 | 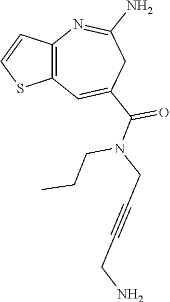 [SMILES: CCCN(CC#CCN)C(=O)C1=Cc2sccc2N=C(N)C1] | 316.4 | >9000 | 4101 |
| TAZ-18 | 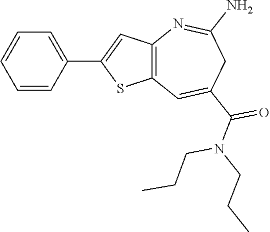 [SMILES: CCCN(CCC)C(=O)C1=Cc2sc(-c3ccccc3)cc2N=C(N)C1] | 367.5 | >9000 | >9000 |
| TAZ-19 | 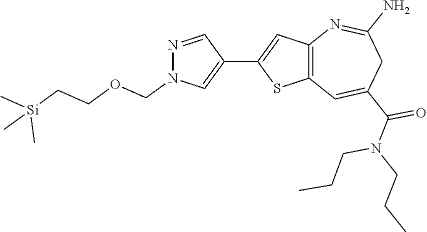 [SMILES: CCCN(CCC)C(=O)C1=Cc2sc(-c3cnn(COCC[Si](C)(C)C)c3)cc2N=C(N)C1] | 487.7 | >9000 | >9000 |
| TAZ-20 | 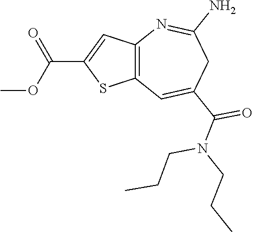 [SMILES: CCCN(CCC)C(=O)C1=Cc2sc(C(=O)OC)cc2N=C(N)C1] | 349.4 | >9000 | >9000 |
| TAZ-21 | 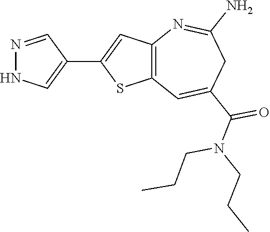 [SMILES: CCCN(CCC)C(=O)C1=Cc2sc(-c3cn[nH]c3)cc2N=C(N)C1] | 357.5 | >9000 | >9000 |
| TAZ-22 | 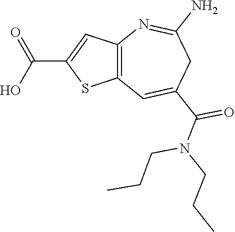 [SMILES: CCCN(CCC)C(=O)C1=Cc2sc(C(=O)O)cc2N=C(N)C1] | 335.4 | >9000 | >9000 |
| TAZ-23 | 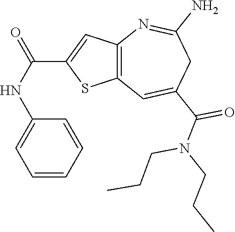 [SMILES: CCCN(CCC)C(=O)C1=Cc2sc(C(=O)Nc3ccccc3)cc2N=C(N)C1] | 410.5 | >9000 | >9000 |
| TAZ-24 | 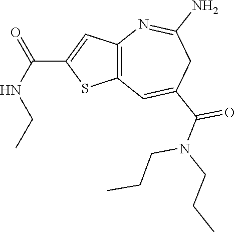 [SMILES: CCCN(CCC)C(=O)C1=Cc2sc(C(=O)NCC)cc2N=C(N)C1] | 362.5 | >9000 | >9000 |
| TAZ-25 | 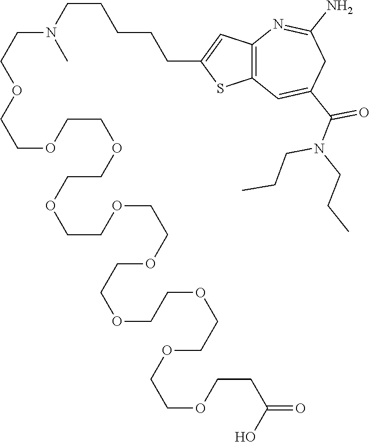 [SMILES: CCCN(CCC)C(=O)C1=Cc2sc(CCCCCN(C)CCOCCOCCOCCOCCOCCOCCOCCOCCOCCOCCC(=O)O)cc2N=C(N)C1] | 903.2 | Not Determined | Not Determined |
| TAZ-26 | 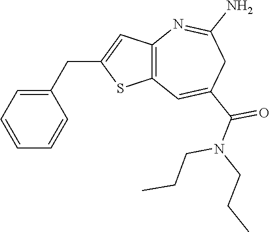 [SMILES: CCCN(CCC)C(=O)C1=Cc2sc(Cc3ccccc3)cc2N=C(N)C1] | 381.5 | >9000 | >9000 |
| TAZ-27 | 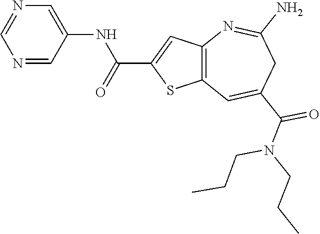 [SMILES: CCCN(CCC)C(=O)C1=Cc2sc(C(=O)Nc3cncnc3)cc2N=C(N)C1] | 412.5 | >9000 | >9000 |
| TAZ-28 | 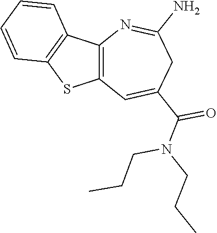 [SMILES: CCCN(CCC)C(=O)C1=Cc2sc3ccccc3c2N=C(N)C1] | 341.5 | >9000 | >9000 |
| TAZ-29 | 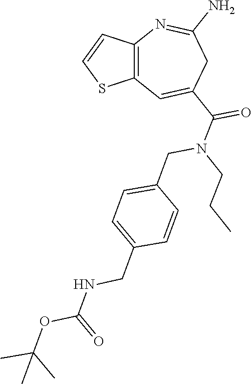 [SMILES: CCCN(Cc1ccc(CNC(=O)OC(C)(C)C)cc1)C(=O)C1=Cc2sccc2N=C(N)C1] | 468.6 | >9000 | >9000 |
| TAZ-30 | 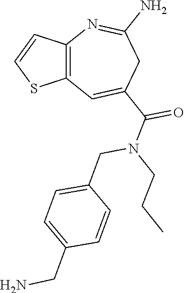 [SMILES: CCCN(Cc1ccc(CN)cc1)C(=O)C1=Cc2sccc2N=C(N)C1] | 368.5 | >9000 | 641 |

TABLE 1b
Thienoazepine compounds (TAZ)

| | | | HEK293 | HEK293 |
|---|---|---|---|---|
| TAZ | | | hTLR7 | hTLR8 |
| No. | Structure | MW | EC50 (nM) | EC50 (nM) |
| TAZ-31 | 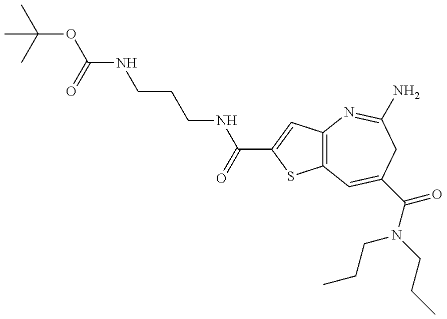 [SMILES: CCCN(CCC)C(=O)C1=Cc2sc(C(=O)NCCCNC(=O)OC(C)(C)C)cc2N=C(N)C1] | 491.65 | >9000 | >9000 |
| TAZ-32 | 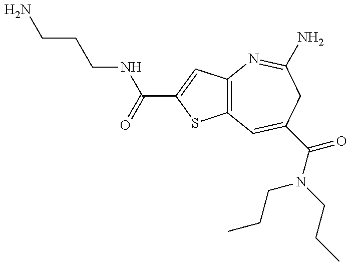 [SMILES: CCCN(CCC)C(=O)C1=Cc2sc(C(=O)NCCCN)cc2N=C(N)C1] | 391.53 | >9000 | >9000 |
| TAZ-33 | 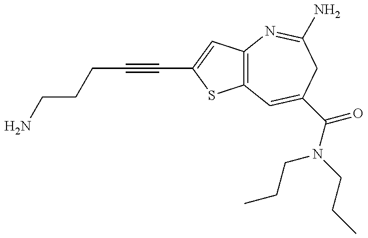 [SMILES: CCCN(CCC)C(=O)C1=Cc2sc(C#CCCCN)cc2N=C(N)C1] | 372.53 | >9000 | >9000 |
| TAZ-34 | 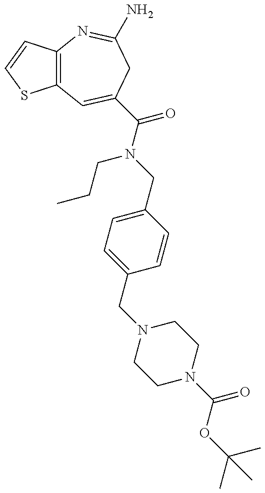 [SMILES: CCCN(Cc1ccc(CN2CCN(C(=O)OC(C)(C)C)CC2)cc1)C(=O)C1=Cc2sccc2N=C(N)C1] | 537.72 | >9000 | 339 |
| TAZ-35 | 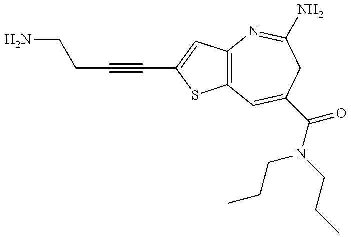 [SMILES: CCCN(CCC)C(=O)C1=Cc2sc(C#CCCN)cc2N=C(N)C1] | 358.5 | >9000 | 2870 |
| TAZ-36 | 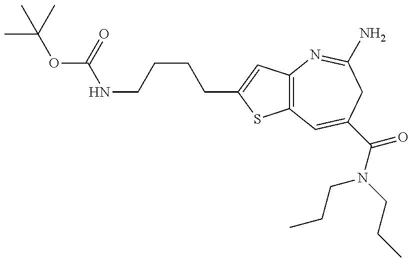 [SMILES: CCCN(CCC)C(=O)C1=Cc2sc(CCCCNC(=O)OC(C)(C)C)cc2N=C(N)C1] | 462.65 | 8524 | >9000 |
| TAZ-37 | 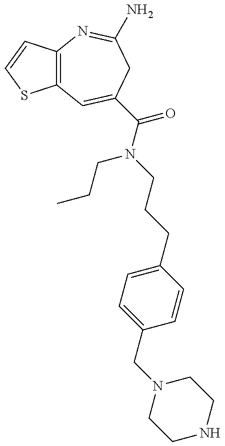 [SMILES: CCCN(CCCc1ccc(CN2CCNCC2)cc1)C(=O)C1=Cc2sccc2N=C(N)C1] | 437.6 | >9000 | 2938 |
| TAZ-38 | 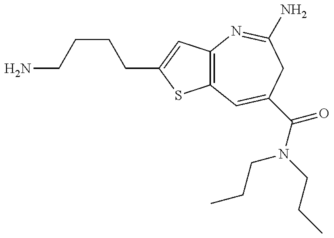 [SMILES: CCCN(CCC)C(=O)C1=Cc2sc(CCCCN)cc2N=C(N)C1] | 362.53 | 4148 | 3752 |
| TAZ-39 | 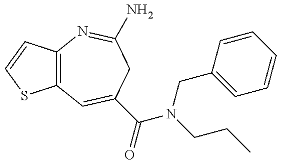 [SMILES: CCCN(Cc1ccccc1)C(=O)C1=Cc2sccc2N=C(N)C1] | 339.46 | >9000 | >9000 |
| TAZ-40 | 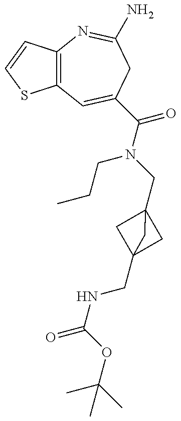 [SMILES: CCCN(CC12CC(CNC(=O)OC(C)(C)C)(C1)C2)C(=O)C1=Cc2sccc2N=C(N)C1] | 458.62 | >9000 | >9000 |
| TAZ-41 | 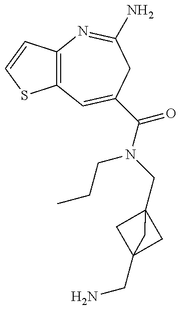 [SMILES: CCCN(CC12CC(CN)(C1)C2)C(=O)C1=Cc2sccc2N=C(N)C1] | 358.5 | >9000 | >9000 |
| TAZ-42 | 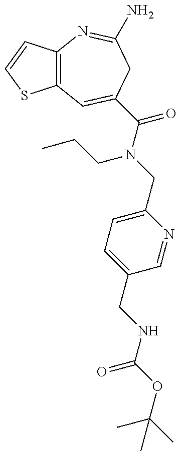 [SMILES: CCCN(Cc1ccc(CNC(=O)OC(C)(C)C)cn1)C(=O)C1=Cc2sccc2N=C(N)C1] | 469.6 | >9000 | >9000 |
| TAZ-43 | 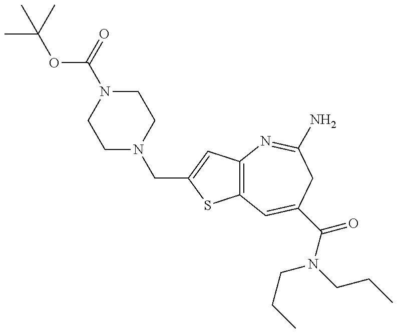 [SMILES: CCCN(CCC)C(=O)C1=Cc2sc(CN3CCN(C(=O)OC(C)(C)C)CC3)cc2N=C(N)C1] | 489.68 | >9000 | >9000 |
| TAZ-44 | 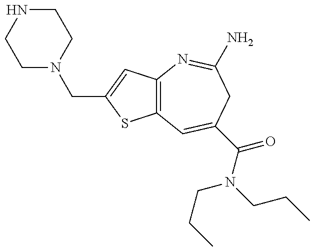 [SMILES: CCCN(CCC)C(=O)C1=Cc2sc(CN3CCNCC3)cc2N=C(N)C1] | 389.56 | 4526 | 593 |
| TAZ-45 | 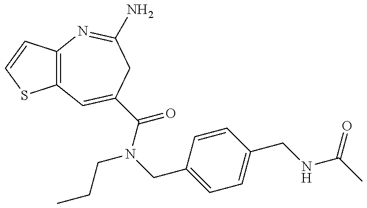 [SMILES: CCCN(Cc1ccc(CNC(C)=O)cc1)C(=O)C1=Cc2sccc2N=C(N)C1] | 410.53 | >9000 | 1779 |
| TAZ-46 | 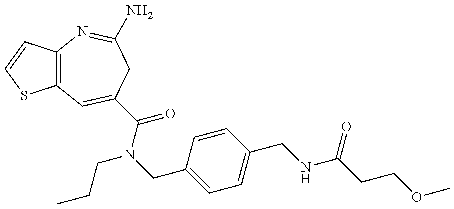 [SMILES: CCCN(Cc1ccc(CNC(=O)CCOC)cc1)C(=O)C1=Cc2sccc2N=C(N)C1] | 454.59 | >9000 | 1576 |
| TAZ-47 | 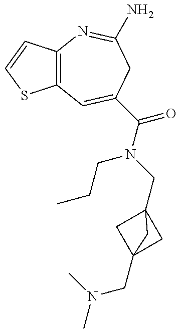 [SMILES: CCCN(CC12CC(CN(C)C)(C1)C2)C(=O)C1=Cc2sccc2N=C(N)C1] | 386.56 | >9000 | >9000 |
| TAZ-48 | 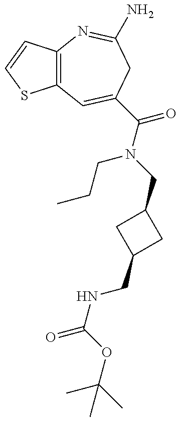 [SMILES: CCCN(C[C@H]1C[C@@H](CNC(=O)OC(C)(C)C)C1)C(=O)C1=Cc2sccc2N=C(N)C1] | 446.61 | >9000 | >9000 |
| TAZ-49 | 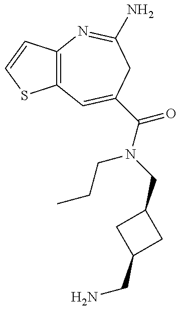 [SMILES: CCCN(C[C@H]1C[C@@H](CN)C1)C(=O)C1=Cc2sccc2N=C(N)C1] | 346.49 | >9000 | >9000 |
| TAZ-50 | 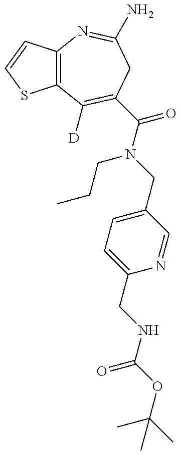 [SMILES: [2H]C1=C(C(=O)N(CCC)Cc2ccc(CNC(=O)OC(C)(C)C)nc2)CC(N)=Nc2ccsc21] | 469.6 | 7125 | 7938 |
| TAZ-51 | 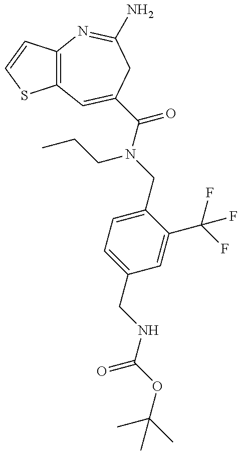 [SMILES: CCCN(Cc1ccc(CNC(=O)OC(C)(C)C)cc1C(F)(F)F)C(=O)C1=Cc2sccc2N=C(N)C1] | 536.61 | >9000 | 5635 |
| TAZ-52 | 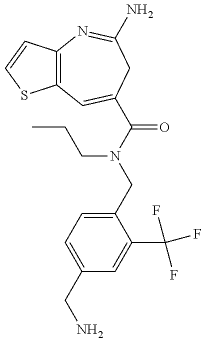 [SMILES: CCCN(Cc1ccc(CN)cc1C(F)(F)F)C(=O)C1=Cc2sccc2N=C(N)C1] | 436.5 | 2705 | 151 |
| TAZ-53 | 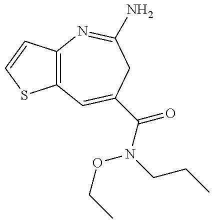 [SMILES: CCCN(OCC)C(=O)C1=Cc2sccc2N=C(N)C1] | 293.39 | 3621 | 181 |
| TAZ-54 | 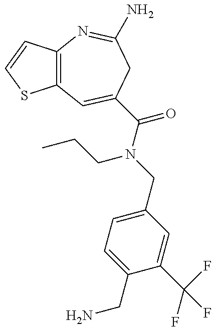 [SMILES: CCCN(Cc1ccc(CN)c(C(F)(F)F)c1)C(=O)C1=Cc2sccc2N=C(N)C1] | 436.5 | 905 | 35 |
| TAZ-55 | 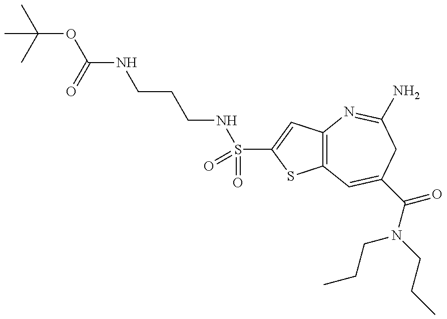 [SMILES: CCCN(CCC)C(=O)C1=Cc2sc(S(=O)(=O)NCCCNC(=O)OC(C)(C)C)cc2N=C(N)C1] | 527.7 | >9000 | >9000 |
| TAZ-56 | 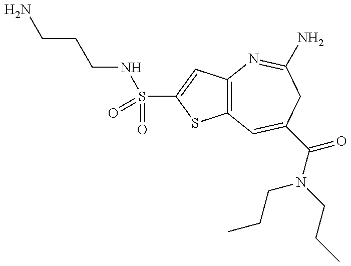 [SMILES: CCCN(CCC)C(=O)C1=Cc2sc(S(=O)(=O)NCCCN)cc2N=C(N)C1] | 427.58 | >9000 | >9000 |
| TAZ-57 | 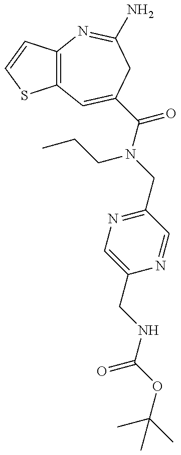 [SMILES: CCCN(Cc1cnc(CNC(=O)OC(C)(C)C)cn1)C(=O)C1=Cc2sccc2N=C(N)C1] | 470.59 | 1053 | 3850 |
| TAZ-58 | 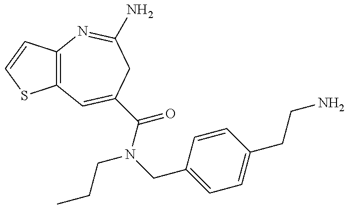 [SMILES: CCCN(Cc1ccc(CCN)cc1)C(=O)C1=Cc2sccc2N=C(N)C1] | 382.52 | >9000 | 1296 |
| TAZ-59 | 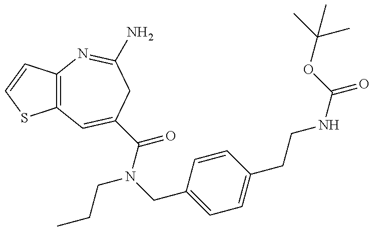 [SMILES: CCCN(Cc1ccc(CCNC(=O)OC(C)(C)C)cc1)C(=O)C1=Cc2sccc2N=C(N)C1] | 482.64 | >9000 | >9000 |
| TAZ-60 | 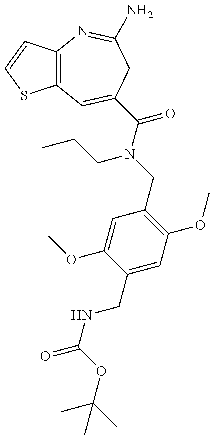 [SMILES: CCCN(Cc1cc(OC)c(CNC(=O)OC(C)(C)C)cc1OC)C(=O)C1=Cc2sccc2N=C(N)C1] | 528.67 | Not available | Not available |
| TAZ-61 | 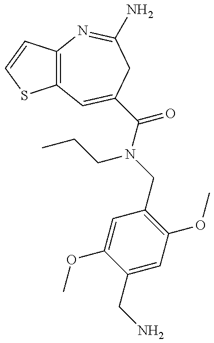 [SMILES: CCCN(Cc1cc(OC)c(CN)cc1OC)C(=O)C1=Cc2sccc2N=C(N)C1] | 428.55 | Not available | Not available |
| TAZ-62 | 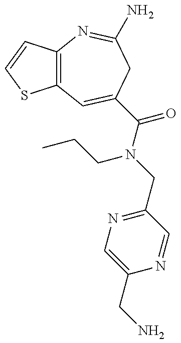 [SMILES: CCCN(Cc1cnc(CN)cn1)C(=O)C1=Cc2sccc2N=C(N)C1] | 370.47 | 8317 | 178 |
| TAZ-63 | 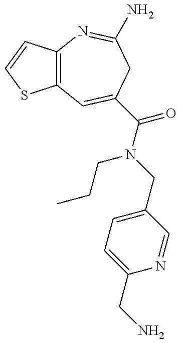 [SMILES: CCCN(Cc1ccc(CN)nc1)C(=O)C1=Cc2sccc2N=C(N)C1] | 369.48 | 4209 | 517 |
| TAZ-64 | 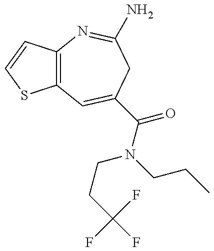 [SMILES: CCCN(CCC(F)(F)F)C(=O)C1=Cc2sccc2N=C(N)C1] | 345.38 | 5057 | 2742 |
| TAZ-65 | 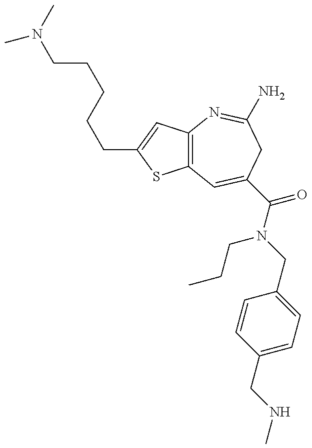 [SMILES: CCCN(Cc1ccc(CNC)cc1)C(=O)C1=Cc2sc(CCCCCN(C)C)cc2N=C(N)C1] | 495.73 | >9000 | 311 |
| TAZ-66 | 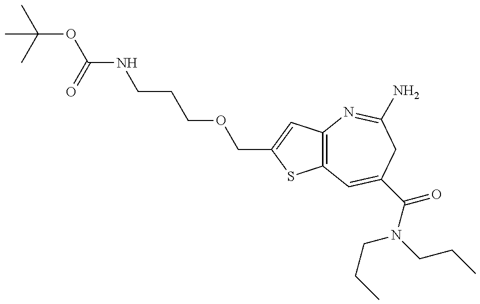 [SMILES: CCCN(CCC)C(=O)C1=Cc2sc(COCCCNC(=O)OC(C)(C)C)cc2N=C(N)C1] | 478.65 | 4423 | 4236 |
| TAZ-67 | 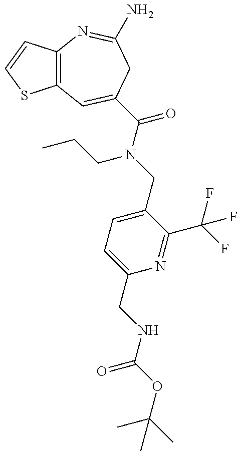 [SMILES: CCCN(Cc1ccc(CNC(=O)OC(C)(C)C)nc1C(F)(F)F)C(=O)C1=Cc2sccc2N=C(N)C1] | 537.6 | Not available | Not available |
| TAZ-68 | 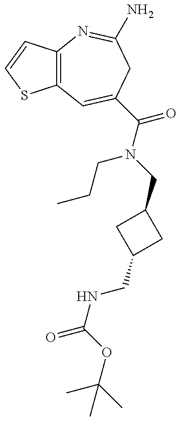 [SMILES: CCCN(C[C@H]1C[C@H](CNC(=O)OC(C)(C)C)C1)C(=O)C1=Cc2sccc2N=C(N)C1] | 446.61 | Not available | Not available |
| TAZ-69 | 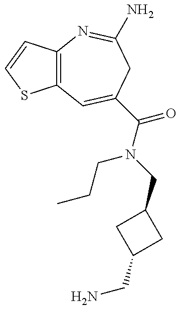 [SMILES: CCCN(C[C@H]1C[C@H](CN)C1)C(=O)C1=Cc2sccc2N=C(N)C1] | 346.49 | >9000 | 4297 |
| TAZ-70 | 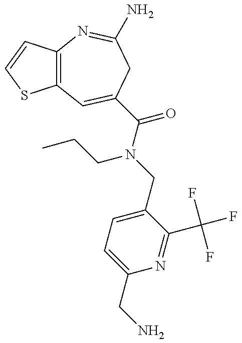 [SMILES: CCCN(Cc1ccc(CN)nc1C(F)(F)F)C(=O)C1=Cc2sccc2N=C(N)C1] | 437.48 | 6613 | 75 |
| TAZ-71 | 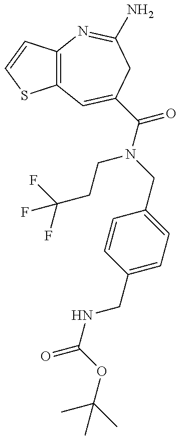 [SMILES: CC(C)(C)OC(=O)NCc1ccc(CN(CCC(F)(F)F)C(=O)C2=Cc3sccc3N=C(N)C2)cc1] | 522.58 | | |
| TAZ-72 | 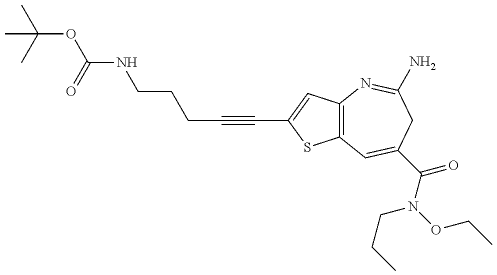 [SMILES: CCCN(OCC)C(=O)C1=Cc2sc(C#CCCCNC(=O)OC(C)(C)C)cc2N=C(N)C1] | 474.62 | 3146 | 2834 |
| TAZ-73 | 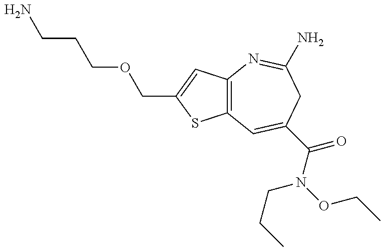 [SMILES: CCCN(OCC)C(=O)C1=Cc2sc(COCCCN)cc2N=C(N)C1] | 378.53 | >9000 | 2985 |
| TAZ-74 | 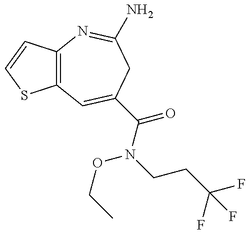 [SMILES: CCON(CCC(F)(F)F)C(=O)C1=Cc2sccc2N=C(N)C1] | 347.36 | 4108 | 301 |
| TAZ-75 | 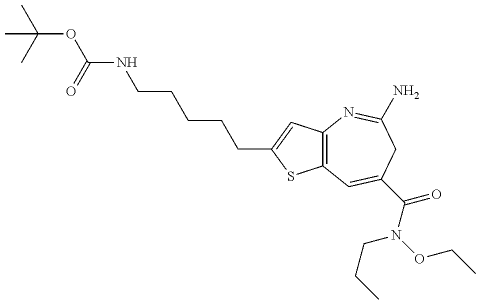 [SMILES: CCCN(OCC)C(=O)C1=Cc2sc(CCCCCNC(=O)OC(C)(C)C)cc2N=C(N)C1] | 478.65 | 5079 | 2025 |
| TAZ-76 | 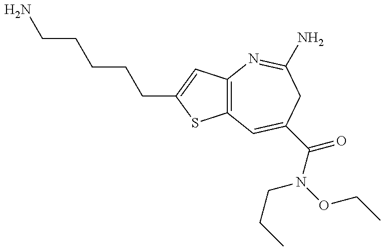 [SMILES: CCCN(OCC)C(=O)C1=Cc2sc(CCCCCN)cc2N=C(N)C1] | 378.53 | 3436 | 1418 |
| TAZ-77 | 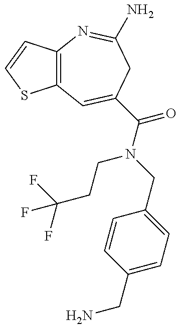 [SMILES: NCc1ccc(CN(CCC(F)(F)F)C(=O)C2=Cc3sccc3N=C(N)C2)cc1] | 422.47 | 3896 | 59 |
| TAZ-78 | 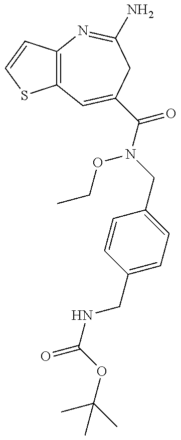 [SMILES: CCON(Cc1ccc(CNC(=O)OC(C)(C)C)cc1)C(=O)C1=Cc2sccc2N=C(N)C1] | 470.59 | Not available | Not available |
| TAZ-79 | 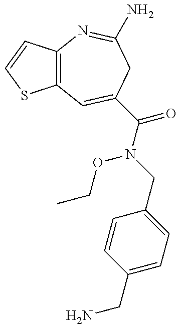 [SMILES: CCON(Cc1ccc(CN)cc1)C(=O)C1=Cc2sccc2N=C(N)C1] | 370.47 | >9000 | 4126 |
| TAZ-80 | 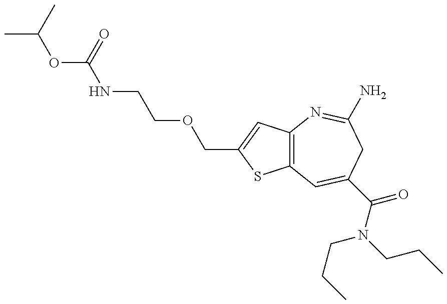 [SMILES: CCCN(CCC)C(=O)C1=Cc2sc(COCCNC(=O)OC(C)C)cc2N=C(N)C1] | 464.62 | 4002 | 3793 |
| TAZ-81 | 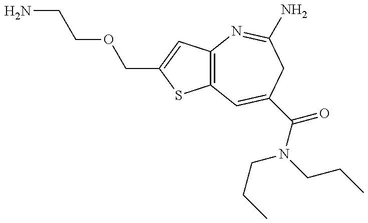 [SMILES: CCCN(CCC)C(=O)C1=Cc2sc(COCCN)cc2N=C(N)C1] | 364.51 | >9000 | 7951 |
| TAZ-82 | 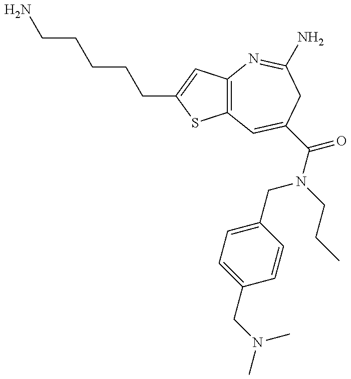 [SMILES: CCCN(Cc1ccc(CN(C)C)cc1)C(=O)C1=Cc2sc(CCCCCN)cc2N=C(N)C1] | 481.7 | 4998 | 2870 |
| TAZ-83 | 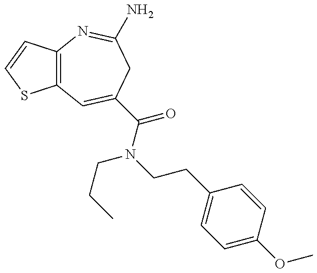 [SMILES: CCCN(CCc1ccc(OC)cc1)C(=O)C1=Cc2sccc2N=C(N)C1] | 383.51 | 3519 | 3369 |
| TAZ-84 | 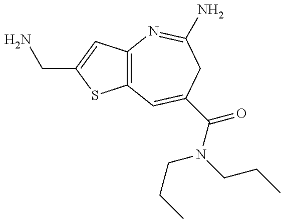 [SMILES: CCCN(CCC)C(=O)C1=Cc2sc(CN)cc2N=C(N)C1] | 320.45 | 4950 | 1373 |
| TAZ-85 | 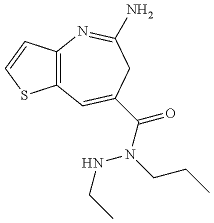 [SMILES: CCCN(NCC)C(=O)C1=Cc2sccc2N=C(N)C1] | 292.4 | >9000 | 4026 |
| TAZ-86 | 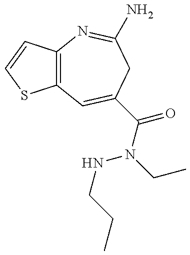 [SMILES: CCCNN(CC)C(=O)C1=Cc2sccc2N=C(N)C1] | 292.4 | >9000 | 4919 |
| TAZ-87 | 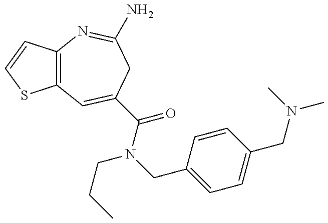 [SMILES: CCCN(Cc1ccc(CN(C)C)cc1)C(=O)C1=Cc2sccc2N=C(N)C1] | 396.55 | 1939 | 214 |
| TAZ-88 | 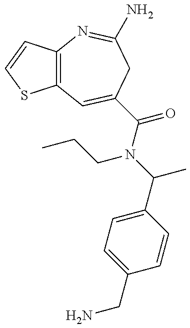 [SMILES: CCCN(C(=O)C1=Cc2sccc2N=C(N)C1)C(C)c1ccc(CN)cc1] | 382.52 | >9000 | 2270 |
| TAZ-89 | 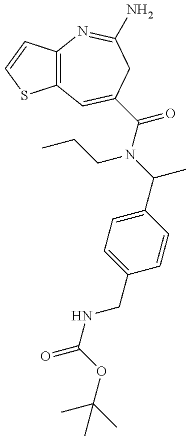 [SMILES: CCCN(C(=O)C1=Cc2sccc2N=C(N)C1)C(C)c1ccc(CNC(=O)OC(C)(C)C)cc1] | 482.64 | Not available | Not available |
| TAZ-90 | 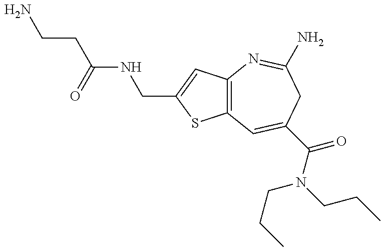 [SMILES: CCCN(CCC)C(=O)C1=Cc2sc(CNC(=O)CCN)cc2N=C(N)C1] | 391.53 | 909 | 3190 |
| TAZ-91 | 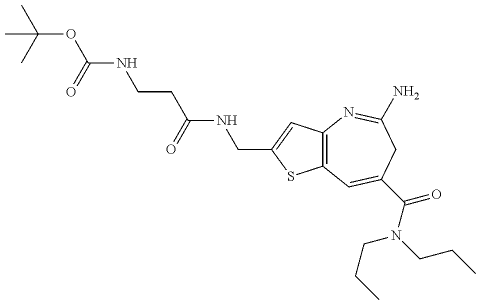 [SMILES: CCCN(CCC)C(=O)C1=Cc2sc(CNC(=O)CCNC(=O)OC(C)(C)C)cc2N=C(N)C1] | 491.65 | 4276 | 4151 |
| TAZ-92 | 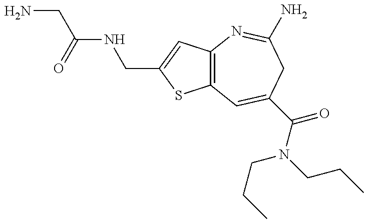 [SMILES: CCCN(CCC)C(=O)C1=Cc2sc(CNC(=O)CN)cc2N=C(N)C1] | 377.51 | >9000 | 2835 |
| TAZ-93 | 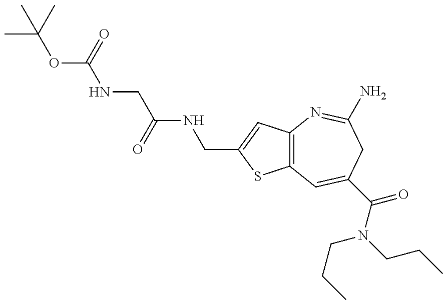 [SMILES: CCCN(CCC)C(=O)C1=Cc2sc(CNC(=O)CNC(=O)OC(C)(C)C)cc2N=C(N)C1] | 477.62 | 4494 | 3134 |
| TAZ-94 | 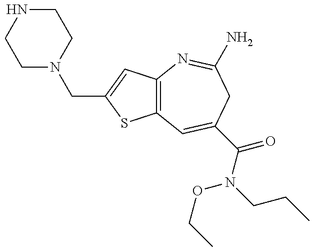 [SMILES: CCCN(OCC)C(=O)C1=Cc2sc(CN3CCNCC3)cc2N=C(N)C1] | 391.53 | 6202 | 101 |
| TAZ-95 | 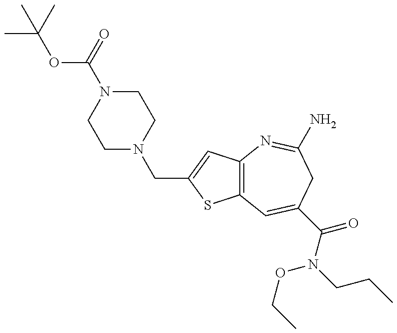 [SMILES: CCCN(OCC)C(=O)C1=Cc2sc(CN3CCN(C(=O)OC(C)(C)C)CC3)cc2N=C(N)C1] | 491.65 | Not available | Not available |
| TAZ-96 | 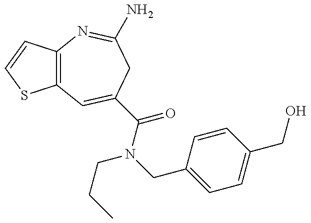 [SMILES: CCCN(Cc1ccc(CO)cc1)C(=O)C1=Cc2sccc2N=C(N)C1] | 369.48 | 2964 | 326 |
| TAZ-97 | 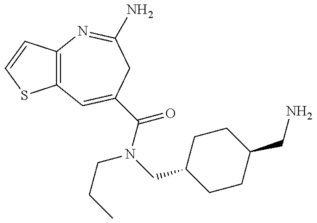 [SMILES: CCCN(C[C@H]1CC[C@H](CN)CC1)C(=O)C1=Cc2sccc2N=C(N)C1] | 374.54 | Not available | >9000 |
| TAZ-98 | 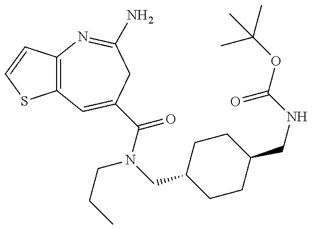 [SMILES: CCCN(C[C@H]1CC[C@H](CNC(=O)OC(C)(C)C)CC1)C(=O)C1=Cc2sccc2N=C(N)C1] | 474.66 | Not available | 631 |
| TAZ-99 | 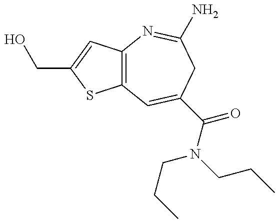 [SMILES: CCCN(CCC)C(=O)C1=Cc2sc(CO)cc2N=C(N)C1] | 321.44 | >9000 | 3622 |
| TAZ-100 | 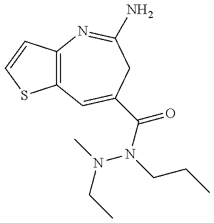 [SMILES: CCCN(C(=O)C1=Cc2sccc2N=C(N)C1)N(C)CC] | 306.43 | Not available | >9000 |
| TAZ-101 | 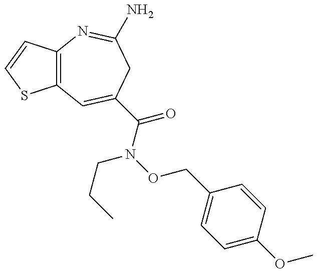 [SMILES: CCCN(OCc1ccc(OC)cc1)C(=O)C1=Cc2sccc2N=C(N)C1] | 385.48 | Not available | 203 |
| TAZ-102 | 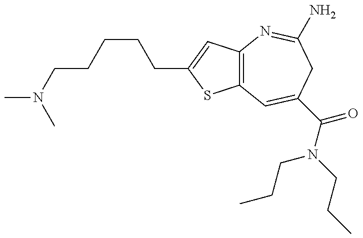 [SMILES: CCCN(CCC)C(=O)C1=Cc2sc(CCCCCN(C)C)cc2N=C(N)C1] | 404.61 | 793 | 384 |
| TAZ-103 | 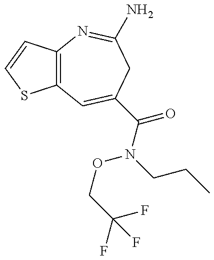 [SMILES: CCCN(OCC(F)(F)F)C(=O)C1=Cc2sccc2N=C(N)C1] | 347.36 | 3824 | 1377 |
| TAZ-104 | 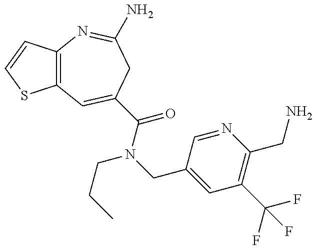 [SMILES: CCCN(Cc1cnc(CN)c(C(F)(F)F)c1)C(=O)C1=Cc2sccc2N=C(N)C1] | 437.48 | 3173 | 1444 |
| TAZ-105 | 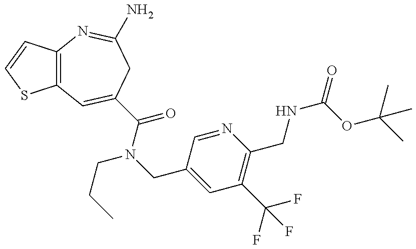 [SMILES: CCCN(Cc1cnc(CNC(=O)OC(C)(C)C)c(C(F)(F)F)c1)C(=O)C1=Cc2sccc2N=C(N)C1] | 537.6 | Not available | Not available |
| TAZ-106 | 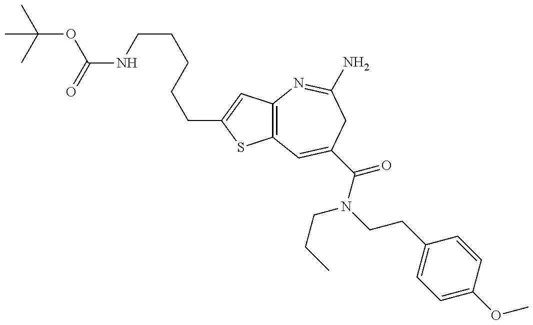 [SMILES: CCCN(CCc1ccc(OC)cc1)C(=O)C1=Cc2sc(CCCCCNC(=O)OC(C)(C)C)cc2N=C(N)C1] | 568.77 | 4732 | >9000 |
| TAZ-107 | 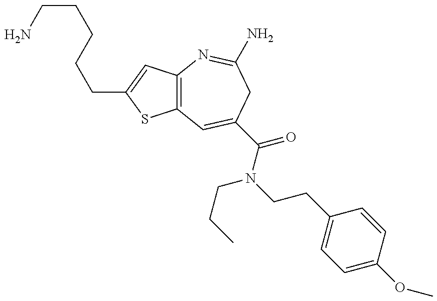 [SMILES: CCCN(CCc1ccc(OC)cc1)C(=O)C1=Cc2sc(CCCCCN)cc2N=C(N)C1] | 468.66 | 3579 | 6984 |
| TAZ-108 | 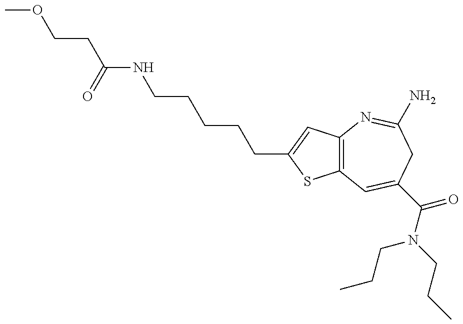 [SMILES: CCCN(CCC)C(=O)C1=Cc2sc(CCCCCNC(=O)CCOC)cc2N=C(N)C1] | 462.65 | 3852 | 3472 |
| TAZ-109 | 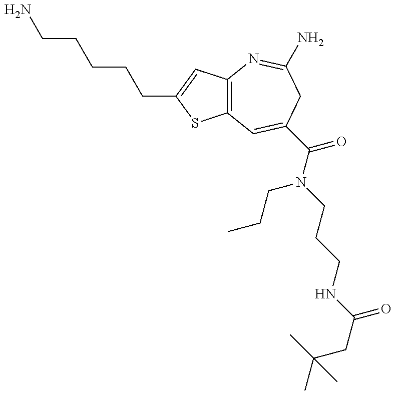 [SMILES: CCCN(CCCNC(=O)CC(C)(C)C)C(=O)C1=Cc2sc(CCCCCN)cc2N=C(N)C1] | 489.72 | | |
| TAZ-110 | 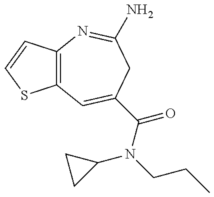 [SMILES: CCCN(C(=O)C1=Cc2sccc2N=C(N)C1)C1CC1] | 289.4 | | |
| TAZ-111 | 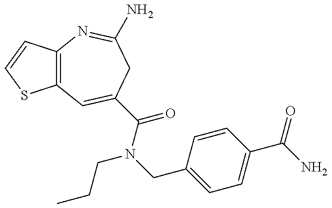 [SMILES: CCCN(Cc1ccc(C(N)=O)cc1)C(=O)C1=Cc2sccc2N=C(N)C1] | 382.48 | | |
| TAZ-112 | 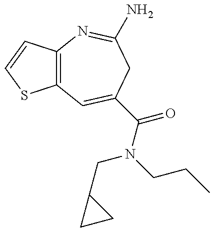 [SMILES: CCCN(CC1CC1)C(=O)C1=Cc2sccc2N=C(N)C1] | 303.42 | | |
| TAZ-113 | 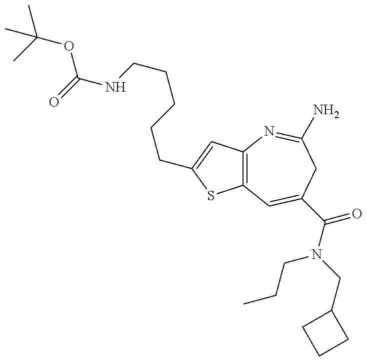 [SMILES: CCCN(CC1CCC1)C(=O)C1=Cc2sc(CCCCCNC(=O)OC(C)(C)C)cc2N=C(N)C1] | 502.71 | | |
| TAZ-114 | 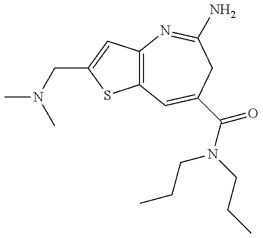 [SMILES: CCCN(CCC)C(=O)C1=Cc2sc(CN(C)C)cc2N=C(N)C1] | 348.51 | | |
| TAZ-115 | 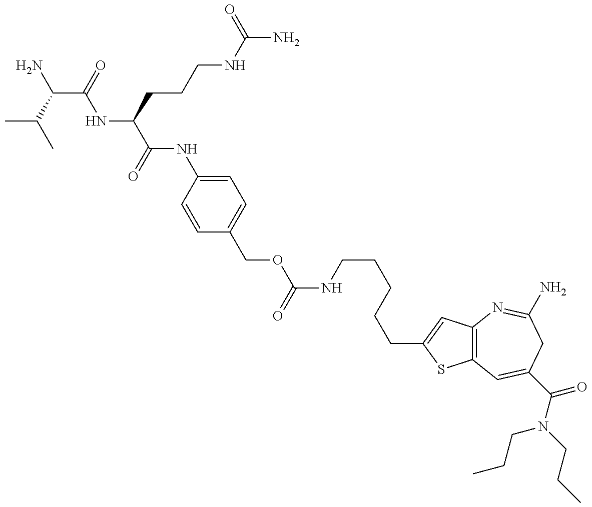 [SMILES: CCCN(CCC)C(=O)C1=Cc2sc(CCCCCNC(=O)OCc3ccc(NC(=O)[C@H](CCCNC(N)=O)NC(=O)[C@@H](N)C(C)C)cc3)cc2N=C(N)C1] | 782.01 | | |

TABLE 1c
Thienoazepine compounds (TAZ)

| | | | HEK293 | |
|---|---|---|---|---|
| | | | hTLR7 | HEK293 |
| TAZ | | | EC50 | hTLR8 |
| No. | Structure | MW | (nM) | EC50 (nM) |
| TAZ- 116 | 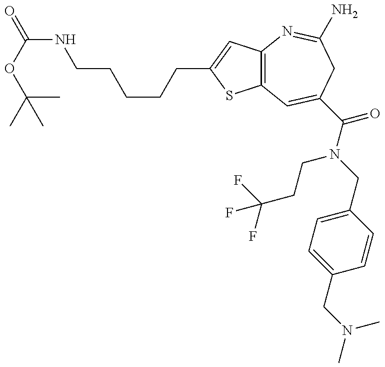 [SMILES: CN(C)Cc1ccc(CN(CCC(F)(F)F)C(=O)C2=Cc3sc(CCCCCNC(=O)OC(C)(C)C)cc3N=C(N)C2)cc1] | 635.8 | 1617 | |
| TAZ- 117 | 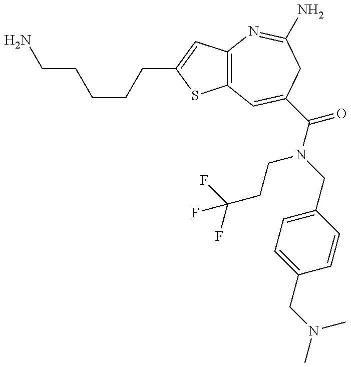 [SMILES: CN(C)Cc1ccc(CN(CCC(F)(F)F)C(=O)C2=Cc3sc(CCCCCN)cc3N=C(N)C2)cc1] | 535.7 | 9000 | 9000 |
| TAZ- 118 | 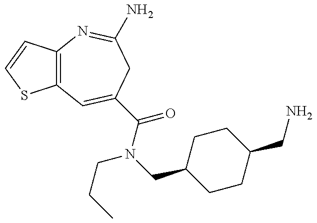 [SMILES: CCCN(C[C@H]1CC[C@@H](CN)CC1)C(=O)C1=Cc2sccc2N=C(N)C1] | 374.5 | 9000 | 4438 |
| TAZ- 119 | 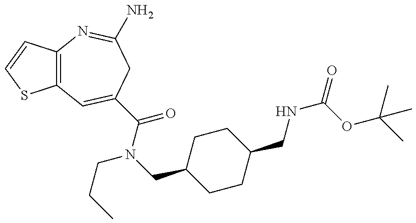 [SMILES: CCCN(C[C@H]1CC[C@@H](CNC(=O)OC(C)(C)C)CC1)C(=O)C1=Cc2sccc2N=C(N)C1] | 474.7 | 2875 | 9000 |
| TAZ- 120 | 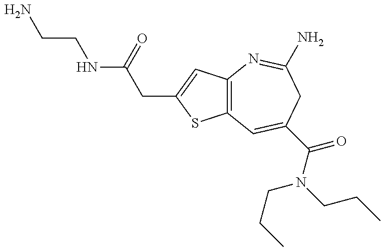 [SMILES: CCCN(CCC)C(=O)C1=Cc2sc(CC(=O)NCCN)cc2N=C(N)C1] | 391.5 | 9000 | 9000 |
| TAZ- 121 | 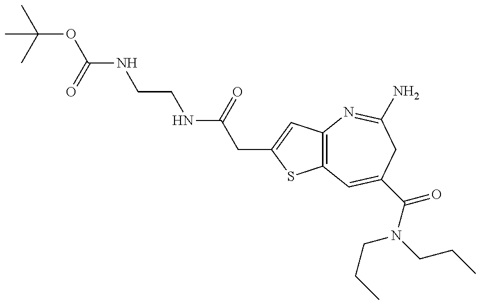 [SMILES: CCCN(CCC)C(=O)C1=Cc2sc(CC(=O)NCCNC(=O)OC(C)(C)C)cc2N=C(N)C1] | 491.7 | | |
| TAZ- 122 | 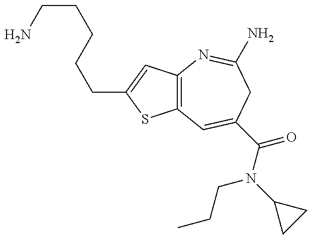 [SMILES: CCCN(C(=O)C1=Cc2sc(CCCCCN)cc2N=C(N)C1)C1CC1] | 374.5 | 3349 | 3244 |
| TAZ- 123 | 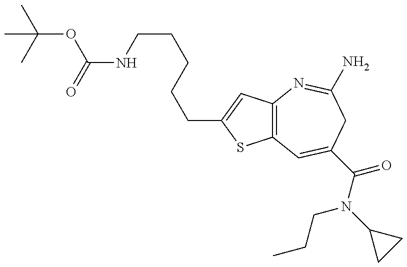 [SMILES: CCCN(C(=O)C1=Cc2sc(CCCCCNC(=O)OC(C)(C)C)cc2N=C(N)C1)C1CC1] | 474.7 | 4629 | 9000 |
| TAZ- 124 | 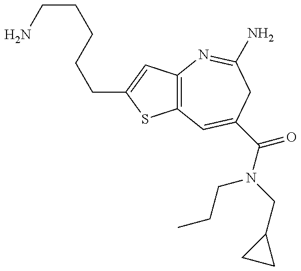 [SMILES: CCCN(CC1CC1)C(=O)C1=Cc2sc(CCCCCN)cc2N=C(N)C1] | 388.6 | 1727 | 1942 |
| TAZ- 125 | 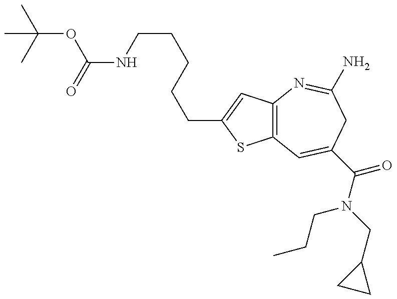 [SMILES: CCCN(CC1CC1)C(=O)C1=Cc2sc(CCCCCNC(=O)OC(C)(C)C)cc2N=C(N)C1] | 488.7 | 2705 | 9000 |
| TAZ- 126 | 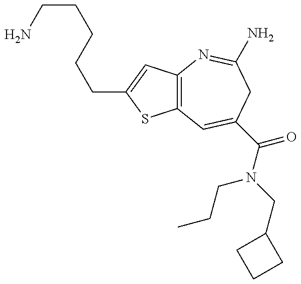 [SMILES: CCCN(CC1CCC1)C(=O)C1=Cc2sc(CCCCCN)cc2N=C(N)C1] | 402.6 | 950 | 633 |
| TAZ- 127 | 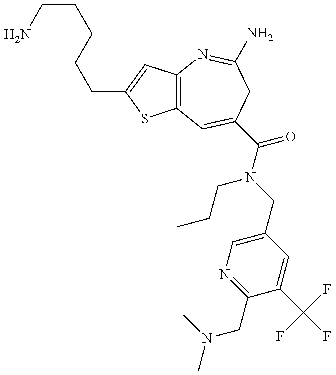 [SMILES: CCCN(Cc1cnc(CN(C)C)c(C(F)(F)F)c1)C(=O)C1=Cc2sc(CCCCCN)cc2N=C(N)C1] | 550.7 | 9000 | |
| TAZ- 128 | 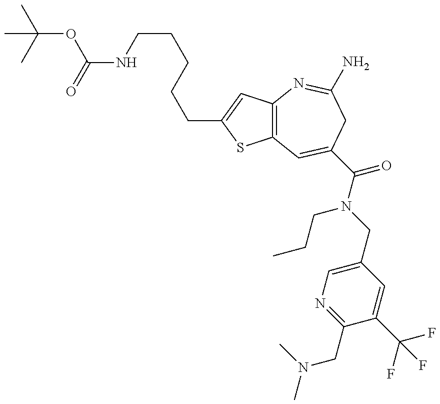 [SMILES: CCCN(Cc1cnc(CN(C)C)c(C(F)(F)F)c1)C(=O)C1=Cc2sc(CCCCCNC(=O)OC(C)(C)C)cc2N=C(N)C1] | 650.8 | 9000 | 9000 |
| TAZ- 129 | 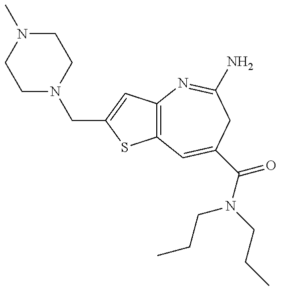 [SMILES: CCCN(CCC)C(=O)C1=Cc2sc(CN3CCN(C)CC3)cc2N=C(N)C1] | 403.6 | 5571 | 848 |
| TAZ- 130 | 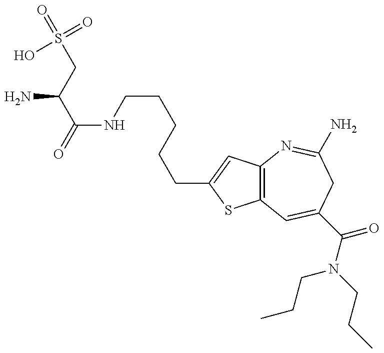 [SMILES: CCCN(CCC)C(=O)C1=Cc2sc(CCCCCNC(=O)[C@@H](N)CS(=O)(=O)O)cc2N=C(N)C1] | 527.7 | | |
| TAZ- 131 | 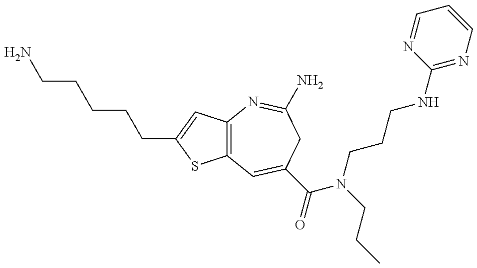 [SMILES: CCCN(CCCNc1ncccn1)C(=O)C1=Cc2sc(CCCCCN)cc2N=C(N)C1] | 469.7 | | |
| TAZ- 132 | 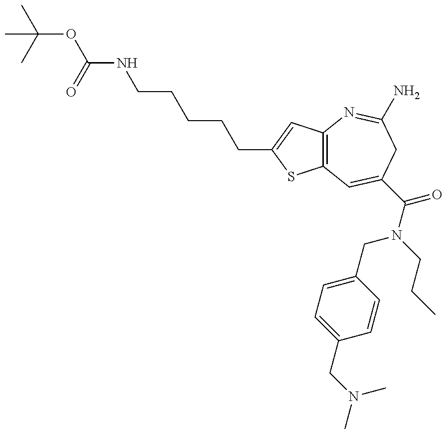 [SMILES: CCCN(Cc1ccc(CN(C)C)cc1)C(=O)C1=Cc2sc(CCCCCNC(=O)OC(C)(C)C)cc2N=C(N)C1] | 581.8 | | |
| TAZ- 133 | 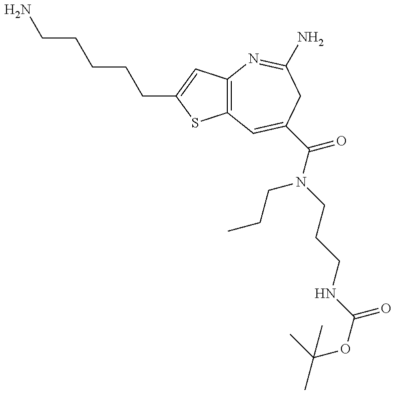 [SMILES: CCCN(CCCNC(=O)OC(C)(C)C)C(=O)C1=Cc2sc(CCCCCN)cc2N=C(N)C1] | 491.7 | 586 | 3008 |
| TAZ- 134 | 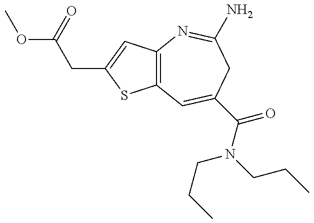 [SMILES: CCCN(CCC)C(=O)C1=Cc2sc(CC(=O)OC)cc2N=C(N)C1] | 363.5 | 4697 | 3420 |
| TAZ- 135 | 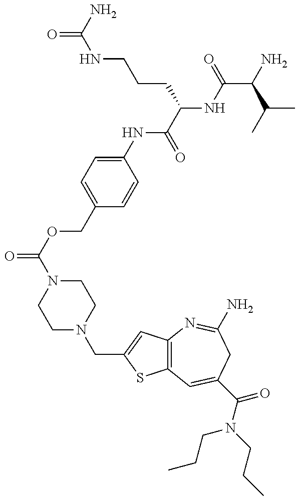 [SMILES: CCCN(CCC)C(=O)C1=Cc2sc(CN3CCN(C(=O)OCc4ccc(NC(=O)[C@H](CCCNC(N)=O)NC(=O)[C@@H](N)C(C)C)cc4)CC3)cc2N=C(N)C1] | 795.0 | | |
| TAZ- 136 | 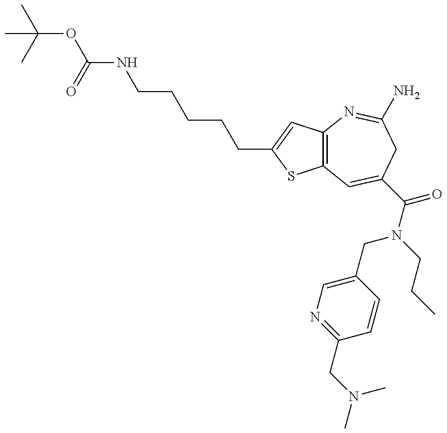 [SMILES: CCCN(Cc1ccc(CN(C)C)nc1)C(=O)C1=Cc2sc(CCCCCNC(=O)OC(C)(C)C)cc2N=C(N)C1] | 582.8 | | |
| TAZ- 137 | 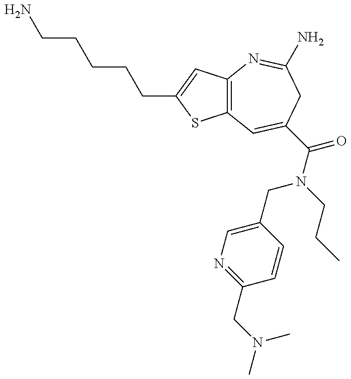 [SMILES: CCCN(Cc1ccc(CN(C)C)nc1)C(=O)C1=Cc2sc(CCCCCN)cc2N=C(N)C1] | 482.7 | | |
| TAZ- 138 | 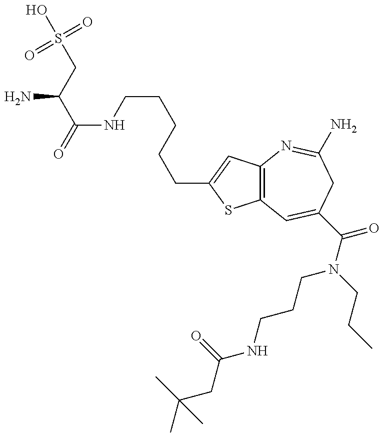 [SMILES: CCCN(CCCNC(=O)CC(C)(C)C)C(=O)C1=Cc2sc(CCCCCNC(=O)[C@@H](N)CS(=O)(=O)O)cc2N=C(N)C1] | 640.9 | | |
| TAZ- 139 | 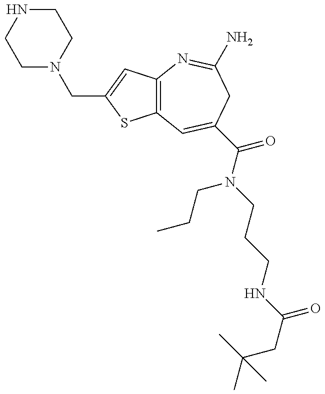 [SMILES: CCCN(CCCNC(=O)CC(C)(C)C)C(=O)C1=Cc2sc(CN3CCNCC3)cc2N=C(N)C1] | 502.7 | 4206 | 9000 |
| TAZ- 140 | 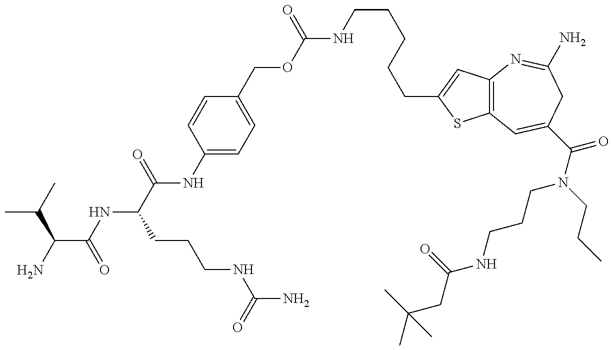 [SMILES: CCCN(CCCNC(=O)CC(C)(C)C)C(=O)C1=Cc2sc(CCCCCNC(=O)OCc3ccc(NC(=O)[C@H](CCCNC(N)=O)NC(=O)[C@@H](N)C(C)C)cc3)cc2N=C(N)C1] | 895.2 | | |
| TAZ- 141 | 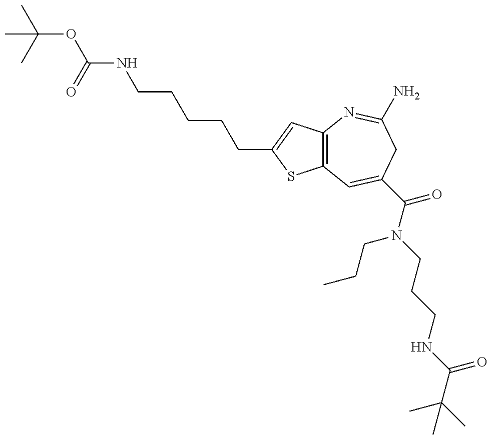 [SMILES: CCCN(CCCNC(=O)C(C)(C)C)C(=O)C1=Cc2sc(CCCCCNC(=O)OC(C)(C)C)cc2N=C(N)C1] | 575.8 | 380 | 3583 |
| TAZ- 142 | 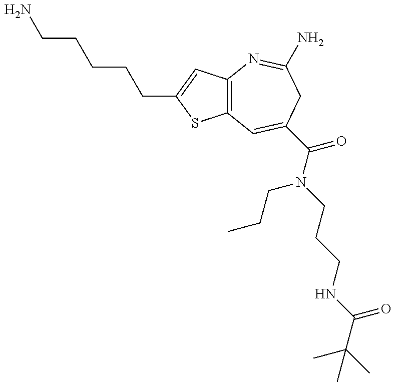 [SMILES: CCCN(CCCNC(=O)C(C)(C)C)C(=O)C1=Cc2sc(CCCCCN)cc2N=C(N)C1] | 475.7 | 827 | 9000 |
| TAZ- 143 | 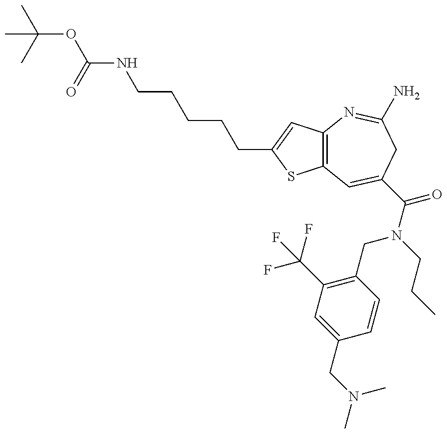 [SMILES: CCCN(Cc1ccc(CN(C)C)cc1C(F)(F)F)C(=O)C1=Cc2sc(CCCCCNC(=O)OC(C)(C)C)cc2N=C(N)C1] | 649.8 | 1884 | 9000 |
| TAZ- 144 | 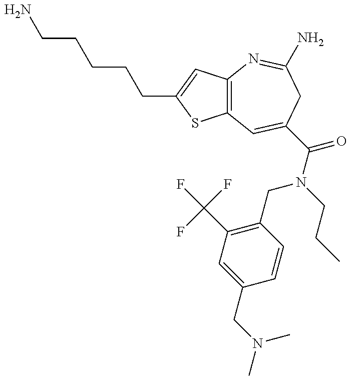 [SMILES: CCCN(Cc1ccc(CN(C)C)cc1C(F)(F)F)C(=O)C1=Cc2sc(CCCCCN)cc2N=C(N)C1] | 549.7 | 1282 | 2855 |
| TAZ- 145 | 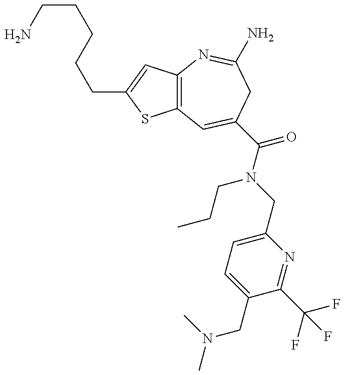 [SMILES: CCCN(Cc1ccc(CN(C)C)c(C(F)(F)F)n1)C(=O)C1=Cc2sc(CCCCCN)cc2N=C(N)C1] | 550.7 | 5940 | 4009 |
| TAZ- 146 | 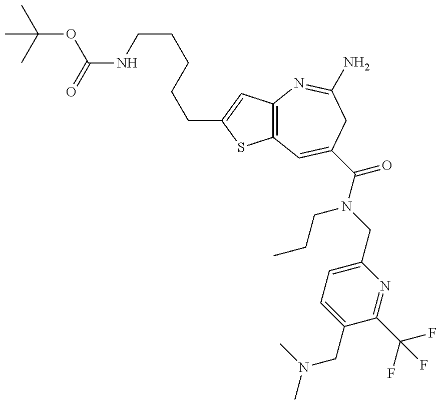 [SMILES: CCCN(Cc1ccc(CN(C)C)c(C(F)(F)F)n1)C(=O)C1=Cc2sc(CCCCCNC(=O)OC(C)(C)C)cc2N=C(N)C1] | 650.8 | 9000 | 9000 |
| TAZ- 147 | 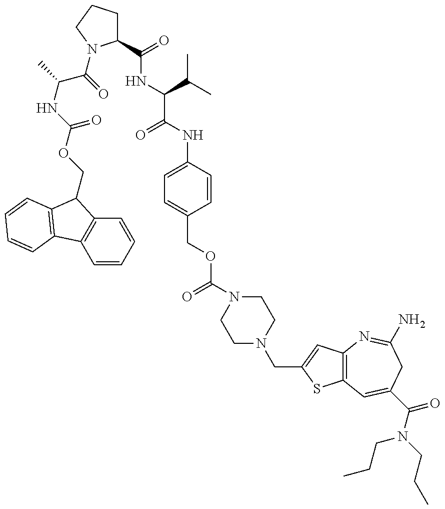 [SMILES: CCCN(CCC)C(=O)C1=Cc2sc(CN3CCN(C(=O)OCc4ccc(NC(=O)[C@@H](NC(=O)[C@@H]5CCCN5C(=O)[C@@H](C)NC(=O)OCC5c6ccccc6-c6ccccc65)C(C)C)cc4)CC3)cc2N=C(N)C1] | 1028.3 | | |
| TAZ- 148 | 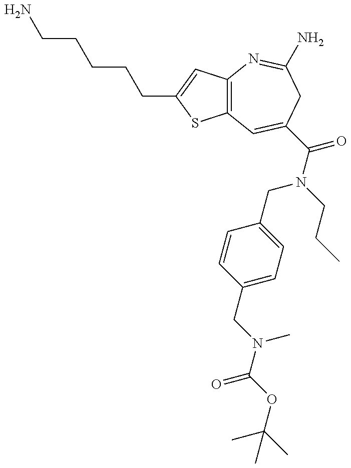 [SMILES: CCCN(Cc1ccc(CN(C)C(=O)OC(C)(C)C)cc1)C(=O)C1=Cc2sc(CCCCCN)cc2N=C(N)C1] | 567.8 | | |
| TAZ- 149 | 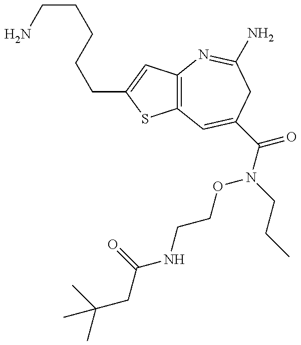 [SMILES: CCCN(OCCNC(=O)CC(C)(C)C)C(=O)C1=Cc2sc(CCCCCN)cc2N=C(N)C1] | 491.7 | 534 | 2237 |
| TAZ- 150 | 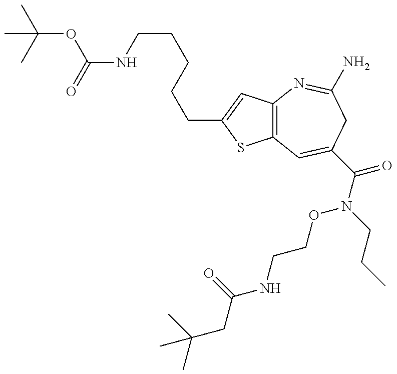 [SMILES: CCCN(OCCNC(=O)CC(C)(C)C)C(=O)C1=Cc2sc(CCCCCNC(=O)OC(C)(C)C)cc2N=C(N)C1] | 591.8 | 1990 | 1668 |
| TAZ- 151 | 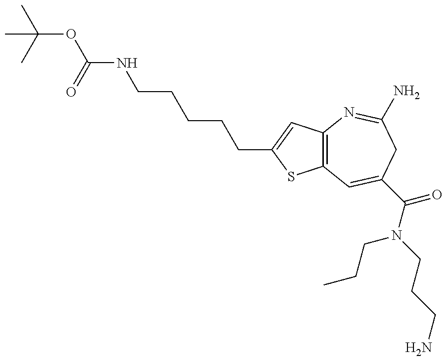 [SMILES: CCCN(CCCN)C(=O)C1=Cc2sc(CCCCCNC(=O)OC(C)(C)C)cc2N=C(N)C1] | 491.7 | | |
| TAZ- 152 | 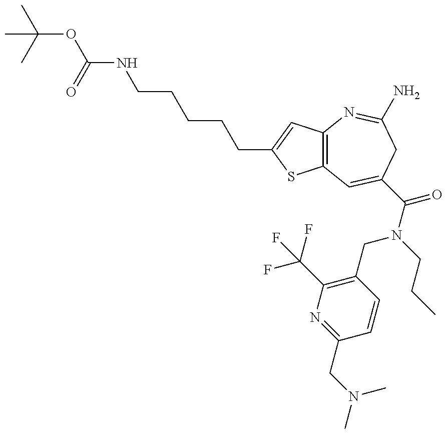 [SMILES: CCCN(Cc1ccc(CN(C)C)nc1C(F)(F)F)C(=O)C1=Cc2sc(CCCCCNC(=O)OC(C)(C)C)cc2N=C(N)C1] | 650.8 | | |
| TAZ- 153 | 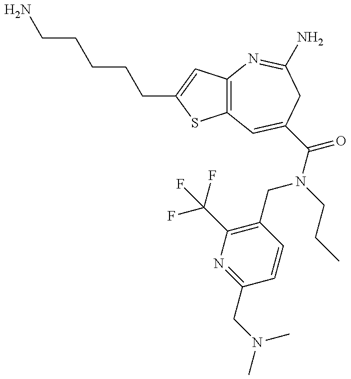 [SMILES: CCCN(Cc1ccc(CN(C)C)nc1C(F)(F)F)C(=O)C1=Cc2sc(CCCCCN)cc2N=C(N)C1] | 550.7 | | |
| TAZ- 154 | 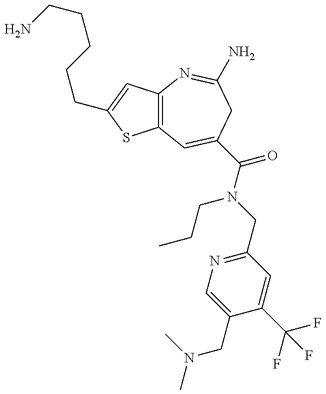 [SMILES: CCCN(Cc1cc(C(F)(F)F)c(CN(C)C)cn1)C(=O)C1=Cc2sc(CCCCCN)cc2N=C(N)C1] | 550.7 | | |
| TAZ- 155 | 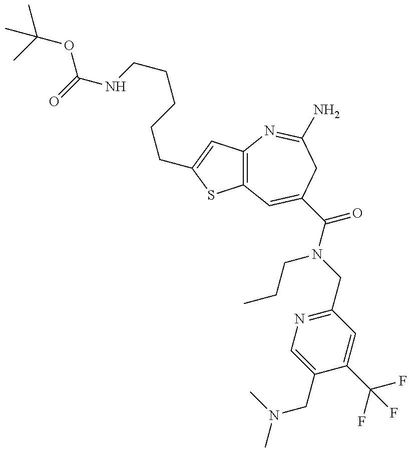 [SMILES: CCCN(Cc1cc(C(F)(F)F)c(CN(C)C)cn1)C(=O)C1=Cc2sc(CCCCCNC(=O)OC(C)(C)C)cc2N=C(N)C1] | 650.8 | | |
| TAZ- 156 | 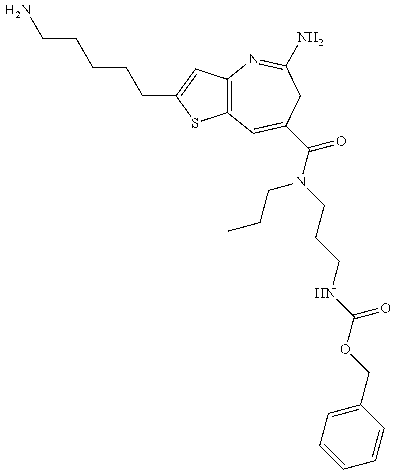 [SMILES: CCCN(CCCNC(=O)OCc1ccccc1)C(=O)C1=Cc2sc(CCCCCN)cc2N=C(N)C1] | 525.7 | | |
| TAZ- 157 | 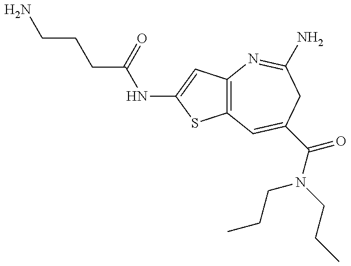 [SMILES: CCCN(CCC)C(=O)C1=Cc2sc(NC(=O)CCCN)cc2N=C(N)C1] | 391.5 | | |
| TAZ- 158 | 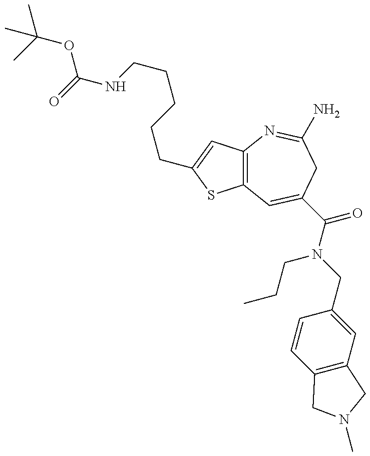 [SMILES: CCCN(Cc1ccc2c(c1)CN(C)C2)C(=O)C1=Cc2sc(CCCCCNC(=O)OC(C)(C)C)cc2N=C(N)C1] | 578.8 | | |
| TAZ- 159 | 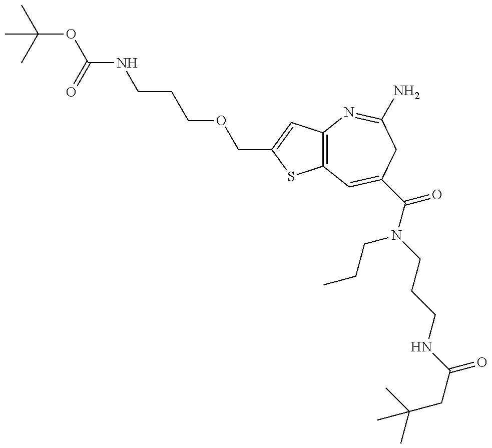 [SMILES: CCCN(CCCNC(=O)CC(C)(C)C)C(=O)C1=Cc2sc(COCCCNC(=O)OC(C)(C)C)cc2N=C(N)C1] | 591.8 | | |
| TAZ- 160 | 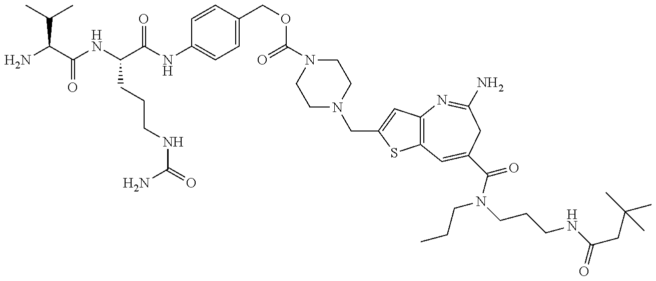 [SMILES: CCCN(CCCNC(=O)CC(C)(C)C)C(=O)C1=Cc2sc(CN3CCN(C(=O)OCc4ccc(NC(=O)[C@H](CCCNC(N)=O)NC(=O)[C@@H](N)C(C)C)cc4)CC3)cc2N=C(N)C1] | 908.2 | | |
| TAZ- 161 | 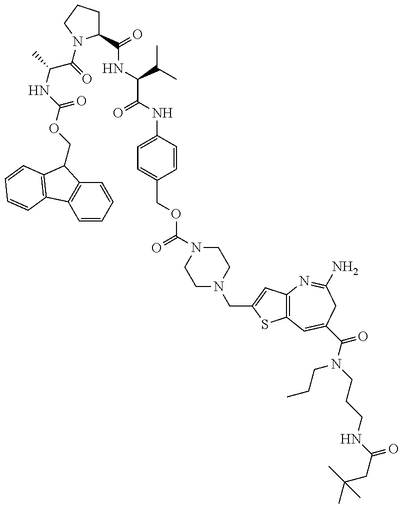 [SMILES: CCCN(CCCNC(=O)CC(C)(C)C)C(=O)C1=Cc2sc(CN3CCN(C(=O)OCc4ccc(NC(=O)[C@@H](NC(=O)[C@@H]5CCCN5C(=O)[C@@H](C)NC(=O)OCC5c6ccccc6-c6ccccc65)C(C)C)cc4)CC3)cc2N=C(N)C1] | 1141.4 | | |
| TAZ- 162 | 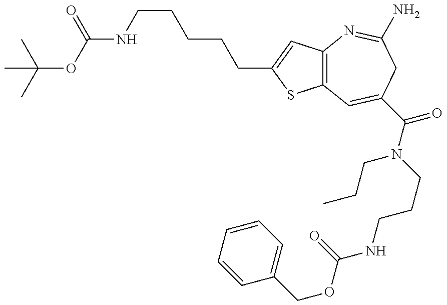 [SMILES: CCCN(CCCNC(=O)OCc1ccccc1)C(=O)C1=Cc2sc(CCCCCNC(=O)OC(C)(C)C)cc2N=C(N)C1] | 625.8 | 1238 | 1247 |
| TAZ- 163 | 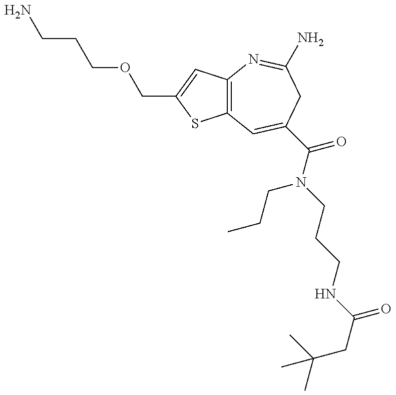 [SMILES: CCCN(CCCNC(=O)CC(C)(C)C)C(=O)C1=Cc2sc(COCCCN)cc2N=C(N)C1] | 491.7 | | |
| TAZ- 164 | 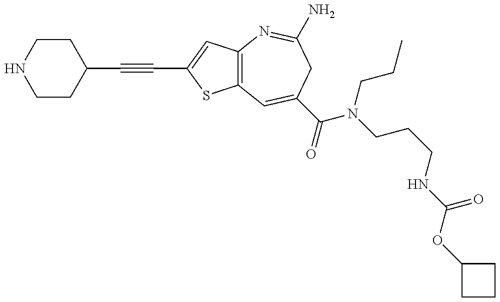 [SMILES: CCCN(CCCNC(=O)OC1CCC1)C(=O)C1=Cc2sc(C#CC3CCNCC3)cc2N=C(N)C1] | 511.7 | 476 | 3889 |
| TAZ- 165 | 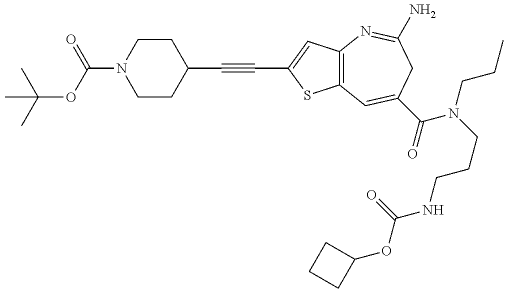 [SMILES: CCCN(CCCNC(=O)OC1CCC1)C(=O)C1=Cc2sc(C#CC3CCN(C(=O)OC(C)(C)C)CC3)cc2N=C(N)C1] | 611.8 | 9000 | |
| TAZ-166 | 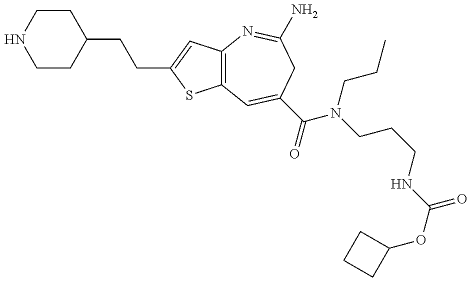 [SMILES: CCCN(CCCNC(=O)OC1CCC1)C(=O)C1=Cc2sc(CCC3CCNCC3)cc2N=C(N)C1] | 515.7 | 802 | 3697 |
| TAZ- 167 | 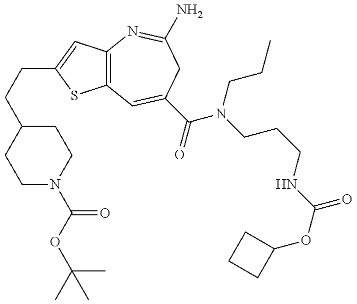 [SMILES: CCCN(CCCNC(=O)OC1CCC1)C(=O)C1=Cc2sc(CCC3CCN(C(=O)OC(C)(C)C)CC3)cc2N=C(N)C1] | 615.8 | 3129 | |
| TAZ- 168 | 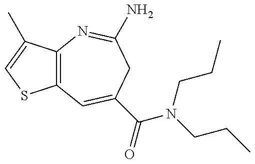 [SMILES: CCCN(CCC)C(=O)C1=Cc2scc(C)c2N=C(N)C1] | 305.4 | 9000 | 3875 |
| TAZ- 169 | 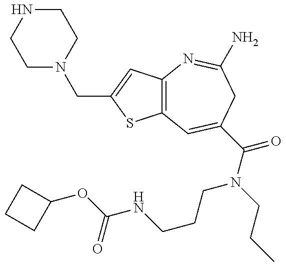 [SMILES: CCCN(CCCNC(=O)OC1CCC1)C(=O)C1=Cc2sc(CN3CCNCC3)cc2N=C(N)C1] | 502.7 | 3174 | 9000 |
| TAZ- 170 | 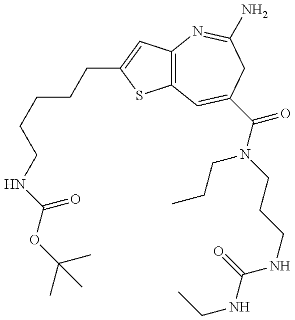 [SMILES: CCCN(CCCNC(=O)NCC)C(=O)C1=Cc2sc(CCCCCNC(=O)OC(C)(C)C)cc2N=C(N)C1] | 562.8 | 282 | 3371 |
| TAZ- 171 | 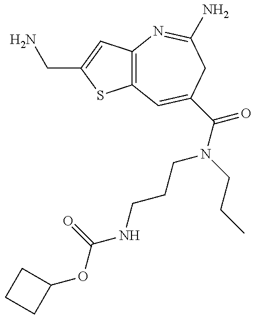 [SMILES: CCCN(CCCNC(=O)OC1CCC1)C(=O)C1=Cc2sc(CN)cc2N=C(N)C1] | 433.6 | 5288 | 3128 |
| TAZ- 172 | 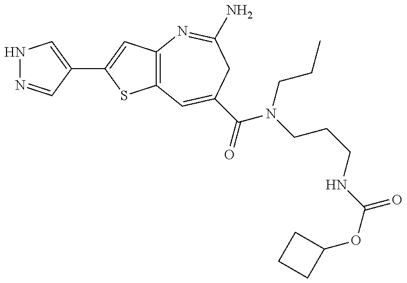 [SMILES: CCCN(CCCNC(=O)OC1CCC1)C(=O)C1=Cc2sc(-c3cn[nH]c3)cc2N=C(N)C1] | 470.6 | 153 | 3695 |
| TAZ- 173 | 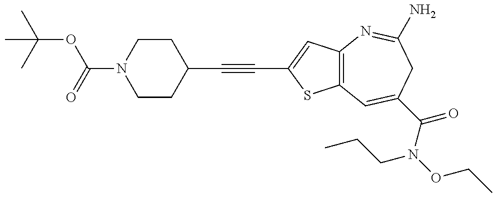 [SMILES: CCCN(OCC)C(=O)C1=Cc2sc(C#CC3CCN(C(=O)OC(C)(C)C)CC3)cc2N=C(N)C1] | 500.7 | 7689 | 9000 |
| TAZ- 174 | 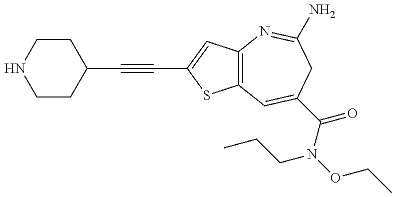 [SMILES: CCCN(OCC)C(=O)C1=Cc2sc(C#CC3CCNCC3)cc2N=C(N)C1] | 400.5 | 5809 | 1641 |
| TAZ- 175 | 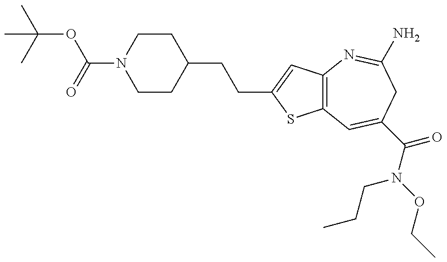 [SMILES: CCCN(OCC)C(=O)C1=Cc2sc(CCC3CCN(C(=O)OC(C)(C)C)CC3)cc2N=C(N)C1] | 504.7 | 4178 | 474 |
| TAZ- 176 | 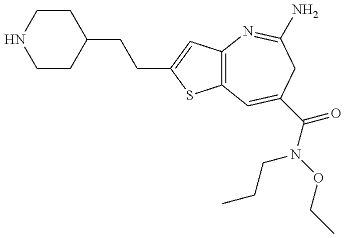 [SMILES: CCCN(OCC)C(=O)C1=Cc2sc(CCC3CCNCC3)cc2N=C(N)C1] | 404.6 | 915 | 249 |
| TAZ- 177 | 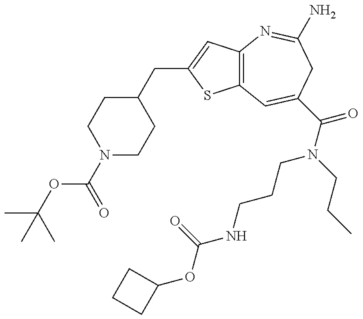 [SMILES: CCCN(CCCNC(=O)OC1CCC1)C(=O)C1=Cc2sc(CC3CCN(C(=O)OC(C)(C)C)CC3)cc2N=C(N)C1] | 601.8 | 882 | 3402 |
| TAZ- 178 | 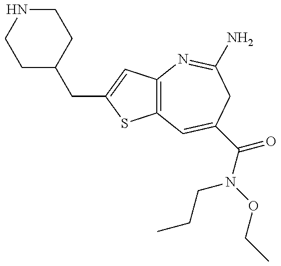 [SMILES: CCCN(OCC)C(=O)C1=Cc2sc(CC3CCNCC3)cc2N=C(N)C1] | 390.5 | 714 | 124 |
| TAZ- 179 | 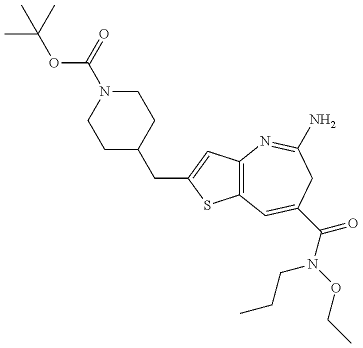 [SMILES: CCCN(OCC)C(=O)C1=Cc2sc(CC3CCN(C(=O)OC(C)(C)C)CC3)cc2N=C(N)C1] | 490.7 | 9000 | 2543 |
| TAZ- 180 | 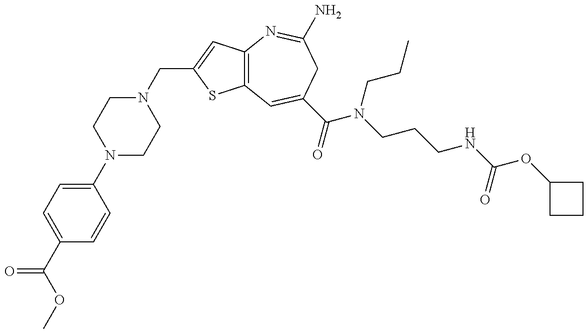 [SMILES: CCCN(CCCNC(=O)OC1CCC1)C(=O)C1=Cc2sc(CN3CCN(c4ccc(C(=O)OC)cc4)CC3)cc2N=C(N)C1] | 636.8 | 3648 | 9000 |
| TAZ-181 | 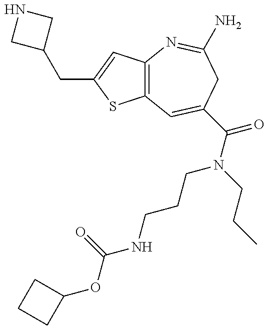 [SMILES: CCCN(CCCNC(=O)OC1CCC1)C(=O)C1=Cc2sc(CC3CNC3)cc2N=C(N)C1] | 473.6 | 2032 | 9000 |
| TAZ- 182 | 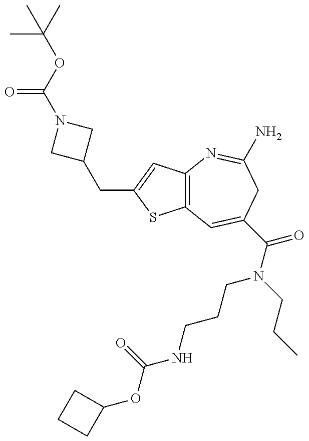 [SMILES: CCCN(CCCNC(=O)OC1CCC1)C(=O)C1=Cc2sc(CC3CN(C(=O)OC(C)(C)C)C3)cc2N=C(N)C1] | 573.8 | 101 | 840 |
| TAZ- 183 | 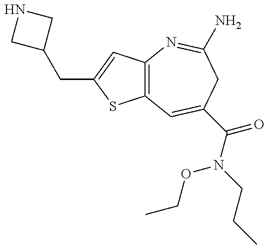 [SMILES: CCCN(OCC)C(=O)C1=Cc2sc(CC3CNC3)cc2N=C(N)C1] | 362.5 | 3560 | 485 |
| TAZ- 184 | 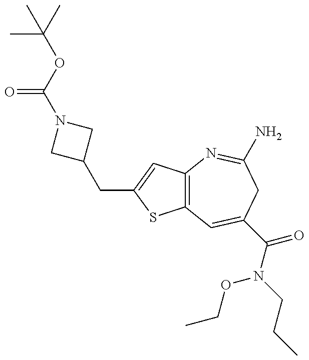 [SMILES: CCCN(OCC)C(=O)C1=Cc2sc(CC3CN(C(=O)OC(C)(C)C)C3)cc2N=C(N)C1] | 462.6 | 922 | 930 |
| TAZ- 185 | 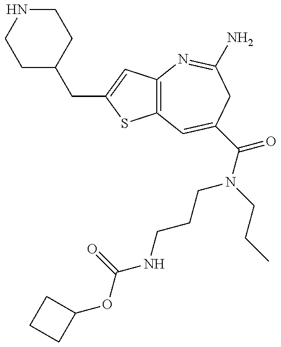 [SMILES: CCCN(CCCNC(=O)OC1CCC1)C(=O)C1=Cc2sc(CC3CCNCC3)cc2N=C(N)C1] | 501.7 | 3704 | 9000 |
| TAZ- 186 | 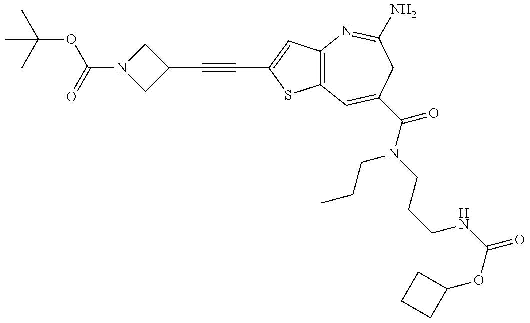 [SMILES: CCCN(CCCNC(=O)OC1CCC1)C(=O)C1=Cc2sc(C#CC3CN(C(=O)OC(C)(C)C)C3)cc2N=C(N)C1] | 583.7 | | |
| TAZ- 187 | 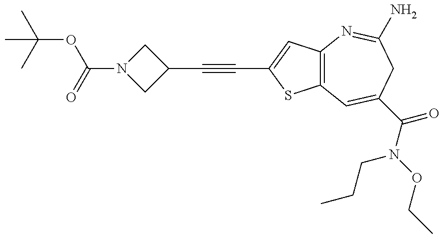 [SMILES: CCCN(OCC)C(=O)C1=Cc2sc(C#CC3CN(C(=O)OC(C)(C)C)C3)cc2N=C(N)C1] | 472.6 | 9000 | 964 |
| TAZ- 188 | 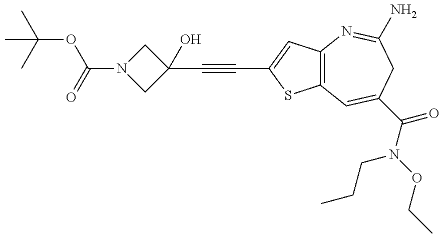 [SMILES: CCCN(OCC)C(=O)C1=Cc2sc(C#CC3(O)CN(C(=O)OC(C)(C)C)C3)cc2N=C(N)C1] | 488.6 | 9000 | 7798 |
| TAZ- 189 | 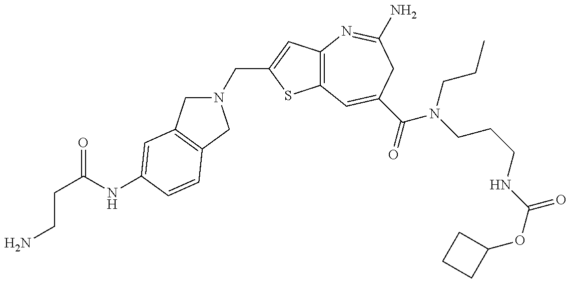 [SMILES: CCCN(CCCNC(=O)OC1CCC1)C(=O)C1=Cc2sc(CN3Cc4ccc(NC(=O)CCN)cc4C3)cc2N=C(N)C1] | 621.8 | 5721 | 3391 |
| TAZ- 190 | 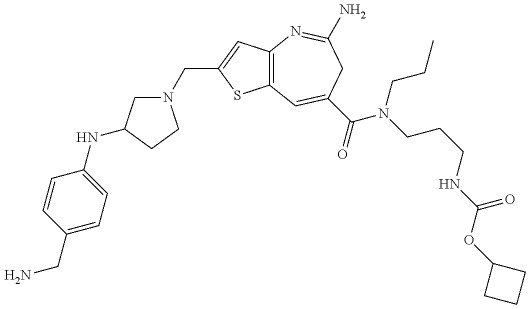 [SMILES: CCCN(CCCNC(=O)OC1CCC1)C(=O)C1=Cc2sc(CN3CCC(Nc4ccc(CN)cc4)C3)cc2N=C(N)C1] | 607.8 | 2988 | 4803 |
| TAZ- 191 | 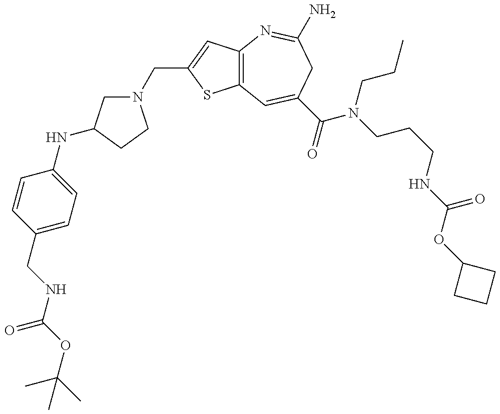 [SMILES: CCCN(CCCNC(=O)OC1CCC1)C(=O)C1=Cc2sc(CN3CCC(Nc4ccc(CNC(=O)OC(C)(C)C)cc4)C3)cc2N=C(N)C1] | 707.9 | | |
| TAZ- 192 | 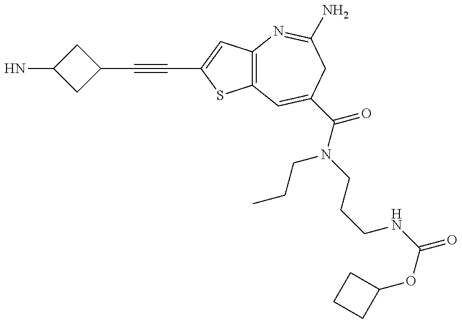 [SMILES: CCCN(CCCNC(=O)OC1CCC1)C(=O)C1=Cc2sc(C#CC3CC(N)C3)cc2N=C(N)C1] | 483.6 | 824 | 3484 |
| TAZ- 193 | 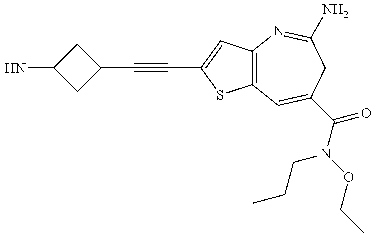 [SMILES: CCCN(OCC)C(=O)C1=Cc2sc(C#CC3CC(N)C3)cc2N=C(N)C1] | 372.5 | 3362 | 1517 |
| TAZ- 194 | 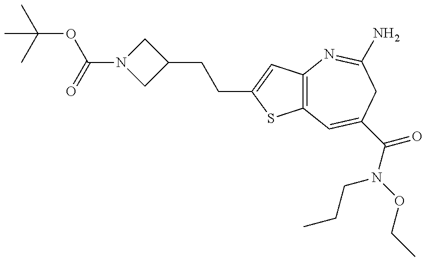 [SMILES: CCCN(OCC)C(=O)C1=Cc2sc(CCC3CN(C(=O)OC(C)(C)C)C3)cc2N=C(N)C1] | 476.6 | 467 | 631 |
| TAZ- 195 | 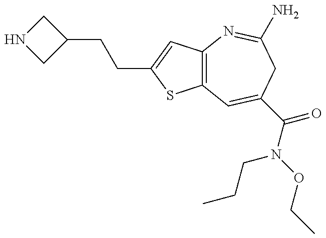 [SMILES: CCCN(OCC)C(=O)C1=Cc2sc(CCC3CNC3)cc2N=C(N)C1] | 376.5 | 6603 | 953 |
| TAZ- 196 | 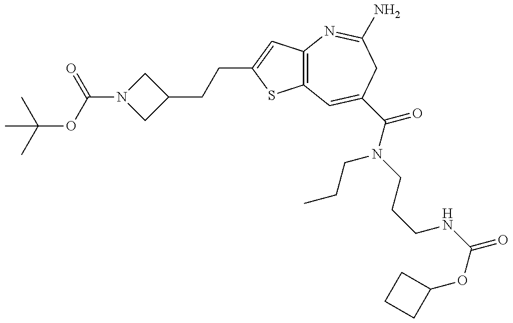 [SMILES: CCCN(CCCNC(=O)OC1CCC1)C(=O)C1=Cc2sc(CCC3CN(C(=O)OC(C)(C)C)C3)cc2N=C(N)C1] | 587.8 | | |
| TAZ- 197 | 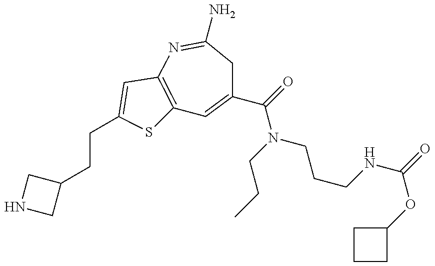 [SMILES: CCCN(CCCNC(=O)OC1CCC1)C(=O)C1=Cc2sc(CCC3CNC3)cc2N=C(N)C1] | 487.7 | 1808 | 9000 |
| TAZ- 198 | 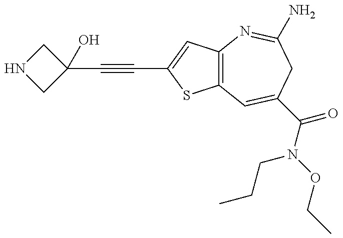 [SMILES: CCCN(OCC)C(=O)C1=Cc2sc(C#CC3(O)CNC3)cc2N=C(N)C1] | 388.5 | 9000 | 4275 |
| TAZ- 199 | 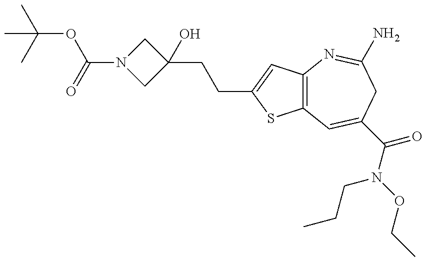 [SMILES: CCCN(OCC)C(=O)C1=Cc2sc(CCC3(O)CN(C(=O)OC(C)(C)C)C3)cc2N=C(N)C1] | 492.6 | 31 | 1614 |
| TAZ- 200 | 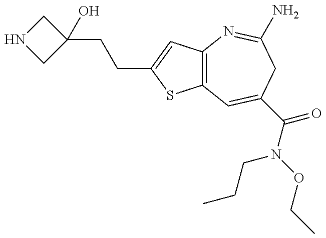 [SMILES: CCCN(OCC)C(=O)C1=Cc2sc(CCC3(O)CNC3)cc2N=C(N)C1] | 392.5 | 2916 | 4071 |
| TAZ- 201 | 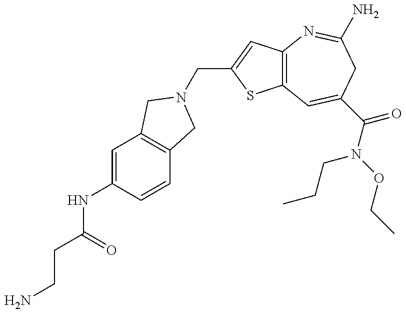 [SMILES: CCCN(OCC)C(=O)C1=Cc2sc(CN3Cc4ccc(NC(=O)CCN)cc4C3)cc2N=C(N)C1] | 510.7 | 9000 | 2957 |
| TAZ- 202 | 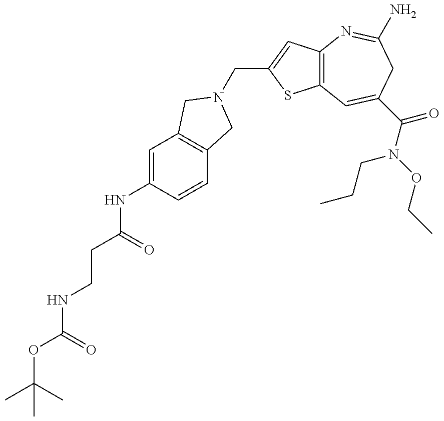 [SMILES: CCCN(OCC)C(=O)C1=Cc2sc(CN3Cc4ccc(NC(=O)CCNC(=O)OC(C)(C)C)cc4C3)cc2N=C(N)C1] | 610.8 | 3680 | 1373 |
| TAZ- 203 | 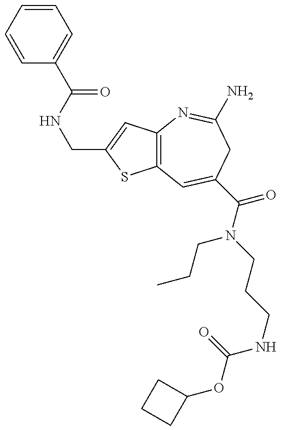 [SMILES: CCCN(CCCNC(=O)OC1CCC1)C(=O)C1=Cc2sc(CNC(=O)c3ccccc3)cc2N=C(N)C1] | 537.7 | 3261 | 3251 |
| TAZ- 204 | 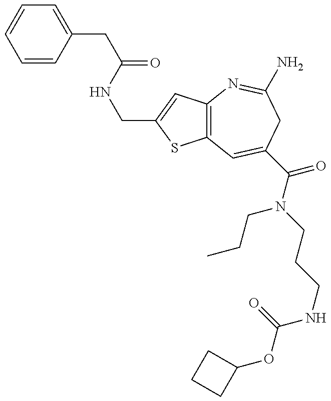 [SMILES: CCCN(CCCNC(=O)OC1CCC1)C(=O)C1=Cc2sc(CNC(=O)Cc3ccccc3)cc2N=C(N)C1] | 551.7 | 2665 | 2250 |
| TAZ- 205 | 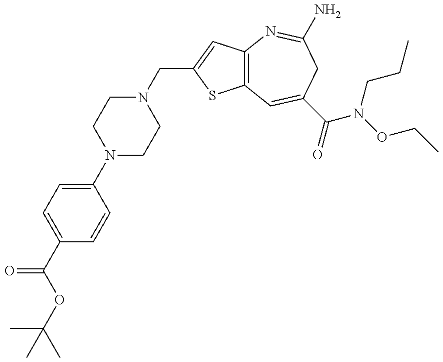 [SMILES: CCCN(OCC)C(=O)C1=Cc2sc(CN3CCN(c4ccc(C(=O)OC(C)(C)C)cc4)CC3)cc2N=C(N)C1] | 567.8 | 9000 | 9000 |
| TAZ- 206 | 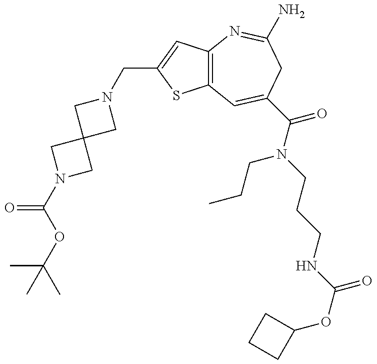 [SMILES: CCCN(CCCNC(=O)OC1CCC1)C(=O)C1=Cc2sc(CN3CC4(C3)CN(C(=O)OC(C)(C)C)C4)cc2N=C(N)C1] | 614.8 | | |
| TAZ- 207 | 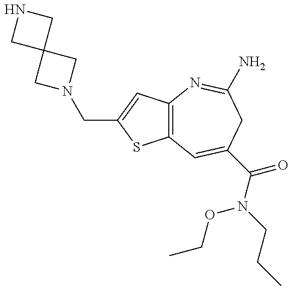 [SMILES: CCCN(OCC)C(=O)C1=Cc2sc(CN3CC4(CNC4)C3)cc2N=C(N)C1] | 403.5 | 9000 | 9000 |
| TAZ- 208 | 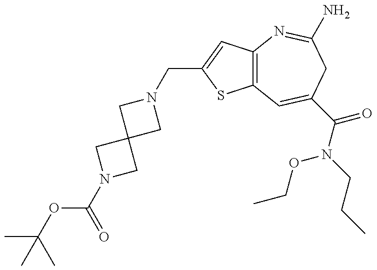 [SMILES: CCCN(OCC)C(=O)C1=Cc2sc(CN3CC4(C3)CN(C(=O)OC(C)(C)C)C4)cc2N=C(N)C1] | 503.7 | 3614 | 8507 |
| TAZ- 209 | 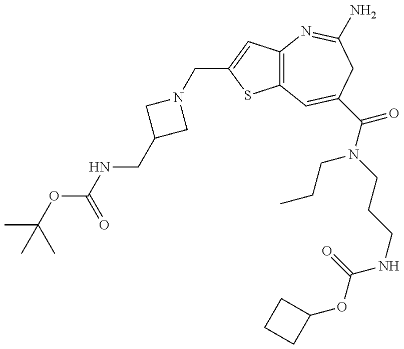 [SMILES: CCCN(CCCNC(=O)OC1CCC1)C(=O)C1=Cc2sc(CN3CC(CNC(=O)OC(C)(C)C)C3)cc2N=C(N)C1] | 602.8 | | |
| TAZ- 210 | 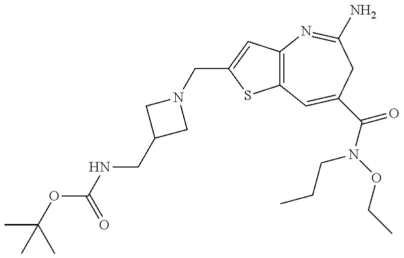 [SMILES: CCCN(OCC)C(=O)C1=Cc2sc(CN3CC(CNC(=O)OC(C)(C)C)C3)cc2N=C(N)C1] | 491.7 | 1546 | 2939 |
| TAZ- 211 | 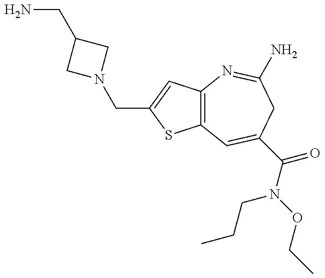 [SMILES: CCCN(OCC)C(=O)C1=Cc2sc(CN3CC(CN)C3)cc2N=C(N)C1] | 391.5 | 9000 | 4316 |
| TAZ- 212 | 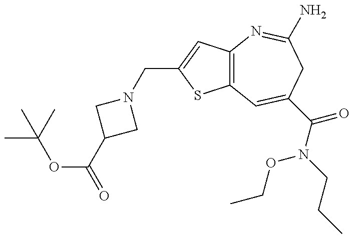 [SMILES: CCCN(OCC)C(=O)C1=Cc2sc(CN3CC(C(=O)OC(C)(C)C)C3)cc2N=C(N)C1] | 462.6 | 1265 | 2542 |
| TAZ- 213 | 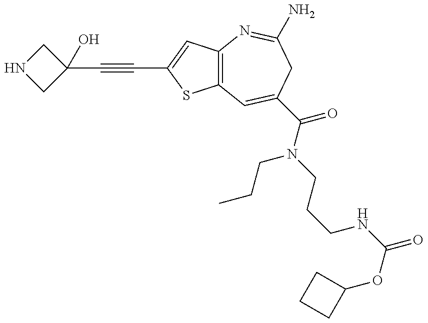 [SMILES: CCCN(CCCNC(=O)OC1CCC1)C(=O)C1=Cc2sc(C#CC3(O)CNC3)cc2N=C(N)C1] | 499.6 | | |
| TAZ- 214 | 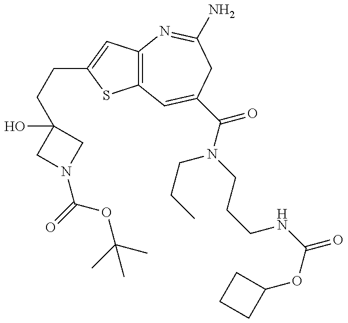 [SMILES: CCCN(CCCNC(=O)OC1CCC1)C(=O)C1=Cc2sc(CCC3(O)CN(C(=O)OC(C)(C)C)C3)cc2N=C(N)C1] | 603.8 | | |
| TAZ- 215 | 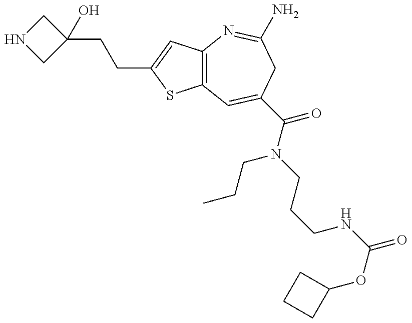 [SMILES: CCCN(CCCNC(=O)OC1CCC1)C(=O)C1=Cc2sc(CCC3(O)CNC3)cc2N=C(N)C1] | 503.7 | | |
| TAZ- 216 | 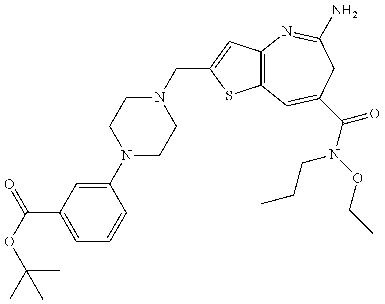 [SMILES: CCCN(OCC)C(=O)C1=Cc2sc(CN3CCN(c4cccc(C(=O)OC(C)(C)C)c4)CC3)cc2N=C(N)C1] | 567.8 | 9000 | |
| TAZ- 217 | 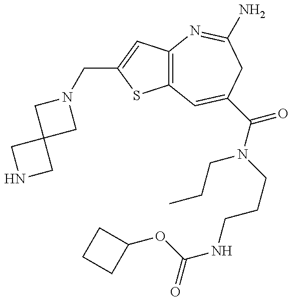 [SMILES: CCCN(CCCNC(=O)OC1CCC1)C(=O)C1=Cc2sc(CN3CC4(CNC4)C3)cc2N=C(N)C1] | 514.7 | | |
| TAZ- 218 | 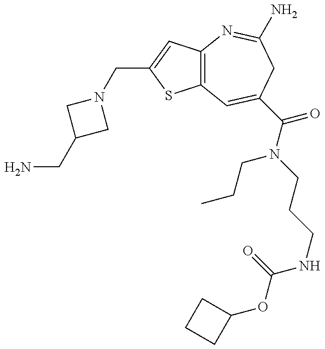 [SMILES: CCCN(CCCNC(=O)OC1CCC1)C(=O)C1=Cc2sc(CN3CC(CN)C3)cc2N=C(N)C1] | 502.7 | | |
| TAZ- 219 | 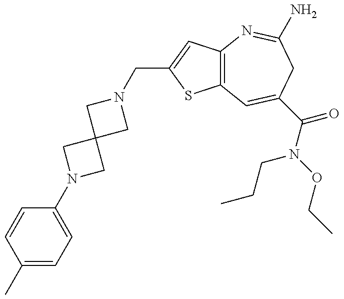 [SMILES: CCCN(OCC)C(=O)C1=Cc2sc(CN3CC4(C3)CN(c3ccc(C)cc3)C4)cc2N=C(N)C1] | 493.7 | 3197 | 847 |
| TAZ- 220 | 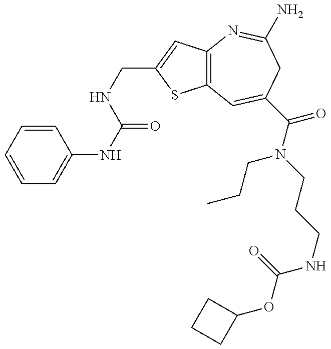 [SMILES: CCCN(CCCNC(=O)OC1CCC1)C(=O)C1=Cc2sc(CNC(=O)Nc3ccccc3)cc2N=C(N)C1] | 552.7 | | |
| TAZ- 221 | 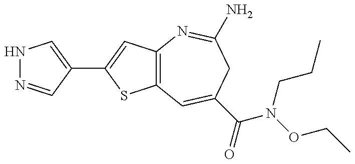 [SMILES: CCCN(OCC)C(=O)C1=Cc2sc(-c3cn[nH]c3)cc2N=C(N)C1] | 359.5 | 6118 | 6698 |
| TAZ- 222 | 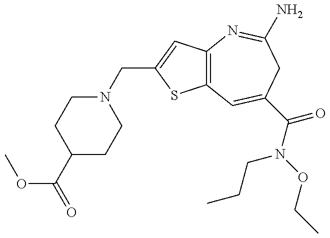 [SMILES: CCCN(OCC)C(=O)C1=Cc2sc(CN3CCC(C(=O)OC)CC3)cc2N=C(N)C1] | 448.6 | 4892 | 2826 |
| TAZ- 223 | 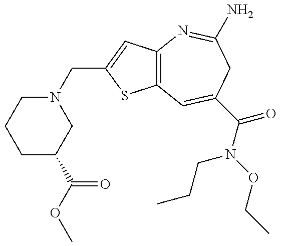 [SMILES: CCCN(OCC)C(=O)C1=Cc2sc(CN3CCC[C@@H](C(=O)OC)C3)cc2N=C(N)C1] | 448.6 | 3803 | 3138 |
| TAZ- 224 | 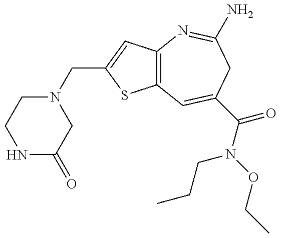 [SMILES: CCCN(OCC)C(=O)C1=Cc2sc(CN3CCNC(=O)C3)cc2N=C(N)C1] | 405.5 | 3410 | 2723 |
| TAZ- 225 | 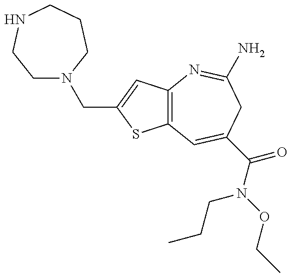 [SMILES: CCCN(OCC)C(=O)C1=Cc2sc(CN3CCCNCC3)cc2N=C(N)C1] | 405.6 | 2560 | 1212 |
| TAZ- 226 | 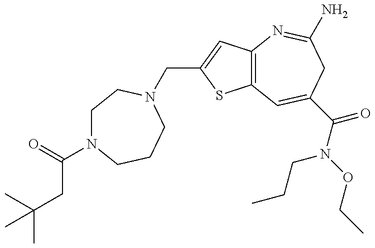 [SMILES: CCCN(OCC)C(=O)C1=Cc2sc(CN3CCCN(C(=O)CC(C)(C)C)CC3)cc2N=C(N)C1] | 505.7 | 436 | 4434 |
| TAZ- 227 | 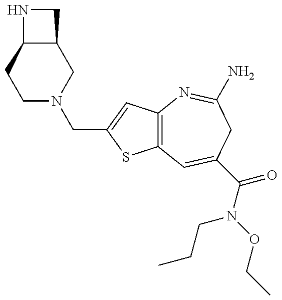 [SMILES: CCCN(OCC)C(=O)C1=Cc2sc(CN3CC[C@H]4NC[C@H]4C3)cc2N=C(N)C1] | 417.6 | 4393 | 2289 |
| TAZ- 228 | 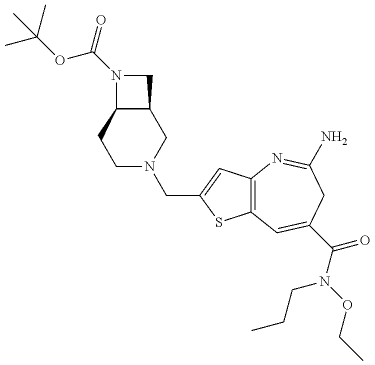 [SMILES: CCCN(OCC)C(=O)C1=Cc2sc(CN3CC[C@@H]4[C@H](C3)CN4C(=O)OC(C)(C)C)cc2N=C(N)C1] | 517.7 | 3759 | 3237 |
| TAZ- 229 | 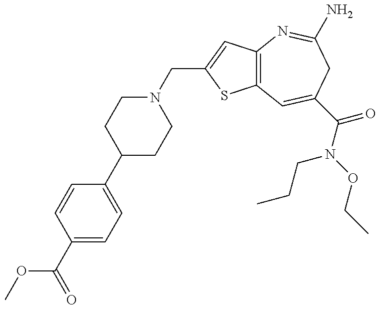 [SMILES: CCCN(OCC)C(=O)C1=Cc2sc(CN3CCC(c4ccc(C(=O)OC)cc4)CC3)cc2N=C(N)C1] | 524.7 | 9000 | 9000 |
| TAZ- 230 | 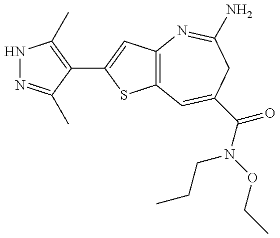 [SMILES: CCCN(OCC)C(=O)C1=Cc2sc(-c3c(C)n[nH]c3C)cc2N=C(N)C1] | 387.5 | | |
| TAZ- 231 | 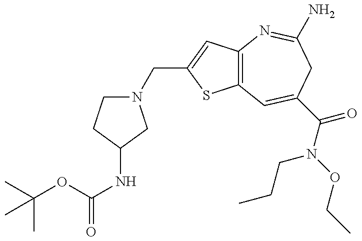 [SMILES: CCCN(OCC)C(=O)C1=Cc2sc(CN3CCC(NC(=O)OC(C)(C)C)C3)cc2N=C(N)C1] | 491.7 | | |
| TAZ- 232 | 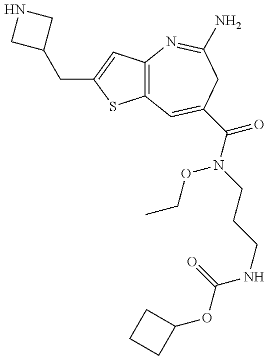 [SMILES: CCON(CCCNC(=O)OC1CCC1)C(=O)C1=Cc2sc(CC3CNC3)cc2N=C(N)C1] | 475.6 | | |
| TAZ- 233 | 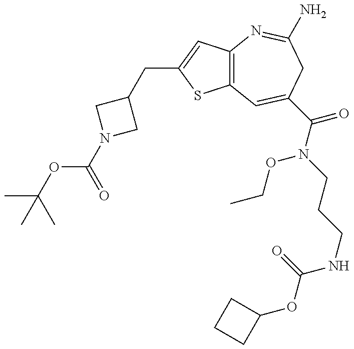 [SMILES: CCON(CCCNC(=O)OC1CCC1)C(=O)C1=Cc2sc(CC3CN(C(=O)OC(C)(C)C)C3)cc2N=C(N)C1] | 575.7 | | |
| TAZ- 234 | 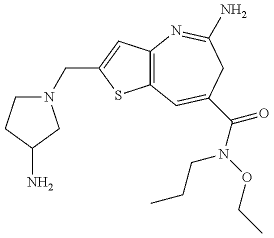 [SMILES: CCCN(OCC)C(=O)C1=Cc2sc(CN3CCC(N)C3)cc2N=C(N)C1] | 391.5 | 3326 | 361 |
| TAZ- 235 | 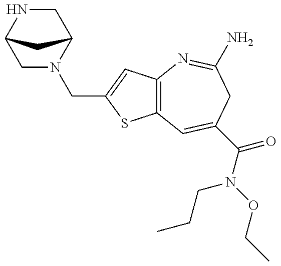 [SMILES: CCCN(OCC)C(=O)C1=Cc2sc(CN3C[C@H]4C[C@@H]3CN4)cc2N=C(N)C1] | 403.5 | 4570 | 3052 |
| TAZ- 236 | 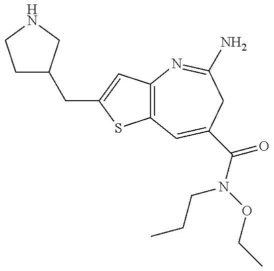 [SMILES: CCCN(OCC)C(=O)C1=Cc2sc(CC3CCNC3)cc2N=C(N)C1] | 376.5 | 2216 | 423 |
| TAZ- 237 | 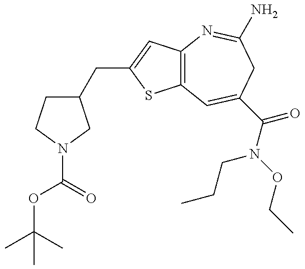 [SMILES: CCCN(OCC)C(=O)C1=Cc2sc(CC3CCN(C(=O)OC(C)(C)C)C3)cc2N=C(N)C1] | 476.6 | | |
| TAZ- 238 | 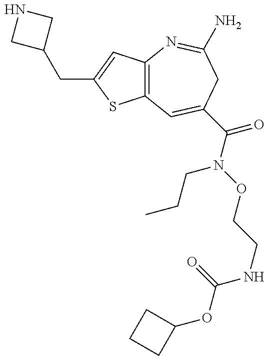 [SMILES: CCCN(OCCNC(=O)OC1CCC1)C(=O)C1=Cc2sc(CC3CNC3)cc2N=C(N)C1] | 475.6 | 112 | 286 |
| TAZ- 239 | 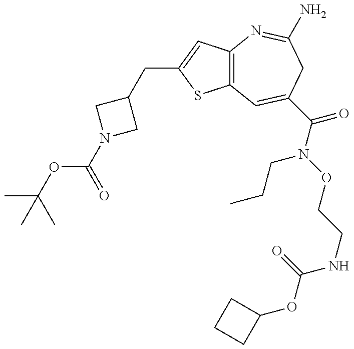 [SMILES: CCCN(OCCNC(=O)OC1CCC1)C(=O)C1=Cc2sc(CC3CN(C(=O)OC(C)(C)C)C3)cc2N=C(N)C1] | 575.7 | | |
| TAZ- 240 | 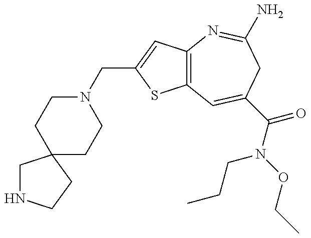 [SMILES: CCCN(OCC)C(=O)C1=Cc2sc(CN3CCC4(CCNC4)CC3)cc2N=C(N)C1] | 445.6 | 4245 | 5461 |
| TAZ- 241 | 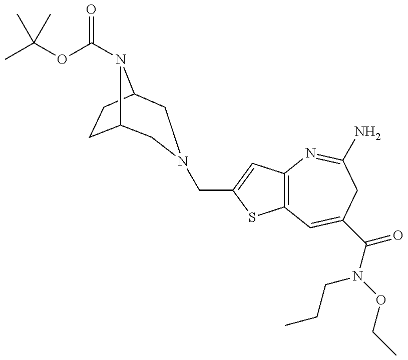 [SMILES: CCCN(OCC)C(=O)C1=Cc2sc(CN3CC4CCC(C3)N4C(=O)OC(C)(C)C)cc2N=C(N)C1] | 517.7 | 2815 | 3061 |
| TAZ- 242 | 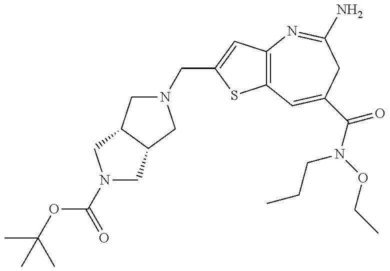 [SMILES: CCCN(OCC)C(=O)C1=Cc2sc(CN3C[C@@H]4CN(C(=O)OC(C)(C)C)C[C@@H]4C3)cc2N=C(N)C1] | 517.7 | 9000 | 3777 |
| TAZ- 243 | 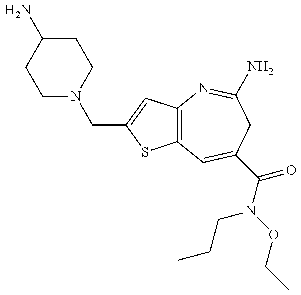 [SMILES: CCCN(OCC)C(=O)C1=Cc2sc(CN3CCC(N)CC3)cc2N=C(N)C1] | 405.6 | 9000 | 164 |
| TAZ- 244 | 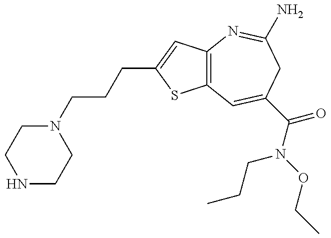 [SMILES: CCCN(OCC)C(=O)C1=Cc2sc(CCCN3CCNCC3)cc2N=C(N)C1] | 419.6 | 2460 | 146 |
| TAZ- 245 | 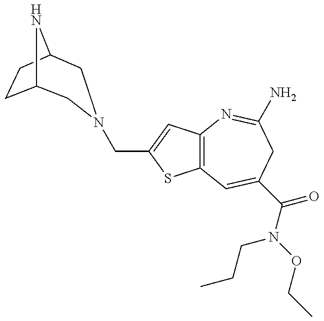 [SMILES: CCCN(OCC)C(=O)C1=Cc2sc(CN3CC4CCC(C3)N4)cc2N=C(N)C1] | 417.6 | 5023 | 145 |
| TAZ- 246 | 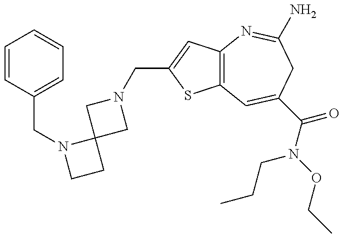 [SMILES: CCCN(OCC)C(=O)C1=Cc2sc(CN3CC4(CCN4Cc4ccccc4)C3)cc2N=C(N)C1] | 493.7 | 9000 | 1107 |
| TAZ- 247 | 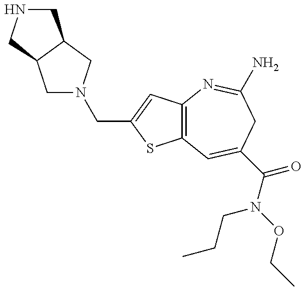 [SMILES: CCCN(OCC)C(=O)C1=Cc2sc(CN3C[C@H]4CNC[C@H]4C3)cc2N=C(N)C1] | 417.6 | 9000 | 418 |
| TAZ- 248 | 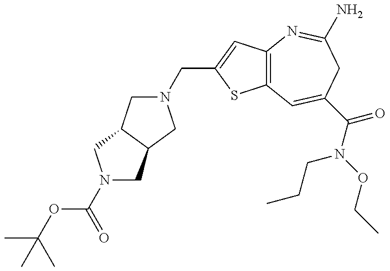 [SMILES: CCCN(OCC)C(=O)C1=Cc2sc(CN3C[C@H]4CN(C(=O)OC(C)(C)C)C[C@@H]4C3)cc2N=C(N)C1] | 517.7 | 9000 | 1114 |
| TAZ- 249 | 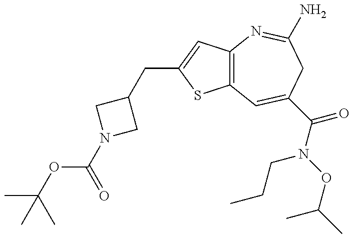 [SMILES: CCCN(OC(C)C)C(=O)C1=Cc2sc(CC3CN(C(=O)OC(C)(C)C)C3)cc2N=C(N)C1] | 476.6 | 362 | 640 |
| TAZ- 250 | 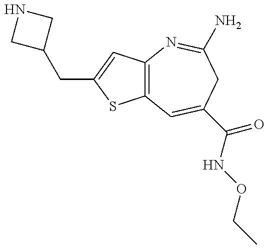 [SMILES: CCONC(=O)C1=Cc2sc(CC3CNC3)cc2N=C(N)C1] | 320.4 | 9000 | 9000 |
| TAZ- 251 | 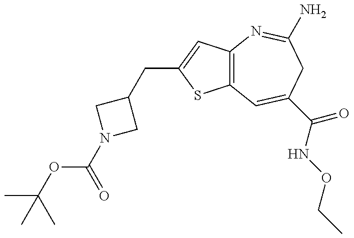 [SMILES: CCONC(=O)C1=Cc2sc(CC3CN(C(=O)OC(C)(C)C)C3)cc2N=C(N)C1] | 420.5 | | |
| TAZ- 252 | 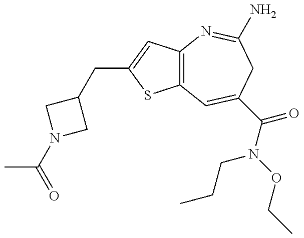 [SMILES: CCCN(OCC)C(=O)C1=Cc2sc(CC3CN(C(C)=O)C3)cc2N=C(N)C1] | 404.5 | 4453 | 868 |
| TAZ- 253 | 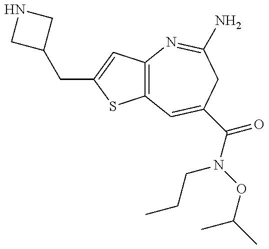 [SMILES: CCCN(OC(C)C)C(=O)C1=Cc2sc(CC3CNC3)cc2N=C(N)C1] | 376.5 | 7166 | 192 |
| TAZ- 254 | 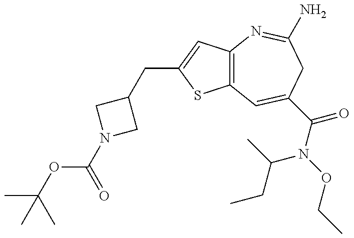 [SMILES: CCON(C(=O)C1=Cc2sc(CC3CN(C(=O)OC(C)(C)C)C3)cc2N=C(N)C1)C(C)CC] | 476.6 | 8133 | 8267 |
| TAZ- 255 | 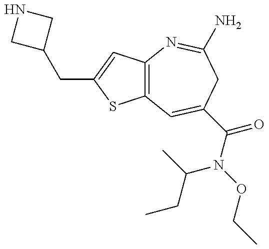 [SMILES: CCON(C(=O)C1=Cc2sc(CC3CNC3)cc2N=C(N)C1)C(C)CC] | 376.5 | | |
| TAZ- 256 | 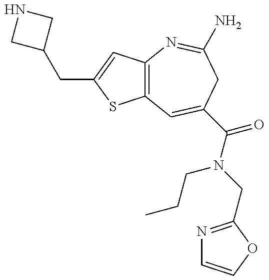 [SMILES: CCCN(Cc1ncco1)C(=O)C1=Cc2sc(CC3CNC3)cc2N=C(N)C1] | 399.5 | 9000 | 3409 |
| TAZ- 257 | 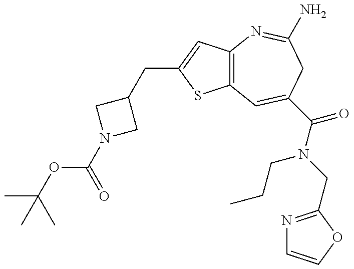 [SMILES: CCCN(Cc1ncco1)C(=O)C1=Cc2sc(CC3CN(C(=O)OC(C)(C)C)C3)cc2N=C(N)C1] | 499.6 | | |
| TAZ- 258 | 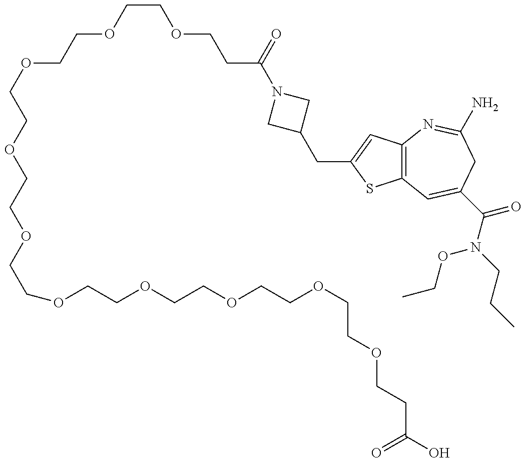 [SMILES: CCCN(OCC)C(=O)C1=Cc2sc(CC3CN(C(=O)CCOCCOCCOCCOCCOCCOCCOCCOCCOCCOCCC(=O)O)C3)cc2N=C(N)C1] | 903.1 | | |
| TAZ- 259 | 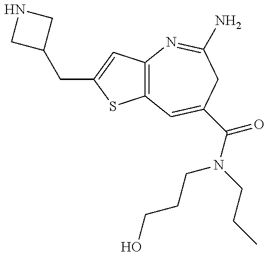 [SMILES: CCCN(CCCO)C(=O)C1=Cc2sc(CC3CNC3)cc2N=C(N)C1] | 376.5 | | |
| TAZ- 260 | 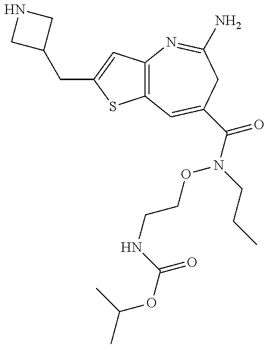 [SMILES: CCCN(OCCNC(=O)OC(C)C)C(=O)C1=Cc2sc(CC3CNC3)cc2N=C(N)C1] | 463.6 | | |
| TAZ- 261 | 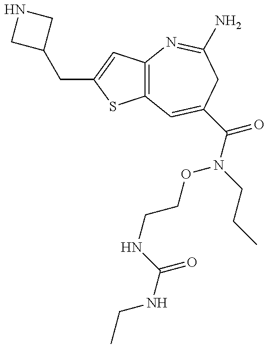 [SMILES: CCCN(OCCNC(=O)NCC)C(=O)C1=Cc2sc(CC3CNC3)cc2N=C(N)C1] | 448.6 | | |
| TAZ- 262 | 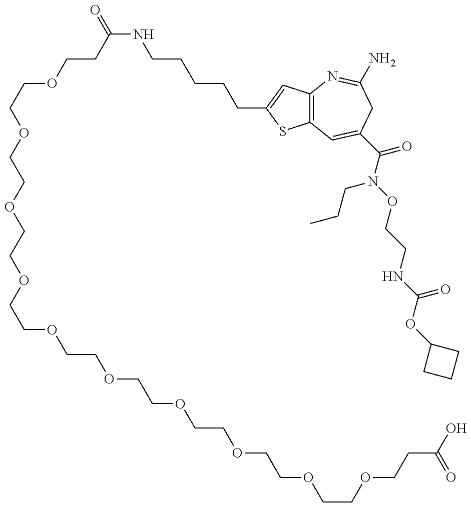 [SMILES: CCCN(OCCNC(=O)OC1CCC1)C(=O)C1=Cc2sc(CCCCCNC(=O)CCOCCOCCOCCOCCOCCOCCOCCOCCOCCOCCC(=O)O)cc2N=C(N)C1] | 1032.2 | | |
| TAZ- 263 | 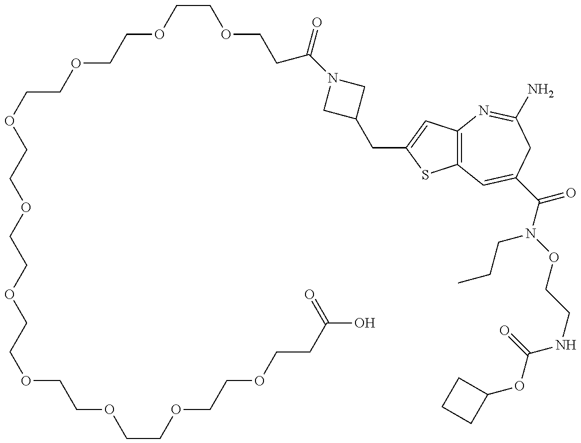 [SMILES: CCCN(OCCNC(=O)OC1CCC1)C(=O)C1=Cc2sc(CC3CN(C(=O)CCOCCOCCOCCOCCOCCOCCOCCOCCOCCOCCC(=O)O)C3)cc2N=C(N)C1] | 1016.2 | | |

### Thienoazepine-Linker Compounds

The macromolecule-supported compounds of the invention are prepared by conjugation of a macromolecular support with a thienoazepine-linker compound. The thienoazepine-linker compounds comprise a thienoazepine (TAZ) moiety covalently attached to a linker unit. The linker units comprise functional groups and subunits which affect stability, permeability, solubility, and other pharmacokinetic, safety, and efficacy properties of the macromolecule-supported compounds. The linker unit includes a reactive functional group which reacts, i.e. conjugates, with a reactive functional group of the macromolecular support. For example, a nucleophilic group such as a lysine side chain amino of the macromolecular support reacts with an electrophilic reactive functional group of the TAZ-linker compound to form the macromolecule-supported compound. Also, for example, a cysteine thiol of the macromolecular support reacts with a maleimide or bromoacetamide group of the TAZ-linker compound to form the macromolecule-supported compound.

Electrophilic reactive functional groups suitable for the TAZ-linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2nd Edition, Academic Press, 2008.

The invention provides solutions to the limitations and challenges to the design, preparation and use of macromolecule-supported compounds. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases macromolecule-supported compound yield and can render process scale-up difficult.

Exemplary embodiments include a 5-amino-thienoazepine-linker compound of Formula II:

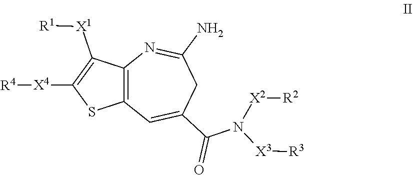

where one of R¹, R², R³, and R⁴ is attached to L;

R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:

—(C₁-C₁₂ alkyldiyl)-N(R⁵)—*;

—(C₁-C₁₂ alkyldiyl)-N(R⁵)₂;

—(C₁-C₁₂ alkyldiyl)-OR⁵;

—(C₃-C₁₂ carbocyclyl);

—(C₃-C₁₂ carbocyclyl)-*;

—(C₃-C₁₂ carbocyclyl)-(C₁-C₁₂ alkyldiyl)-NR⁵—*;

—(C₃-C₁₂ carbocyclyl)-(C₁-C₁₂ alkyldiyl)-N(R⁵)₂;

—(C₃-C₁₂ carbocyclyl)-NR⁵—C(═NR⁵)NR⁵—*;

—(C₆-C₂₀ aryl);

—(C₆-C₂₀ aryl)-*;

—(C₆-C₂₀ aryldiyl)-N(R⁵)—*;

—(C₆-C₂₀ aryldiyl)-(C₁-C₁₂ alkyldiyl)-N(R⁵)—*;

—(C₆-C₂₀ aryldiyl)-(C₁-C₁₂ alkyldiyl)-(C₂-C₂₀ heterocyclyldiyl)-*;

—(C₆-C₂₀ aryldiyl)-(C₁-C₁₂ alkyldiyl)-N(R⁵)₂;

—(C₆-C₂₀ aryldiyl)-(C₁-C₁₂ alkyldiyl)-NR⁵—C(═NR^{5a})N(R⁵)—*;

—(C₂-C₂₀ heterocyclyl);

—(C₂-C₂₀ heterocyclyl)-*;

—(C₂-C₉ heterocyclyl)-(C₁-C₁₂ alkyldiyl)-NR⁵—*;

—(C₂-C₉ heterocyclyl)-(C₁-C₁₂ alkyldiyl)-N(R⁵)₂;

—(C₂-C₉ heterocyclyl)-C(═O)—(C₁-C₁₂ alkyldiyl)-N(R⁵)—*;

—(C₂-C₉ heterocyclyl)-NR⁵—C(═NR^{5a})NR⁵—*;

—(C₂-C₉ heterocyclyl)-NR⁵—(C₆-C₂₀ aryldiyl)-(C₁-C₁₂ alkyldiyl)-N(R⁵)—*;

—(C₂-C₉ heterocyclyl)-(C₆-C₂₀ aryldiyl)-*;

—(C₁-C₂₀ heteroaryl);

—(C₁-C₂₀ heteroaryl)-*;

—(C₁-C₂₀ heteroaryl)-(C₁-C₁₂ alkyldiyl)-N(R⁵)—*;

—(C₁-C₂₀ heteroaryl)-(C₁-C₁₂ alkyldiyl)-N(R⁵)₂;

—(C₁-C₂₀ heteroaryl)-NR⁵—C(═NR^{5a})N(R⁵)—*;

—(C₁-C₂₀ heteroaryl)-N(R⁵)C(═O)—(C₁-C₁₂ alkyldiyl)-N(R⁵)—*;

—C(═O)—*;

—C(═O)—(C₁-C₁₂ alkyldiyl)-N(R⁵)—*;

—C(═O)—(C₂-C₂₀ heterocyclyldiyl)-*;

—C(═O)N(R⁵)₂;

—C(═O)N(R⁵)—*;

—C(═O)N(R⁵)—(C₁-C₁₂ alkyldiyl)-N(R⁵)C(═O)R⁵;

—C(═O)N(R⁵)—(C₁-C₁₂ alkyldiyl)-N(R⁵)C(═O)N(R⁵)₂;

—C(═O)NR⁵—(C₁-C₁₂ alkyldiyl)-N(R⁵)CO₂R⁵;

—C(═O)NR⁵—(C₁-C₁₂ alkyldiyl)-N(R⁵)C(═NR^{5a})N(R⁵)₂;

—C(═O)NR⁵—(C₁-C₁₂ alkyldiyl)-NR⁵C(═NR^{5a})R⁵;

—C(═O)NR⁵—(C₁-C₈ alkyldiyl)-NR⁵(C₂-C₅ heteroaryl);

—C(═O)NR⁵—(C₁-C₂₀ heteroaryldiyl)-N(R⁵)—*;

—C(═O)NR⁵—(C₁-C₂₀ heteroaryldiyl)-*;

—C(═O)NR⁵—(C₁-C₂₀ heteroaryldiyl)-(C₁-C₁₂ alkyldiyl)-N(R⁵)₂;

—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;

—N(R⁵)₂;

—N(R⁵)—*;

—N(R⁵)C(═O)R⁵;

—N(R⁵)C(═O)—*;

—N(R⁵)C(═O)N(R⁵)₂;

—N(R⁵)C(═O)N(R⁵)—*;

—N(R⁵)CO₂R⁵;

—NR⁵C(═NR^{5a})N(R⁵)₂;

—NR⁵C(═NR^{5a})N(R⁵)—*;

—NR⁵C(═NR^{5a})R⁵;

—N(R⁵)C(═O)—(C₁-C₁₂ alkyldiyl)-N(R⁵)—*;

—N(R⁵)—(C₂-C₅ heteroaryl);

—N(R⁵)—S(═O)₂—(C₁-C₁₂ alkyl);

—O—(C₁-C₁₂ alkyl);

—O—(C₁-C₁₂ alkyldiyl)-N(R⁵)₂;

—O—(C₁-C₁₂ alkyldiyl)-N(R⁵)—*;

—S(═O)₂—(C₂-C₂₀ heterocyclyldiyl)-*;

—S(═O)₂—(C₂-C₂₀ heterocyclyldiyl)-(C₁-C₁₂ alkyldiyl)-N(R⁵)₂;

—S(═O)₂—(C₂-C₂₀ heterocyclyldiyl)-(C₁-C₁₂ alkyldiyl)-NR⁵—*; and

—S(═O)₂—(C₂-C₂₀ heterocyclyldiyl)-(C₁-C₁₂ alkyldiyl)-OH;

or R² and R³ together form a 5- or 6-membered heterocyclyl ring;

X¹, X², X³, and X⁴ are independently selected from the group consisting of a bond, C(═O), C(═O)N(R⁵), O, N(R⁵), S, S(O)₂, and S(O)₂N(R⁵); R⁵ is selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;

R^{5a} is selected from the group consisting of C₆-C₂₀ aryl and C₁-C₂₀ heteroaryl;

where the asterisk * indicates the attachment site of L, and where one of R¹, R², R³ and R⁴ is attached to L;

L is the linker selected from the group consisting of:

Q-C(═O)-(PEG)-;

Q-C(═O)-(PEG)-C(═O)—;

Q-C(═O)-(PEG)-O—;

Q-C(═O)-(PEG)-C(═O)-(PEP)-;

Q-C(═O)-(PEG)-C(═O)N(R⁵)—(C₁-C₁₂ alkyldiyl)-;

Q-C(═O)-(PEG)-C(═O)N(R⁵)—(C₁-C₁₂ alkyldiyl)-N(R⁵)C(═O)—(C₂-C₅ monoheterocyclyldiyl)-;

Q-C(═O)-(PEG)-C(═O)N(R⁵)—(C₁-C₁₂ alkyldiyl)-(MCgluc)-;

Q-C(═O)-(PEG)-C(═O)-(MCgluc)-;

Q-C(═O)-(PEG)-C(═O)-(PEP)-N(R⁵)—(C₁-C₁₂ alkyldiyl)-;

Q-C(═O)-(PEG)-C(═O)-(PEP)-N(R⁵)—(C₁-C₁₂ alkyldiyl)-N(R⁵)C(═O)—(C₂-C₅ monoheterocyclyldiyl)-;

Q-C(═O)-(PEG)-N(R⁵)—;

Q-C(═O)-(PEG)-N(R⁵)C(═O)—;

Q-C(═O)-(PEG)-N(R⁵)-(PEG)-C(═O)-(PEP)-;

Q-C(═O)-(PEG)-N⁺(R⁵)₂-(PEG)-C(═O)-(PEP)-;

Q-C(═O)-(PEG)-C(═O)—N(R⁵)CH(AA₁)C(═O)-(PEG)-C(═O)-(PEP)-;

Q-C(═O)-(PEG)-C(═O)—N(R⁵)CH(AA₁)C(═O)—N(R⁵)—(C₁-C₁₂ alkyldiyl)-;

Q-C(═O)-(PEG)-SS-(C₁-C₁₂ alkyldiyl)-OC(═O)—;

Q-C(═O)-(PEG)-SS-(C₁-C₁₂ alkyldiyl)-C(═O)—;

Q-C(═O)—(C₁-C₁₂ alkyldiyl)-C(═O)-(PEP)-;

Q-C(═O)—(C₁-C₁₂ alkyldiyl)-C(═O)-(PEP)-N(R⁵)—(C₁-C₁₂ alkyldiyl)-;

Q-C(═O)—(C₁-C₁₂ alkyldiyl)-C(═O)-(PEP)-N(R⁵)—(C₁-C₁₂ alkyldiyl)-N(R⁵)—C(═O);

Q-C(═O)—(C₁-C₁₂ alkyldiyl)-C(═O)-(PEP)-N(R⁵)—(C₁-C₁₂ alkyldiyl)-N(R⁵)C(═O)—(C₂-C₅ monoheterocyclyldiyl)-;

Q-C(═O)—CH₂CH₂OCH₂CH₂—(C₁-C₂₀ heteroaryldiyl)-CH₂O-(PEG)-C(═O)-(MCgluc)-;

Q-C(═O)—CH₂CH₂OCH₂CH₂—(C₁-C₂₀ heteroaryldiyl)-CH₂O-(PEG)-C(═O)-(MCgluc)-N(R⁵)—(C₁-C₁₂ alkyldiyl)-N(R⁵)C(═O)—(C₂-C₅ monoheterocyclyldiyl)-; and

Q-(CH₂)ₘ—C(═O)-(PEP)-N(R⁵)—(C₁-C₁₂ alkyldiyl)-N(R⁵)C(═O)—(C₂-C₅ monoheterocyclyldiyl)-;

where PEG has the formula: —(CH₂CH₂O)n-(CH₂)m-; m is an integer from 1 to 5, and n is an integer from 2 to 50;

PEP has the formula:

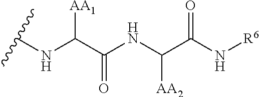

where AA₁ and AA₂ are independently selected from an amino acid side chain, or AA₁ or AA₂ and an adjacent nitrogen atom form a 5-membered ring proline amino acid, and the wavy line indicates a point of attachment and;

R⁶ is selected from the group consisting of C₆-C₂₀ aryldiyl and C₁-C₂₀ heteroaryldiyl, substituted with —CH₂O—C(═O)— and optionally with:

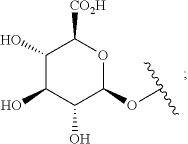 [SMILES: *O[C@@H]1O[C@H](C(=O)O)[C@@H](O)[C@H](O)[C@H]1O]

and

MCgluc is selected from the groups:

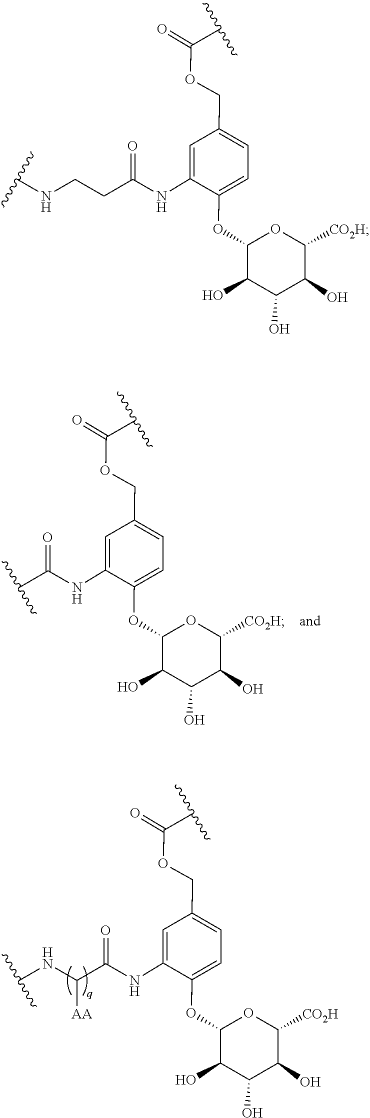

where q is 1 to 8, and AA is an amino acid side chain; and

Q is selected from the group consisting of N-hydroxysuccinimidyl, N-hydroxysulfosuccinimidyl, maleimide, and phenoxy substituted with one or more groups independently selected from F, Cl, NO₂, and SO₃⁻;

where alkyl, alkyldiyl, alkenyl, alkenyldiyl, alkynyl, alkynyldiyl, aryl, aryldiyl carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the thienoazepine-linker compound of Formula II includes wherein PEP has the formula:

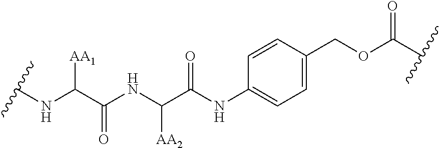

wherein AA₁ and AA₂ are independently selected from a side chain of a naturally-occurring amino acid.

An exemplary embodiment of the thienoazepine-linker compound of Formula II includes wherein AA₁ or AA₂ with an adjacent nitrogen atom form a 5-membered ring to form a proline amino acid.

An exemplary embodiment of the thienoazepine-linker compound of Formula II includes wherein PEP has the formula:

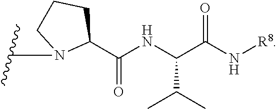

An exemplary embodiment of the thienoazepine-linker compound of Formula II includes wherein MCgluc has the formula:

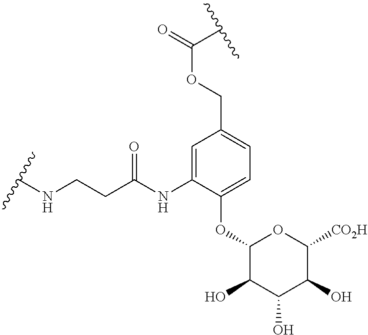 [SMILES: *NCCC(=O)Nc1cc(COC(*)=O)ccc1O[C@@H]1O[C@H](C(=O)O)[C@@H](O)[C@H](O)[C@H]1O]

An exemplary embodiment of the thienoazepine-linker compound of Formula II includes wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

An exemplary embodiment of the thienoazepine-linker compound of Formula II includes wherein AA₁ is —CH(CH₃)₂, and AA₂ is —CH₂CH₂CH₂NHC(O)NH₂.

An exemplary embodiment of the thienoazepine-linker compound of Formula II includes wherein AA₁ and AA₂ are independently selected from GlcNAc aspartic acid, —CH₂SO₃H, and —CH₂OPO₃H.

An exemplary embodiment of the thienoazepine-linker compound of Formula II includes wherein X¹ is a bond, and R¹ is H.

An exemplary embodiment of the thienoazepine-linker compound of Formula II includes wherein X² is a bond, and R² is C₁-C₈ alkyl.

An exemplary embodiment of the thienoazepine-linker compound of Formula II includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

An exemplary embodiment of the thienoazepine-linker compound of Formula II includes wherein R² and R³ are each independently selected from —CH₂CH₂CH₃, —OCH₂CH₃, —CH₂CH₂CF₃, and —CH₂CH₂CH₂OH.

An exemplary embodiment of the thienoazepine-linker compound of Formula II includes wherein R² is C₁-C₈ alkyl and R³ is —(C₁-C₈ alkyldiyl)-N(R⁵)CO₂R⁴.

An exemplary embodiment of the thienoazepine-linker compound of Formula II includes wherein R² is —CH₂CH₂CH₃ and R³ is —CH₂CH₂CH₂NHCO₂(t-Bu).

An exemplary embodiment of the thienoazepine-linker compound of Formula II includes wherein R² and R³ are each —CH₂CH₂CH₃.

An exemplary embodiment of the thienoazepine-linker compound of Formula II includes wherein X³—R³ is selected from the group consisting of:

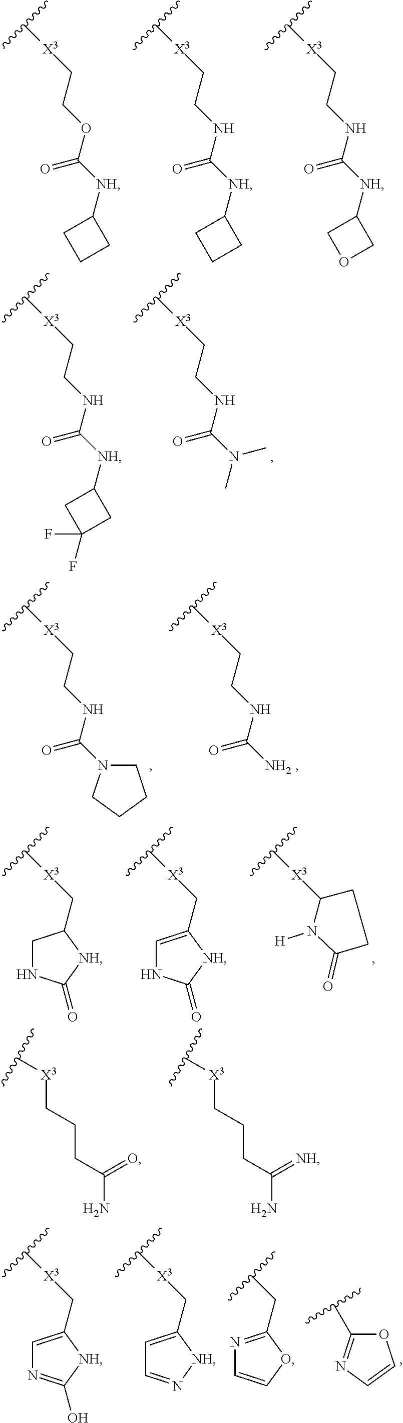

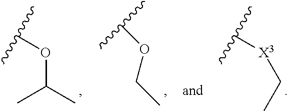

An exemplary embodiment of the thienoazepine-linker compound of Formula II includes wherein one of R² and R³ is selected from:

—(C₁-C₁₂ alkyldiyl)-N(R⁵)—*;

—(C₁-C₁₂ alkyldiyl)-O—(C₁-C₁₂ alkyldiyl)-N(R⁵)—*;

—(C₁-C₁₂ alkyldiyl)-N(R⁵)C(═NR⁵)—N(R⁵)—*;

—(C₁-C₁₂ alkyldiyl)-(C₆-C₂₀ aryldiyl)-(C₁-C₁₂ alkyldiyl)-N(R⁵)—*;

—(C₁-C₁₂ alkyldiyl)-(C₆-C₂₀ aryldiyl)-(C₁-C₁₂ alkyldiyl)-N(R⁵)—C(═NR⁵)N(R⁵)—*;

—(C₂-C₆ alkynyldiyl)-N(R⁵)—*; and

—(C₂-C₆ alkynyldiyl)-N(R⁵)C(═NR⁵)N(R⁵)—*;

X² and X³ are a bond, and where the asterisk * indicates the attachment site of L.

An exemplary embodiment of the thienoazepine-linker compound of Formula II includes wherein L is selected from the group consisting of:

Q-C(═O)-(PEG)-;

Q-C(═O)-(PEG)-C(═O)—;

Q-C(═O)-(PEG)-O—;

Q-C(═O)-(PEG)-N(R⁵)—; and

Q-C(═O)-(PEG)-N(R⁵)C(═O)—.

An exemplary embodiment of the thienoazepine-linker compound of Formula II is selected from Formulae IIa-IIc:

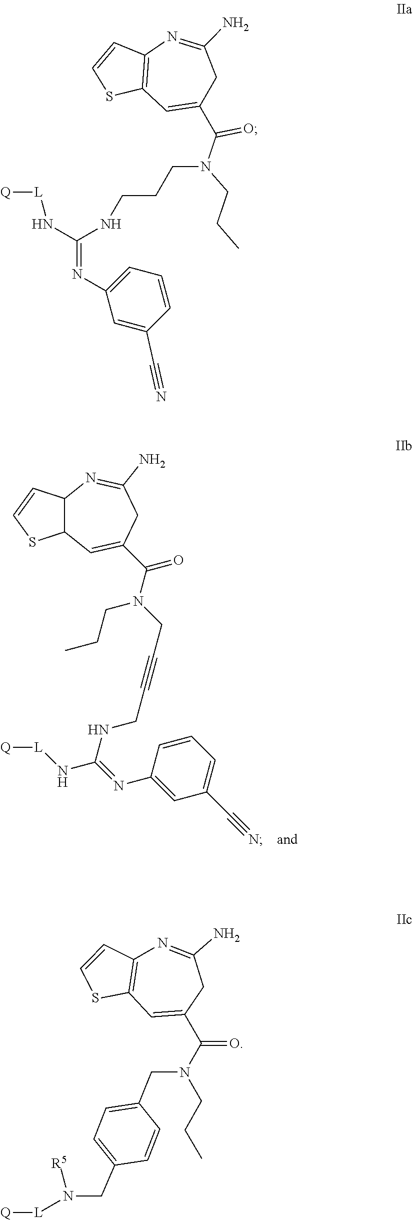

An exemplary embodiment of the thienoazepine-linker compound of Formula II is selected from Formulae IId-IIh:

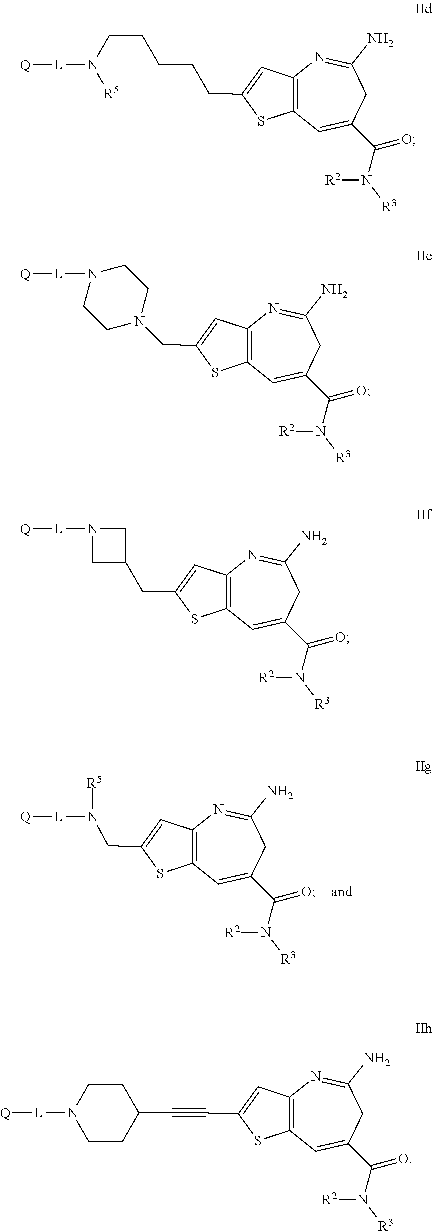

An exemplary embodiment of the thienoazepine-linker compound of Formula II includes wherein Q is selected from:

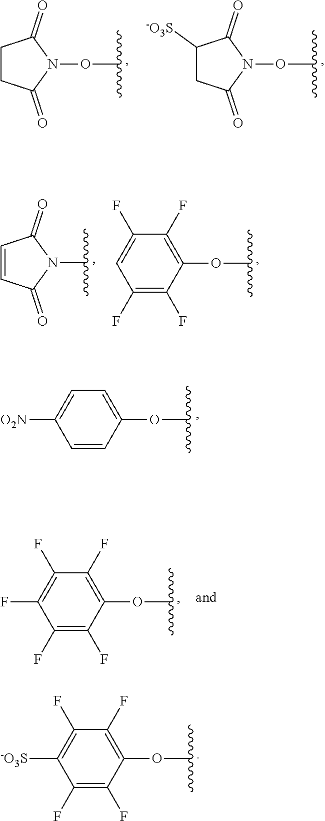

An exemplary embodiment of the thienoazepine-linker compound of Formula II includes wherein Q is phenoxy substituted with one or more F.

An exemplary embodiment of the thienoazepine-linker compound of Formula II includes wherein Q is 2,3,5,6-tetrafluorophenoxy.

An exemplary embodiment of the thienoazepine-linker (TAZ-L) compound is selected from Tables 2a-c. Each compound was synthesized, purified, and characterized by mass spectrometry and shown to have the mass indicated. Additional experimental procedures are found in the Examples. The thienoazepine-linker compounds of Tables 2a-c demonstrate the surprising and unexpected property of TLR8 agonist selectivity which may predict useful therapeutic activity to treat cancer and other disorders. The thienoazepine-linker compounds of Tables 2a-c are used in conjugation with macromolecules by the methods of Example 201 to form the macromolecular-supported compounds (MSC) of Table 3.

TABLE 2a
Thienoazepine-linker (TAZ-L) Formula II compounds

| TAZ-L No. | Structure | MW |
|---|---|---|
| TAZ-L-1 | 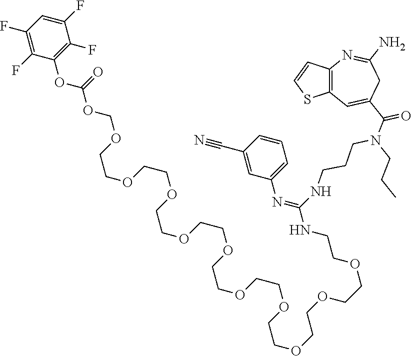 [SMILES: CCCN(CCCN/C(=N\c1cccc(C#N)c1)NCCOCCOCCOCCOCCOCCOCCOCCOCCOCCOCOC(=O)Oc1c(F)c(F)cc(F)c1F)C(=O)C1=Cc2sccc2N=C(N)C1] | 1110.2 |
| TAZ-L-2 | 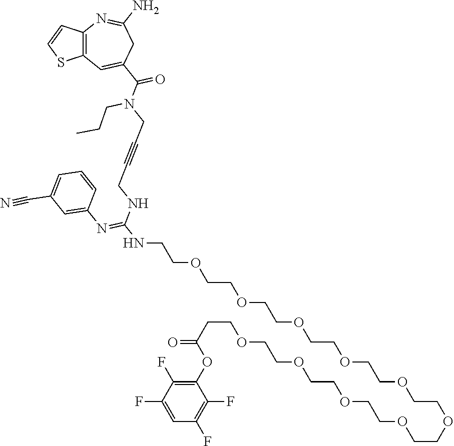 [SMILES: CCCN(CC#CCN/C(=N\c1cccc(C#N)c1)NCCOCCOCCOCCOCCOCCOCCOCCOCCOCCOCCC(=O)Oc1c(F)c(F)cc(F)c1F)C(=O)C1=Cc2sccc2N=C(N)C1] | 1120.2 |
| TAZ-L-3 | 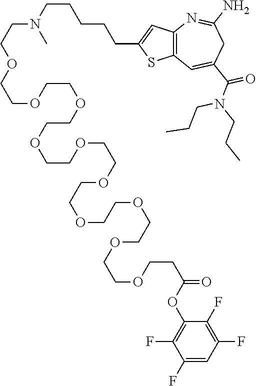 [SMILES: CCCN(CCC)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1051.2 |
| TAZ-L-4 | 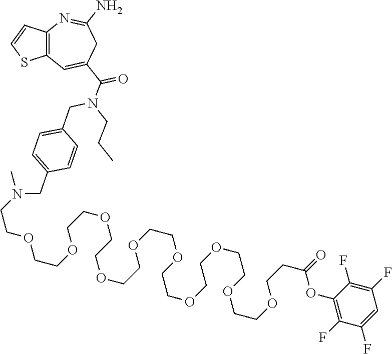 [SMILES: CCCN(Cc1ccc(CN(C)CCOCCOCCOCCOCCOCCOCCOCCOCCOCCOCCC(=O)Oc2c(F)c(F)cc(F)c2F)cc1)C(=O)C1=Cc2sccc2N=C(N)C1] | 1043.2 |

TABLE 2b
Thienoazepine-linker (TAZ-L) Formula II compounds

| TAZ-L No. | Structure | MW |
|---|---|---|
| TAZ-L-5 | 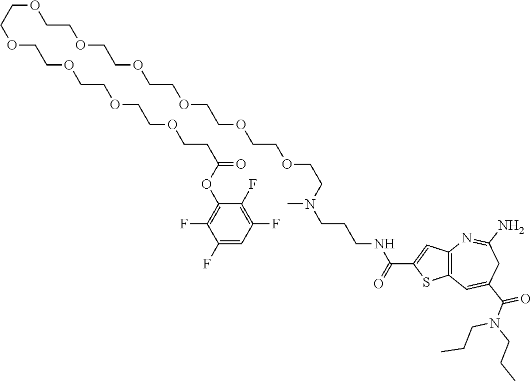 [SMILES: CCCN(CCC)C(=O)C1=Cc2sc(C(=O)NCCCN(C)CCOCCOCCOCCOCCOCCOCCOCCOCCOCCOCCC(=O)Oc3c(F)c(F)cc(F)c3F)cc2N=C(N)C1] | 1066.2 |
| TAZ-L-6 | 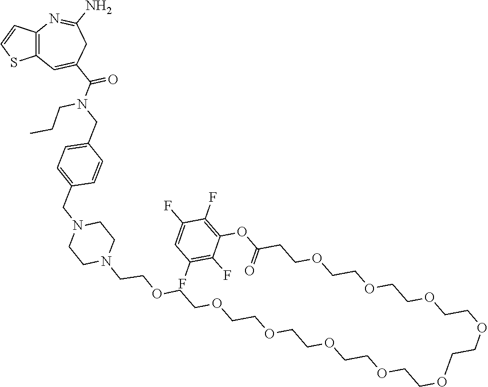 [SMILES: CCCN(Cc1ccc(CN2CCN(CCOCCOCCOCCOCCOCCOCCOCCOCCOCCOCCC(=O)Oc3c(F)c(F)cc(F)c3F)CC2)cc1)C(=O)C1=Cc2sccc2N=C(N)C1] | 1098.3 |
| TAZ-L-7 | 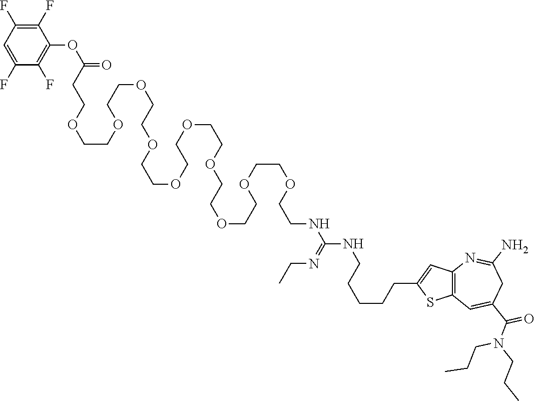 [SMILES: CCCN(CCC)C(=O)C1=Cc2sc(CCCCCN/C(=N/CC)NCCOCCOCCOCCOCCOCCOCCOCCOCCOCCOCCC(=O)Oc3c(F)c(F)cc(F)c3F)cc2N=C(N)C1] | 1107.3 |
| TAZ-L-8 | 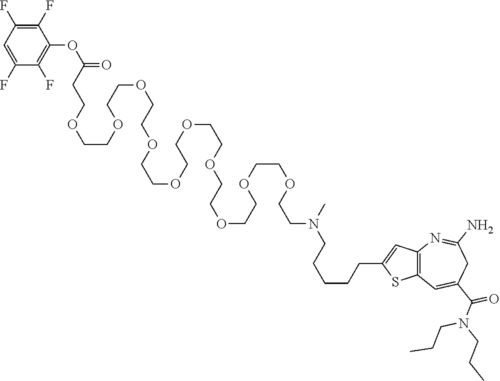 [SMILES: CCCN(CCC)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1037.2 |
| TAZ-L-9 | 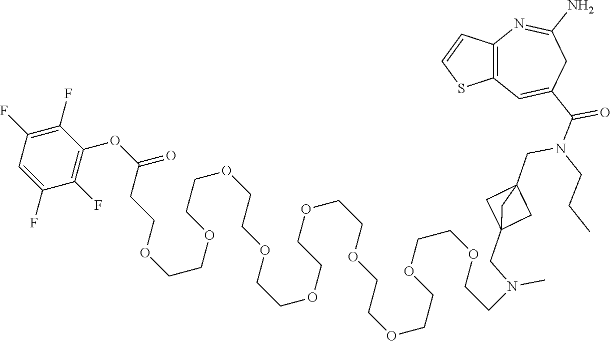 [SMILES: CCCN(CC12CC(CN(C)CCOCCOCCOCCOCCOCCOCCOCCOCCOCCOCCC(=O)Oc3c(F)c(F)cc(F)c3F)(C1)C2)C(=O)C1=Cc2sccc2N=C(N)C1] | 1033.2 |
| TAZ-L-10 | 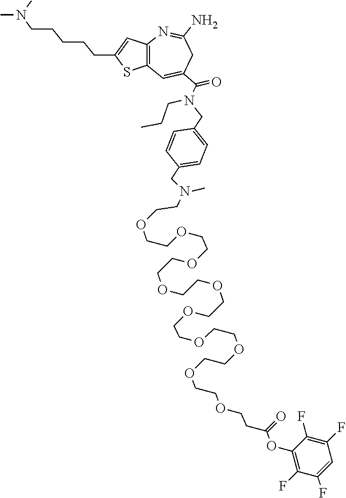 [SMILES: CCCN(Cc1ccc(CN(C)CCOCCOCCOCCOCCOCCOCCOCCOCCOCCOCCC(=O)Oc2c(F)c(F)cc(F)c2F)cc1)C(=O)C1=Cc2sc(CCCCCN(C)C)cc2N=C(N)C1] | 1156.4 |
| TAZ-L-11 | 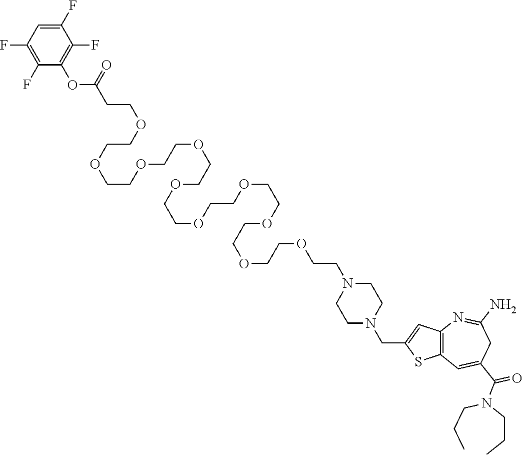 [SMILES: CCCN(CCC)C(=O)C1=Cc2sc(CN3CCN(CCOCCOCCOCCOCCOCCOCCOCCOCCOCCOCCC(=O)Oc4c(F)c(F)cc(F)c4F)CC3)cc2N=C(N)C1] | 1050.2 |
| TAZ-L-12 | 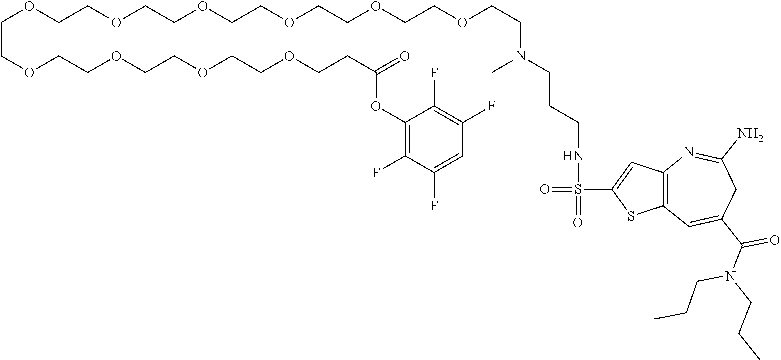 [SMILES: CCCN(CCC)C(=O)C1=Cc2sc(S(=O)(=O)NCCCN(C)CCOCCOCCOCCOCCOCCOCCOCCOCCOCCOCCC(=O)Oc3c(F)c(F)cc(F)c3F)cc2N=C(N)C1] | 1102.3 |
| TAZ-L-13 | 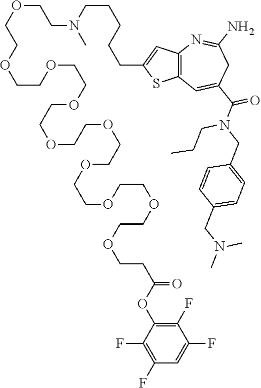 [SMILES: CCCN(Cc1ccc(CN(C)C)cc1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1156.4 |
| TAZ-L-14 | 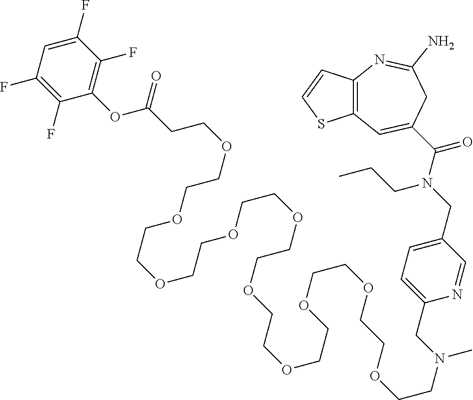 [SMILES: CCCN(Cc1ccc(CN(C)CCOCCOCCOCCOCCOCCOCCOCCOCCOCCOCCC(=O)Oc2c(F)c(F)cc(F)c2F)nc1)C(=O)C1=Cc2sccc2N=C(N)C1] | 1044.2 |
| TAZ-L-15 | 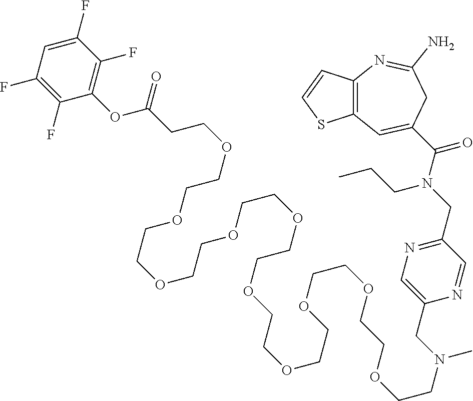 [SMILES: CCCN(Cc1cnc(CN(C)CCOCCOCCOCCOCCOCCOCCOCCOCCOCCOCCC(=O)Oc2c(F)c(F)cc(F)c2F)cn1)C(=O)C1=Cc2sccc2N=C(N)C1] | 1045.1 |
| TAZ-L-16 | 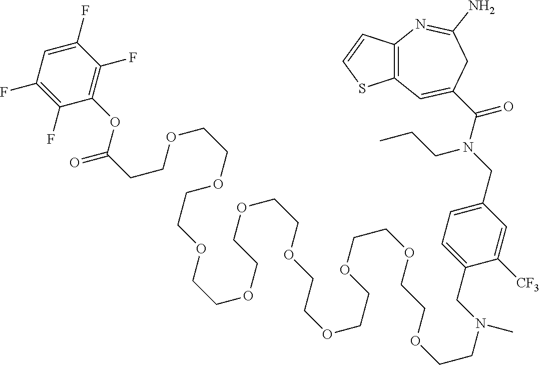 [SMILES: CCCN(Cc1ccc(CN(C)CCOCCOCCOCCOCCOCCOCCOCCOCCOCCOCCC(=O)Oc2c(F)c(F)cc(F)c2F)c(C(F)(F)F)c1)C(=O)C1=Cc2sccc2N=C(N)C1] | 1111.2 |
| TAZ-L-17 | 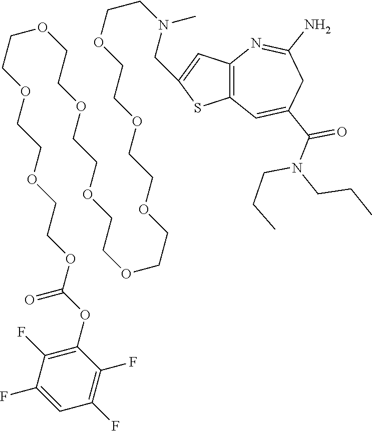 [SMILES: CCCN(CCC)C(=O)C1=Cc2sc(CN(C)CCOCCOCCOCCOCCOCCOCCOCCOCCOCCOC(=O)Oc3c(F)c(F)cc(F)c3F)cc2N=C(N)C1] | 995.13 |
| TAZ-L-18 | 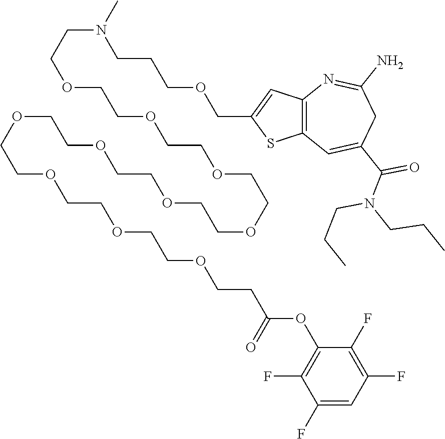 [SMILES: CCCN(CCC)C(=O)C1=Cc2sc(COCCCN(C)CCOCCOCCOCCOCCOCCOCCOCCOCCOCCOCCC(=O)Oc3c(F)c(F)cc(F)c3F)cc2N=C(N)C1] | 1053.2 |
| TAZ-L-19 | 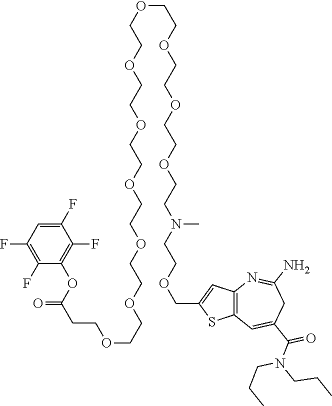 [SMILES: CCCN(CCC)C(=O)C1=Cc2sc(COCCN(C)CCOCCOCCOCCOCCOCCOCCOCCOCCOCCOCCC(=O)Oc3c(F)c(F)cc(F)c3F)cc2N=C(N)C1] | 1039.2 |
| TAZ-L-20 | 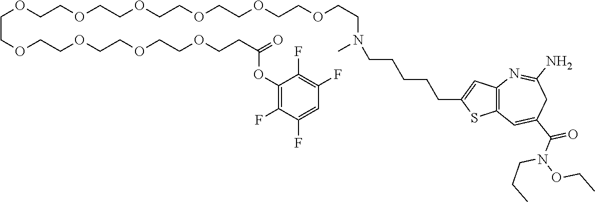 [SMILES: CCCN(OCC)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1053.2 |
| TAZ-L-21 | 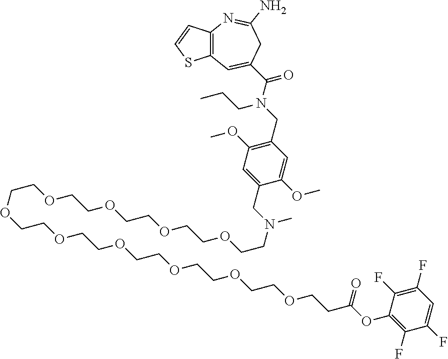 [SMILES: CCCN(Cc1cc(OC)c(CN(C)CCOCCOCCOCCOCCOCCOCCOCCOCCOCCOCCC(=O)Oc2c(F)c(F)cc(F)c2F)cc1OC)C(=O)C1=Cc2sccc2N=C(N)C1] | 1103.2 |
| TAZ-L-22 | 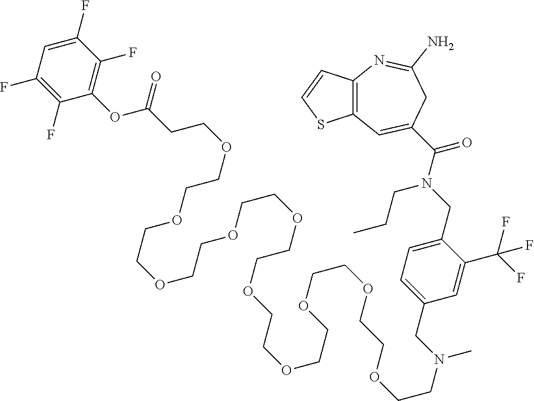 [SMILES: CCCN(Cc1ccc(CN(C)CCOCCOCCOCCOCCOCCOCCOCCOCCOCCOCCC(=O)Oc2c(F)c(F)cc(F)c2F)cc1C(F)(F)F)C(=O)C1=Cc2sccc2N=C(N)C1] | 1111.2 |
| TAZ-L-23 | 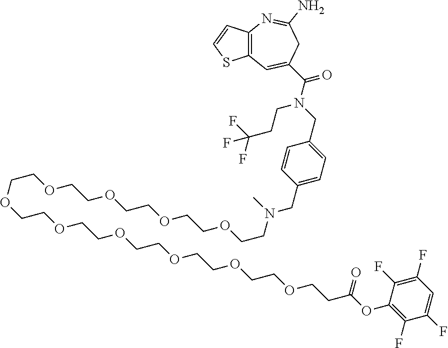 [SMILES: CN(CCOCCOCCOCCOCCOCCOCCOCCOCCOCCOCCC(=O)Oc1c(F)c(F)cc(F)c1F)Cc1ccc(CN(CCC(F)(F)F)C(=O)C2=Cc3sccc3N=C(N)C2)cc1] | 1097.1 |
| TAZ-L-24 | 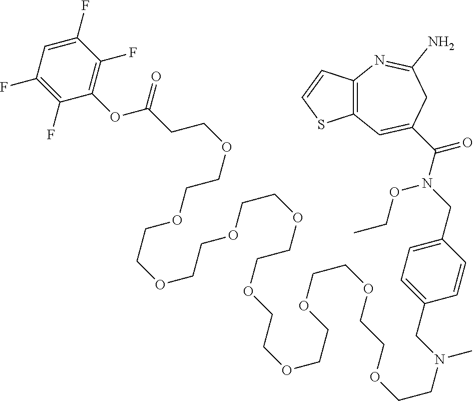 [SMILES: CCON(Cc1ccc(CN(C)CCOCCOCCOCCOCCOCCOCCOCCOCCOCCOCCC(=O)Oc2c(F)c(F)cc(F)c2F)cc1)C(=O)C1=Cc2sccc2N=C(N)C1] | 1045.1 |
| TAZ-L-25 | 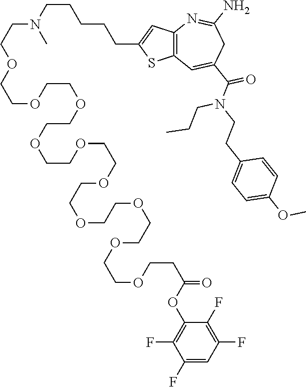 [SMILES: CCCN(CCc1ccc(OC)cc1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1143.3 |
| TAZ-L-26 | 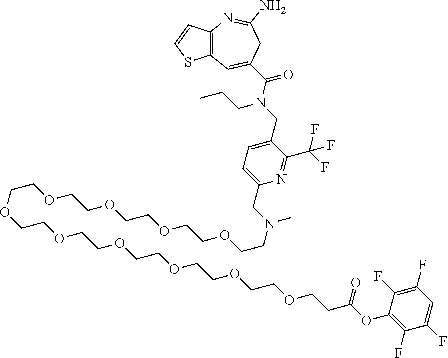 [SMILES: CCCN(Cc1ccc(CN(C)CCOCCOCCOCCOCCOCCOCCOCCOCCOCCOCCC(=O)Oc2c(F)c(F)cc(F)c2F)nc1C(F)(F)F)C(=O)C1=Cc2sccc2N=C(N)C1] | 1112.2 |
| TAZ-L-27 | 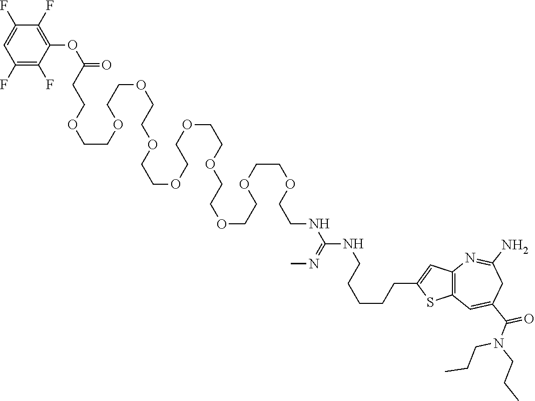 [SMILES: CCCN(CCC)C(=O)C1=Cc2sc(CCCCCN/C(=N/C)NCCOCCOCCOCCOCCOCCOCCOCCOCCOCCOCCC(=O)Oc3c(F)c(F)cc(F)c3F)cc2N=C(N)C1] | 1093.3 |
| TAZ-L-28 | 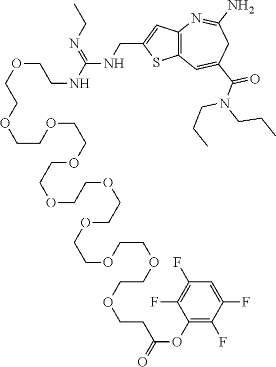 [SMILES: CCCN(CCC)C(=O)C1=Cc2sc(CN/C(=N\CC)NCCOCCOCCOCCOCCOCCOCCOCCOCCOCCOCCC(=O)Oc3c(F)c(F)cc(F)c3F)cc2N=C(N)C1] | 1051.2 |
| TAZ-L-29 | 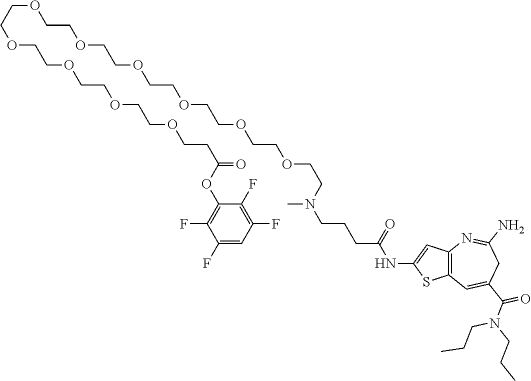 [SMILES: CCCN(CCC)C(=O)C1=Cc2sc(NC(=O)CCCN(C)CCOCCOCCOCCOCCOCCOCCOCCOCCOCCOCCC(=O)Oc3c(F)c(F)cc(F)c3F)cc2N=C(N)C1] | 1066.2 |
| TAZ-L-30 | 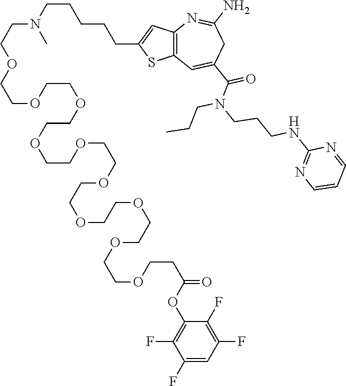 [SMILES: CCCN(CCCNc1ncccn1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1144.3 |
| TAZ-L-31 | 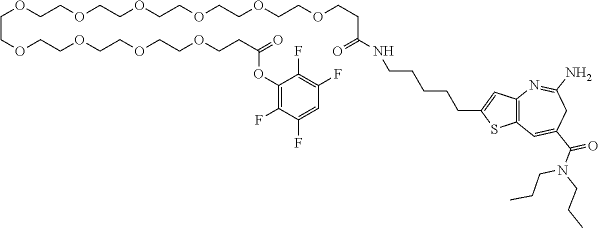 [SMILES: CCCN(CCC)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1065.2 |
| TAZ-L-32 | 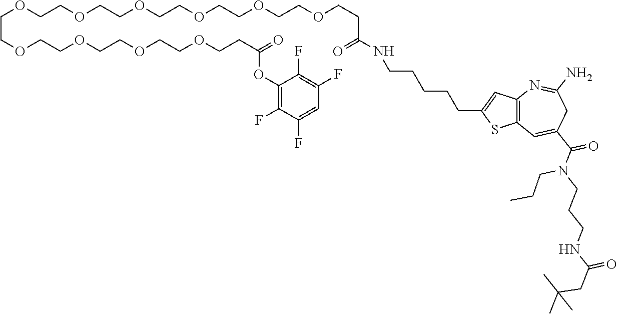 [SMILES: CCCN(CCCNC(=O)CC(C)(C)C)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1178.4 |
| TAZ-L-33 | 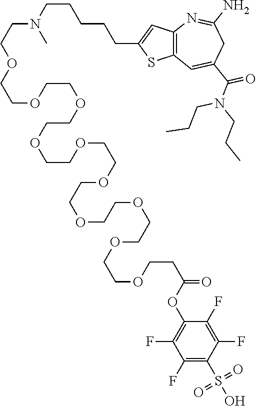 [SMILES: CCCN(CCC)C(=O)C1=Cc2sc(CCCCCN(C)CCOCCOCCOCCOCCOCCOCCOCCOCCOCCOCCC(=O)Oc3c(F)c(F)c(S(=O)(=O)O)c(F)c3F)cc2N=C(N)C1] | 1131.3 |
| TAZ-L-34 | 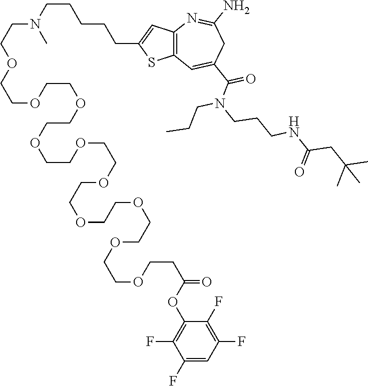 [SMILES: CCCN(CCCNC(=O)CC(C)(C)C)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1164.4 |
| TAZ-L-35 | 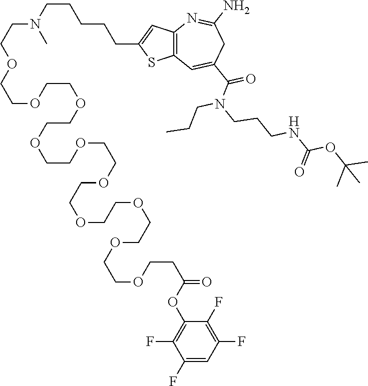 [SMILES: CCCN(CCCNC(=O)OC(C)(C)C)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1166.4 |
| TAZ-L-36 | 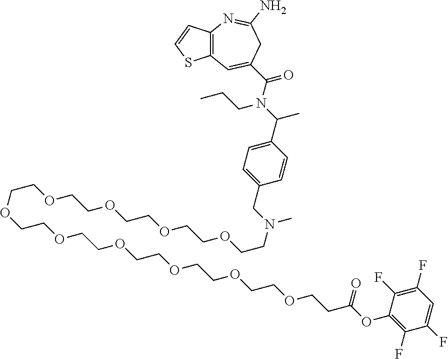 [SMILES: CCCN(C(=O)C1=Cc2sccc2N=C(N)C1)C(C)c1ccc(CN(C)CCOCCOCCOCCOCCOCCOCCOCCOCCOCCOCCC(=O)Oc2c(F)c(F)cc(F)c2F)cc1] | 1057.2 |
| TAZ-L-37 | 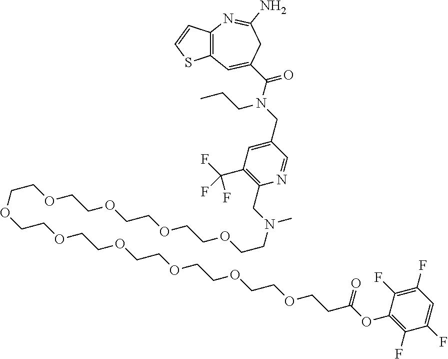 [SMILES: CCCN(Cc1cnc(CN(C)CCOCCOCCOCCOCCOCCOCCOCCOCCOCCOCCC(=O)Oc2c(F)c(F)cc(F)c2F)c(C(F)(F)F)c1)C(=O)C1=Cc2sccc2N=C(N)C1] | 1112.2 |

TABLE 2c
Thienoazepine-linker (TAZ-L) Formula II compounds

| TAZ-L No. | Structure | MW |
|---|---|---|
| TAZ-L-38 | 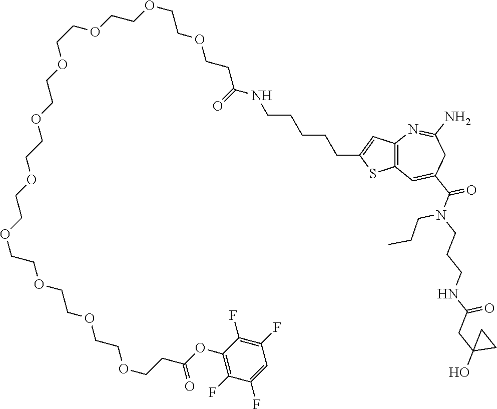 [SMILES: CCCN(CCCNC(=O)CC1(O)CC1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1178.3 |
| TAZ-L-39 | 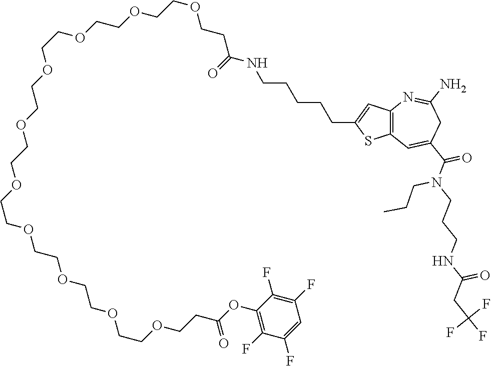 [SMILES: CCCN(CCCNC(=O)CC(F)(F)F)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1190.3 |
| TAZ-L-40 | 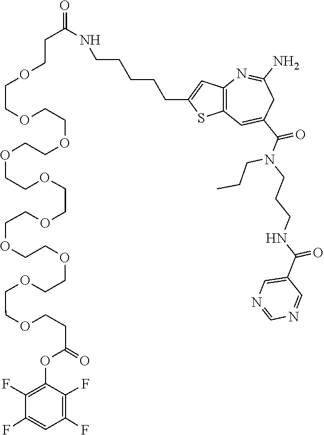 [SMILES: CCCN(CCCNC(=O)c1cncnc1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1186.3 |
| TAZ-L-41 | 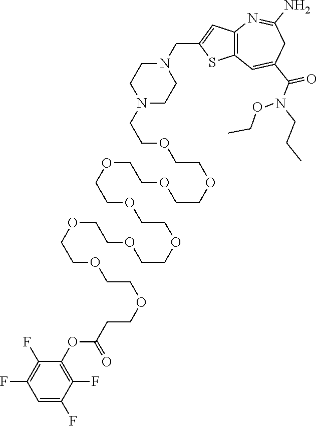 [SMILES: CCCN(OCC)C(=O)C1=Cc2sc(CN3CCN(CCOCCOCCOCCOCCOCCOCCOCCOCCOCCOCCC(=O)Oc4c(F)c(F)cc(F)c4F)CC3)cc2N=C(N)C1] | 1052.2 |
| TAZ-L-42 | 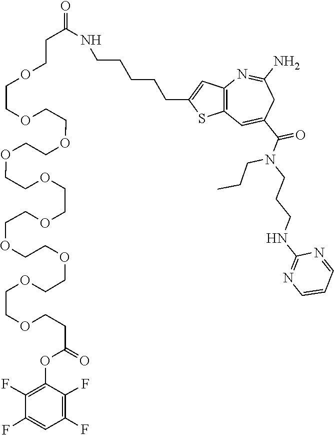 [SMILES: CCCN(CCCNc1ncccn1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1158.3 |
| TAZ-L-43 | 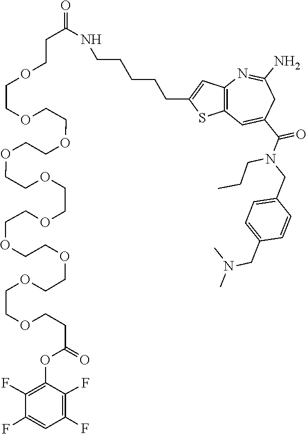 [SMILES: CCCN(Cc1ccc(CN(C)C)cc1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1170.4 |
| TAZ-L-44 | 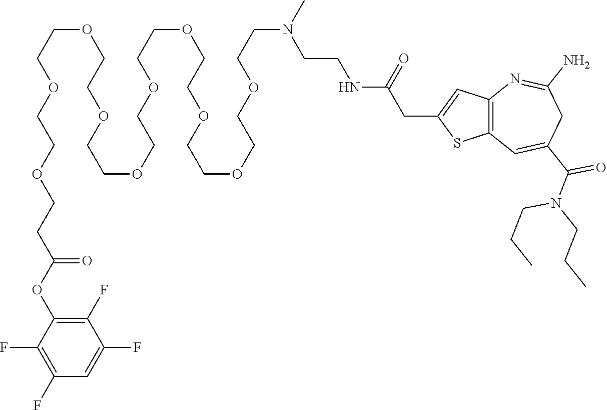 [SMILES: CCCN(CCC)C(=O)C1=Cc2sc(CC(=O)NCCN(C)CCOCCOCCOCCOCCOCCOCCOCCOCCOCCOCCC(=O)Oc3c(F)c(F)cc(F)c3F)cc2N=C(N)C1] | 1066.2 |
| TAZ-L-45 | 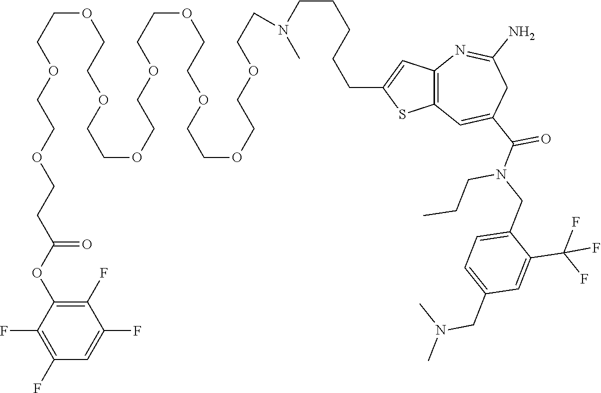 [SMILES: CCCN(Cc1ccc(CN(C)C)cc1C(F)(F)F)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1224.4 |
| TAZ-L-46 | 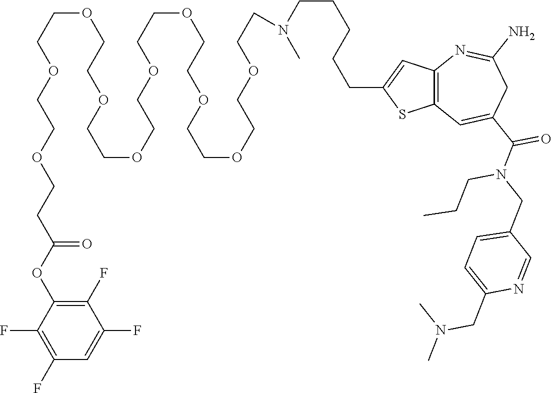 [SMILES: CCCN(Cc1ccc(CN(C)C)nc1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1157.4 |
| TAZ-L-47 | 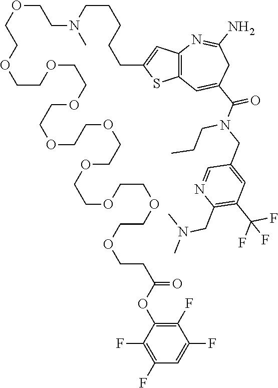 [SMILES: CCCN(Cc1cnc(CN(C)C)c(C(F)(F)F)c1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1225.4 |
| TAZ-L-48 | 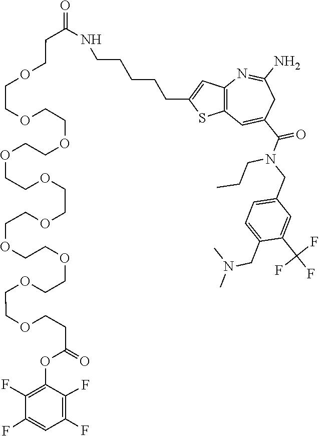 [SMILES: CCCN(Cc1ccc(CN(C)C)c(C(F)(F)F)c1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1238.4 |
| TAZ-L-49 | 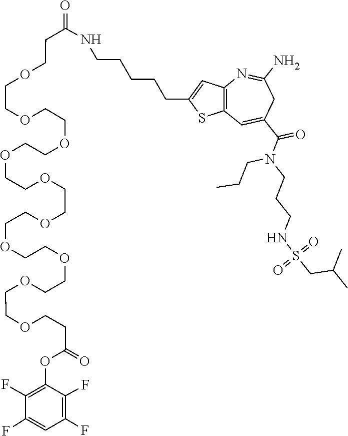 [SMILES: CCCN(CCCNS(=O)(=O)CC(C)C)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1200.4 |
| TAZ-L-50 | 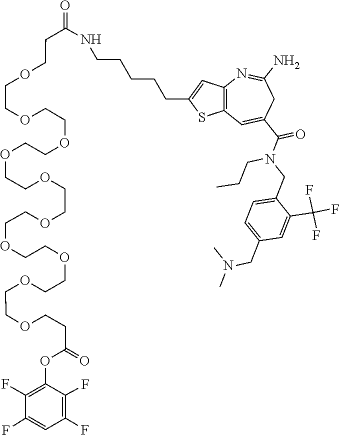 [SMILES: CCCN(Cc1ccc(CN(C)C)cc1C(F)(F)F)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1238.4 |
| TAZ-L-51 | 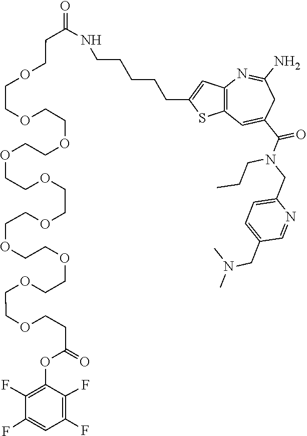 [SMILES: CCCN(Cc1ccc(CN(C)C)cn1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1171.3 |
| TAZ-L-52 | 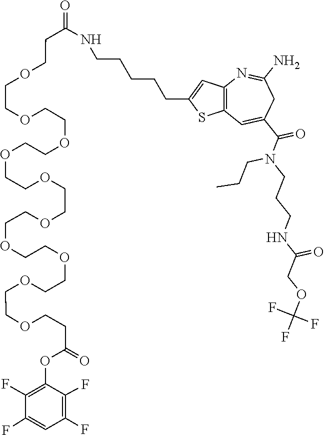 [SMILES: CCCN(CCCNC(=O)COC(F)(F)F)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1206.3 |
| TAZ-L-53 | 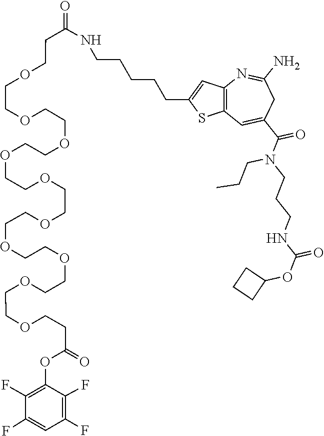 [SMILES: CCCN(CCCNC(=O)OC1CCC1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1178.3 |
| TAZ-L-54 | 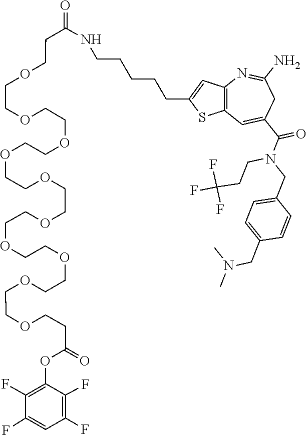 [SMILES: CN(C)Cc1ccc(CN(CCC(F)(F)F)C(=O)C2=Cc3sc(CCCCCNC(=O)CCOCCOCCOCCOCCOCCOCCOCCOCCOCCOCCC(=O)Oc4c(F)c(F)cc(F)c4F)cc3N=C(N)C2)cc1] | 1224.3 |
| TAZ-L-55 | 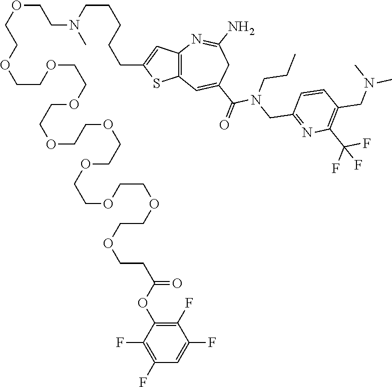 [SMILES: CCCN(Cc1ccc(CN(C)C)c(C(F)(F)F)n1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1225.4 |
| TAZ-L-56 | 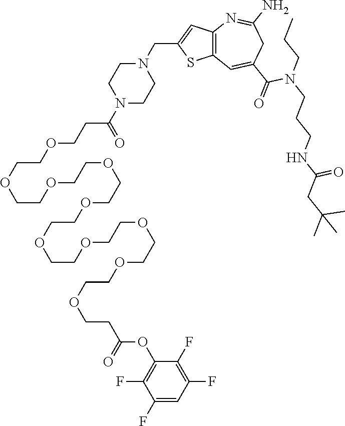 [SMILES: CCCN(CCCNC(=O)CC(C)(C)C)C(=O)C1=Cc2sc(CN3CCN(C(=O)CCOCCOCCOCCOCCOCCOCCOCCOCCOCCOCCC(=O)Oc4c(F)c(F)cc(F)c4F)CC3)cc2N=C(N)C1] | 1191.4 |
| TAZ-L-57 | 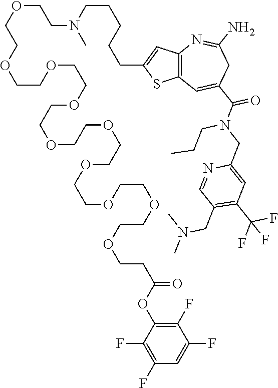 [SMILES: CCCN(Cc1cc(C(F)(F)F)c(CN(C)C)cn1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1225.4 |
| TAZ-L-58 | 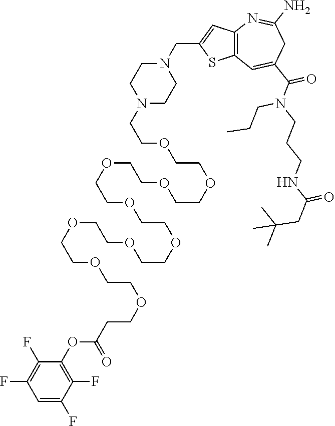 [SMILES: CCCN(CCCNC(=O)CC(C)(C)C)C(=O)C1=Cc2sc(CN3CCN(CCOCCOCCOCCOCCOCCOCCOCCOCCOCCOCCC(=O)Oc4c(F)c(F)cc(F)c4F)CC3)cc2N=C(N)C1] | 1163.4 |
| TAZ-L-59 | 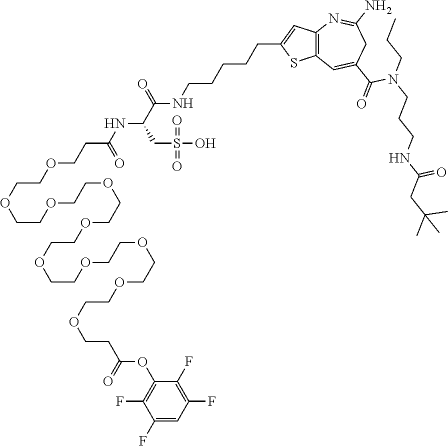 [SMILES: CCCN(CCCNC(=O)CC(C)(C)C)C(=O)C1=Cc2sc(CCCCCNC(=O)[C@H](CS(=O)(=O)O)NC(=O)CCOCCOCCOCCOCCOCCOCCOCCOCCOCCOCCC(=O)Oc3c(F)c(F)cc(F)c3F)cc2N=C(N)C1] | 1329.5 |
| TAZ-L-60 | 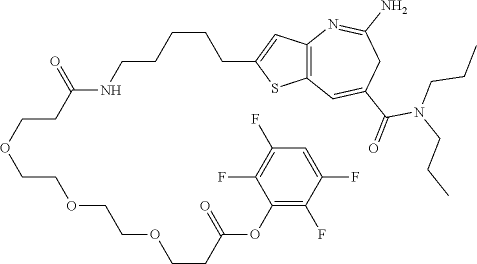 [SMILES: CCCN(CCC)C(=O)C1=Cc2sc(CCCCCNC(=O)CCOCCOCCOCCC(=O)Oc3c(F)c(F)cc(F)c3F)cc2N=C(N)C1] | 756.9 |
| TAZ-L-61 | 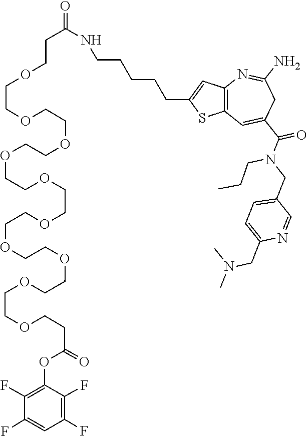 [SMILES: CCCN(Cc1ccc(CN(C)C)nc1)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1171.3 |
| TAZ-L-62 | 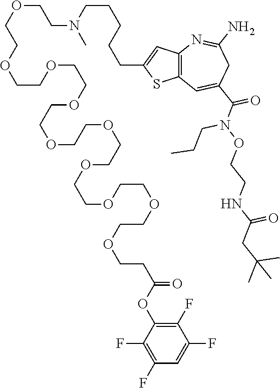 [SMILES: CCCN(OCCNC(=O)CC(C)(C)C)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1166.4 |
| TAZ-L-63 | 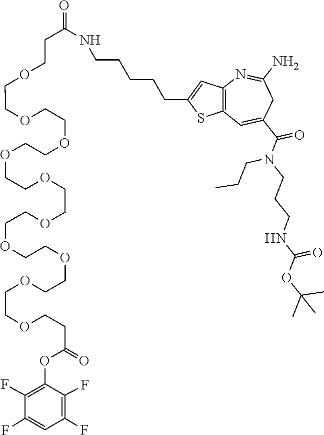 [SMILES: CCCN(CCCNC(=O)OC(C)(C)C)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1180.4 |
| TAZ-L-64 | 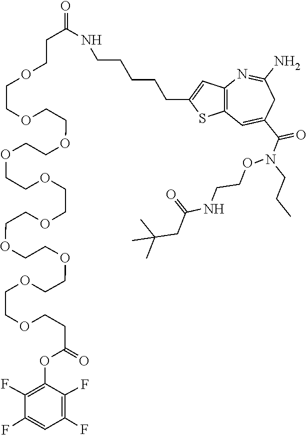 [SMILES: CCCN(OCCNC(=O)CC(C)(C)C)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1180.4 |
| TAZ-L-65 | 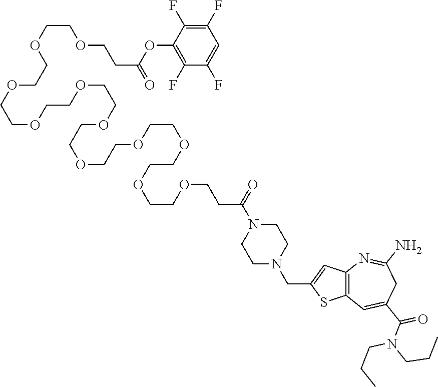 [SMILES: CCCN(CCC)C(=O)C1=Cc2sc(CN3CCN(C(=O)CCOCCOCCOCCOCCOCCOCCOCCOCCOCCOCCOCCC(=O)Oc4c(F)c(F)cc(F)c4F)CC3)cc2N=C(N)C1] | 1122.3 |
| TAZ-L-66 | 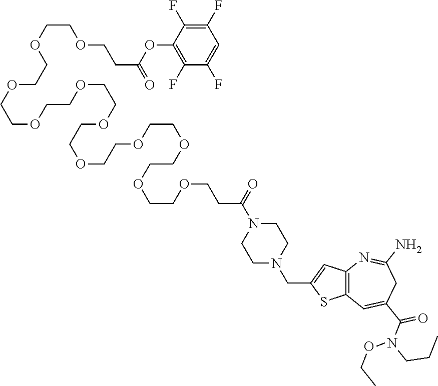 [SMILES: CCCN(OCC)C(=O)C1=Cc2sc(CN3CCN(C(=O)CCOCCOCCOCCOCCOCCOCCOCCOCCOCCOCCOCCC(=O)Oc4c(F)c(F)cc(F)c4F)CC3)cc2N=C(N)C1] | 1124.2 |
| TAZ-L-67 | 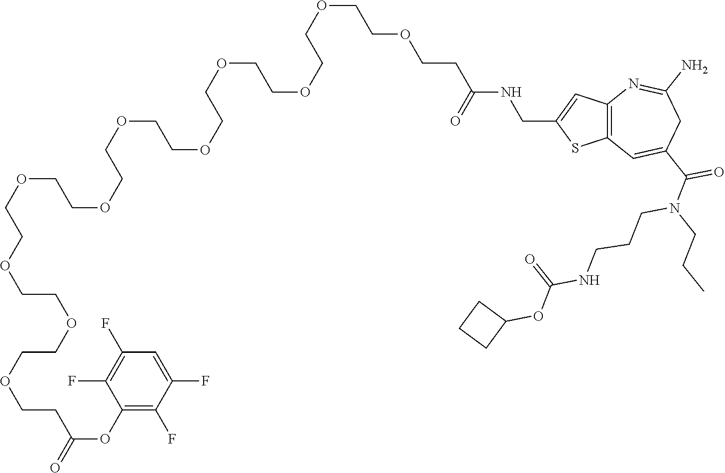 [SMILES: CCCN(CCCNC(=O)OC1CCC1)C(=O)C1=Cc2sc(CNC(=O)CCOCCOCCOCCOCCOCCOCCOCCOCCOCCOCCOCCC(=O)Oc3c(F)c(F)cc(F)c3F)cc2N=C(N)C1] | 1166.3 |
| TAZ-L-68 | 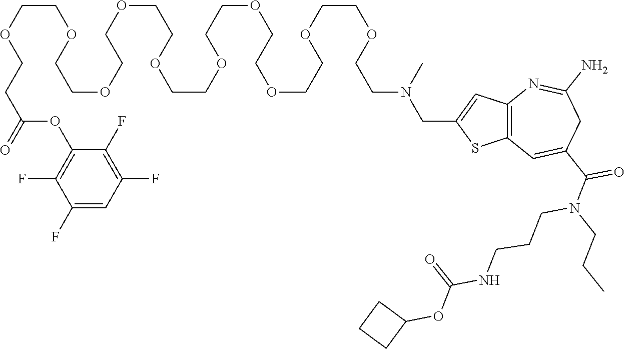 [SMILES: CCCN(CCCNC(=O)OC1CCC1)C(=O)C1=Cc2sc(CN(C)CCOCCOCCOCCOCCOCCOCCOCCOCCOCCOCCC(=O)Oc3c(F)c(F)cc(F)c3F)cc2N=C(N)C1] | 1108.2 |
| TAZ-L-69 | 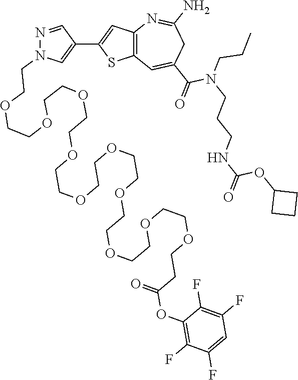 [SMILES: CCCN(CCCNC(=O)OC1CCC1)C(=O)C1=Cc2sc(-c3cnn(CCOCCOCCOCCOCCOCCOCCOCCOCCOCCOCCC(=O)Oc4c(F)c(F)cc(F)c4F)c3)cc2N=C(N)C1] | 1131.2 |
| TAZ-L-70 | 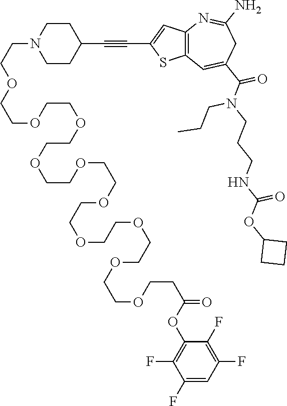 [SMILES: CCCN(CCCNC(=O)OC1CCC1)C(=O)C1=Cc2sc(C#CC3CCN(CCOCCOCCOCCOCCOCCOCCOCCOCCOCCOCCC(=O)Oc4c(F)c(F)cc(F)c4F)CC3)cc2N=C(N)C1] | 1172.3 |
| TAZ-L-71 | 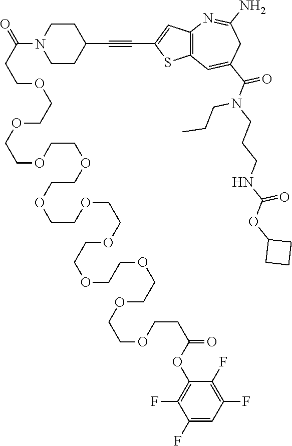 [SMILES: CCCN(CCCNC(=O)OC1CCC1)C(=O)C1=Cc2sc(C#CC3CCN(C(=O)CCOCCOCCOCCOCCOCCOCCOCCOCCOCCOCCOCCC(=O)Oc4c(F)c(F)cc(F)c4F)CC3)cc2N=C(N)C1] | 1244.4 |
| TAZ-L-72 | 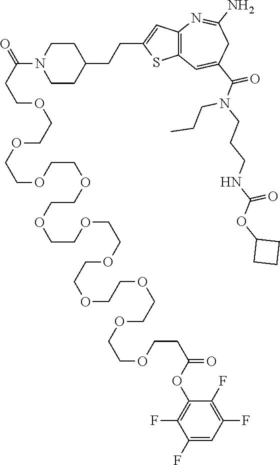 [SMILES: CCCN(CCCNC(=O)OC1CCC1)C(=O)C1=Cc2sc(CCC3CCN(C(=O)CCOCCOCCOCCOCCOCCOCCOCCOCCOCCOCCOCCC(=O)Oc4c(F)c(F)cc(F)c4F)CC3)cc2N=C(N)C1] | 1248.4 |
| TAZ-L-73 | 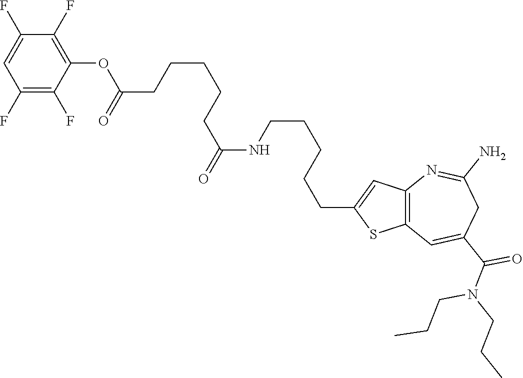 [SMILES: CCCN(CCC)C(=O)C1=Cc2sc(CCCCCNC(=O)CCCCCC(=O)Oc3c(F)c(F)cc(F)c3F)cc2N=C(N)C1] | 666.8 |
| TAZ-L-74 | 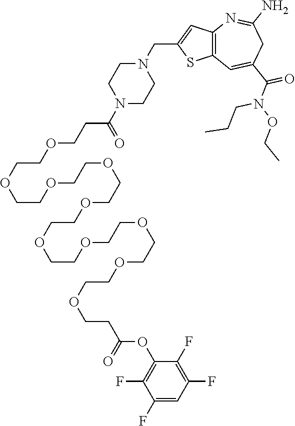 [SMILES: CCCN(OCC)C(=O)C1=Cc2sc(CN3CCN(C(=O)CCOCCOCCOCCOCCOCCOCCOCCOCCOCCOCCC(=O)Oc4c(F)c(F)cc(F)c4F)CC3)cc2N=C(N)C1] | 1080.2 |
| TAZ-L-75 | 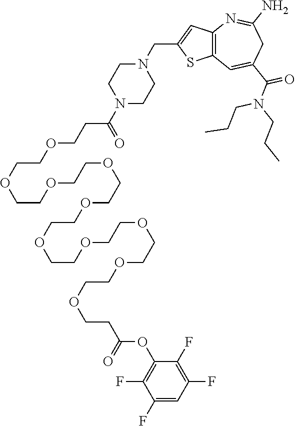 [SMILES: CCCN(CCC)C(=O)C1=Cc2sc(CN3CCN(C(=O)CCOCCOCCOCCOCCOCCOCCOCCOCCOCCOCCC(=O)Oc4c(F)c(F)cc(F)c4F)CC3)cc2N=C(N)C1] | 1078.2 |
| TAZ-L-76 | 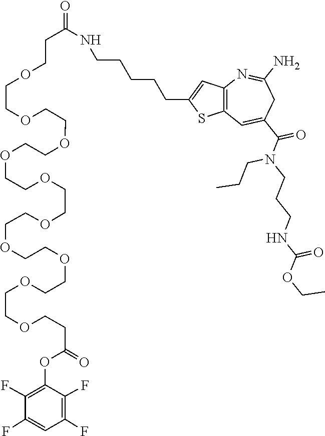 [SMILES: CCCN(CCCNC(=O)OCC)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1152.3 |
| TAZ-L-77 | 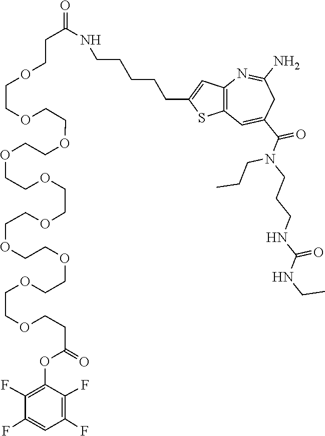 [SMILES: CCCN(CCCNC(=O)NCC)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1] | 1151.3 |
| TAZ-L-78 | 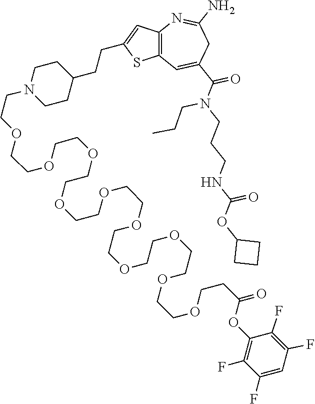 [SMILES: CCCN(CCCNC(=O)OC1CCC1)C(=O)C1=Cc2sc(CCC3CCN(CCOCCOCCOCCOCCOCCOCCOCCOCCOCCOCCC(=O)Oc4c(F)c(F)cc(F)c4F)CC3)cc2N=C(N)C1] | 1176.4 |
| TAZ-L-79 | 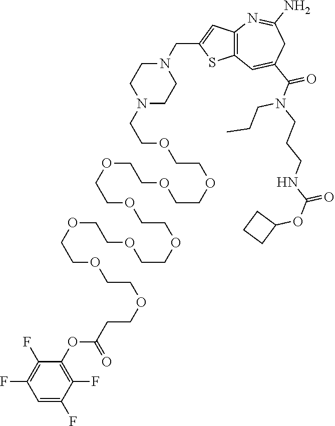 [SMILES: CCCN(CCCNC(=O)OC1CCC1)C(=O)C1=Cc2sc(CN3CCN(CCOCCOCCOCCOCCOCCOCCOCCOCCOCCOCCC(=O)Oc4c(F)c(F)cc(F)c4F)CC3)cc2N=C(N)C1] | 1163.3 |
| TAZ-L-80 | 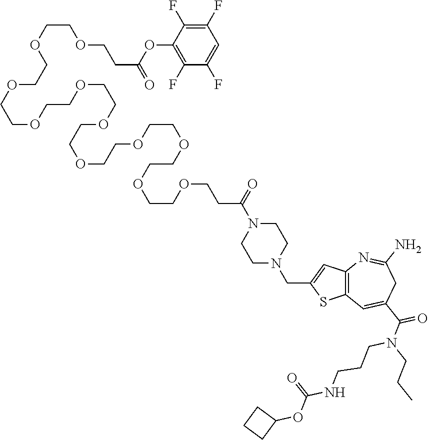 [SMILES: CCCN(CCCNC(=O)OC1CCC1)C(=O)C1=Cc2sc(CN3CCN(C(=O)CCOCCOCCOCCOCCOCCOCCOCCOCCOCCOCCOCCC(=O)Oc4c(F)c(F)cc(F)c4F)CC3)cc2N=C(N)C1] | 1235.4 |
| TAZ-L-81 | 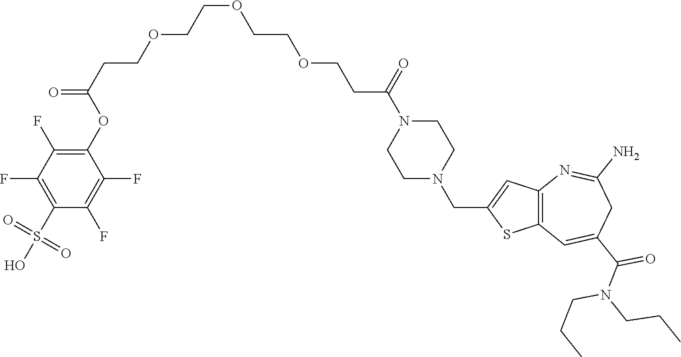 [SMILES: CCCN(CCC)C(=O)C1=Cc2sc(CN3CCN(C(=O)CCOCCOCCOCCC(=O)Oc4c(F)c(F)c(S(=O)(=O)O)c(F)c4F)CC3)cc2N=C(N)C1] | 849.9 |
| TAZ-L-82 | 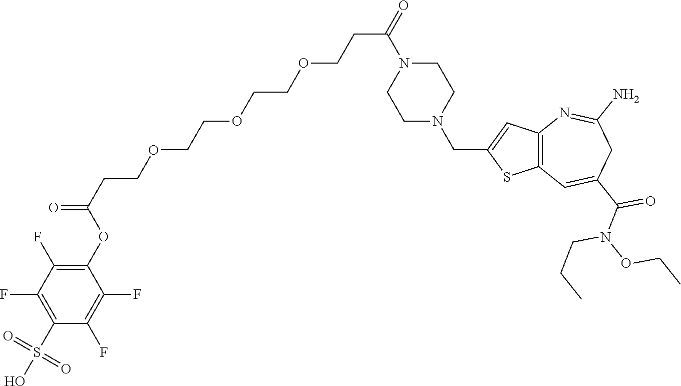 [SMILES: CCCN(OCC)C(=O)C1=Cc2sc(CN3CCN(C(=O)CCOCCOCCOCCC(=O)Oc4c(F)c(F)c(S(=O)(=O)O)c(F)c4F)CC3)cc2N=C(N)C1] | 851.9 |
| TAZ-L-83 | 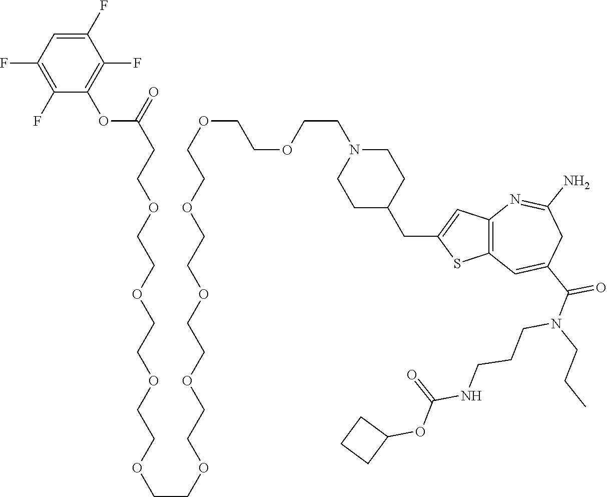 [SMILES: CCCN(CCCNC(=O)OC1CCC1)C(=O)C1=Cc2sc(CC3CCN(CCOCCOCCOCCOCCOCCOCCOCCOCCOCCOCCC(=O)Oc4c(F)c(F)cc(F)c4F)CC3)cc2N=C(N)C1] | 1162.3 |
| TAZ-L-84 | 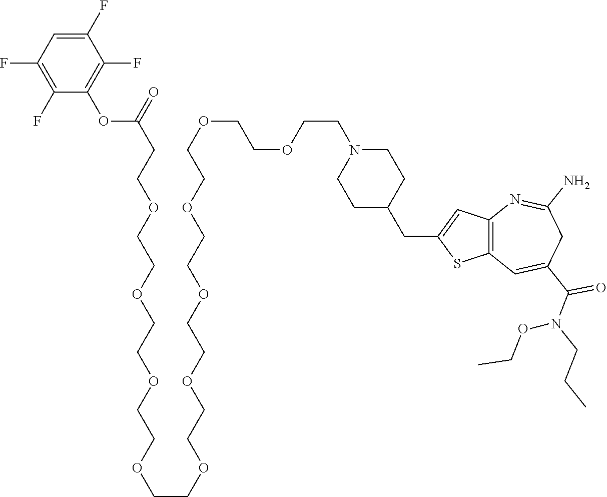 [SMILES: CCCN(OCC)C(=O)C1=Cc2sc(CC3CCN(CCOCCOCCOCCOCCOCCOCCOCCOCCOCCOCCC(=O)Oc4c(F)c(F)cc(F)c4F)CC3)cc2N=C(N)C1] | 1051.2 |
| TAZ-L-85 | 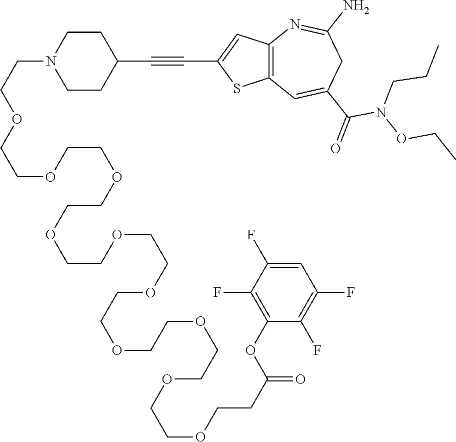 [SMILES: CCCN(OCC)C(=O)C1=Cc2sc(C#CC3CCN(CCOCCOCCOCCOCCOCCOCCOCCOCCOCCOCCC(=O)Oc4c(F)c(F)cc(F)c4F)CC3)cc2N=C(N)C1] | 1061.2 |
| TAZ-L-86 | 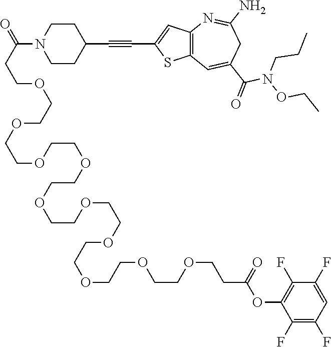 [SMILES: CCCN(OCC)C(=O)C1=Cc2sc(C#CC3CCN(C(=O)CCOCCOCCOCCOCCOCCOCCOCCOCCOCCOCCC(=O)Oc4c(F)c(F)cc(F)c4F)CC3)cc2N=C(N)C1] | 1089.2 |
| TAZ-L-87 | 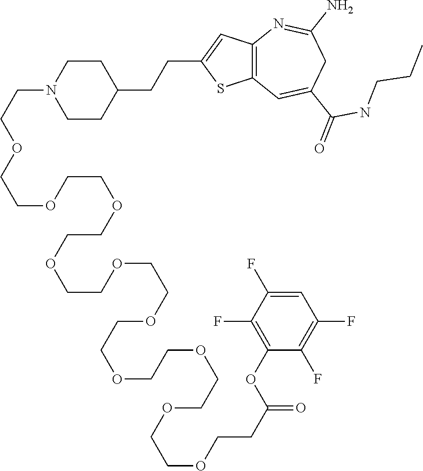 | 1065.2 |
| TAZ-L-88 | 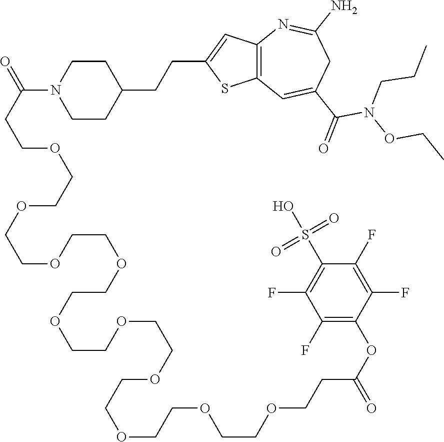 | 1173.3 |
| TAZ-L-89 | 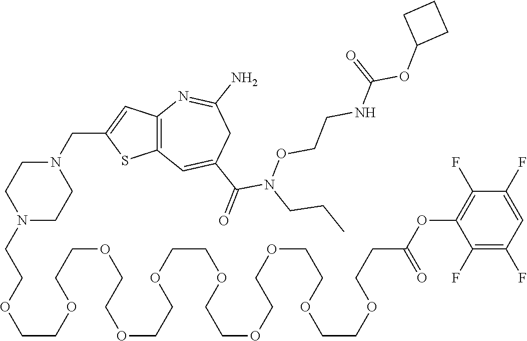 [SMILES: CCCN(OCCNC(=O)OC1CCC1)C(=O)C1=Cc2sc(CN3CCN(CCOCCOCCOCCOCCOCCOCCOCCOCCOCCOCCC(=O)Oc4c(F)c(F)cc(F)c4F)CC3)cc2N=C(N)C1] | 1165.3 |
| TAZ-L-90 | 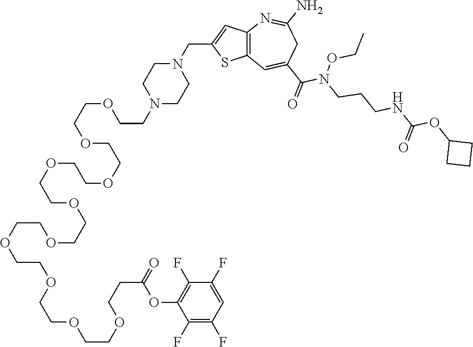 [SMILES: CCON(CCCNC(=O)OC1CCC1)C(=O)C1=Cc2sc(CN3CCN(CCOCCOCCOCCOCCOCCOCCOCCOCCOCCOCCC(=O)Oc4c(F)c(F)cc(F)c4F)CC3)cc2N=C(N)C1] | 1165.3 |
| TAZ-L-91 | 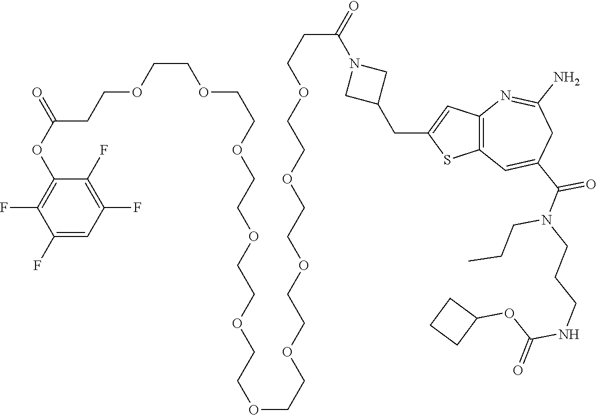 [SMILES: CCCN(CCCNC(=O)OC1CCC1)C(=O)C1=Cc2sc(CC3CN(C(=O)CCOCCOCCOCCOCCOCCOCCOCCOCCOCCOCCC(=O)Oc4c(F)c(F)cc(F)c4F)C3)cc2N=C(N)C1] | 1162.3 |
| TAZ-L-92 | 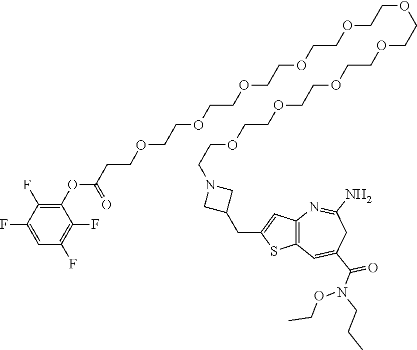 [SMILES: CCCN(OCC)C(=O)C1=Cc2sc(CC3CN(CCOCCOCCOCCOCCOCCOCCOCCOCCOCCOCCC(=O)Oc4c(F)c(F)cc(F)c4F)C3)cc2N=C(N)C1] | 1023.1 |
| TAZ-L-93 | 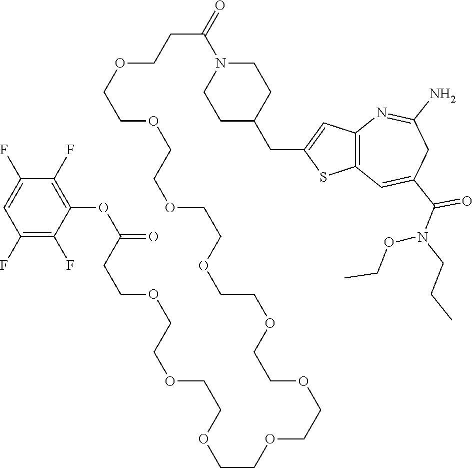 [SMILES: CCCN(OCC)C(=O)C1=Cc2sc(CC3CCN(C(=O)CCOCCOCCOCCOCCOCCOCCOCCOCCOCCOCCC(=O)Oc4c(F)c(F)cc(F)c4F)CC3)cc2N=C(N)C1] | 1079.2 |
| TAZ-L-94 | 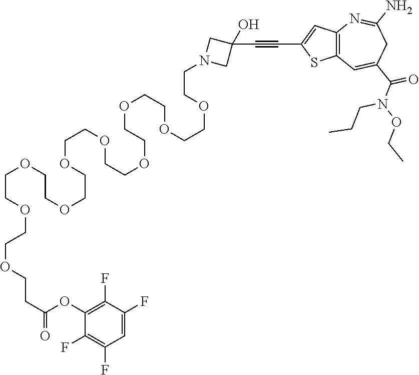 | 1049.1 |
| TAZ-L-95 | 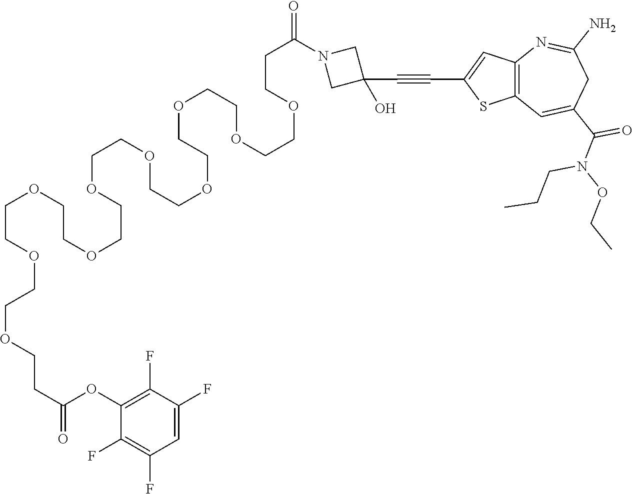 [SMILES: CCCN(OCC)C(=O)C1=Cc2sc(C#CC3(O)CN(C(=O)CCOCCOCCOCCOCCOCCOCCOCCOCCOCCOCCC(=O)Oc4c(F)c(F)cc(F)c4F)C3)cc2N=C(N)C1] | 1077.1 |
| TAZ-L-96 | 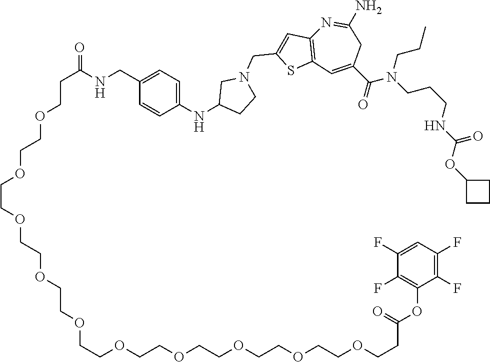 [SMILES: CCCN(CCCNC(=O)OC1CCC1)C(=O)C1=Cc2sc(CN3CCC(Nc4ccc(CNC(=O)CCOCCOCCOCCOCCOCCOCCOCCOCCOCCOCCC(=O)Oc5c(F)c(F)cc(F)c5F)cc4)C3)cc2N=C(N)C1] | 1296.5 |
| TAZ-L-97 | 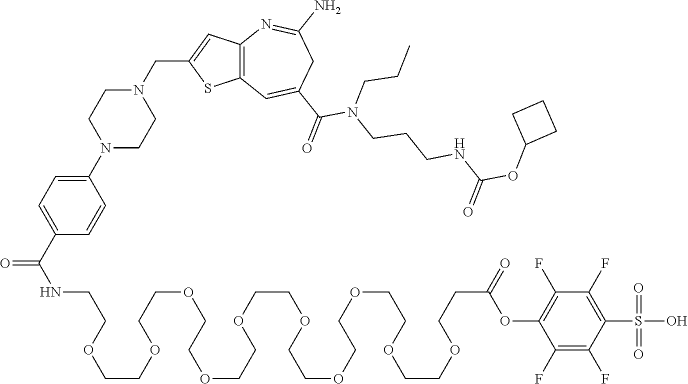 [SMILES: CCCN(CCCNC(=O)OC1CCC1)C(=O)C1=Cc2sc(CN3CCN(c4ccc(C(=O)NCCOCCOCCOCCOCCOCCOCCOCCOCCOCCOCCC(=O)Oc5c(F)c(F)c(S(=O)(=O)O)c(F)c5F)cc4)CC3)cc2N=C(N)C1] | 1362.5 |
| TAZ-L-98 | 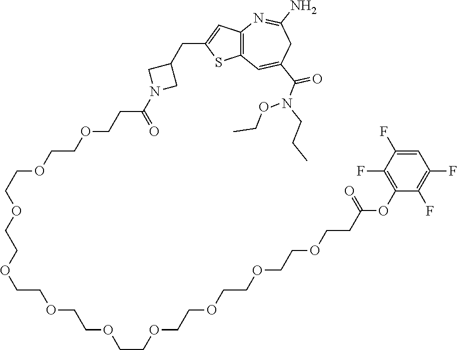 [SMILES: CCCN(OCC)C(=O)C1=Cc2sc(CC3CN(C(=O)CCOCCOCCOCCOCCOCCOCCOCCOCCOCCOCCC(=O)Oc4c(F)c(F)cc(F)c4F)C3)cc2N=C(N)C1] | 1051.2 |
| TAZ-L-99 | 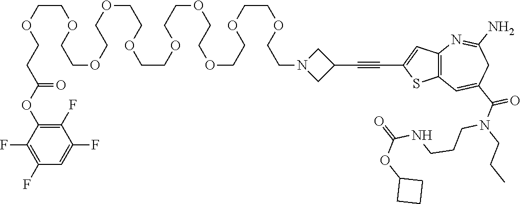 [SMILES: CCCN(CCCNC(=O)OC1CCC1)C(=O)C1=Cc2sc(C#CC3CN(CCOCCOCCOCCOCCOCCOCCOCCOCCOCCOCCC(=O)Oc4c(F)c(F)cc(F)c4F)C3)cc2N=C(N)C1] | 1144.3 |
| TAZ-L-100 | 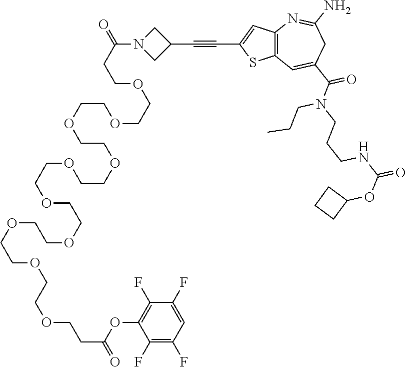 [SMILES: CCCN(CCCNC(=O)OC1CCC1)C(=O)C1=Cc2sc(C#CC3CN(C(=O)CCOCCOCCOCCOCCOCCOCCOCCOCCOCCOCCC(=O)Oc4c(F)c(F)cc(F)c4F)C3)cc2N=C(N)C1] | 1172.3 |
| TAZ-L-101 | 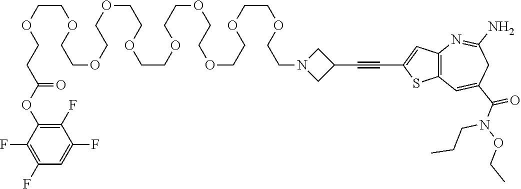 [SMILES: CCCN(OCC)C(=O)C1=Cc2sc(C#CC3CN(CCOCCOCCOCCOCCOCCOCCOCCOCCOCCOCCC(=O)Oc4c(F)c(F)cc(F)c4F)C3)cc2N=C(N)C1] | 1033.1 |
| TAZ-L-102 | 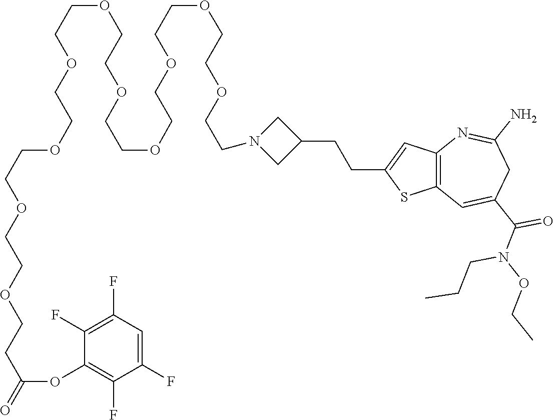 [SMILES: CCCN(OCC)C(=O)C1=Cc2sc(CCC3CN(CCOCCOCCOCCOCCOCCOCCOCCOCCOCCOCCC(=O)Oc4c(F)c(F)cc(F)c4F)C3)cc2N=C(N)C1] | 1037.2 |
| TAZ-L-103 | 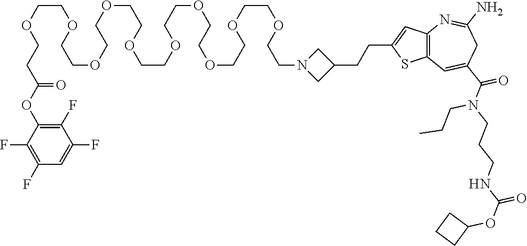 [SMILES: CCCN(CCCNC(=O)OC1CCC1)C(=O)C1=Cc2sc(CCC3CN(CCOCCOCCOCCOCCOCCOCCOCCOCCOCCOCCC(=O)Oc4c(F)c(F)cc(F)c4F)C3)cc2N=C(N)C1] | 1148.3 |
| TAZ-L-104 | 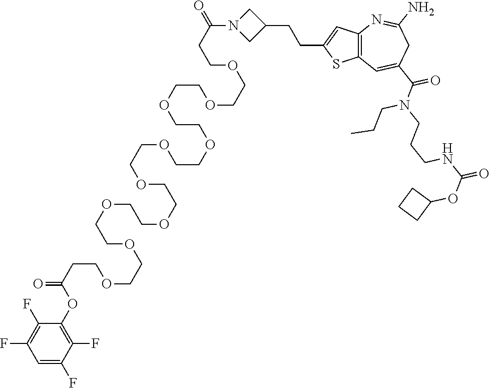 [SMILES: CCCN(CCCNC(=O)OC1CCC1)C(=O)C1=Cc2sc(CCC3CN(C(=O)CCOCCOCCOCCOCCOCCOCCOCCOCCOCCOCCC(=O)Oc4c(F)c(F)cc(F)c4F)C3)cc2N=C(N)C1] | 1176.3 |
| TAZ-L-105 | 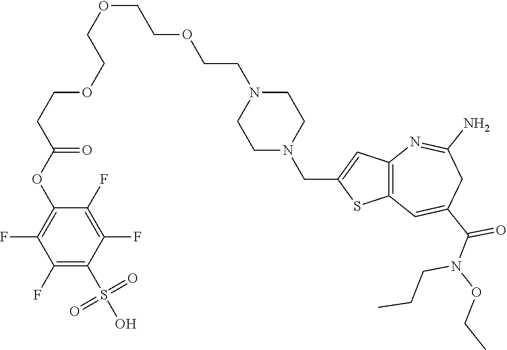 [SMILES: CCCN(OCC)C(=O)C1=Cc2sc(CN3CCN(CCOCCOCCOCCC(=O)Oc4c(F)c(F)c(S(=O)(=O)O)c(F)c4F)CC3)cc2N=C(N)C1] | 823.9 |
| TAZ-L-106 | 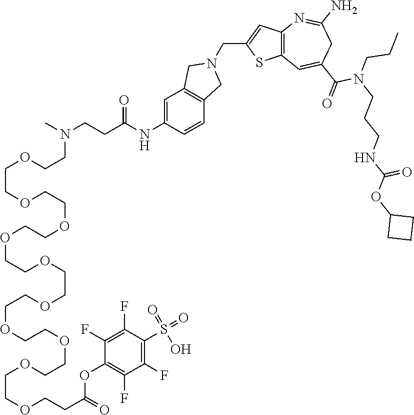 [SMILES: CCCN(CCCNC(=O)OC1CCC1)C(=O)C1=Cc2sc(CN3Cc4ccc(NC(=O)CCN(C)CCOCCOCCOCCOCCOCCOCCOCCOCCOCCOCCC(=O)Oc5c(F)c(F)c(S(=O)(=O)O)c(F)c5F)cc4C3)cc2N=C(N)C1] | 1376.5 |
| TAZ-L-107 | 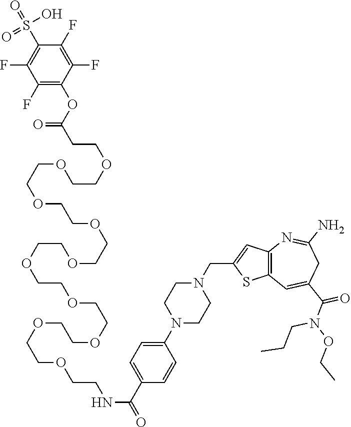 | 1251.4 |
| TAZ-L-108 | 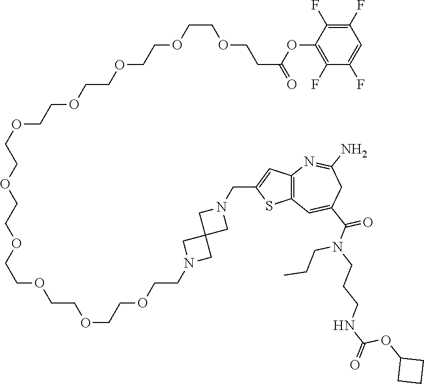 [SMILES: CCCN(CCCNC(=O)OC1CCC1)C(=O)C1=Cc2sc(CN3CC4(CN(CCOCCOCCOCCOCCOCCOCCOCCOCCOCCOCCC(=O)Oc5c(F)c(F)cc(F)c5F)C4)C3)cc2N=C(N)C1] | 1175.3 |
| TAZ-L-109 | 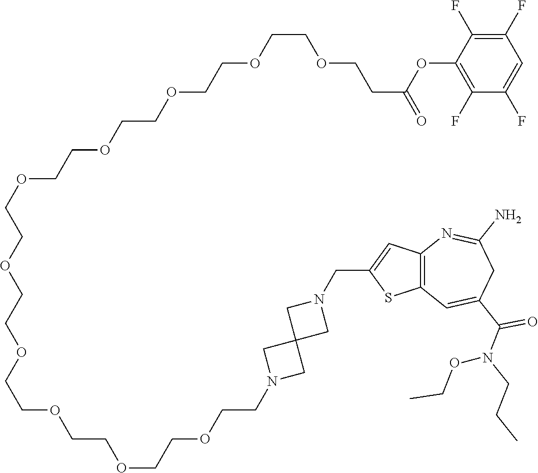 [SMILES: CCCN(OCC)C(=O)C1=Cc2sc(CN3CC4(CN(CCOCCOCCOCCOCCOCCOCCOCCOCCOCCOCCC(=O)Oc5c(F)c(F)cc(F)c5F)C4)C3)cc2N=C(N)C1] | 1064.2 |
| TAZ-L-110 | 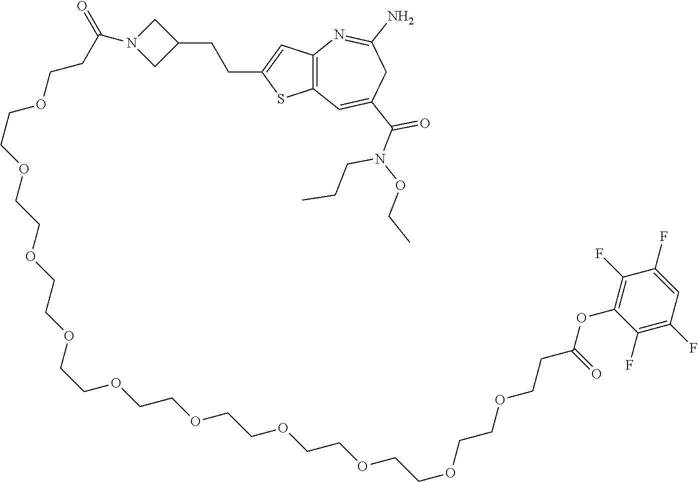 [SMILES: CCCN(OCC)C(=O)C1=Cc2sc(CCC3CN(C(=O)CCOCCOCCOCCOCCOCCOCCOCCOCCOCCOCCC(=O)Oc4c(F)c(F)cc(F)c4F)C3)cc2N=C(N)C1] | 1065.2 |
| TAZ-L-111 | 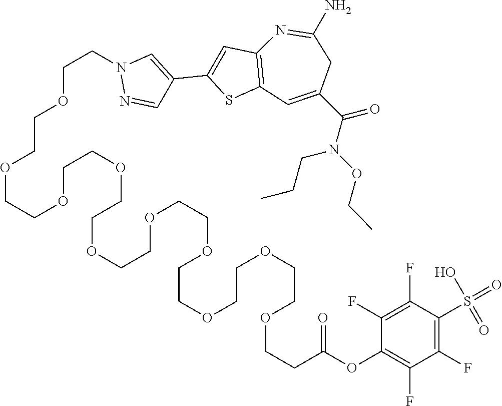 | 1100.2 |
| TAZ-L-112 | 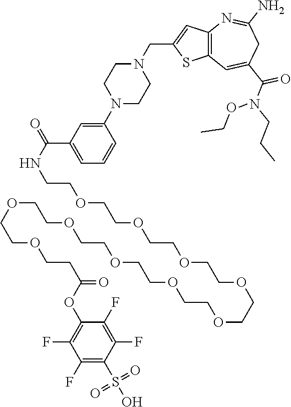 | 1251.4 |
| TAZ-L-113 | 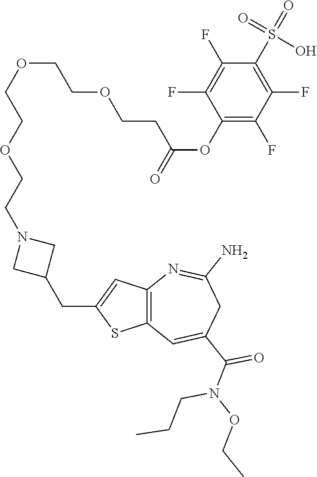 | 794.8 |
| TAZ-L-114 | 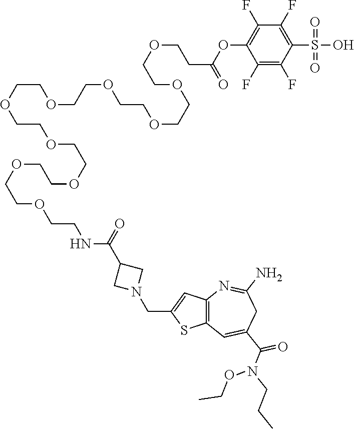 [SMILES: CCCN(OCC)C(=O)C1=Cc2sc(CN3CC(C(=O)NCCOCCOCCOCCOCCOCCOCCOCCOCCOCCOCCC(=O)Oc4c(F)c(F)c(S(=O)(=O)O)c(F)c4F)C3)cc2N=C(N)C1] | 1146.2 |
| TAZ-L-115 | 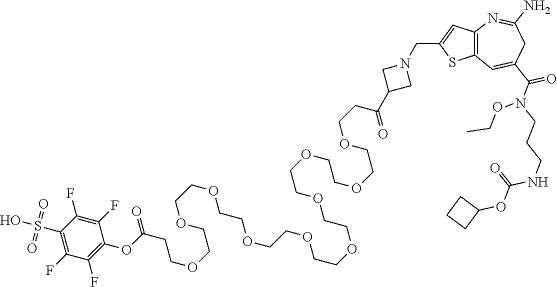 [SMILES: CCON(CCCNC(=O)OC1CCC1)C(=O)C1=Cc2sc(CN3CC(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1244.3 |
| TAZ-L-116 | 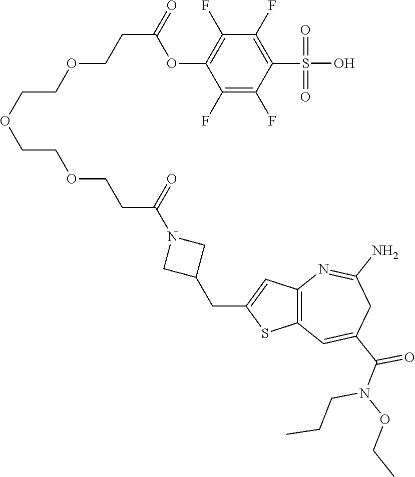 [SMILES: CCCN(OCC)C(=O)C1=Cc2sc(CC3CN(C(=O)CCOCCOCCOCCC(=O)Oc4c(F)c(F)c(S(=O)(=O)O)c(F)c4F)C3)cc2N=C(N)C1] | 822.8 |
| TAZ-L-117 | 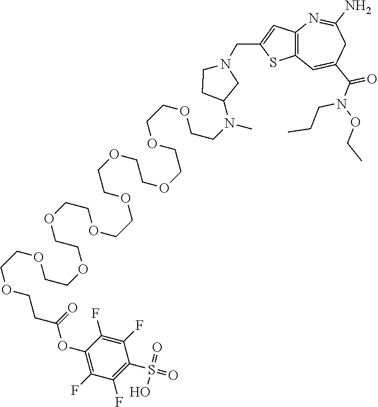 [SMILES: CCCN(OCC)C(=O)C1=Cc2sc(CN3CCC(N(C)CCOCCOCCOCCOCCOCCOCCOCCOCCOCCOCCC(=O)Oc4c(F)c(F)c(S(=O)(=O)O)c(F)c4F)C3)cc2N=C(N)C1] | 1146.3 |
| TAZ-L-118 | 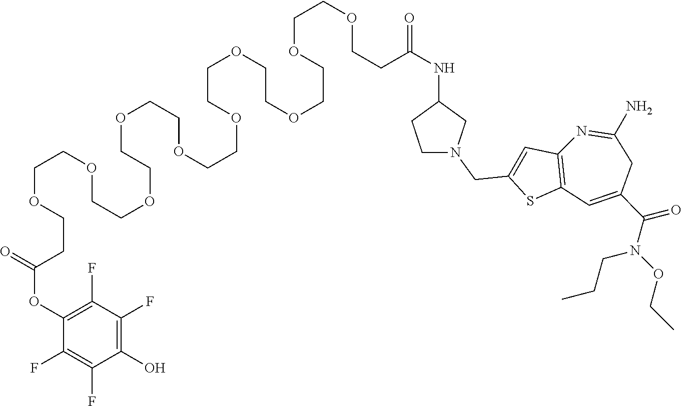 [SMILES: CCCN(OCC)C(=O)C1=Cc2sc(CN3CCC(NC(=O)CCOCCOCCOCCOCCOCCOCCOCCOCCOCCOCCC(=O)Oc4c(F)c(F)c(O)c(F)c4F)C3)cc2N=C(N)C1] | 1160.3 |
| TAZ-L-119 | 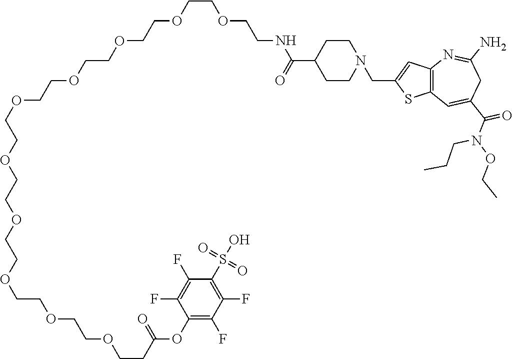 [SMILES: CCCN(OCC)C(=O)C1=Cc2sc(CN3CCC(C(=O)NCCOCCOCCOCCOCCOCCOCCOCCOCCOCCOCCC(=O)Oc4c(F)c(F)c(S(=O)(=O)O)c(F)c4F)CC3)cc2N=C(N)C1] | 1174.3 |
| TAZ-L-120 | 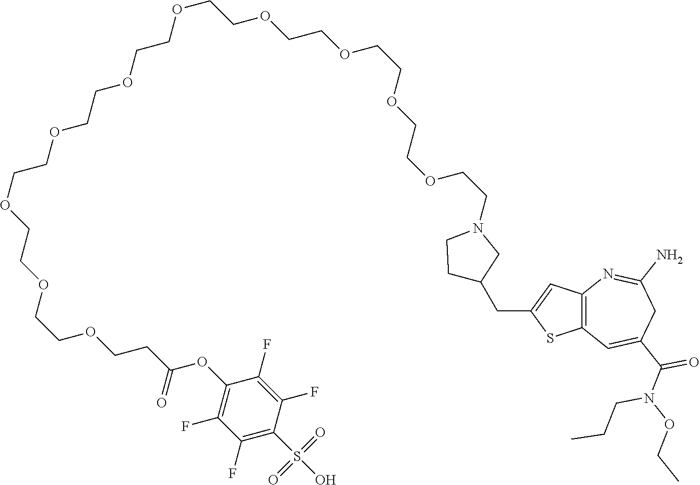 [SMILES: CCCN(OCC)C(=O)C1=Cc2sc(CC3CCN(CCOCCOCCOCCOCCOCCOCCOCCOCCOCCOCCC(=O)Oc4c(F)c(F)c(S(=O)(=O)O)c(F)c4F)C3)cc2N=C(N)C1] | 1117.2 |
| TAZ-L-121 | 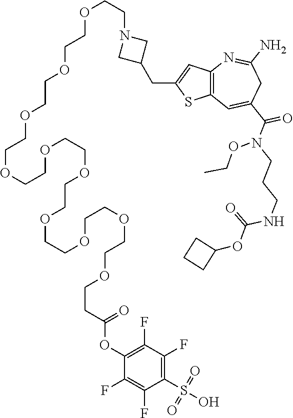 [SMILES: CCON(CCCNC(=O)OC1CCC1)C(=O)C1=Cc2sc(CC3CN(CCOCCOCCOCCOCCOCCOCCOCCOCCOCCOCCC(=O)Oc4c(F)c(F)c(S(=O)(=O)O)c(F)c4F)C3)cc2N=C(N)C1] | 1216.3 |
| TAZ-L-122 | 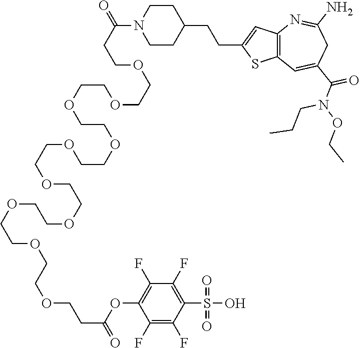 | 1173.3 |
| TAZ-L-123 | 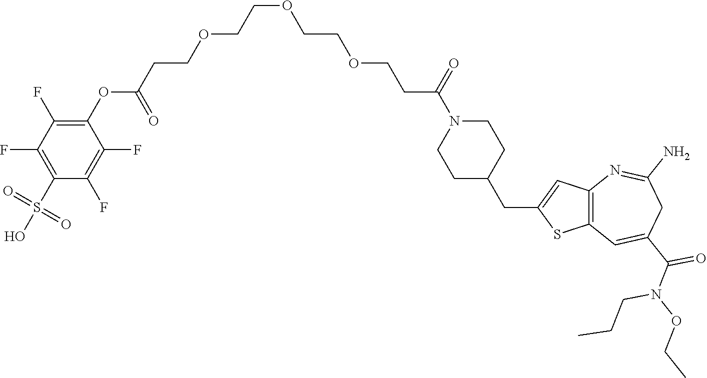 [SMILES: CCCN(OCC)C(=O)C1=Cc2sc(CC3CCN(C(=O)CCOCCOCCOCCC(=O)Oc4c(F)c(F)c(S(=O)(=O)O)c(F)c4F)CC3)cc2N=C(N)C1] | 850.9 |
| TAZ-L-124 | 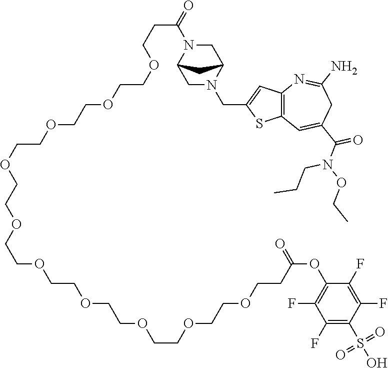 | 1172.3 |
| TAZ-L-125 | 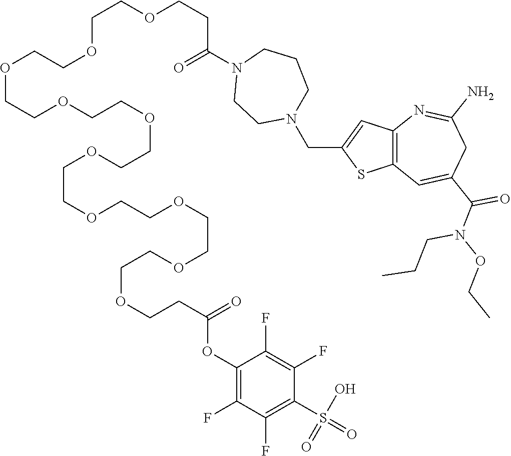 [SMILES: CCCN(OCC)C(=O)C1=Cc2sc(CN3CCCN(C(=O)CCOCCOCCOCCOCCOCCOCCOCCOCCOCCOCCC(=O)Oc4c(F)c(F)c(S(=O)(=O)O)c(F)c4F)CC3)cc2N=C(N)C1] | 1174.3 |
| TAZ-L-126 | 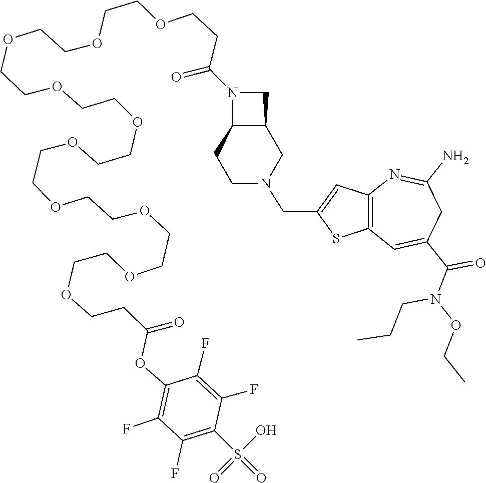 [SMILES: CCCN(OCC)C(=O)C1=Cc2sc(CN3CC[C@@H]4[C@H](C3)CN4C(=O)CCOCCOCCOCCOCCOCCOCCOCCOCCOCCOCCC(=O)Oc3c(F)c(F)c(S(=O)(=O)O)c(F)c3F)cc2N=C(N)C1] | 1186.3 |
| TAZ-L-127 | 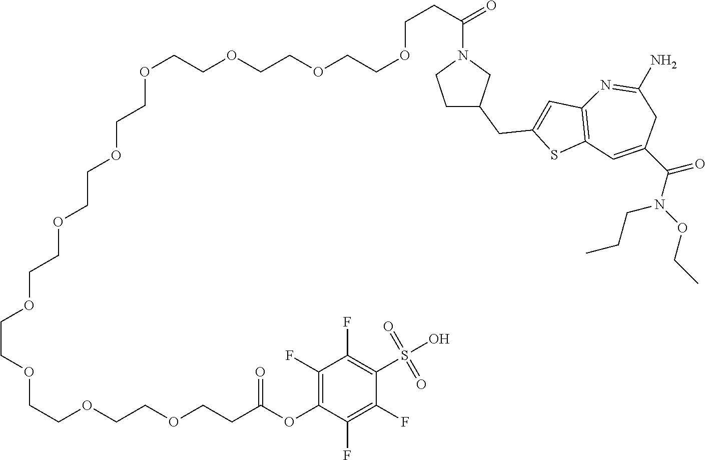 [SMILES: CCCN(OCC)C(=O)C1=Cc2sc(CC3CCN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1145.2 |
| TAZ-L-128 | 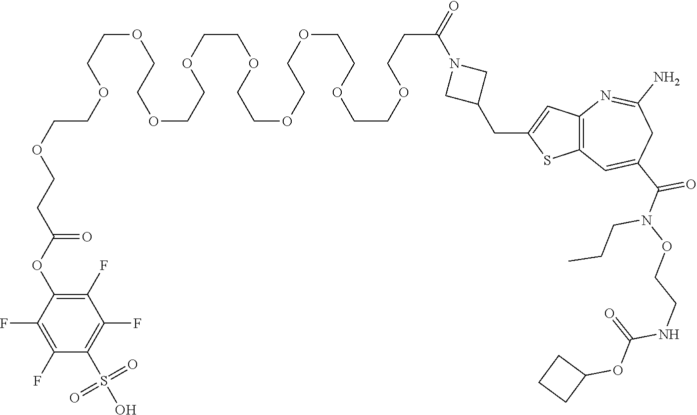 [SMILES: CCCN(OCCNC(=O)OC1CCC1)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1244.3 |
| TAZ-L-129 | 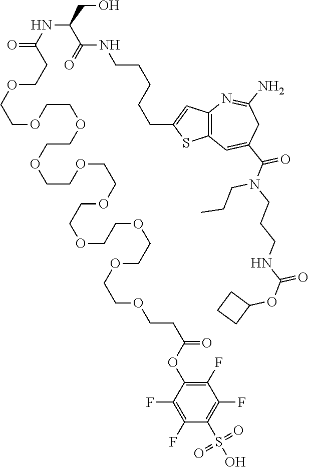 | 1409.5 |
| TAZ-L-130 | 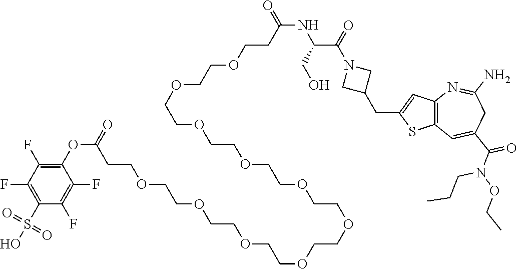 [SMILES: CCCN(OCC)C(=O)C1=Cc2sc(CC3CN(C(=O)[C@H](CO)NC(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1282.4 |
| TAZ-L-131 | 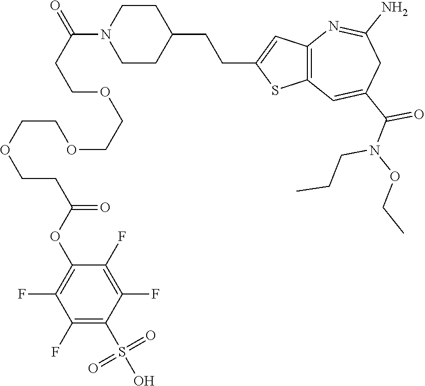 | 864.9 |
| TAZ-L-132 | 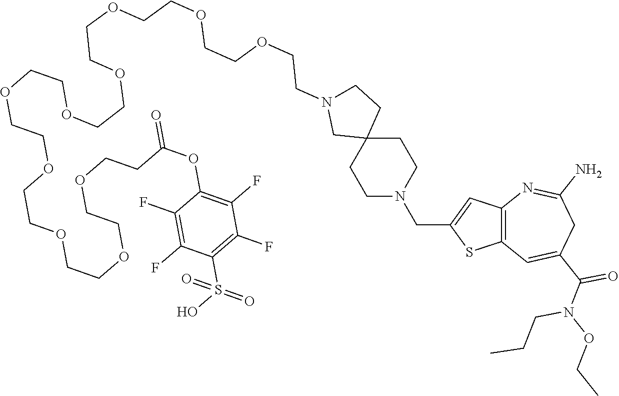 | 1186.3 |
| TAZ-L-133 | 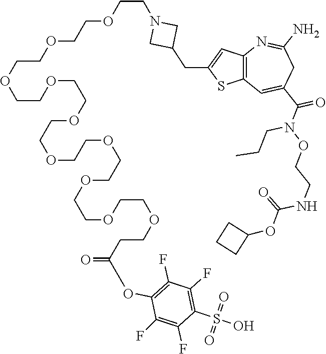 | 1216.3 |
| TAZ-L-134 | 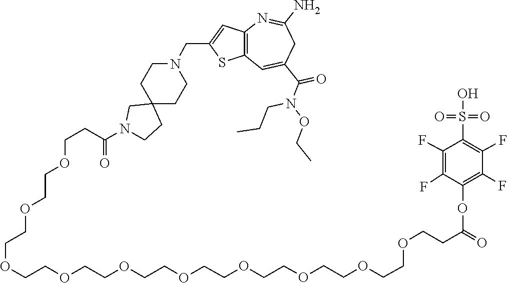 [SMILES: CCCN(OCC)C(=O)C1=Cc2sc(CN3CCC4(CC3)CCN(C(=O)CCOCCOCCOCCOCCOCCOCCOCCOCCOCCOCCC(=O)Oc3c(F)c(F)c(S(=O)(=O)O)c(F)c3F)C4)cc2N=C(N)C1] | 1214.3 |
| TAZ-L-135 | 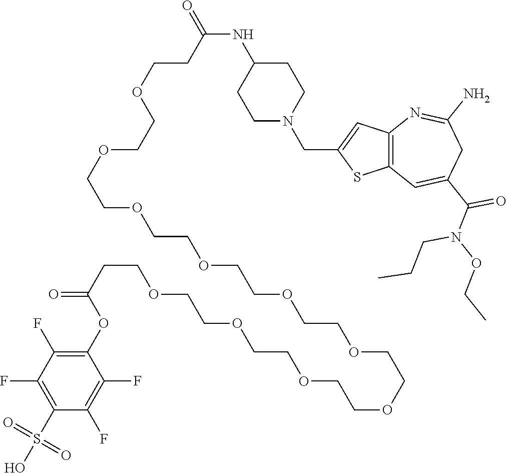 [SMILES: CCCN(OCC)C(=O)C1=Cc2sc(CN3CCC(NC(=O)CCOCCOCCOCCOCCOCCOCCOCCOCCOCCOCCC(=O)Oc4c(F)c(F)c(S(=O)(=O)O)c(F)c4F)CC3)cc2N=C(N)C1] | 1174.3 |
| TAZ-L-136 | 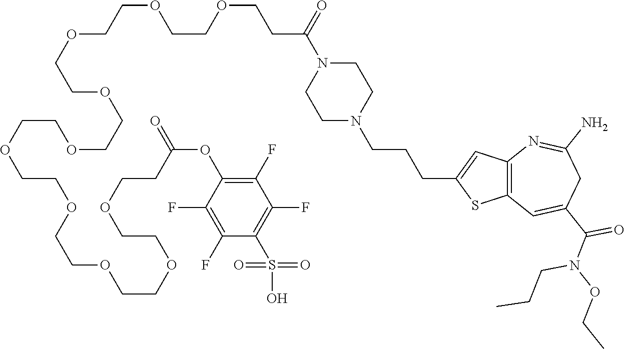 [SMILES: CCCN(OCC)C(=O)C1=Cc2sc(CCCN3CCN(C(=O)CCOCCOCCOCCOCCOCCOCCOCCOCCOCCOCCC(=O)Oc4c(F)c(F)c(S(=O)(=O)O)c(F)c4F)CC3)cc2N=C(N)C1] | 1188.3 |
| TAZ-L-137 | 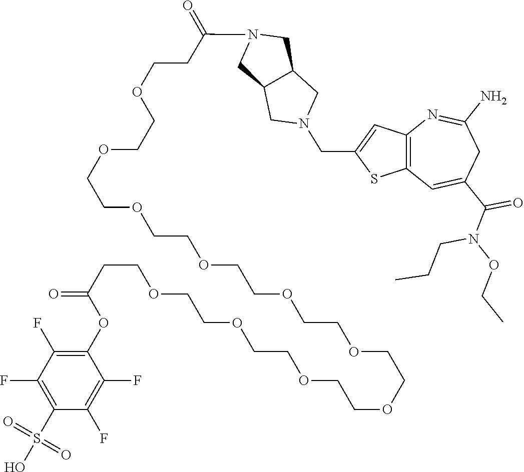 [SMILES: CCCN(OCC)C(=O)C1=Cc2sc(CN3C[C@@H]4CN(C(=O)CCOCCOCCOCCOCCOCCOCCOCCOCCOCCOCCC(=O)Oc5c(F)c(F)c(S(=O)(=O)O)c(F)c5F)C[C@@H]4C3)cc2N=C(N)C1] | 1186.3 |
| TAZ-L-138 | 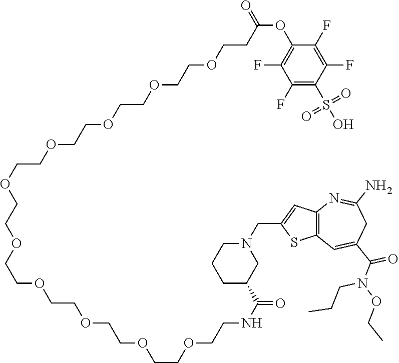 | 1174.3 |
| TAZ-L-139 | 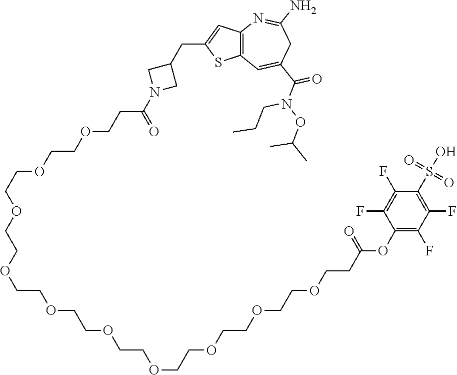 [SMILES: CCCN(OC(C)C)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1145.2 |
| TAZ-L-140 | 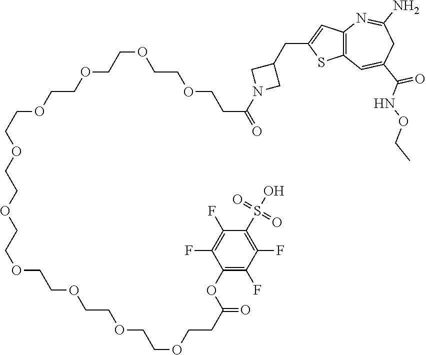 [SMILES: CCONC(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1089.1 |
| TAZ-L-141 | 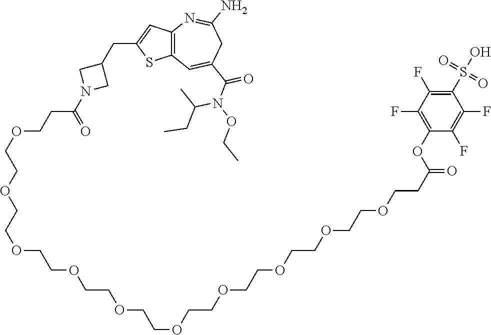 | 1145.2 |
| TAZ-L-142 | 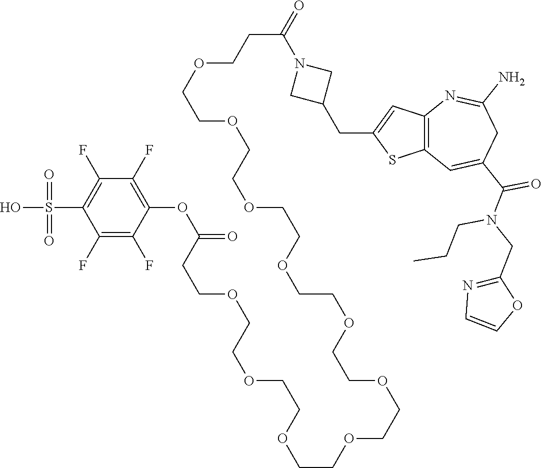 [SMILES: CCCN(Cc1ncco1)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1168.2 |
| TAZ-L-143 | 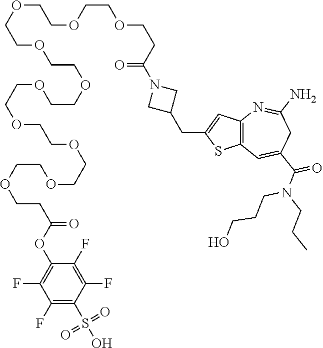 [SMILES: CCCN(CCCO)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1145.2 |
| TAZ-L-144 | 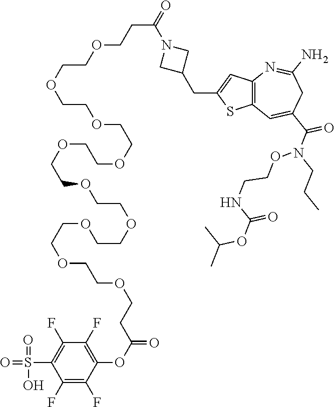 | 1232.3 |
| TAZ-L-145 | 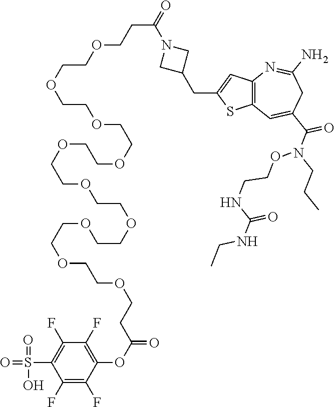 [SMILES: CCCN(OCCNC(=O)NCC)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1217.3 |
| TAZ-L-146 | 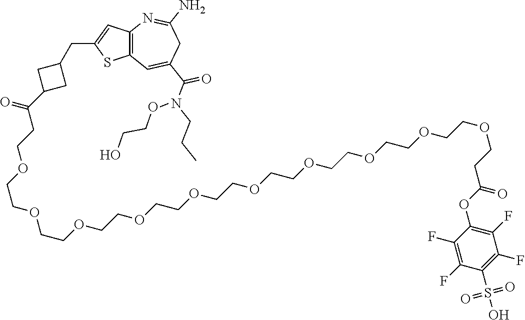 [SMILES: CCCN(OCCO)C(=O)C1=Cc2sc(CC3CC(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1] | 1147.2 |
| TAZ-L-147 | 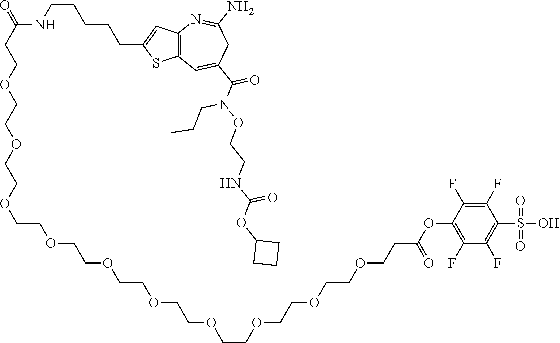 | 1260.4 |
| TAZ-L-148 | 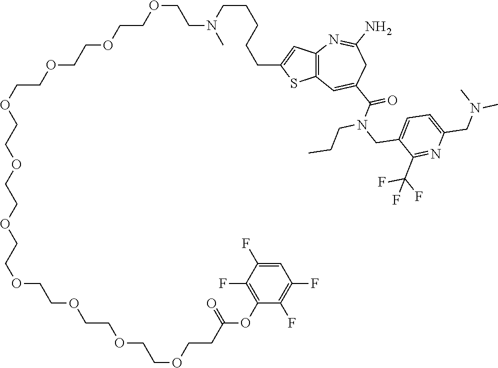 [SMILES: CCCN(Cc1ccc(CN(C)C)nc1C(F)(F)F)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1] | 1225.4 |
| TAZ-L-149 | 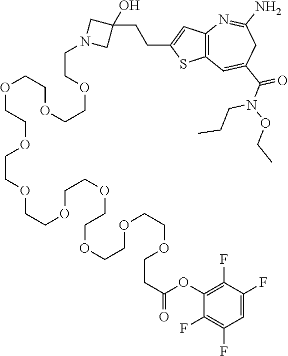 [SMILES: CCCN(OCC)C(=O)C1=Cc2sc(CCC3(O)CN(CCOCCOCCOCCOCCOCCOCCOCCOCCOCCOCCC(=O)Oc4c(F)c(F)cc(F)c4F)C3)cc2N=C(N)C1] | 1053.2 |
| TAZ-L-150 | 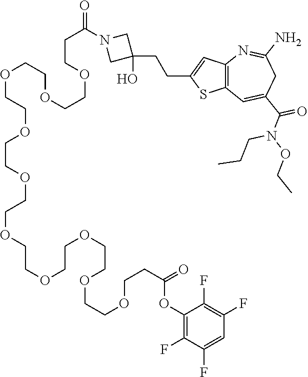 [SMILES: CCCN(OCC)C(=O)C1=Cc2sc(CCC3(O)CN(C(=O)CCOCCOCCOCCOCCOCCOCCOCCOCCOCCOCCC(=O)Oc4c(F)c(F)cc(F)c4F)C3)cc2N=C(N)C1] | 1081.2 |

### Macromolecule-Supported Compounds

Exemplary embodiments of macromolecule-supported compounds comprise a macromolecular support covalently attached to one or more thienoazepine (TAZ) moieties by a linker, and having Formula L:

Ms-[L-TAZ]ₚ I

or a pharmaceutically acceptable salt thereof,

wherein:

Ms is the macromolecular support;

p is an integer from 1 to 50;

TAZ is the 5-amino-thienoazepine moiety having the formula:

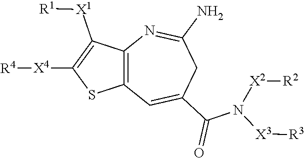

R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:

—(C₁-C₁₂ alkyldiyl)-N(R⁵)—*;

—(C₁-C₁₂ alkyldiyl)-N(R⁵)₂;

—(C₁-C₁₂ alkyldiyl)-OR⁵;

—(C₃-C₁₂ carbocyclyl);

—(C₃-C₁₂ carbocyclyl)-*;

—(C₃-C₁₂ carbocyclyl)-(C₁-C₁₂ alkyldiyl)-NR⁵—*;

—(C₃-C₁₂ carbocyclyl)-(C₁-C₁₂ alkyldiyl)-N(R⁵)₂;

—(C₃-C₁₂ carbocyclyl)-NR⁵—C(═NR⁵)NR⁵—*;

—(C₆-C₂₀ aryl);

—(C₆-C₂₀ aryl)-*;

—(C₆-C₂₀ aryldiyl)-N(R⁵)—*;

—(C₆-C₂₀ aryldiyl)-(C₁-C₁₂ alkyldiyl)-N(R⁵)—*;

—(C₆-C₂₀ aryldiyl)-(C₁-C₁₂ alkyldiyl)-(C₂-C₂₀ heterocyclyldiyl)-*;

—(C₆-C₂₀ aryldiyl)-(C₁-C₁₂ alkyldiyl)-N(R⁵)₂;

—(C₆-C₂₀ aryldiyl)-(C₁-C₁₂ alkyldiyl)-NR⁵—C(═NR^{5a})N(R⁵)—*;

—(C₂-C₂₀ heterocyclyl);

—(C₂-C₂₀ heterocyclyl)-*;

—(C₂-C₉ heterocyclyl)-(C₁-C₁₂ alkyldiyl)-NR⁵—*;

—(C₂-C₉ heterocyclyl)-(C₁-C₁₂ alkyldiyl)-N(R⁵)₂;

—(C₂-C₉ heterocyclyl)-C(═O)—(C₁-C₁₂ alkyldiyl)-N(R⁵)—*;

—(C₂-C₉ heterocyclyl)-NR⁵—C(═NR^{5a})NR⁵—*;

—(C₂-C₉ heterocyclyl)-NR⁵—(C₆-C₂₀ aryldiyl)-(C₁-C₁₂ alkyldiyl)-N(R⁵)—*;

—(C₂-C₉ heterocyclyl)-(C₆-C₂₀ aryldiyl)-*;

—(C₁-C₂₀ heteroaryl);

—(C₁-C₂₀ heteroaryl)-*;

—(C₁-C₂₀ heteroaryl)-(C₁-C₁₂ alkyldiyl)-N(R⁵)—*;

—(C₁-C₂₀ heteroaryl)-(C₁-C₁₂ alkyldiyl)-N(R⁵)₂;

—(C₁-C₂₀ heteroaryl)-NR⁵—C(═NR^{5a})N(R⁵)—*;

—(C₁-C₂₀ heteroaryl)-N(R⁵)C(═O)—(C₁-C₁₂ alkyldiyl)-N(R⁵)—*;

—C(═O)—*;

—C(═O)—(C₁-C₁₂ alkyldiyl)-N(R⁵)—*;

—C(═O)—(C₂-C₂₀ heterocyclyldiyl)-*;

—C(═O)N(R⁵)₂;

—C(═O)N(R⁵)—*;

—C(═O)N(R⁵)—(C₁-C₁₂ alkyldiyl)-N(R⁵)C(═O)R⁵;

—C(═O)N(R⁵)—(C₁-C₁₂ alkyldiyl)-N(R⁵)C(═O)N(R⁵)₂;

—C(═O)NR⁵—(C₁-C₁₂ alkyldiyl)-N(R⁵)CO₂R⁵;

—C(═O)NR⁵—(C₁-C₁₂ alkyldiyl)-N(R⁵)C(═NR^{5a})N(R⁵)₂;

—C(═O)NR⁵—(C₁-C₁₂ alkyldiyl)-NR⁵C(═NR^{5a})R⁵;

—C(═O)NR⁵—(C₁-C₈ alkyldiyl)-NR⁵(C₂-C₅ heteroaryl);

—C(═O)NR⁵—(C₁-C₂₀ heteroaryldiyl)-N(R⁵)—*;

—C(═O)NR⁵—(C₁-C₂₀ heteroaryldiyl)-*;

—C(═O)NR⁵—(C₁-C₂₀ heteroaryldiyl)-(C₁-C₁₂ alkyldiyl)-N(R⁵)₂;

—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;

—N(R⁵)₂;

—N(R⁵)—*;

—N(R⁵)C(═O)R⁵;

—N(R⁵)C(═O)N(R⁵)₂;

—N(R⁵)C(═O)N(R⁵)—*;

—N(R⁵)CO₂R⁵;

—NR⁵C(═NR^{5a})N(R⁵)₂;

—NR⁵C(═NR^{5a})N(R⁵)—*;

—NR⁵C(═NR^{5a})R⁵;

—N(R⁵)C(═O)—(C₁-C₁₂ alkyldiyl)-N(R⁵)—*;

—N(R⁵)—(C₂-C₅ heteroaryl);

—N(R⁵)—S(═O)₂—(C₁-C₁₂ alkyl);

—O—(C₁-C₁₂ alkyl);

—O—(C₁-C₁₂ alkyldiyl)-N(R⁵)₂;

—O—(C₁-C₁₂ alkyldiyl)-N(R⁵)—*;

—S(═O)₂—(C₂-C₂₀ heterocyclyldiyl)-*;

—S(═O)₂—(C₂-C₂₀ heterocyclyldiyl)-(C₁-C₁₂ alkyldiyl)-N(R⁵)₂;

—S(═O)₂—(C₂-C₂₀ heterocyclyldiyl)-(C₁-C₁₂ alkyldiyl)-NR⁵—*; and

—S(═O)₂—(C₂-C₂₀ heterocyclyldiyl)-(C₁-C₁₂ alkyldiyl)-OH; or R² and R³ together form a 5- or 6-membered heterocyclyl ring;

X¹, X², X³, and X⁴ are independently selected from the group consisting of a bond, C(═O), C(═O)N(R⁵), O, N(R⁵), S, S(O)₂, and S(O)₂N(R⁵);

R⁵ is selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;

R⁵, is selected from the group consisting of C₆-C₂₀ aryl and C₁-C₂₀ heteroaryl;

where the asterisk * indicates the attachment site of L, and where one of R¹, R², R³ and R⁴ is attached to L;

L is the linker selected from the group consisting of: —C(═O)-(PEG)-;

—C(═O)-(PEG)-C(═O)—;

—C(═O)-(PEG)-O—;

—C(═O)-(PEG)-C(═O)-(PEP)-;

—C(═O)-(PEG)-C(═O)N(R⁵)—(C₁-C₁₂ alkyldiyl)-;

—C(═O)-(PEG)-C(═O)N(R⁵)—(C₁-C₁₂ alkyldiyl)-N(R⁵)C(═O)—(C₂-C₅ monoheterocyclyldiyl)-;

—C(═O)-(PEG)-C(═O)N(R⁵)—(C₁-C₁₂ alkyldiyl)-(MCgluc)-;

—C(═O)-(PEG)-C(═O)-(MCgluc)-;

—C(═O)-(PEG)-C(═O)-(PEP)-N(R⁵)—(C₁-C₁₂ alkyldiyl)-;

—C(═O)-(PEG)-C(═O)-(PEP)-N(R⁵)—(C₁-C₁₂ alkyldiyl)-N(R⁵)C(═O)—(C₂-C₅ monoheterocyclyldiyl)-;

—C(═O)-(PEG)-N(R⁵)—;

—C(═O)-(PEG)-N(R⁵)C(═O)—;

—C(═O)-(PEG)-N(R⁵)-(PEG)-C(═O)-(PEP)-;

—C(═O)-(PEG)-N⁺(R⁵)₂-(PEG)-C(═O)-(PEP)-;

—C(═O)-(PEG)-C(═O)—N(R⁵)CH(AA₁)C(═O)-(PEG)-C(═O)-(PEP)-;

—C(═O)-(PEG)-C(═O)—N(R⁵)CH(AA₁)C(═O)—N(R⁵)—(C₁-C₁₂ alkyldiyl)-;

—C(═O)-(PEG)-SS-(C₁-C₁₂ alkyldiyl)-OC(═O)—;

—C(═O)-(PEG)-SS-(C₁-C₁₂ alkyldiyl)-C(═O)—;

—C(═O)—(C₁-C₁₂ alkyldiyl)-C(═O)-(PEP)-;

—C(═O)—(C₁-C₁₂ alkyldiyl)-C(═O)-(PEP)-N(R⁵)—(C₁-C₁₂ alkyldiyl)-;

—C(═O)—(C₁-C₁₂ alkyldiyl)-C(═O)-(PEP)-N(R⁵)—(C₁-C₁₂ alkyldiyl)-N(R⁵)—C(═O);

—C(═O)—(C₁-C₁₂ alkyldiyl)-C(═O)-(PEP)-N(R⁵)—(C₁-C₁₂ alkyldiyl)-N(R⁵)C(═O)—(C₂-C₅ monoheterocyclyldiyl)-;

—C(═O)—CH₂CH₂OCH₂CH₂—(C₁-C₂₀ heteroaryldiyl)-CH₂O-(PEG)-C(═O)-(MCgluc)-;

—C(═O)—CH₂CH₂OCH₂CH₂—(C₁-C₂₀ heteroaryldiyl)-CH₂O-(PEG)-C(═O)-(MCgluc)-N(R⁵)—(C₁-C₁₂ alkyldiyl)-N(R⁵)C(═O)—(C₂-C₅ monoheterocyclyldiyl)-; and -(succinimidyl)-(CH₂)m-C(═O)-(PEP)-N(R⁵)—(C₁-C₁₂ alkyldiyl)-N(R⁵)C(═O)—(C₂-C₅ monoheterocyclyldiyl)-;

PEG has the formula: —(CH₂CH₂O)ₙ—(CH₂)m-; m is an integer from 1 to 5, and n is an integer from 2 to 50;

PEP has the formula:

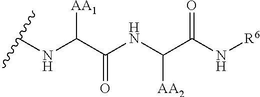

where AA₁ and AA₂ are independently selected from an amino acid side chain, or AA₁ or AA₂ and an adjacent nitrogen atom form a 5-membered ring proline amino acid, and the wavy line indicates a point of attachment;

R⁶ is selected from the group consisting of C₆-C₂₀ aryldiyl and C₁-C₂₀ heteroaryldiyl, substituted with —CH₂O—C(═O)— and optionally with:

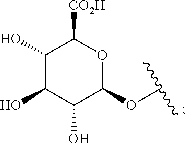 [SMILES: *O[C@@H]1O[C@H](C(=O)O)[C@@H](O)[C@H](O)[C@H]1O]

and

MCgluc is selected from the groups:

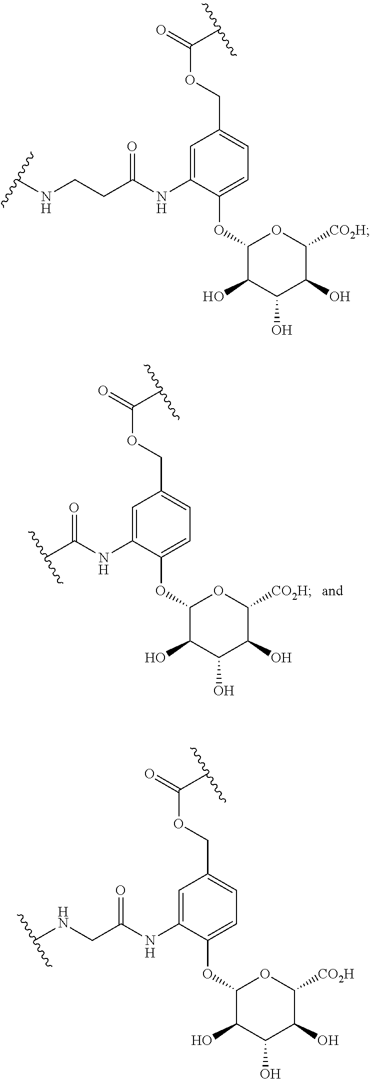

where q is 1 to 8, and AA is an amino acid side chain; and

alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of the macromolecule-supported compound of Formula I includes wherein subscript p is an integer from 1 to 25.

An exemplary embodiment of the macromolecule-supported compound of Formula I includes wherein subscript p is an integer from 1 to 6.

An exemplary embodiment of the macromolecule-supported compound of Formula I includes wherein the macromolecular support is a peptide.

An exemplary embodiment of the macromolecule-supported compound of Formula I includes wherein the macromolecular support is a nucleotide.

An exemplary embodiment of the macromolecule-supported compound of Formula I includes wherein the macromolecular support is a carbohydrate.

An exemplary embodiment of the macromolecule-supported compound of Formula I includes wherein the macromolecular support is a lipid.

An exemplary embodiment of the macromolecule-supported compound of Formula I includes wherein the macromolecular support is an antibody construct.

An exemplary embodiment of the macromolecule-supported compound of Formula I includes wherein the macromolecular support is a biopolymer.

An exemplary embodiment of the macromolecule-supported compound of Formula I includes wherein the macromolecular support is a nanoparticle.

An exemplary embodiment of the macromolecule-supported compound of Formula I includes wherein the macromolecular support is an immune checkpoint inhibitor.

An exemplary embodiment of the macromolecule-supported compound of Formula I includes wherein PEP has the formula:

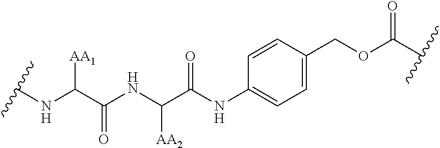

wherein AA₁ and AA₂ are independently selected from a side chain of a naturally-occurring amino acid.

An exemplary embodiment of the macromolecule-supported compound of Formula I includes wherein AA₁ or AA₂ with an adjacent nitrogen atom form a 5-membered ring proline amino acid.

An exemplary embodiment of the macromolecule-supported compound of Formula I includes wherein PEP has the formula:

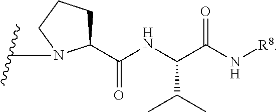

An exemplary embodiment of the macromolecule-supported compound of Formula I includes wherein MCgluc has the formula:

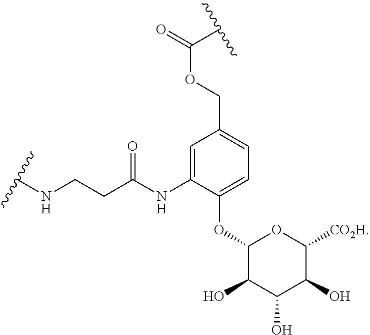 [SMILES: *NCCC(=O)Nc1cc(COC(*)=O)ccc1O[C@@H]1O[C@H](C(=O)O)[C@@H](O)[C@H](O)[C@H]1O]

An exemplary embodiment of the macromolecule-supported compound of Formula I includes wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

An exemplary embodiment of the macromolecule-supported compound of Formula I includes wherein AA₁ is —CH(CH₃)₂, and AA₂ is —CH₂CH₂CH₂NHC(O)NH₂.

An exemplary embodiment of the macromolecule-supported compound of Formula I includes wherein AA₁ and AA₂ are independently selected from GlcNAc aspartic acid, —CH₂SO₃H, and —CH₂OPO₃H.

An exemplary embodiment of the macromolecule-supported compound of Formula I includes wherein X¹ is a bond, and R¹ is H.

An exemplary embodiment of the macromolecule-supported compound of Formula I includes wherein X² is a bond, and R² is C₁-C₈ alkyl.

An exemplary embodiment of the macromolecule-supported compound of Formula I includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

An exemplary embodiment of the macromolecule-supported compound of Formula I includes wherein R² and R³ are each independently selected from —CH₂CH₂CH₃, —OCH₂CH₃, —CH₂CH₂CF₃, and —CH₂CH₂CH₂OH.

An exemplary embodiment of the macromolecule-supported und of Formula I includes wherein R² is C₁-C₈ alkyl and R³ is —(C₁-C₈ alkyldiyl)-N(R⁵)CO₂R⁴.

An exemplary embodiment of the macromolecule-supported compound of Formula I includes wherein R² is —CH₂CH₂CH₃ and R³ is —CH₂CH₂CH₂NHCO₂(t-Bu).

An exemplary embodiment of the macromolecule-supported compound of Formula I includes wherein R² and R³ are each —CH₂CH₂CH₃.

An exemplary embodiment of the macromolecule-supported compound of Formula I includes wherein X³—R³ is selected from the group consisting of:

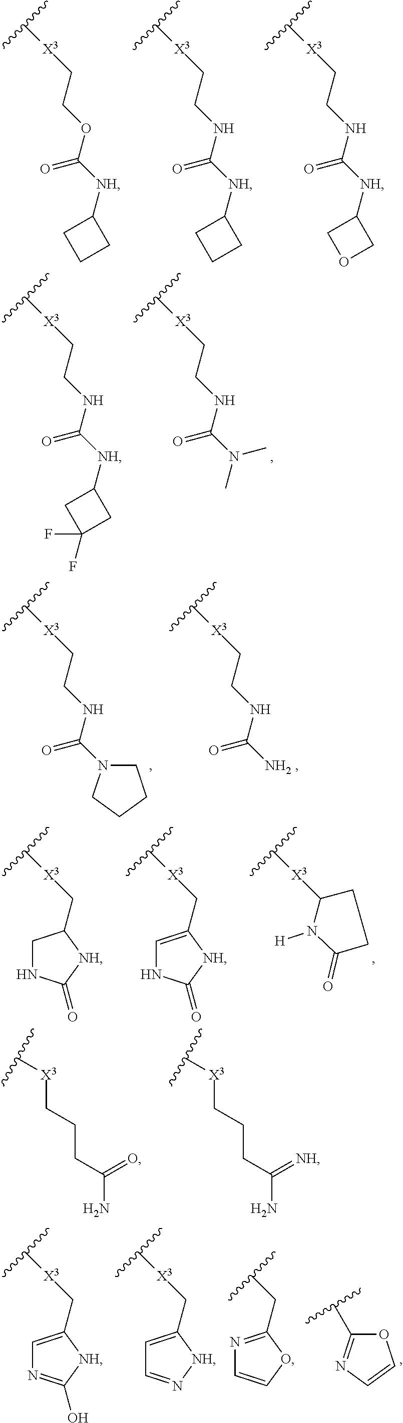

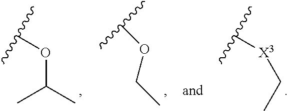

An exemplary embodiment of the macromolecule-supported compound of Formula I includes wherein one of R² and R³ is selected from:

—(C₁-C₁₂ alkyldiyl)-N(R⁵)—*;

—(C₁-C₁₂ alkyldiyl)-O—(C₁-C₁₂ alkyldiyl)-N(R⁵)—*;

—(C₁-C₁₂ alkyldiyl)-N(R⁵)C(═NR⁵)—N(R⁵)—*;

—(C₁-C₁₂ alkyldiyl)-(C₆-C₂₀ aryldiyl)-(C₁-C₁₂ alkyldiyl)-N(R⁵)—*;

—(C₁-C₁₂ alkyldiyl)-(C₆-C₂₀ aryldiyl)-(C₁-C₁₂ alkyldiyl)-N(R⁵)—C(═NR⁵)N(R⁵)—*;

—(C₂-C₆ alkynyldiyl)-N(R⁵)—*; and

—(C₂-C₆ alkynyldiyl)-N(R⁵)C(═NR⁵)N(R⁵)—*;

X² and X³ are a bond, and where the asterisk * indicates the attachment site of L.

An exemplary embodiment of the macromolecule-supported compound of Formula I includes wherein L is selected from the group consisting of: —C(═O)-(PEG)-;

—C(═O)-(PEG)-C(═O)—;

—C(═O)-(PEG)-O—;

—C(═O)-(PEG)-N(R⁵)—; and

—C(═O)-(PEG)-N(R⁵)C(═O)—.

An exemplary embodiment of the macromolecule-supported compound of Formula I is selected from Formulae Ia-Ic:

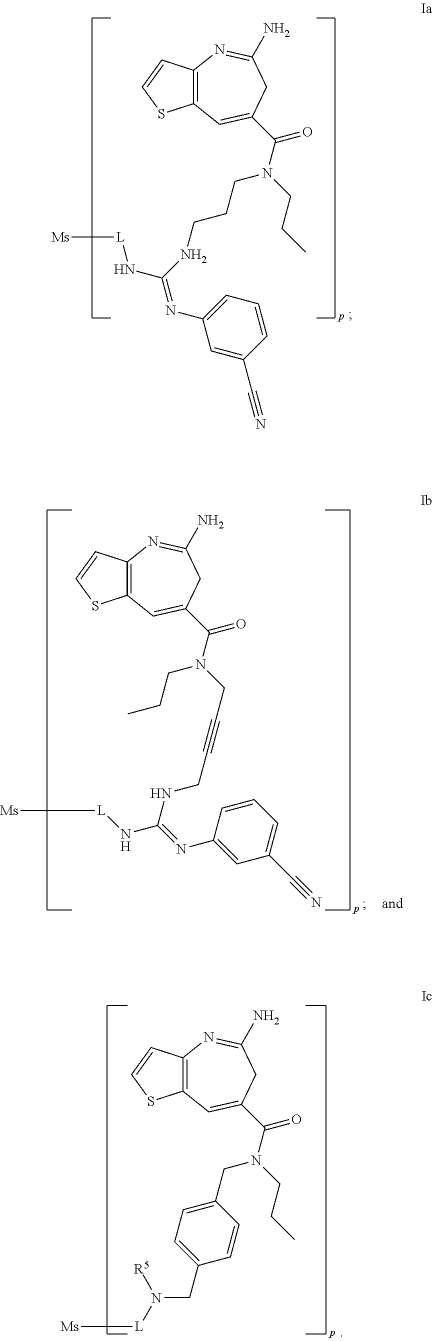

An exemplary embodiment of the macromolecule-supported compound of Formula I is selected from Formulae Id-Ih:

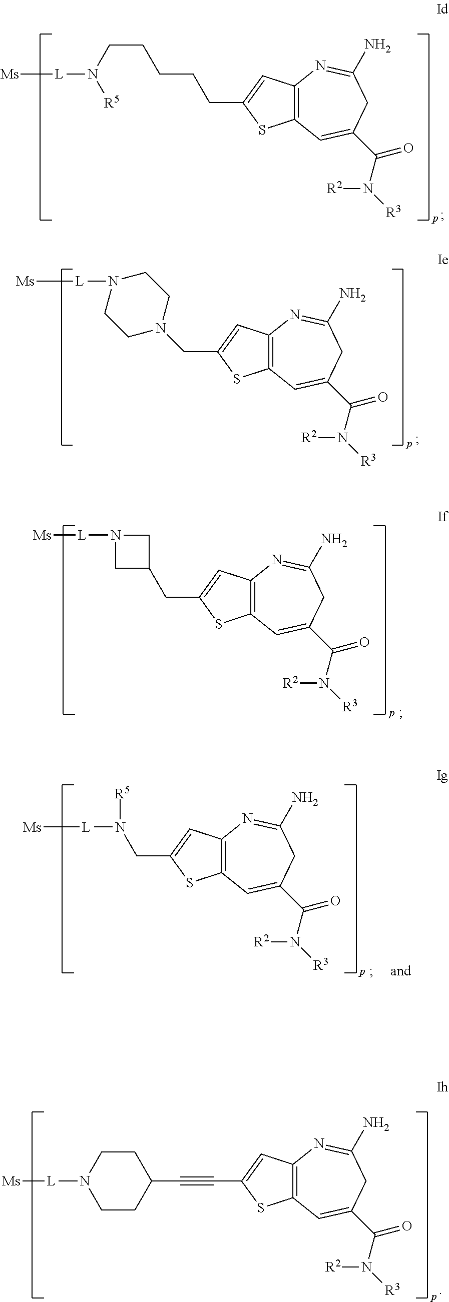

The invention includes all reasonable combinations, and permutations of the features, of the Formula I embodiments.

In certain embodiments, the macromolecule-supported compounds of the invention include those with immunostimulatory activity. The antibody-drug conjugates of the invention selectively deliver an effective dose of a thienoazepine drug to tumor tissue, whereby greater selectivity (i.e., a lower efficacious dose) may be achieved while increasing the therapeutic index (“therapeutic window”) relative to unconjugated thienoazepine.

Drug loading is represented by p, the number of TAZ moieties per antibody in a macromolecule-supported compound of Formula I. Drug (TAZ) loading may range from 1 to about 50 drug (adjuvant) moieties (D) per antibody. Macromolecule-supported compounds of Formula I include mixtures or collections of macromolecular supports conjugated with a range of drug moieties, from 1 to about 25, or 1 to 6. In some embodiments, the number of drug moieties that can be conjugated to a macromolecular support is limited by the number of reactive or available nucleophiles such as amino acid side chain residues like lysine and cysteine. In some embodiments, free cysteine residues are introduced into an antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p=n=1, 2, 3, 4, 5, 6, 7, or 8, or some range there between). Exemplary macromolecule-supported compounds of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intrachain disulfide bonds, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

For some macromolecule-supported compounds, p may be limited by the number of attachment sites on the macromolecular support. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with an TAZ-linker compound of Formula II. In certain embodiments, higher drug loading, e.g. p>5, may cause aggregation, insolubility, toxicity, or loss of cellular permeability of certain macromolecule-support compounds. In certain embodiments, the average drug loading for a macromolecule-supported compound ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of a macromolecule-supported compound may be controlled in different ways, and for example, by: (i) limiting the molar excess of the TAZ-linker intermediate compound relative to macromolecular support, (ii) limiting the conjugation reaction time or temperature, and (iii) partial or limiting reductive denaturing conditions for optimized reactivity.

It is to be understood that where more than one nucleophilic group of the macromolecular support reacts with a drug, then the resulting product is a mixture of macromolecule-support compounds with a distribution of one or more TAZ drug moieties attached to the macromolecular support. The average number of drugs per antibody may be calculated from the mixture by a dual ELISA antibody assay, which is specific for antibody and specific for the drug. Individual macromolecule-supported compound molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous macromolecule-supported compound with a single loading value may be isolated from the conjugation mixture by electrophoresis or chromatography.

An exemplary embodiment of the macromolecule-supported compounds of Formula I is selected from Table 3. BSA (bovine serum albumin) has numerous biochemical applications including ELISAs (Enzyme-Linked Immunosorbent Assay), immunoblots, and immunohistochemistry. Because BSA is a small, stable, moderately non-reactive protein, it is often used as a blocker in immunohistochemistry. KLH peptide (Thermo Scientific Imject™ Mariculture KLH) is a purified keyhole limpet hemocyanin carrier protein that enables simple preparation of highly effective immunogens with peptide antigens.

TABLE 3
Macromolecule-supported compounds (MSC)

| | TAZ- | | |
|---|---|---|---|
| | linker | | |
| MSC No. | Table 2a/b | Macromolecule | DAR |
| MSC-1 | TAZ-L-53 | BSA monomer | 2.53 |
| MSC-2 | TAZ-L-59 | BSA monomer | 2.4 |
| MSC-3 | TAZ-L-73 | BSA monomer | 1.27 |
| MSC-4 | TAZ-L-60 | KLH peptide | 1 |
| MSC-5 | TAZ-L-98 | BSA monomer | 2.28 |

### Compositions of Macromolecule-Supported Compounds

The invention provides a composition, e.g., a pharmaceutically or pharmacologically acceptable composition or formulation, comprising a plurality of macromolecule-supported compounds as described herein and optionally a carrier therefor, e.g., a pharmaceutically or pharmacologically acceptable carrier. The macromolecule-supported compounds can be the same or different in the composition, i.e., the composition can comprise macromolecule-supported compounds that have the same number of adjuvants linked to the same positions on the antibody construct and/or macromolecule-supported compounds that have the same number of TAZ adjuvants linked to different positions on the antibody construct, that have different numbers of adjuvants linked to the same positions on the antibody construct, or that have different numbers of adjuvants linked to different positions on the antibody construct.

In an exemplary embodiment, a composition comprising the macromolecule-supported compound comprises a mixture of the macromolecule-supported compounds, wherein the average drug (TAZ) loading per macromolecular support in the mixture of macromolecule-supported compounds is about 2 to about 5.

A composition of macromolecule-supported compounds of the invention can have an average adjuvant to macromolecular support ratio (DAR) of about 0.4 to about 10. A skilled artisan will recognize that the number of thienoazepine adjuvants conjugated to the macromolecular support, e.g. antibody construct, may vary from macromolecule-supported compound to macromolecule-supported compound in a composition comprising multiple macromolecule-supported compounds of the invention, and, thus, the adjuvant to antibody construct (e.g., antibody) ratio can be measured as an average, which may be referred to as the drug to antibody ratio (DAR). The adjuvant to antibody construct (e.g., antibody) ratio can be assessed by any suitable means, many of which are known in the art.

The average number of adjuvant moieties per macromolecule-support (DAR) in preparations of macromolecule-supported compounds from conjugation reactions may be characterized by conventional means such as mass spectrometry, ELISA assay, and HPLC. The quantitative distribution of macromolecule-supported compounds in a composition in terms of p may also be determined. In some instances, separation, purification, and characterization of homogeneous macromolecule-supported compounds where p is a certain value from macromolecule-supported compounds with other drug loadings may be achieved by means such as reverse phase HPLC or electrophoresis.

In some embodiments, the macromolecule-supported compound further comprises one or more pharmaceutically or pharmacologically acceptable excipients. For example, the macromolecule-supported compounds of the invention can be formulated for parenteral administration, such as IV administration or administration into a body cavity or lumen of an organ. Alternatively, the macromolecule-supported compounds can be injected intra-tumorally. Compositions for injection will commonly comprise a solution of the macromolecule-supported compound dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the macromolecule-supported compound. The concentration of the macromolecule-supported compound in the composition can vary widely, and will be selected primarily based on fluid volumes, viscosities, body weight, and the like, in accordance with the particular mode of administration selected and the patient's needs. In certain embodiments, the concentration of a macromolecule-supported compound in a solution formulation for injection will range from about 0.1% (w/w) to about 10% (w/w).

Method of Treating Cancer with Macromolecule-Supported Compounds

The invention provides a method for treating cancer. The method includes administering a therapeutically effective amount of a macromolecule-supported compound as described herein (e.g., as a composition as described herein) to a subject in need thereof, e.g., a subject that has cancer and is in need of treatment for the cancer. The method includes administering a therapeutically effective amount of a macromolecule-supported compound.

It is contemplated that a macromolecule-supported compound of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, a macromolecule-supported compound for use as a medicament is provided. In certain embodiments, the invention provides a macromolecule-supported compound for use in a method of treating an individual comprising administering to the individual an effective amount of a macromolecule-supported compound. In one such embodiment, the method further comprises administering to the individual an effective amount of at least one additional therapeutic agent, e.g., as described herein.

In a further aspect, the invention provides for the use of a macromolecule-supported compound in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering a macromolecule-supported compound containing an antibody construct that is capable of binding PD-L1 (e.g., atezolizumab, durvalumab, avelumab, biosimilars thereof, or biobetters thereof). In some embodiments, methods for treating breast cancer include administering a macromolecule-supported compound containing an antibody construct that is capable of binding PD-L1 (e.g., atezolizumab, durvalumab, avelumab, biosimilars thereof, or biobetters thereof). In some embodiments, methods for treating triple-negative breast cancer include administering a macromolecule-supported compound containing an antibody construct that is capable of binding PD-L1 (e.g., atezolizumab, durvalumab, avelumab, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering a macromolecule-supported compound containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Macromolecule-supported compounds of the invention can be used either alone or in combination with other agents in a therapy. For instance, a macromolecule-supported compound may be co-administered with at least one additional therapeutic agent, such as a chemotherapeutic agent. Such combination therapies encompass combined administration (where two or more therapeutic agents are included in the same or separate formulations), and separate administration, in which case, administration of the macromolecule-supported compound can occur prior to, simultaneously, and/or following, administration of the additional therapeutic agent and/or adjuvant. Macromolecule-supported compounds can also be used in combination with radiation therapy.

The macromolecule-supported compounds of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Atezolizumab, durvalumab, avelumab, biosimilars thereof, and biobetters thereof are known to be useful in the treatment of cancer, particularly breast cancer, especially triple negative (test negative for estrogen receptors, progesterone receptors, and excess HER2 protein) breast cancer, bladder cancer, and Merkel cell carcinoma. The macromolecule-supported compound described herein can be used to treat the same types of cancers as atezolizumab, durvalumab, avelumab, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

The macromolecule-supported compound is administered to a subject in need thereof in any therapeutically effective amount using any suitable dosing regimen, such as the dosing regimens utilized for atezolizumab, durvalumab, avelumab, biosimilars thereof, and biobetters thereof. For example, the methods can include administering the macromolecule-supported compound to provide a dose of from about 100 ng/kg to about 50 mg/kg to the subject. The macromolecule-supported compound dose can range from about 5 mg/kg to about 50 mg/kg, from about 10 μg/kg to about 5 mg/kg, or from about 100 μg/kg to about 1 mg/kg. The macromolecule-supported compound dose can be about 100, 200, 300, 400, or 500 μg/kg. The macromolecule-supported compound dose can be about 1, 2, 3, 4, 5, 6, 7, 8, 9, or 10 mg/kg. The macromolecule-supported compound dose can also be outside of these ranges, depending on the particular conjugate as well as the type and severity of the cancer being treated. Frequency of administration can range from a single dose to multiple doses per week, or more frequently. In some embodiments, the macromolecule-supported compound is administered from about once per month to about five times per week. In some embodiments, the macromolecule-supported compound is administered once per week.

In another aspect, the invention provides a method for preventing cancer. The method comprises administering a therapeutically effective amount of a macromolecule-supported compound (e.g., as a composition as described above) to a subject. In certain embodiments, the subject is susceptible to a certain cancer to be prevented. For example, the methods can include administering the macromolecule-supported compound to provide a dose of from about 100 ng/kg to about 50 mg/kg to the subject. The macromolecule-supported compound dose can range from about 5 mg/kg to about 50 mg/kg, from about 10 μg/kg to about 5 mg/kg, or from about 100 μg/kg to about 1 mg/kg. The macromolecule-supported compound dose can be about 100, 200, 300, 400, or 500 μg/kg. The macromolecule-supported compound dose can be about 1, 2, 3, 4, 5, 6, 7, 8, 9, or 10 mg/kg. The macromolecule-supported compound dose can also be outside of these ranges, depending on the particular conjugate as well as the type and severity of the cancer being treated. Frequency of administration can range from a single dose to multiple doses per week, or more frequently. In some embodiments, the macromolecule-supported compound is administered from about once per month to about five times per week. In some embodiments, the macromolecule-supported compound is administered once per week.

Some embodiments of the invention provide methods for treating cancer as described above, wherein the cancer is breast cancer. Breast cancer can originate from different areas in the breast, and a number of different types of breast cancer have been characterized. For example, the macromolecule-supported compounds of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering a macromolecule-supported compound containing an antibody construct that is capable of binding HER2 (e.g. trastuzumab, pertuzumab, biosimilars, or biobetters thereof) and PD-L1 (e.g., atezolizumab, durvalumab, avelumab, biosimilars, or biobetters thereof). In some embodiments, methods for treating colon cancer lung cancer, renal cancer, pancreatic cancer, gastric cancer, and esophageal cancer include administering a macromolecule-supported compound containing an antibody construct that is capable of binding CEA, or tumors over-expressing CEA (e.g. labetuzumab, biosimilars, or biobetters thereof). In some embodiments, the cancer is susceptible to a pro-inflammatory response induced by TLR7 and/or TLR8.

### EXAMPLES

### Preparation of Thienoazepine Compounds (TAZ) and Intermediates

### Example 1 Synthesis of 5-amino-2-bromo-N,N-dipropyl-6H-thieno[3,2-b]azepine-7-carboxamide, TAZ-1

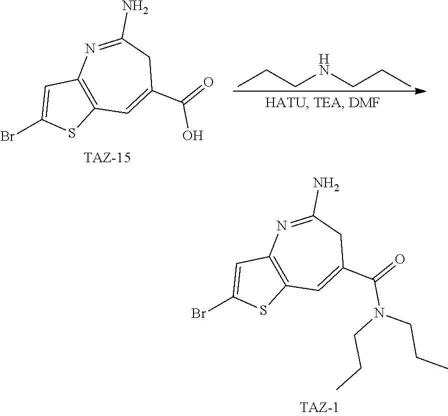

To a solution of 5-amino-2-bromo-6H-thieno[3,2-b]azepine-7-carboxylic acid, TAZ-15 (70 mg, 244 μmol (micromoles), 1 eq) in DMF (1 mL) was added 1-[Bis(dimethylamino)methylene]-11H-1,2,3-triazolo[4,5-b]pyridinium 3-oxide hexafluorophosphate, Hexafluorophosphate Azabenzotriazole Tetramethyl Uronium, HATU (110 mg, 293 μmol, 1.2 eq), N-propylpropan-1-amine (74.0 mg, 731 μmol, 100 μL (microliters), 3 eq) and triethylamine, Et₃N (49.0 mg, 488 μmol, 67.8 μL, 2 eq). The mixture was stirred at 25° C. for 1 h. The mixture was filtered and purified by prep-HPLC (column: Nano-micro Kromasil C18 100*30 mm 8 um; mobile phase: [water(0.1% TFA)−ACN]; B %: 20%-50%, 10 min) to give TAZ-1 (27 mg, 72.91 μmol, 29.9% yield) as white solid. ¹H NMR (CDCl₃, 400 MHz) δ11.66 (s, 1H), 7.75 (s, 1H), 7.22 (s, 1H), 6.79 (s, 1H), 3.40 (s, 4H), 3.28 (s, 2H), 1.68-1.57 (m, 4H), 0.91 (t, J=7.2 Hz, 6H). LC/MS [M+H] 370.0 (calculated); LC/MS [M+H] 370.0 (observed).

### Example 2 Synthesis of 5-amino-N,N-dipropyl-6H-thieno[3,2-b]azepine-7-carboxamide, TAZ-2

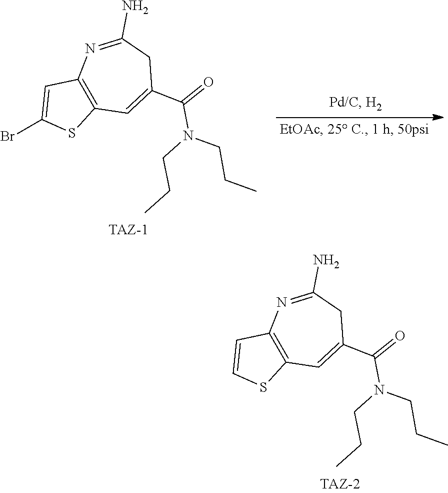 [SMILES: CCCN(CCC)C(=O)C1=Cc2sc(Br)cc2N=C(N)C1 ; CCCN(CCC)C(=O)C1=Cc2sccc2N=C(N)C1]

To a solution of 5-amino-2-bromo-N,N-dipropyl-6H-thieno[3,2-b]azepine-7-carboxamide, TAZ-1 (70 mg, 189 μmol, 1 eq) in ethylacetate, EtOAc (5 mL) was added palladium on carbon, Pd/C (10 mg, 189 μmol, 20% purity, 1 eq) The suspension was degassed under vacuum and purged with H₂ several time and then stirred under H₂ (50 psi) at 25° C. for 1 h. The mixture was filtered and concentrated. The residue was purified by prep-HPLC (column: Nano-micro Kromasil C18 100*30 mm 8 um; mobile phase: [water(0.1% TFA)-ACN]; B %: 15%-40%, 10 min) to give TAZ-2 (12 mg, 41.18 μmol, 21.78% yield) as white solid. ¹H NMR (MeOD-d₄, 400 MHz) δ7.71 (d, J=5.6 Hz, 1H), 7.15-7.09 (m, 2H), 3.44-3.40 (m, 4H), 3.36 (s, 2H), 1.72-1.61 (m, 4H), 0.98-0.84 (m, 6H). LC/MS [M+H] 292.1 (calculated); LC/MS [M+H] 292.1 (observed)

### Example 3 Synthesis of tert-butyl (2-(1-(5-(5-amino-7-(dipropylcarbamoyl)-6H-thieno[3,2-b]azepine-2-carboxamido)pyridin-2-yl)piperidine-4-carboxamido)ethyl)carbamate, TAZ-3

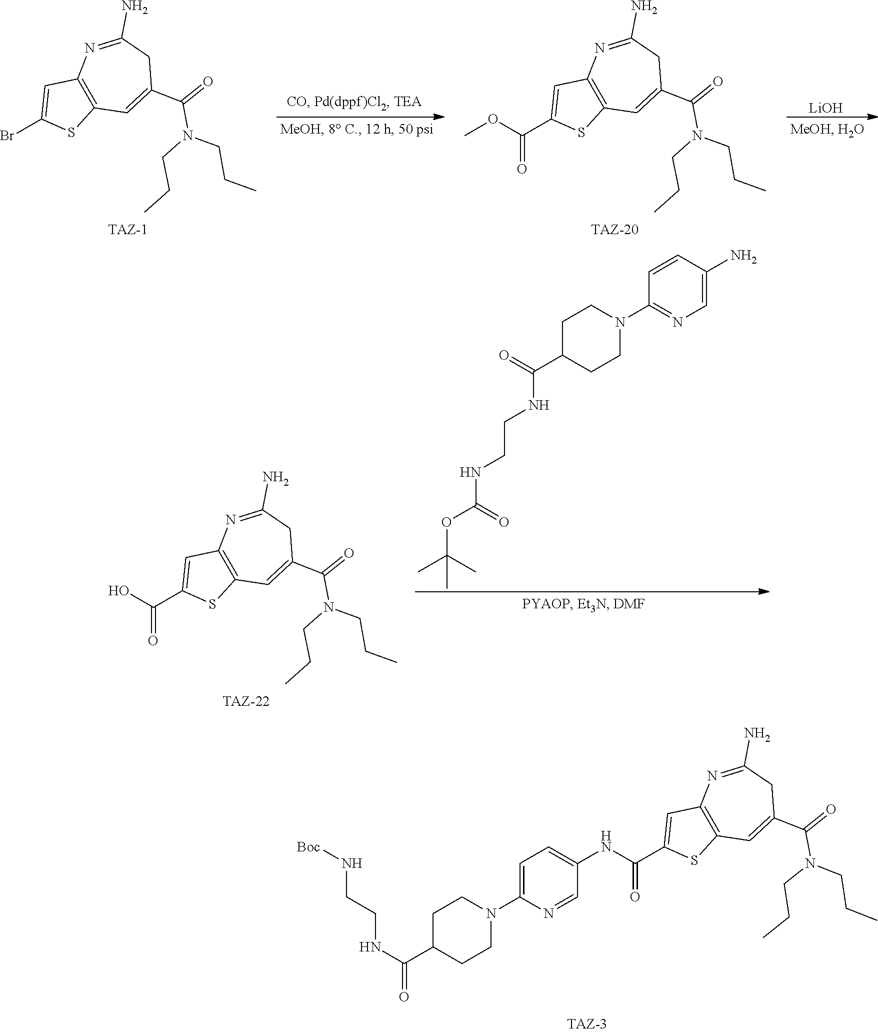 [SMILES: CCCN(CCC)C(=O)C1=Cc2sc(Br)cc2N=C(N)C1 ; CCCN(CCC)C(=O)C1=Cc2sc(C(=O)OC)cc2N=C(N)C1 ; "LiOH" ; CCCN(CCC)C(=O)C1=Cc2sc(C(=O)O)cc2N=C(N)C1 ; CC(C)(C)OC(=O)NCCNC(=O)C1CCN(c2ccc(N)cn2)CC1 ; CCCN(CCC)C(=O)C1=Cc2sc(C(=O)Nc3ccc(N4CCC(C(=O)NCCNC(=O)OC(C)(C)C)CC4)nc3)cc2N=C(N)C1]

### Preparation of methyl 5-amino-7-(dipropylcarbamoyl)-6H-thieno [3,2-b]azepine-2-carboxylate, TAZ-20

To a solution of 5-amino-2-bromo-N,N-dipropyl-6H-thieno[3,2-b]azepine-7-carboxamide, TAZ-1 (0.5 g, 1.30 mmol, 1 eq) in MeOH (5 mL) was added Et₃N (409 mg, 4.05 mmol, 564 μL, 3 eq) and [1,1′-bis(diphenylphosphino)ferrocene]palladium(II) dichloride, Pd(dppf)Cl₂ (99.0 mg, 135 μmol, 0.1 eq) under N₂. The suspension was degassed under vacuum and purged with carbon monoxide, CO several times. The mixture was stirred under CO (50 psi) at 80° C. for 12 h. The mixture was filtered and concentrated to give TAZ-20 (0.5 g, crude) as red solid. ¹H NMR (MeOD-d₄, 400 MHz) δ7.51 (s, 1H), 6.91 (s, 1H), 3.87 (s, 3H), 3.43-3.35 (m, 4H), 3.35 (s, 2H), 1.73-1.60 (m, 4H), 0.97-0.83 (m, 6H).

### Preparation of 5-amino-7-(dipropylcarbamoyl)-6H-thieno[3,2-b] azepine-2-carboxylic acid, TAZ-22

To a solution of methyl 5-amino-7-(dipropylcarbamoyl)-6H-thieno[3,2-b] azepine-2-carboxylate, TAZ-20 (450 mg, 1.29 mmol, 1 eq) in MeOH (10 mL) and H₂O (10 mL) was added LiOH·H₂O (270 mg, 6.44 mmol, 5 eq), and then stirred at 25° C. for 2 h. The reaction mixture was concentrated under reduced pressure to remove MeOH. The residue was diluted with H₂O 30 mL and extracted with EtOAc (10 mL×2). The aqueous phase pH was adjusted to about 4 with aq (aqueous) HCl (1M) and extracted with EtOAc (10 mL×3). The organic layer was washed with brine (10 mL), dried over Na₂SO₄, filtered and concentrated to give TAZ-22 (0.2 g, 596.27 μmol, 46.30% yield) as light yellow solid. ¹H NMR (DMSO-d₆, 400 MHz) δ7.49 (s, 1H), 6.99 (s, 1H), 3.32-3.28 (m, 4H), 3.16 (s, 2H), 1.61-1.46 (m, 4H), 0.82 (br s, 6H).

### Preparation of tert-butyl (2-(1-(5-(5-amino-7-(dipropylcarbamoyl)-6H-thieno[3,2-b]azepine-2-carboxamido)pyridin-2-yl)piperidine-4-carboxamido)ethyl)carbamate, TAZ-3

To a solution of 5-amino-7-(dipropylcarbamoyl)-6H-thieno[3,2-b]azepine-2-carboxylic acid (150 mg, 447 μmol, 1 eq) in DMF (2 mL) was added 7-Aza-benzotriazol-1-yloxy-tripyrrolidino-phosphonium hexafluorophosphate, PYAOP (256 mg, 491.9 μmol, 1.1 eq), Et₃N (45.0 mg, 447.2 μmol, 62.25 μL, 1 eq) and tert-butyl N-[2-[[1-(5-amino-2-pyridyl)piperidine-4-carbonyl]amino]ethyl]carbamate (195. mg, 536.6 μmol, 1.2 eq) and it was stirred at 25° C. for 12 h. The mixture was filtered and purified by prep-HPLC (column: Welch Xtimate C18 150*25 mm*5 um; mobile phase: [water(10 mM NH4HCO3)−ACN]; B %: 30%-60%, 10.5 min) to give TAZ-3 (62 mg, 91.06 μmol, 20.36% yield) as grayness solid. ¹H NMR (MeOD-d₄, 400 MHz) δ8.35 (d, J=2.0 Hz, 1H), 7.89-7.82 (m, 1H), 7.60 (s, 1H), 7.51 (s, 1H), 6.93 (s, 1H), 6.91-6.84 (m, 1H), 4.28 (d, J=12.8 Hz, 2H), 3.44-3.35 (m, 4H), 3.27-3.21 (m, 2H), 3.18-3.12 (m, 2H), 2.97 (s, 2H), 2.88 (t, J=11.6 Hz, 2H), 2.48-2.32 (m, 1H), 1.90-1.80 (m, 2H), 1.79-1.56 (m, 6H), 1.43 (s, 9H), 0.90 (s, 6H). LC/MS [M+H] 681.3 (calculated); LC/MS [M+H] 681.4 (observed).

### Example 4 Synthesis of 5-amino-2-[3-[3-(hydroxymethyl)azetidin-1-yl]sulfonylphenyl]-N,N-dipropyl-6H-thieno[3,2-b]azepine-7-carboxamide, TAZ-4

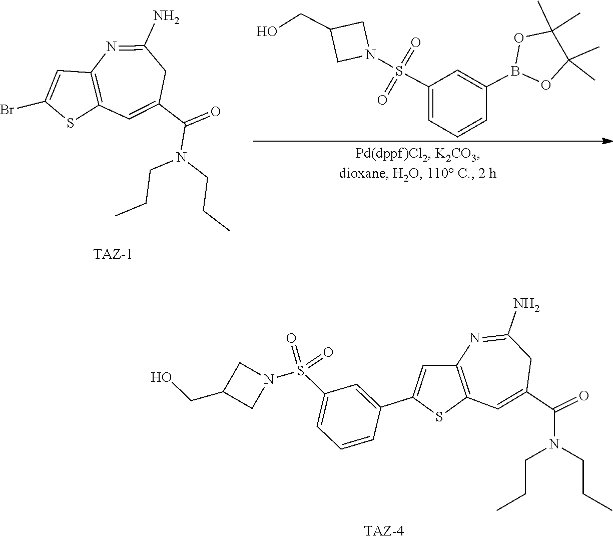 [SMILES: CCCN(CCC)C(=O)C1=Cc2sc(Br)cc2N=C(N)C1 ; CC1(C)OB(c2cccc(S(=O)(=O)N3CC(CO)C3)c2)OC1(C)C ; CCCN(CCC)C(=O)C1=Cc2sc(-c3cccc(S(=O)(=O)N4CC(CO)C4)c3)cc2N=C(N)C1]

To a solution of 5-amino-2-bromo-N,N-dipropyl-6H-thieno[3,2-b]azepine-7-carboxamide, TAZ-1 (71.0 mg, 192 μmol, 1.1 eq) in dioxane (1 mL) and H₂O (0.5 mL) was added [1-[3-(4,4,5,5-tetramethyl-1,3,2-dioxaborolan-2-yl)phenyl]sulfonylazetidin-3-yl]methanol (62.0 mg, 174 μmol, 1 eq), K₂CO₃ (48.0 mg, 348 μmol, 2 eq) and [1,1′-bis(diphenylphosphino)ferrocene]palladium(II) dichloride, Pd(dppf)Cl₂ (6.0 mg, 8.7 μmol, 0.05 eq) at 25° C. under N₂ and then stirred at 110° C. for 2 h. The mixture was filtered and concentrated. The residue was purified by prep-HPLC (column: Welch Xtimate C18 150*25 mm*5 um; mobile phase: [water(10 mM NH4HCO3)−ACN]; B %: 30%-60%, 10.5 min) to give TAZ-4 (22 mg, 42.58 μmol, 24.45% yield) as light yellow solid. ¹H NMR (MeOD-d₄, 400 MHz) δ8.04-7.97 (m, 2H), 7.79-7.75 (m, 1H), 7.73-7.67 (m, 1H), 7.31 (s, 1H), 6.92 (s, 1H), 3.85 (t, J=8.2 Hz, 2H), 3.62-3.56 (m, 2H), 3.45-3.37 (m, 6H), 2.99 (s, 2H), 2.63-2.52 (m, 1H), 1.71-1.59 (m, 4H), 0.99-0.83 (m, 6H). LC/MS [M+H] 517.2 (calculated); LC/MS [M+H] 517.2 (observed)

### Example 5 Synthesis of [4-[[(2S)-2-[[(2S)-2-(9H-fluoren-9-ylmethoxycarbonylamino)-3-methyl-butanoyl]amino]-5-ureido-pentanoyl]amino]phenyl]methyl N-[2-[[1-[5-[[5-amino-7-(dipropylcarbamoyl)-6H-thieno [3,2-b]azepine-2-carbonyl]amino]-2-pyridyl]piperidine-4-carbonyl]amino]ethyl]carbamate, TAZ-5

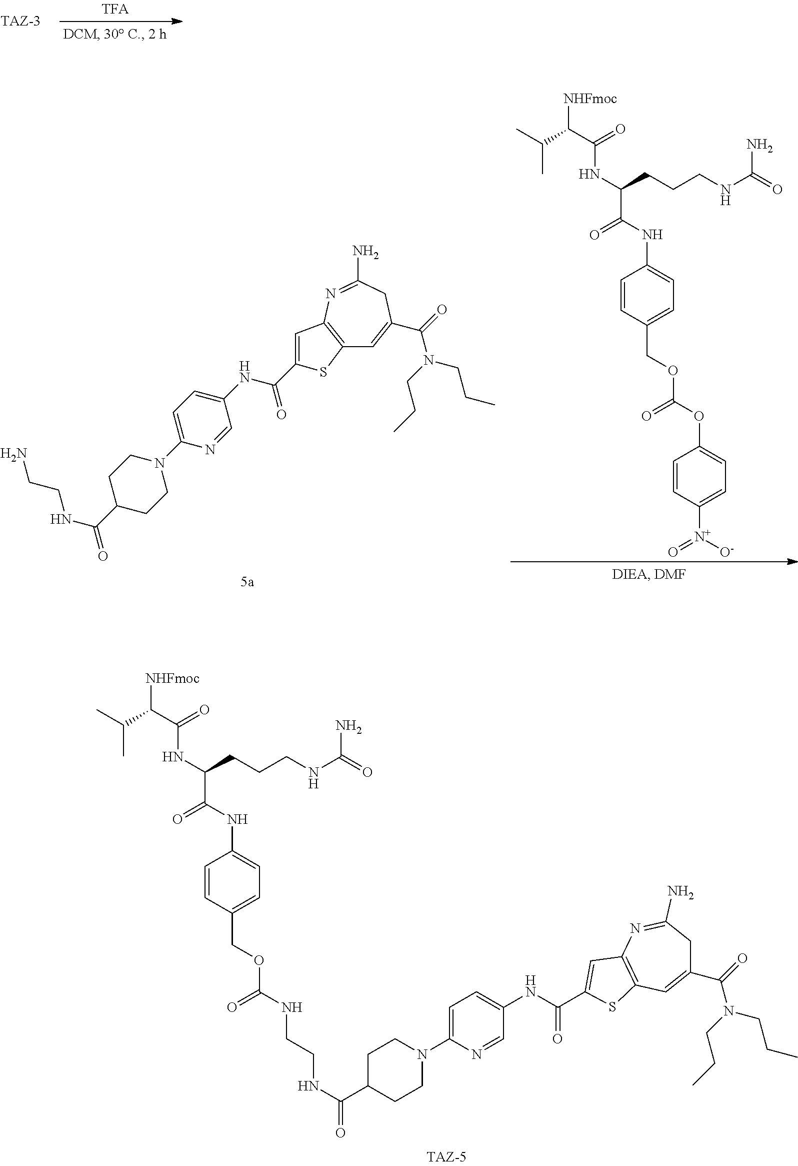

### Preparation of 5-amino-N2-[6-[4-(2-aminoethylcarbamoyl)-1-piperidyl]-3-pyridyl]-N7,N7-dipropyl-6H-thieno[3,2-b]azepine-2,7-dicarboxamide, 5a

To a solution of tert-butyl N-[2-[[1-[5-[[5-amino-7-(dipropylcarbamoyl)-6H-thieno[3,2-b] azepine-2-carbonyl]amino]-2-pyridyl]piperidine-4-carbonyl]amino]ethyl]carbamate, TAZ-3 (50 mg, 73.4 μmol, 1 eq) in DCM (1 mL) was added TFA (84.0 mg, 734 μmol, 54.0 μL, 10 eq). The mixture was stirred at 30° C. for 2 h. The mixture was filtered and concentrated and then lyophilization to give 5a (59 mg, 72.95 μmol, 99.34% yield, 2TFA) as grayness solid. ¹H NMR (MeOD-d₄, 400 MHz) δ8.62 (d, J=2.4 Hz, 1H), 8.17 (dd, J=9.6, 2.8 Hz, 1H), 7.89 (s, 1H), 7.43 (d, J=9.6 Hz, 1H), 7.16 (s, 1H), 4.21 (d, J=13.6 Hz, 2H), 3.52-3.39 (m, 8H), 3.36-3.31 (m, 2H), 3.07 (t, J=6.0 Hz, 2H), 2.68-2.61 (m, 1H), 2.07-2.00 (m, 2H), 1.90-1.78 (m, 2H), 1.67 (dq, J=14.8, 7.2 Hz, 4H), 0.93 (s, 6H).

### Preparation of TAZ-5

To a solution of 5a (50 mg, 61.8 μmol, 1 eq, 2TFA) in DMF (1 mL) was added DIEA (32.0 mg, 247.2 μmol, 43.0 μL, 4 eq) and [4-[[(2S)-2-[[(2S)-2-(9H-fluoren-9-ylmethoxycarbonylamino)-3-methyl-butanoyl]amino]-5-ureido-pentanoyl]amino]phenyl]methyl (4-nitrophenyl) carbonate (52.0 mg, 68.0 μmol, 1.1 eq) and then stirred at 25° C. for 1 h. The mixture was filtered and purified by prep-HPLC (column: Nano-micro Kromasil C18 100*30 mm 8 um; mobile phase: [water(0.1% TFA)−ACN]; B %: 25%-45%, 10 min) to give TAZ-5 (36 mg, 27.2 μmol, 44.03% yield, TFA) as light yellow solid. ¹H NMR (MeOD-d₄, 400 MHz) δ8.58 (s, 1H), 8.07 (br d, J=10.4 Hz, 1H), 7.93 (br s, 1H), 7.86-7.72 (m, 3H), 7.63 (br t, J=7.2 Hz, 2H), 7.57 (br d, J=8.2 Hz, 2H), 7.41-7.34 (m, 2H), 7.33-7.25 (m, 4H), 7.13 (s, 1H), 5.03 (br s, 2H), 4.41-4.29 (m, 3H), 4.23-4.15 (m, 1H), 4.13-4.03 (m, 2H), 3.98-3.91 (m, 1H), 3.51-3.34 (m, 5H), 3.25-3.07 (m, 9H), 2.52-2.31 (m, 1H), 2.08 (br d, J=7.6 Hz, 1H), 1.99-1.36 (m, 12H), 1.04-0.82 (m, 12H). LC/MS [M+H] 1208.6 (calculated); LC/MS [M+H] 1208.5 (observed).

### Example 6 Synthesis of tert-butyl N-[5-[5-amino-7-(dipropylcarbamoyl)-6H-thieno[3,2-b]azepin-2-yl]pent-4-ynyl] carbamate, TAZ-6

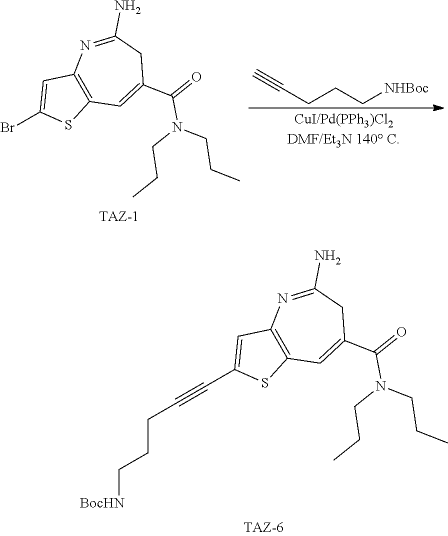 [SMILES: CCCN(CCC)C(=O)C1=Cc2sc(Br)cc2N=C(N)C1 ; C#CCCCNC(=O)OC(C)(C)C ; CCCN(CCC)C(=O)C1=Cc2sc(C#CCCCNC(=O)OC(C)(C)C)cc2N=C(N)C1]

A mixture of 5-amino-2-bromo-N,N-dipropyl-6H-thieno[3,2-b]azepine-7-carboxamide, TAZ-1 (20 mg, 54.0 μmol, 1 eq), tert-butyl N-pent-4-ynylcarbamate (29.69 mg, 162 μmol, 3 eq), Pd(PPh₃)₂Cl₂ (1.90 mg, 2.70 μmol, 0.05 eq), CuI (2.06 mg, 10.8 μmol, 0.2 eq) and PPh₃ (2.83 mg, 10.8 μmol, 0.2 eq) in TEA (0.2 mL) and DMF (0.6 mL) was degassed and purged with N₂ for 3 times, and then stirred at 140° C. for 3 h under N₂. The reaction mixture was quenched by addition of H₂O (5 mL) at 0° C., and then extracted with EtOAc (5 mL×3). The combined organic layers were washed with brine(5 mL×3), dried over Na₂SO₄, filtered and concentrated under reduced pressure. The residue was purified by prep-TLC (SiO₂, EtOAc:MeOH=10:1) to give TAZ-6 (8 mg, 16.9 μmol, 31.34% yield) as yellow solid. ¹H NMR (MeOD-d₄, 400 MHz) δ6.87 (s, 1H), 6.79 (s, 1H), 3.42-3.34 (m, 4H), 3.31 (s, 2H), 3.17 (t, J=6.8 Hz, 2H), 2.48 (t, J=7.2 Hz, 2H), 1.75 (q, J=7.2 Hz, 2H), 1.69-1.56 (m, 4H), 1.44 (s, 9H), 0.92-0.87 (m, 6H). LC/MS [M+H] 473.2 (calculated); LC/MS [M+H] 473.2 (observed)

### Example 7 Synthesis of 5-amino-2-methyl-N,N-dipropyl-6H-thieno[3,2-b]azepine-7-carboxamide, TAZ-7

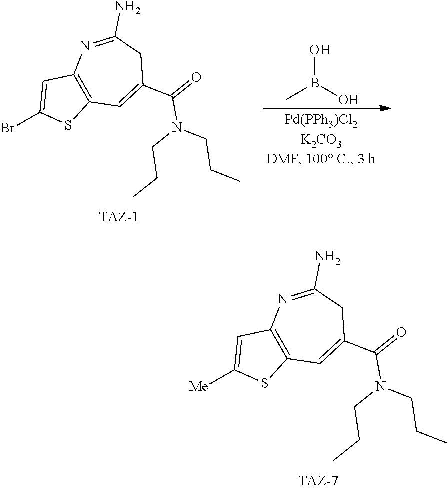

To a solution of 5-amino-2-bromo-N,N-dipropyl-6H-thieno[3,2-b]azepine-7-carboxamide, TAZ-1 (30 mg, 81.0 μmol, 1 eq) in DMF (1 mL) was added methylboronic acid (73.0 mg, 1.22 mmol, 15 eq), K₂CO₃ (22.0 mg, 162.03 μmol, 2 eq) and [1,1′-bis(diphenylphosphino)ferrocene]palladium(II) dichloride, Pd(dppf)Cl₂ (2.96 mg, 4.05 μmol, 0.05 eq) under N₂ and then stirred at 100° C. for 3 h. The mixture was filtered and concentrated. The residue was purified by prep-HPLC (column: Nano-micro Kromasil C18 100*30 mm 8 um; mobile phase: [water(0.1% TFA)−ACN]; B %: 10%-40%, 10 min) to give TAZ-7 (8 mg, 26.19 μmol, 32.33% yield) as white solid. ¹H NMR (MeOD-d₄, 400 MHz) δ7.00 (s, 1H) 6.83 (s, 1H) 3.43 (br t, J=7.2 Hz, 4H) 3.34 (s, 2H) 2.53 (s, 3H) 1.69-1.60 (m, 4H) 0.98-0.85 (m, 6H). LC/MS [M+H] 306.2 (calculated); LC/MS [M+H] 306.2 (observed).

### Example 8 Synthesis of tert-butyl N-[3-[(5-amino-6H-thieno[3,2-b]azepine-7-carbonyl)-propyl-amino]propyl]carbamate, TAZ-8

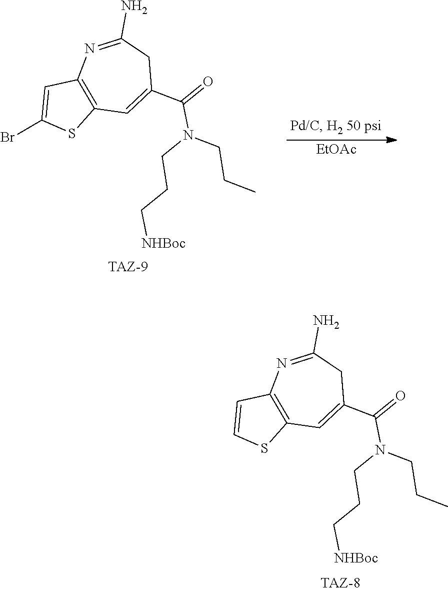 [SMILES: "EtOAc" ; CCCN(CCCNC(=O)OC(C)(C)C)C(=O)C1=Cc2sc(Br)cc2N=C(N)C1 ; CCCN(CCCNC(=O)OC(C)(C)C)C(=O)C1=Cc2sccc2N=C(N)C1]

To a solution of tert-butyl N-[3-[(5-amino-2-bromo-6H-thieno[3,2-b]azepine-7-carbonyl)-propyl-amino]propyl]carbamate, TAZ-9 (0.72 g, 1.48 mmol, 1 eq) in EtOAc (10 mL) was added palladium on carbon, Pd/C (10%, 0.2 g) under N₂. The suspension was degassed under vacuum and purged with H₂ several times, and then stirred under hydrogen gas, H₂ (50 psi) at 25° C. for 12 h. The reaction mixture was filtered and concentrated under reduced pressure. The residue was purified by column chromatography (SiO₂, EtOAc:EtOH=1:0 to 3:1) to give TAZ-8 (0.4 g, 984 μmol, 66.34% yield) as a light yellow solid. ¹H NMR (MeOD, 400 MHz) δ7.67 (d, J=5.6 Hz, 1H), 7.13-7.05 (m, 2H), 3.50 (t, J=7.6 Hz, 2H), 3.44 (t, J=7.6 Hz, 2H), 3.30-3.24 (m, 2H), 3.07 (s, 2H), 1.84-1.78 (m, 2H), 1.72-1.61 (m, 2H), 1.41 (s, 9H), 0.93-0.88 (m, 3H). LC/MS [M+H] 407.2 (calculated); LC/MS [M+H] 407.2 (observed).

### Example 9 Synthesis of tert-butyl N-[3-[(5-amino-2-bromo-6H-thieno[3,2-b]azepine-7-carbonyl)-propyl-amino]propyl]carbamate, TAZ-9

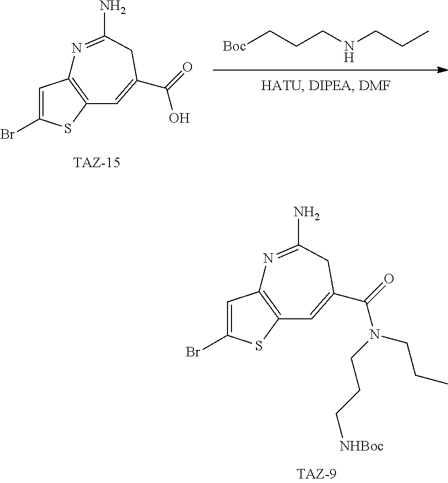 [SMILES: NC1=Nc2cc(Br)sc2C=C(C(=O)O)C1 ; CCCNCCCC(=O)OC(C)(C)C ; CCCN(CCCNC(=O)OC(C)(C)C)C(=O)C1=Cc2sc(Br)cc2N=C(N)C1]

To a mixture of 5-amino-2-bromo-6H-thieno[3,2-b]azepine-7-carboxylic acid, TAZ-15 (0.5 g, 1.74 mmol, 1 eq) in DMF (5 mL) was added 1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium, HATU (795 mg, 2.09 mmol, 1.2 eq) and DIPEA (675 mg, 5.22 mmol, 910 μL, 3 eq) at 25° C. After 15 min, tert-butyl N-[3-(propylamino)propyl]carbamate (489.70 mg, 2.26 mmol, 1.3 eq) was added at 25° C., and then stirred for 1 h. The reaction mixture was quenched by addition of H₂O (30 mL) at 0° C., and then extracted with EtOAc(15 mL×3). The combined organic layers were washed with brine (10 mL×3), dried over Na₂SO₄, filtered and concentrated under reduced pressure. The residue was purified by column chromatography (SiO₂, Petroleum ether/Ethyl acetate=1/0 to 0/1) and (SiO₂, EtOAc:MeOH=1:0 to 5:1) to give TAZ-9 (0.73 g, 1.50 mmol, 86.36% yield) as a light yellow solid. ¹H NMR (MeOD, 400 MHz) δ6.95 (s, 1H), 6.86 (s, 1H), 3.50-3.43 (m, 2H), 3.42-3.35 (m, 2H), 3.07-3.01 (m, 4H), 1.84-1.74 (m, 2H), 1.69-1.57 (m, 2H), 1.41 (s, 9H), 0.91-0.86 (m, 3H). LC/MS [M+H] 485.1 (calculated); LC/MS [M+H] 485.1 (observed).

### Example 10 Synthesis of tert-butyl N-[4-[(5-amino-2-bromo-6H-thieno[3,2-b]azepine-7-carbonyl)-propyl-amino]but-2-ynyl]carbamate, TAZ-10

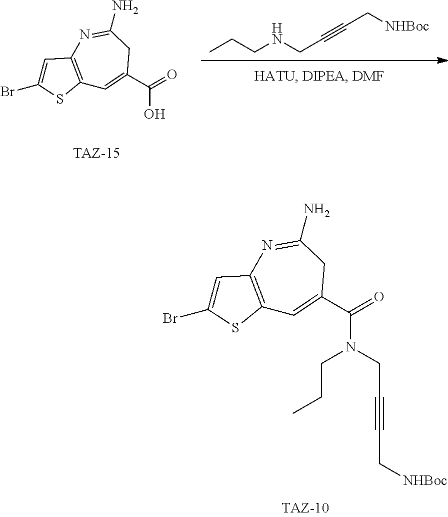 [SMILES: NC1=Nc2cc(Br)sc2C=C(C(=O)O)C1 ; CCCNCC#CCNC(=O)OC(C)(C)C ; CCCN(CC#CCNC(=O)OC(C)(C)C)C(=O)C1=Cc2sc(Br)cc2N=C(N)C1]

To a solution of 5-amino-2-bromo-6H-thieno[3,2-b]azepine-7-carboxylic acid, TAZ-15 (0.2 g, 697 μmol, 1 eq) in DMF (4 mL) was added HATU (318 mg, 836 μmol, 1.2 eq) and DIPEA (270 mg, 2.09 mmol, 3 eq) at 25° C. After 15 min, tert-butyl N-[4-(propylamino)but-2-ynyl]carbamate (205 mg, 906 μmol, 1.3 eq) was added at 25° C. and then stirred at 25° C. for 1 h. The reaction mixture was quenched by addition of H₂O (20 mL) at 0° C., and then extracted with EtOAc(10 mL×3). The combined organic layers were washed with brine (5 mL×3), dried over Na₂SO₄, filtered and concentrated under reduced pressure. The residue was purified by column chromatography (SiO₂, Petroleum ether/Ethyl acetate=1/0 to 0/1) to give TAZ-10 (150 mg, 302.77 μmol, 43.47% yield) as a yellow solid. ¹H NMR (MeOD, 400 MHz) δ7.20-7.18 (m, 2H), 4.29 (s, 2H), 3.84 (s, 2H), 3.58-3.50 (m, 2H), 3.40 (s, 2H), 1.76-1.66 (m, 2H), 1.43 (s, 9H), 0.94 (t, J=7.2 Hz, 3H). LC/MS [M+H] 495.1 (calculated); LC/MS [M+H] 495.1 (observed).

### Example 11 Synthesis of 5-amino-N-(3-aminopropyl)-N-propyl-6H-thieno[3,2-b]azepine-7-carboxamide, TAZ-11

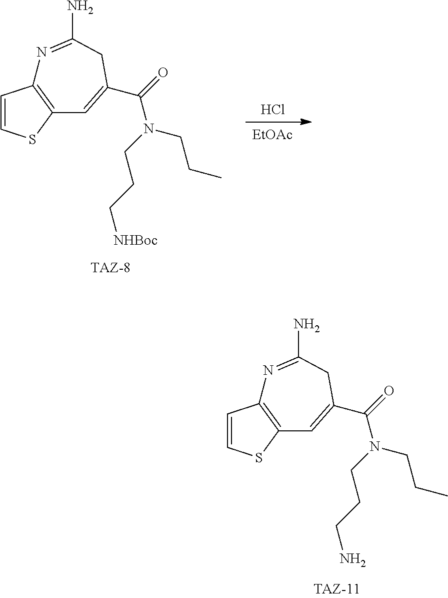 [SMILES: "EtOAc" ; CCCN(CCCNC(=O)OC(C)(C)C)C(=O)C1=Cc2sccc2N=C(N)C1 ; Cl ; CCCN(CCCN)C(=O)C1=Cc2sccc2N=C(N)C1]

To a solution of tert-butyl N-[3-[(5-amino-6H-thieno[3,2-b]azepine-7-carbonyl)-propyl-amino]propyl]carbamate, TAZ-8 (1.4 g, 3.44 mmol, 1 eq) in EtOAc (10 mL) and MeOH (1 mL) was added HCl/EtOAc (4 M, 20 mL, 23.23 eq) at 25° C. and then stirred for 0.5 h. The reaction mixture was concentrated under reduced pressure to give TAZ-11 (1.28 g, crude, HCl) as a light yellow solid. ¹H NMR (MeOD-d₄, 400 MHz) δ7.74 (d, J=5.6 Hz, 1H), 7.20 (s, 1H), 7.15 (d, J=5.6 Hz, 1H), 3.59 (t, J=6.8 Hz, 2H), 3.49 (t, J=7.2 Hz, 2H), 3.41 (s, 2H), 3.00 (t, J=7.2 Hz, 2H), 2.08-1.97 (m, 2H), 1.75-1.62 (m, 2H), 0.91 (t, J=7.2 Hz, 3H). LC/MS [M+H] 307.2 (calculated); LC/MS [M+H] 307.1 (observed).

### Example 12 Synthesis of 5-amino-2-[1-[3-(hydroxymethyl)azetidin-1-yl]sulfonylpyrazol-4-yl]-N,N-dipropyl-6H-thieno[3,2-b]azepine-7-carboxamide,, TAZ-12

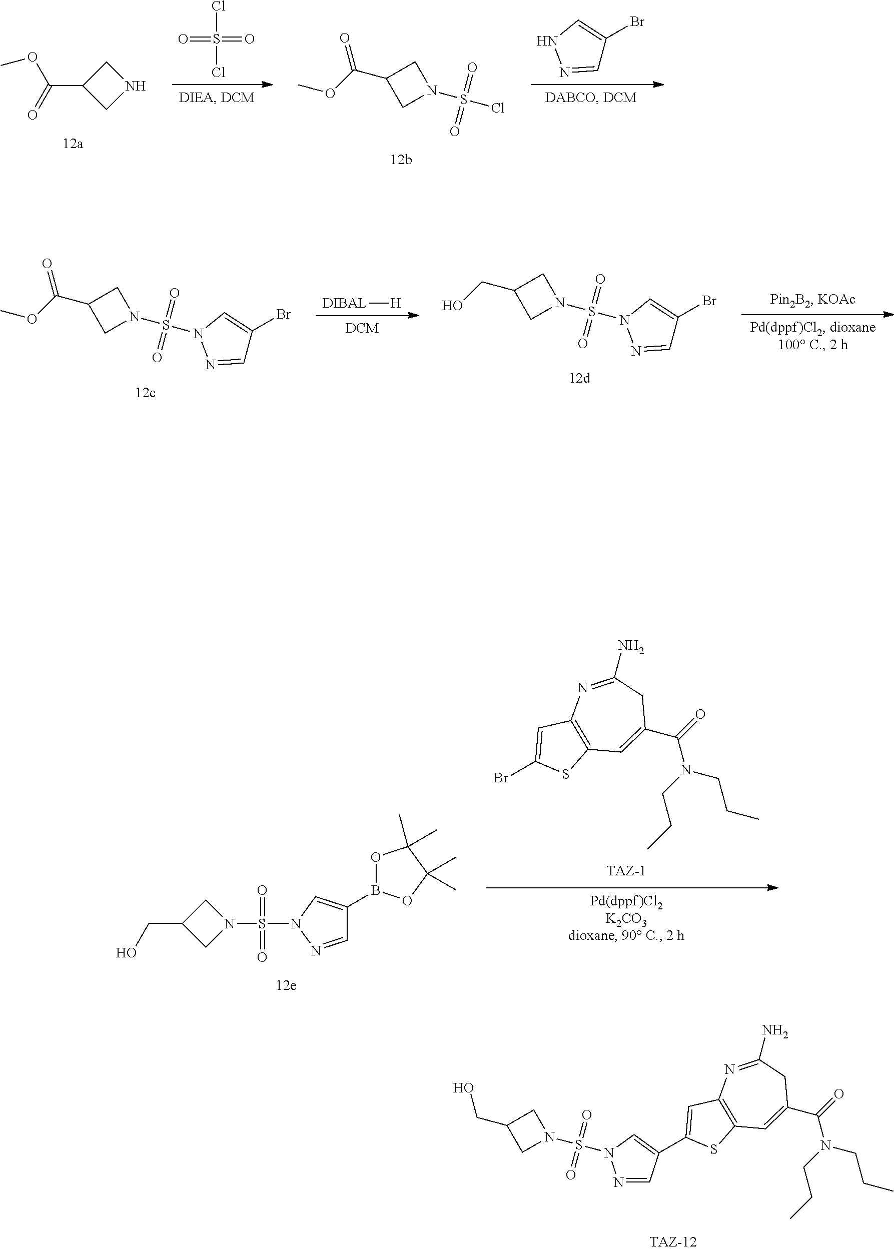

Preparation of methyl 1-chlorosulfonylazetidine-3-carboxylate, 12b To a mixture of sulfuryl chloride (3.34 g, 24.7 mmol, 2.47 mL, 1.5 eq) in DCM (50 mL) was added a solution of methyl azetidine-3-carboxylate, 12a (2.5 g, 16.49 mmol, 1 eq, HCl) and DIEA (8.53 g, 65.97 mmol, 11.49 mL, 4 eq) in DCM (30 mL) at −78° C. and then stirred for 2 h at this temperature. The mixture was diluted with water and extracted with EtOAc (60 mL×3). The organic layer was washed with the brine, dried over Na₂SO₄, filtered and concentrated. The residue was purified by silica gel chromatography (Petroleum ether/Ethyl acetate=1/0, 0/1) to afford 12b (2.75 g, 12.87 mmol, 78.05% yield) as colorless oil. ¹H NMR (CDCl₃, 400 MHz) δ4.36-4.25 (m, 4H), 3.80 (s, 3H), 3.57-3.47 (m, 1H).

### Preparation of methyl 1-(4-bromopyrazol-1-yl)sulfonylazetidine-3-carboxylate, 12c

To a mixture of 4-bromo-1H-pyrazole (1.58 g, 10.77 mmol, 1.0 eq) in DCM (40 mL) was added DABCO (1.57 g, 14.0 mmol, 1.54 mL, 1.3 eq) and 12b (2.3 g, 10.8 mmol, 1.0 eq) in one portion at 25° C. and it was stirred for 2 h. The mixture was diluted with water and extracted with EtOAc (50 mL×3). The organic layer was washed with brine, dried over Na₂SO₄, filtered and concentrated. The residue was purified by silica gel chromatography (Petroleum ether/Ethyl acetate=1/0, 3/1) to afford 12c (3 g, 9.25 mmol, 85.97% yield) as yellow oil. ¹H NMR (CDCl₃, 400 MHz) δ8.03 (s, 1H), 7.77 (s, 1H), 4.32-4.27 (m, 4H), 3.73 (s, 3H), 3.41-3.34 (m, 1H).

### Preparation of [1-(4-bromopyrazol-1-yl)sulfonylazetidin-3-yl]methanol, 12d

To a solution of 12c (3.3 g, 10.2 mmol, 1 eq) in DCM (50 mL) was added DIBAL-H (1 M, 40.7 mL, 4 eq) slowly at 0° C. under N₂, and then stirred at this temperature for 2 h. The mixture was quenched with water (1.5 mL) and dried over Na₂SO₄, filtered and concentrated to obtain 12d (1.19 g, crude) as yellow oil. ¹H NMR (CDCl₃, 400 MHz) δ8.04 (s, 1H), 7.77 (s, 1H), 4.18-4.14 (t, J=8.4 Hz, 2H), 3.96 (dd, J=5.6, 8.4 Hz, 2H), 3.66 (d, J=5.6 Hz, 2H).

### Preparation of [1-[4-(4,4,5,5-tetramethyl-1,3,2-dioxaborolan-2-yl)pyrazol-1-yl]sulfonylazetidin-3-yl]methanol, 12e

To a mixture of 12d (0.1 g, 338 μmol, 1.0 eq) in dioxane (2 mL) was added Pin₂B₂(129 mg, 507 μmol, 1.5 eq), potassium acetate, KOAc (66.3 mg, 675 μmol, 2.0 eq) and Pd(dppf)Cl₂ (12.4 mg, 16.9 μmol, 0.05 eq) in one portion at 25° C. under N₂ and it was stirred at 100° C. for 2 h. Then the mixture was diluted with water and extracted with EtOAc (10 mL×3). The organic layer was washed with brine, dried over Na₂SO₄, filtered and concentrated to give 12e (0.1 g, crude) as black oil.

### Preparation of TAZ-12

To a mixture of 5-amino-2-bromo-N,N-dipropyl-6H-thieno[3,2-b]azepine-7-carboxamide, TAZ-1 (54 mg, 146 μmol, 1.0 eq) and 12e (50 mg, 146 μmol, 1.0 eq) in dioxane (2 mL) and H₂O (0.2 mL) was added K₂CO₃ (60.4 mg, 437 μmol, 3.0 eq) and Pd(dppf)Cl₂ (5.3 mg, 7.28 μmol, 0.05 eq) in one portion at 25° C. under N₂. The mixture was stirred at 90° C. for 2 h. Then the reaction was diluted with water and extracted with EtOAc (10 mL×3). The organic layer was washed with brine, dried over Na₂SO₄, filtered and concentrated. The residue was further purification by prep-HPLC (column: Phenomenex Gemini-NX C18 75*30 mm*3 um; mobile phase: [water(10 mM NH₄HCO₃)−ACN]; B %: 30%-50%, 10.5 min) to give 5-amino-2-[1-[3-(hydroxymethyl)azetidin-1-yl]sulfonylpyrazol-4-yl]-N,N-dipropyl-6H-thieno[3,2-b]azepine-7-carboxamide (9 mg, 17.8 μmol, 12.19% yield) as white solid. ¹H NMR (MeOD, 400 MHz) δ8.56 (s, 1H), 8.28 (s, 1H), 7.42 (s, 1H), 7.38-7.35 (m, 2H), 6.85 (s, 1H), 4.13 (t, J=8.8 Hz, 2H), 3.89 (dd, J=6.0, 8.4 Hz, 2H), 3.49 (d, J=6.0 Hz, 2H), 3.43-3.37 (m, 4H), 2.97 (s, 2H), 2.72-2.67 (m, 1H), 1.69-1.61 (m, 4H), 0.93-0.87 (m, 6H). LC/MS [M+H] 507.2 (calculated); LC/MS [M+H] 507.2 (observed).

### Example 13 Synthesis of tert-butyl N-[5-[5-amino-7-(dipropylcarbamoyl)-6H-thieno[3,2-b]azepin-2-yl]pentyl]carbamate, TAZ-13

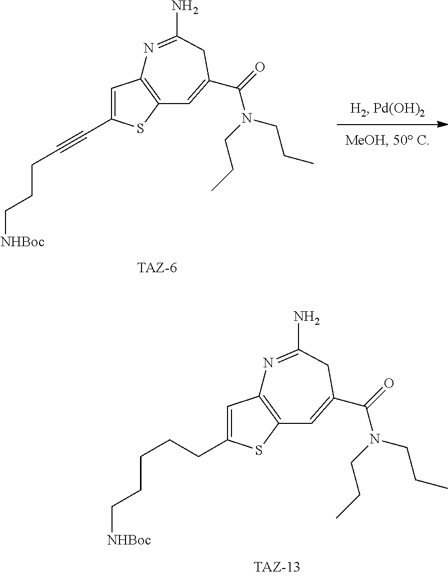 [SMILES: CCCN(CCC)C(=O)C1=Cc2sc(C#CCCCNC(=O)OC(C)(C)C)cc2N=C(N)C1 ; CCCN(CCC)C(=O)C1=Cc2sc(CCCCCNC(=O)OC(C)(C)C)cc2N=C(N)C1]

To a solution of tert-butyl N-[5-[5-amino-7-(dipropylcarbamoyl)-6H-thieno[3,2-b]azepin-2-yl] pent-4-ynyl]carbamate, TAZ-6 (0.8 g, 1.69 mmol, 1.0 eq) in MeOH (30 mL) was added Pd(OH)₂/C (10%, 0.3 g) under N₂. The suspension was degassed under vacuum and purged with H₂ several times. The mixture was stirred under H₂ (50 psi) at 25° C. for 12 hours. The reaction mixture was filtered and concentrated. The residue was purified by flash silica gel chromatography (ISCO®; 20 g SepaFlash® Silica Flash Column, Eluent of 0˜100% Ethyl acetate/Petroleum ether gradient @ 60 mL/min) to give TAZ-13 (0.6 g, 1.26 mmol, 74.37% yield) as yellow oil. ¹H NMR (MeOD, 400 MHz) δ6.80 (s, 1H), 6.62 (s, 1H), 3.43-3.35 (m, 4H), 3.03 (t, J=7.2 Hz, 2H), 2.91 (s, 2H), 2.78 (t, J=7.2 Hz, 2H), 1.69-1.60 (m, 6H), 1.50-1.40 (m, 13H), 0.92-0.87 (m, 6H). LC/MS [M+H] 477.3 (calculated); LC/MS [M+H] 477.3 (observed)

### Example 14 Synthesis of 5-amino-2-(5-aminopentyl)-N,N-dipropyl-6H-thieno [3,2-b]azepine-7-carboxamide, TAZ-14

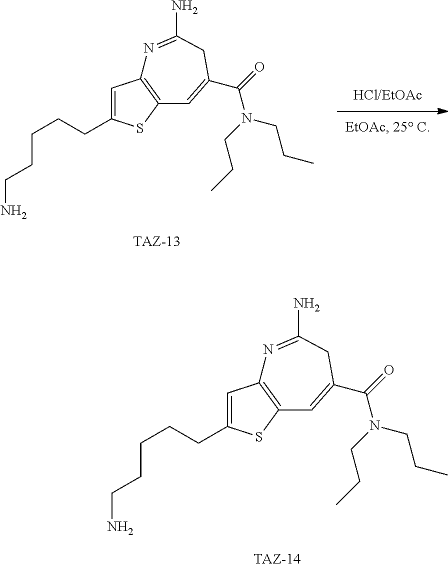 [SMILES: CCCN(CCC)C(=O)C1=Cc2sc(CCCCCN)cc2N=C(N)C1 ; CCCN(CCC)C(=O)C1=Cc2sc(CCCCCN)cc2N=C(N)C1]

To a solution of tert-butyl N-[5-[5-amino-7-(dipropylcarbamoyl)-6H-thieno[3,2-b]azepin-2-yl]pentyl]carbamate, TAZ-13 (0.31 g, 650 μmol, 1.0 eq) in EtOAc (10 mL) was added HCl/EtOAc (4 M, 4.88 mL, 30.0 eq) at 25° C. and then stirred for 1 hour at this temperature. The mixture was concentrated under reduced pressure to give TAZ-14 (0.26 g, 630 μmol, 96.80% yield, HCl) as yellow solid. ¹HNMR (MeOD, 400 MHz) δ7.02 (s, 1H), 6.92 (s, 1H), 3.45-3.43 (m, 4H), 3.35 (s, 2H), 2.98-2.86 (m, 4H), 1.82-1.62 (m, 8H), 1.55-1.45 (m, 2H), 0.92-0.87 (m, 6H). LC/MS [M+H] 377.2 (calculated); LC/MS [M+H] 377.2 (observed).

### Example 15 Synthesis of 5-amino-2-bromo-6H-thieno[3,2-b]azepine-7-carboxylic acid, TAZ-15

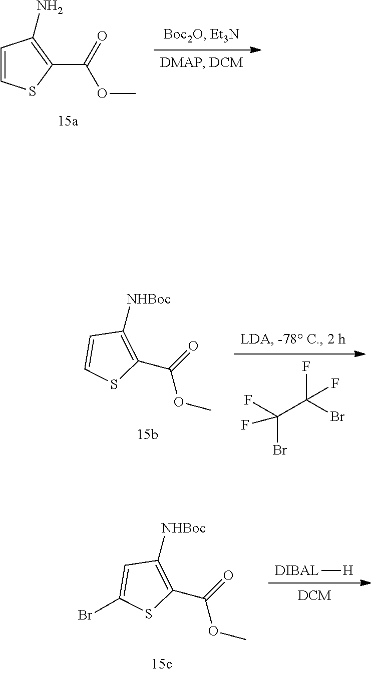

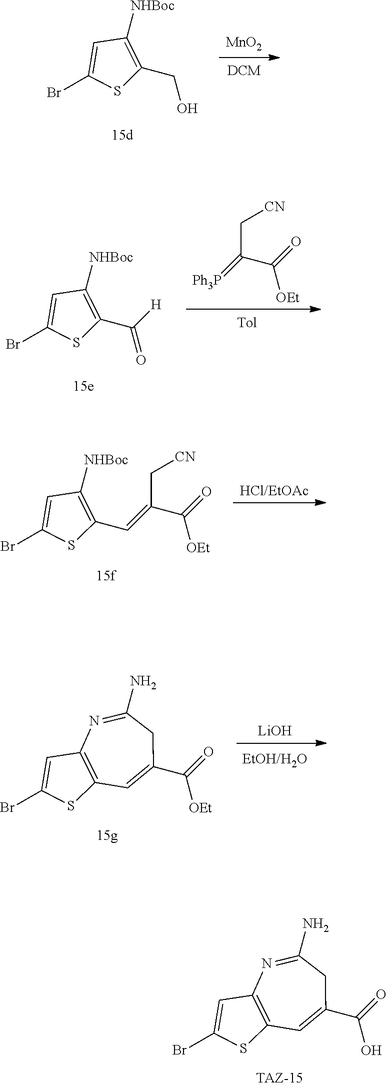

### Preparation of methyl 3-(tert-butoxycarbonylamino)thiophene-2-carboxylate, 15b

To a solution of methyl 3-aminothiophene-2-carboxylate, 15a (19 g, 121 mmol, 1 eq) and Et₃N (14.7 g, 145 mmol, 20.2 mL, 1.2 eq) in DCM (100 mL) was added Boc₂O (29.0 g, 133 mmol, 30.5 mL, 1.1 eq) in DCM (50 mL) dropwise at 25° C., then DMAP (738 mg, 6.0 mmol, 0.05 eq) was added to the mixture. The resulting mixture was stirred at 25° C. for 3 h. The mixture was diluted with water (100 mL) and extracted with DCM (50 mL×3). The organic layer was washed with brine (30 mL), dried over Na₂SO₄, filtered and concentrated. The residue was purified by flash silica gel chromatography (ISCO®; 20 g SepaFlash® Silica Flash Column, Eluent of 0˜20% Ethyl acetate/Petroleum ether gradient @ 45 mL/min) to give 15b (12 g, 46.64 mmol, 38.58% yield) as white solid. ¹H NMR (CDCl₃, 400 MHz) δ9.36 (s, 1H), 7.89 (d, J=5.6 Hz 1H), 7.42 (d, J=5.6 Hz, 1H), 3.88 (s, 3H), 1.53 (s, 9H).

### Preparation of methyl 5-bromo-3-(tert-butoxycarbonylamino)thiophene-2-carboxylate, 15c

To a solution of 15b (8.9 g, 34.6 mmol, 1 eq) in THE (50 mL) was added LDA (2 M, 60.0 mL, 3.5 eq) at −78° C., the mixture was stirred for 1 h at −78° C., then 1,2-dibromo-1,1,2,2-tetrafluoro-ethane (53.92 g, 207.54 mmol, 6 eq) was added and then stirred for 1 h at this temperature. The mixture was poured into cold ammonium chloride solution (100 mL) while stirring vigorously, and extracted with EtOAc (50 mL×3). The organic layer was washed with brine (50 mL), dried over Na₂SO₄, filtered and concentrated. The residue was purified by flash silica gel chromatography (ISCO®; 10 g SepaFlash® Silica Flash Column, Eluent of 0˜20% Ethyl acetate/Petroleum ether gradient @ 45 mL/min) to give 15c (4 g, 11.90 mmol, 34.40% yield) as off-white solid. ¹H NMR (CDCl₃, 400 MHz) δ9.33 (s, 1H), 7.97 (s, 1H), 3.86 (s, 3H), 1.54 (s, 9H)

### Preparation of tert-butyl N-[5-bromo-2-(hydroxymethyl)-3-thienyl]carbamate, 15d

To a solution of 15c (4 g, 11.9 mmol, 1 eq) in DCM (60 mL) was added DIBAL-H (1 M, 59.0 mL, 5 eq) at 0° C. under N₂ and then stirred at 25° C. for 2 h. The reaction mixture was quenched by addition of H₂O 1 mL at 0° C., and then added 15% NaOH (0.5 mL) and H₂O (1 mL) at 0° C. The mixture was stirred for 30 min at 25° C., filtered and the cake was washed with EtOAc (50 mL), the filtrate was concentrated to give 15d (3 g, 9.7 mmol, 81.82% yield) as yellow solid. ¹H NMR (CDCl₃, 400 MHz) δ7.21 (s, 1H), 6.68 (s, 1H), 4.60 (s, 2H), 1.51 (s, 9H).

### Preparation of tert-butyl N-(5-bromo-2-formyl-3-thienyl)carbamate, 15e

To a solution of 15d (3 g, 9.73 mmol, 1 eq) in DCM (30 mL) was added MnO₂ (8.5 g, 97.34 mmol, 10 eq) at 25° C. The mixture was stirred at 50° C. for 12 h. The mixture was filtered and concentrated to give 15e (1.5 g, 4.90 mmol, 50.33% yield) as yellow solid. ¹H NMR (CDCl₃, 400 MHz) δ9.82 (s, 1H), 9.51 (s, 1H), 8.03 (s, 1H), 1.53 (s, 9H).

### Preparation of ethyl (E)-3-[5-bromo-3-(tert-butoxycarbonylamino)-2-thienyl]-2-(cyanomethyl)prop-2-enoate, 15f

To a solution of 15e (1.5 g, 4.90 mmol, 1 eq) in toluene (15 mL) was added ethyl 3-cyano-2-(triphenyl-phosphanylidene)propanoate (2.5 g, 6.4 mmol, 1.3 eq) at 25° C., and then stirred at 75° C. for 2 h. The mixture was concentrated and the residue was purified by flash silica gel chromatography (ISCO®; 2 g SepaFlash® Silica Flash Column, Eluent of 0˜80% Ethyl acetate/Petroleum ether gradient @ 45 mL/min) to give 15f (1.65 g, 3.97 mmol, 81.10% yield) as yellow solid. ¹H NMR (CDCl₃, 400 MHz) δ7.81 (s, 1H), 7.73 (s, 1H), 6.72 (s, 1H), 4.35 (q, J=7.2 Hz, 2H), 3.68 (s, 2H), 1.54 (s, 9H), 1.39 (t, J=7.2 Hz, 3H).

### Preparation of ethyl 5-amino-2-bromo-6H-thieno[3,2-b]azepine-7-carboxylate, 15g

To a solution of 15f (1.3 g, 3.13 mmol, 1 eq) in EtOAc (10 mL) was added HCl/EtOAc (4 M, 13.00 mL, 16.6 eq) at 25° C. The mixture was stirred at 25° C. for 2 h and then concentrated to give 15g (1 g, crude) was obtained as yellow solid. ¹H NMR (DMSO-d₆, 400 MHz) δ10.13 (s, 1H), 9.29 (s, 1H), 7.91 (s, 1H), 7.37 (s, 1H), 4.24 (q, J=7.2 Hz, 2H), 3.52 (s, 2H), 1.28 (t, J=7.2 Hz, 3H).

### Preparation of 5-amino-2-bromo-6H-thieno[3,2-b]azepine-7-carboxylic acid, TAZ-15

To a solution of 15g (1 g, 3.17 mmol, 1 eq) in EtOH (10 mL) and H₂O (1 mL) was added LiOH·H₂O (665 mg, 15.8 mmol, 5 eq) and then stirred at 25° C. for 12 h. The reaction mixture was concentrated under reduced pressure to remove EtOH. The residue was diluted with H₂O (30 mL), and then the pH of mixture was adjusted to 4 by aq HCl (1 M), and extracted with EtOAc (20 mL×3). The organic layer was washed with brine (10 mL), dried over Na₂SO₄, filtered and concentrated to give TAZ-15 (0.85 g, 2.96 mmol, 93.30% yield) as yellow solid. ¹H NMR (DMSO-d₆, 400 MHz) δ7.65 (s, 1H), 7.34 (br s, 2H), 6.97 (s, 1H), 2.97 (s, 2H).

### Example 16 Synthesis of tert-butyl N-[4-[(5-amino-6H-thieno[3,2-b]azepine-7-carbonyl)-propyl-amino]but-2-ynyl]carbamate, TAZ-16

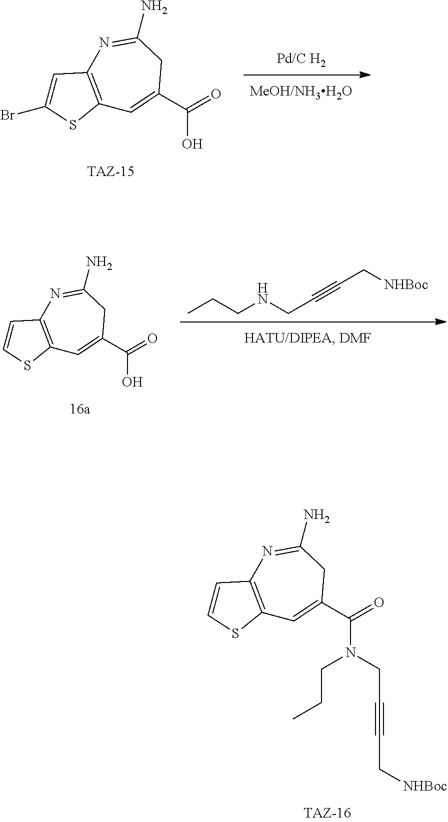

### Preparation of 5-amino-6H-thieno[3,2-b]azepine-7-carboxylic acid, 16a

To a solution of 5-amino-2-bromo-6H-thieno[3,2-b]azepine-7-carboxylic acid, TAZ-15 (1 g, 3.48 mmol, 1 eq) in MeOH (20 mL) was added Pd/C (10%, 0.2 g) and aqueous ammonium hydroxide, NH₃·H₂O (4.88 g, 34.8 mmol, 5.37 mL, 25% purity, 10 eq) under N₂. The suspension was degassed under vacuum and purged with H₂ several times, and then stirred under H₂ (50 psi) at 25° C. for 12 h. The reaction mixture was filtered through Celite® (Johns Manville) and the pH of filtrate was adjusted to˜6 with 2 N HCl at 0° C., and then concentrated under reduced pressure to remove MeOH. The solid was filtered and dried under reduced pressure to give 16a (0.54 g, 2.59 mmol, 74.46% yield) as a light yellow solid. ¹H NMR (DMSO-d₆, 400 MHz) δ7.71 (s, 1H), 7.61 (d, J=5.2 Hz, 1H), 6.98 (br s, 2H), 6.83 (d, J=5.2 Hz, 1H), 2.91 (s, 2H).

### Preparation of TAZ-16

To a solution of 16a (0.33 g, 1.58 mmol, 1 eq) in DMF (4 mL) was added HATU (662.82 mg, 1.74 mmol, 1.1 eq) and DIPEA (1.02 g, 7.92 mmol, 1.38 mL, 5 eq) at 0° C. After 10 min, tert-butyl N-[4-(propylamino)but-2-ynyl]carbamate (394.51 mg, 1.74 mmol, 1.1 eq) was added at 0° C., and then the resulting mixture was stirred at 25° C. for 30 min. The reaction mixture was quenched by addition of H₂O (30 mL) at 0° C., and then extracted with EtOAc(15 mL×3). The combined organic layers were washed with brine (5 mL×3), dried over Na₂SO₄, filtered and concentrated under reduced pressure. The residue was purified by prep-HPLC (TFA condition; column: Phenomenex Luna C18 100*30 mm*5 um; mobile phase: [water(0.1% TFA)−ACN]; B %: 15%-45%, 10 min) to give TAZ-16 (0.185 g, 444.14 μmol, 28.03% yield) as a light yellow solid. ¹H NMR (MeOD, 400 MHz) δ7.74 (d, J=5.6 Hz, 1H), 7.29 (s, 1H), 7.13 (d, J=5.6 Hz, 1H), 4.30 (s, 2H), 3.84 (s, 2H), 3.54-3.52 (m, 2H), 3.38 (s, 2H), 1.76-1.67 (m, 2H), 1.43 (s, 9H), 0.94 (t, J=7.6 Hz, 3H). LC/MS [M+H] 417.2 (calculated); LC/MS [M+H] 417.2 (observed).

### Example 17 Synthesis of 5-amino-N-(4-aminobut-2-ynyl)-N-propyl-6H-thieno[3,2-b]azepine-7-carboxamide, TAZ-17

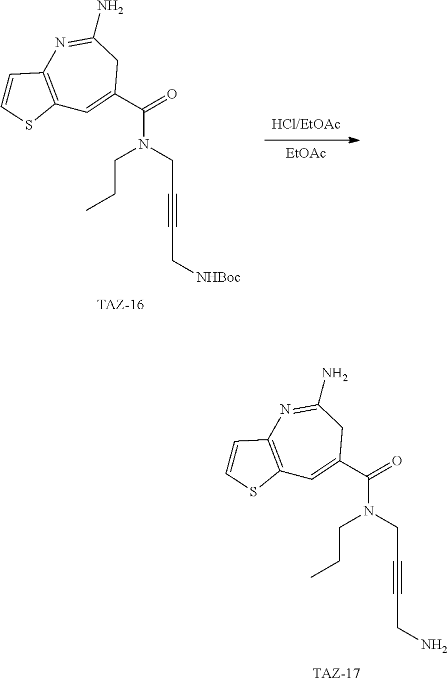 [SMILES: "EtOAc" ; CCCN(CC#CCNC(=O)OC(C)(C)C)C(=O)C1=Cc2sccc2N=C(N)C1 ; CCCN(CC#CCN)C(=O)C1=Cc2sccc2N=C(N)C1]

To a solution of tert-butyl N-[4-[(5-amino-6H-thieno[3,2-b]azepine-7-carbonyl)-propyl-amino]but-2-ynyl]carbamate, TAZ-16 (0.45 g, 1.08 mmol, 1 eq) in EtOAc (2 mL) was added HCl/EtOAc (4 M, 15.00 mL, 55 eq) at 25° C. and then stirred for 0.5 h at this temperature. The reaction mixture was concentrated under reduced pressure to give TAZ-17 (496 mg, crude, HCl) as a light yellow solid. ¹H NMR (MeOD, 400 MHz) δ7.76 (d, J=5.6 Hz, 1H), 7.23 (s, 1H), 7.15 (d, J=5.6 Hz, 1H), 4.40 (s, 2H), 3.87 (s, 2H), 3.57 (t, J=7.2 Hz, 2H), 3.40 (s, 2H), 1.81-1.65 (m, 2H), 0.95 (t, J=7.6 Hz, 3H). LC/MS [M+H] 317.1(calculated); LC/MS [M+H] 317.1 (observed).

### Example 18 Synthesis of 5-amino-2-phenyl-N,N-dipropyl-6H-thieno[3,2-b]azepine-7-carboxamide, TAZ-18

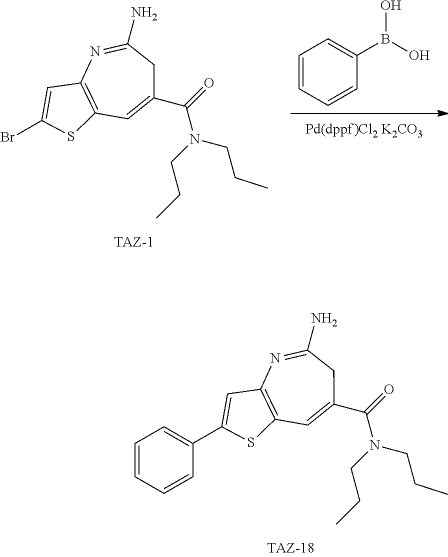 [SMILES: CCCN(CCC)C(=O)C1=Cc2sc(Br)cc2N=C(N)C1 ; OB(O)c1ccccc1 ; CCCN(CCC)C(=O)C1=Cc2sc(-c3ccccc3)cc2N=C(N)C1]

To a mixture of phenylboronic acid (34.5 mg, 283 μmol, 1.5 eq), K₂CO₃ (52.0 mg, 378 μmol, 2.0 eq) and 5-amino-2-bromo-N,N-dipropyl-6H-thieno[3,2-b]azepine-7-carboxamide, TAZ-1 (70.0 mg, 190 μmol, 1.0 eq) in dioxane (2 mL) and H₂O (0.2 mL) was added Pd(dppf)Cl₂ (7.0 mg, 9.45 μmol, 0.05 eq) at 25° C. under N₂ and then stirred at 100° C. for 1 hours. The mixture was filtered and purified by prep-HPLC (column: Nano-micro Kromasil C18 100*30 mm 8 um; mobile phase: [water(0.1% TFA)−ACN]; B %: 25%-50%, 10 min) to afford TAZ-18 (54 mg, 147 μmol, 77.7% yield) as white solid. ¹H NMR (MeOD, 400 MHz) δ7.69 (d, J=7.2 Hz, 2H), 7.49-7.37 (m, 4H), 7.11 (s, 1H), 3.54-3.38 (m, 6H), 1.68 (sxt, J=7.4 Hz, 4H), 0.99-0.92 (m, 6H). LC/MS [M+H] 368.2 (calculated); LC/MS [M+H] 368.1 (observed).

### Example 19 Synthesis of 5-amino-N,N-dipropyl-2-(1-((2-(trimethylsilyl)ethoxy)methyl)-1H-pyrazol-4-yl)-6H-thieno[3,2-b]azepine-7-carboxamide, TAZ-19

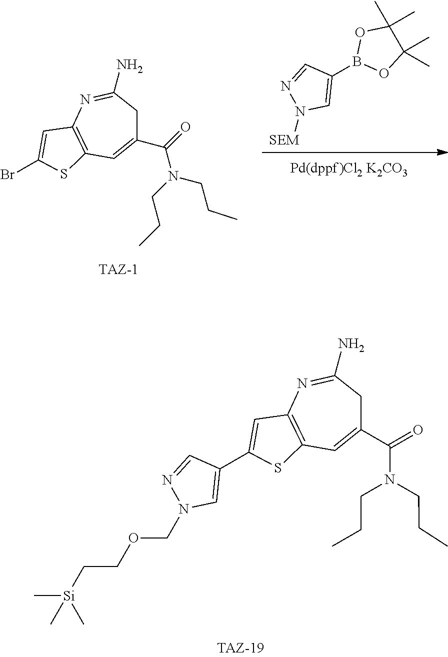 [SMILES: CCCN(CCC)C(=O)C1=Cc2sc(Br)cc2N=C(N)C1 ; CC1(C)OB(c2cnn(COCC[Si](C)(C)C)c2)OC1(C)C ; CCCN(CCC)C(=O)C1=Cc2sc(-c3cnn(COCC[Si](C)(C)C)c3)cc2N=C(N)C1]

To a mixture of 5-amino-2-bromo-N,N-dipropyl-6H-thieno[3,2-b]azepine-7-carboxamide, TAZ-1 (100 mg, 270 μmol, 1.0 eq), K₂CO₃ (75.0 mg, 540 μmol, 2.0 eq) and trimethyl-[2-[[4-(4,4,5,5-tetramethyl-1,3,2-dioxaborolan-2-yl)pyrazol-1-yl]methoxy]ethyl]silane (96 mg, 297 μmol, 1.1 eq) in dioxane (3 mL) and H₂O (0.2 mL) was added Pd(dppf)Cl₂ (9.88 mg, 13.50 μmol, 0.05 eq) at 25° C. under N₂ and then stirred at 95° C. for 1 hours. The mixture was filtered and concentrated in vacuum. The residue was purified by prep-HPLC (column: Phenomenex Gemini-NX C18 75*30 mm*3 um; mobile phase: [water(10 mM NH₄HCO₃)-ACN]; B %: 40%-60%, 10.5 min) to afford TAZ-19 (80 mg, 164 μmol, 60.7% yield) as light yellow solid. ¹H NMR (MeOD, 400 MHz) δ8.09 (s, 1H), 7.80 (s, 1H), 6.97 (s, 1H), 6.85 (s, 1H), 5.45 (s, 2H), 3.61 (t, J=8.0 Hz, 2H), 3.46-3.36 (m, 4H), 2.96 (s, 2H), 1.72-1.57 (m, 4H), 0.90 (t, J=8.0 Hz, 8H), 0.00 (s, 9H). LC/MS [M+H] 488.2 (calculated); LC/MS [M+H] 488.2 (observed).

### Example 20 Synthesis of methyl 5-amino-7-(dipropylcarbamoyl)-6H-thieno[3,2-b]azepine-2-carboxylate, TAZ-20

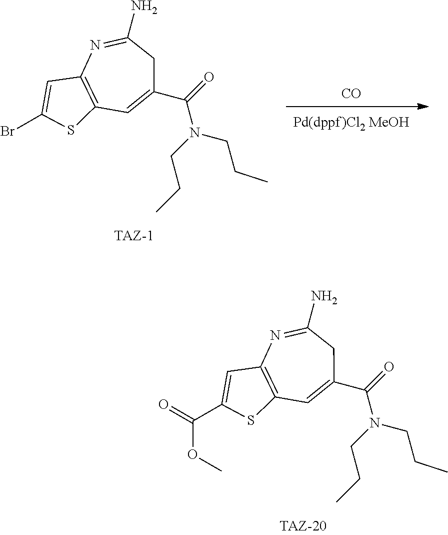 [SMILES: CCCN(CCC)C(=O)C1=Cc2sc(Br)cc2N=C(N)C1 ; "CO" ; CCCN(CCC)C(=O)C1=Cc2sc(C(=O)OC)cc2N=C(N)C1]

To a solution of 5-amino-2-bromo-N,N-dipropyl-6H-thieno[3,2-b]azepine-7-carboxamide, TAZ-1 (1.2 g, 3.24 mmol, 1.0 eq) and Et₃N (984 mg, 9.72 mmol, 1.35 mL, 3 eq) in MeOH (20 mL) was added Pd(dppf)Cl₂ (118.56 mg, 162.03 μmol, 0.05 eq) under N₂. The suspension was degassed under vacuum and purged with CO several times. The mixture was stirred under CO (50 psi) at 80° C. for 12 hours. The mixture was filtered and concentrated in vacuum to afford TAZ-20 (1.1 g, 3.15 mmol, 97.14% yield) as white solid. ¹H NMR (MeOD, 400 MHz) δ7.72 (s, 1H), 7.14 (s, 1H), 3.92 (s, 3H), 3.58-3.37 (m, 6H), 1.69-1.62 (m, 4H), 0.99-0.90 (m, 6H). LC/MS [M+H] 350.2 (calculated); LC/MS [M+H] 350.2 (observed).

### Example 21 Synthesis of 5-amino-N,N-dipropyl-2-(1H-pyrazol-4-yl)-6H-thieno[3,2-b]azepine-7-carboxamide, TAZ-21

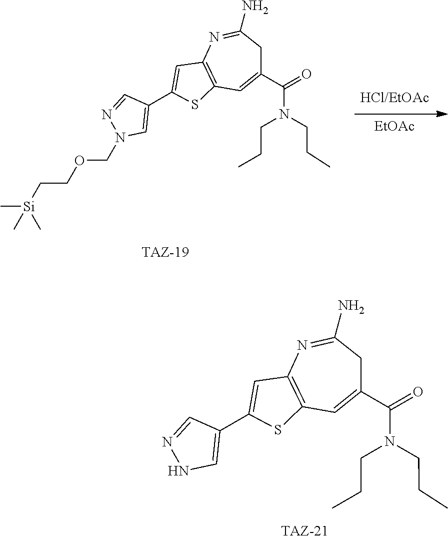 [SMILES: "EtOAc" ; CCCN(CCC)C(=O)C1=Cc2sc(-c3cnn(COCC[Si](C)(C)C)c3)cc2N=C(N)C1 ; CCCN(CCC)C(=O)C1=Cc2sc(-c3cn[nH]c3)cc2N=C(N)C1]

To a mixture of 5-amino-N,N-dipropyl-2-[1-(2-trimethylsilylethoxymethyl) pyrazol-4-yl]-6H-thieno[3,2-b]azepine-7-carboxamide, TAZ-19 (66.0 mg, 135 μmol, 1.0 eq) in MeOH (4 mL) was added HCl/MeOH (4 M, 338 μL, 10.0 eq) at 25° C. and then stirred for 12 hours at this temperature. The mixture was concentrated in vacuum, and the residue was purified by prep-HPLC (column: Phenomenex Gemini-NX C18 75*30 mm*3 um; mobile phase: [water(10 mM NH₄HCO₃)−ACN]; B %: 20%-50%, 10.5 min) to afford TAZ-21 (22 mg, 61.5 μmol, 45.5% yield) as white solid. ¹H NMR (MeOD, 400 MHz) δ7.87 (s, 2H), 6.95 (s, 1H), 6.85 (s, 1H), 3.44-3.35 (m, 4H), 2.96 (s, 2H), 1.66-1.60 (m, 4H), 0.99-0.89 (m, 6H). LC/MS [M+H] 358.2 (calculated); LC/MS [M+H] 358.2 (observed).

### Example 22 Synthesis of 5-amino-7-(dipropylcarbamoyl)-6H-thieno[3,2-b]azepine-2-carboxylic acid, TAZ-22

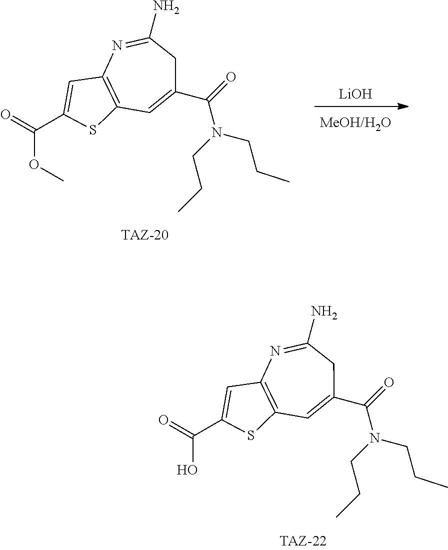 [SMILES: CCCN(CCC)C(=O)C1=Cc2sc(C(=O)OC)cc2N=C(N)C1 ; "LiOH" ; CCCN(CCC)C(=O)C1=Cc2sc(C(=O)O)cc2N=C(N)C1]

To a mixture of methyl 5-amino-7-(dipropylcarbamoyl)-6H-thieno[3,2-b]azepine-2-carboxylate, TAZ-20 (1.1 g, 3.15 mmol, 1.0 eq) in MeOH (20 mL) was added LiOH H₂O (396 mg, 9.44 mmol, 3.0 eq) in H₂O (5 mL) at 25° C. and then stirred for 2 hours at this temperature. The mixture was quenched with aq HCl (4 M) until pH to 5, off-white solid precipitated from the mixture and then filtered to give TAZ-22 (0.85 g, 2.53 mmol, 80.5% yield) as off white solid. ¹H NMR (MeOD, 400 MHz) δ7.41 (s, 1H), 6.92 (s, 1H), 3.37-3.25 (m, 4H), 3.05 (s, 2H), 1.61-1.45 (m, 4H), 0.86-0.75 (m, 6H). LC/MS [M+H] 336.1 (calculated); LC/MS [M+H] 336.1 (observed).

### Example 23 Synthesis of 5-amino-N2-phenyl-N7,N7-dipropyl-6H-thieno [3,2-b]azepine-2,7-dicarboxamide, TAZ-23

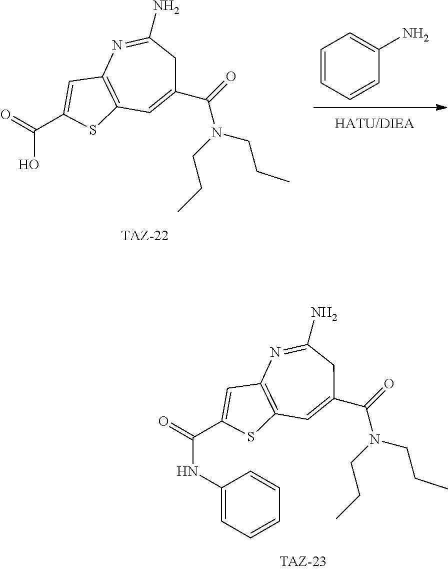 [SMILES: CCCN(CCC)C(=O)C1=Cc2sc(C(=O)O)cc2N=C(N)C1 ; Nc1ccccc1 ; CCCN(CCC)C(=O)C1=Cc2sc(C(=O)Nc3ccccc3)cc2N=C(N)C1]

To a mixture of 5-amino-7-(dipropylcarbamoyl)-6H-thieno[3,2-b]azepine-2-carboxylic acid, TAZ-22 (50 mg, 150 μmol, 1.0 eq) HATU (62 mg, 164 μmol, 1.1 eq) and DIEA (58 mg, 447 μmol, 3.0 eq) in DMF (1 mL) was added aniline (28 mg, 298 μmol, 2.0 eq) at 25° C. and then stirred at 25° C. for 30 min. The mixture was filtered and purified by prep-HPLC (column: Nano-micro Kromasil C18 100*30 mm 8 um; mobile phase: [water(0.1% TFA)−ACN]; B %: 10%-40%, 10 min) to give TAZ-23 (34 mg, 82.8 μmol, 55.6% yield) as white solid. ¹H NMR (MeOD, 400 MHz) δ7.87 (s, 1H), 7.67 (d, J=8.0 Hz, 2H), 7.38 (t, J=8.0 Hz, 2H), 7.18 (d, J=8.0 Hz, 1H), 7.15 (s, 1H), 3.49-3.42 (m, 6H), 1.70-1.65 (m, 4H), 0.98-0.90 (m, 6H). LC/MS [M+H] 411.2 (calculated); LC/MS [M+H] 411.1 (observed).

### Example 24 Synthesis of 5-amino-N2-ethyl-N7,N7-dipropyl-6H-thieno[3,2-b]azepine-2,7-dicarboxamide, TAZ-24

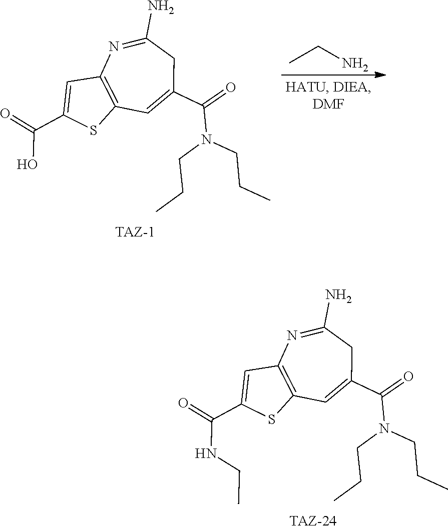

To a mixture of 5-amino-7-(dipropylcarbamoyl)-6H-thieno[3,2-b]azepine-2-carboxylic acid, TAZ-1 (50 mg, 149 μmol, 1.0 eq), HATU (62.4 mg, 164 μmol, 1.1 eq) and DIEA (58 mg, 447 μmol, 3.0 eq) in DMF (1 mL) was added ethanamine (20 mg, 298 μmol, 2.0 eq) at 25° C. and then stirred for 0.5 hours at this temperature. The mixture was filtered and purified by prep-HPLC (column: Nano-micro Kromasil C18 100*30 mm 8 um; mobile phase: [water(0.1% TFA)-ACN]; B %: 5%-35%, 10 min). Afforded TAZ-24 (28 mg, 77.2 μmol, 51.8% yield) as white solid. ¹H NMR (MeOD, 400 MHz) δ7.62 (s, 1H), 7.13 (s, 2H), 3.54-3.35 (m, 8H), 1.73-1.61 (m, 4H), 1.23 (t, J=7.2 Hz, 3H), 0.97-0.90 (m, 6H). LC/MS [M+H] 363.2 (calculated); LC/MS [M+H] 363.2 (observed).

### Example 25 Synthesis of 3-[2-[2-[2-[2-[2-[2-[2-[2-[2-[2-[5-[5-amino-7-(dipropylcarbamoyl)-6H-thieno[3,2-b]azepin-2-yl]pentyl-methyl-amino]ethoxy]ethoxy]ethoxy]ethoxy]ethoxy]ethoxy]ethoxy]ethoxy]ethoxy]ethoxy]propanoic acid, TAZ-25

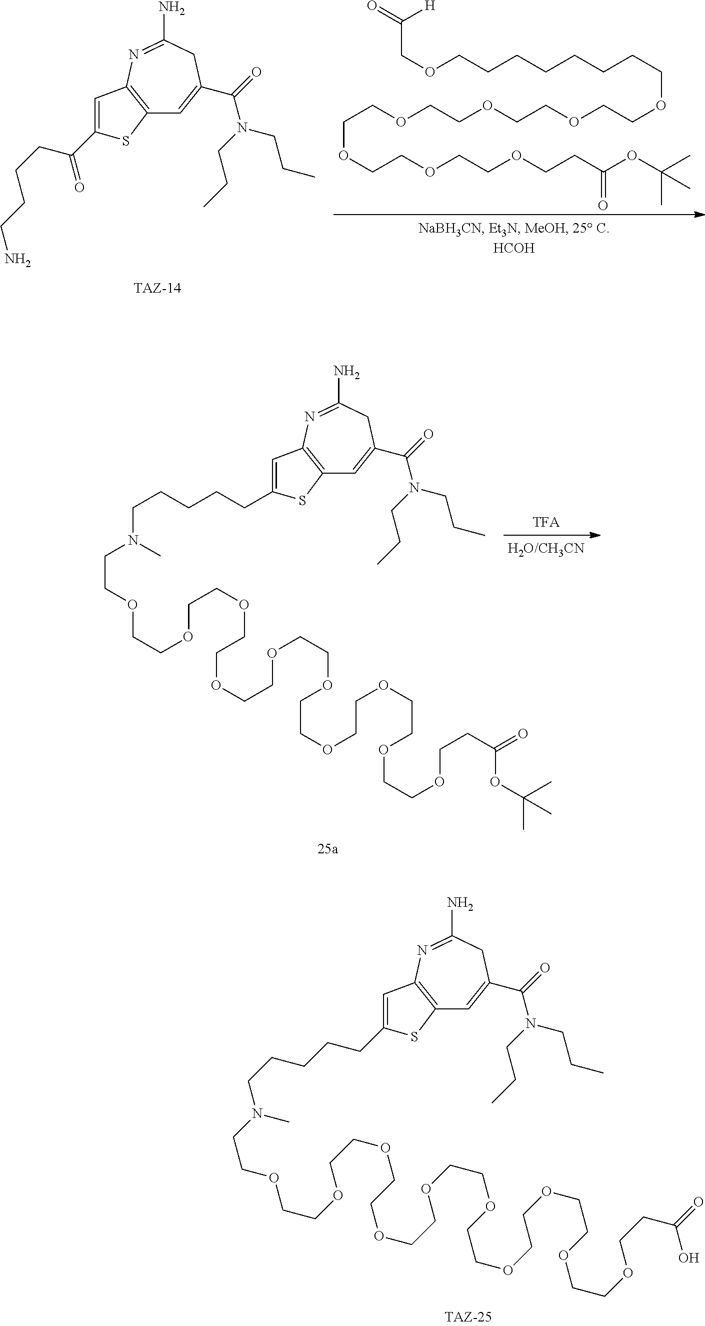

### Preparation of tert-butyl 3-[2-[2-[2-[2-[2-[2-[2-[2-[2-[2-[5-[5-amino-7-(dipropylcarbamoyl)-6H-thieno[3,2-b]azepin-2-yl]pentyl-methyl-amino]ethoxy]ethoxy]ethoxy]ethoxy]ethoxy]ethoxy]ethoxy]ethoxy]ethoxy]ethoxy]propanoate, 25a

To a solution of 5-amino-2-(5-aminopentyl)-N,N-dipropyl-6H-thieno[3,2-b]azepine-7-carboxamide, TAZ-14 (0.2 g, 484 μmol, 1.0 eq, HCl) in MeOH (80 mL) was added Et₃N (73.5 mg, 726 μmol, 101 μL, 1.5 eq) and tert-butyl 3-[2-[2-[2-[2-[2-[2-[2-[2-[2-(2-oxoethoxy) ethoxy]ethoxy]ethoxy]ethoxy]ethoxy]ethoxy]ethoxy]ethoxy]ethoxy]propanoate (368.1 mg, 629 μmol, 1.3 eq) at 25° C. The mixture was stirred at 25° C. for 10 min, then NaBH₃CN (60.8 mg, 968 μmol, 2.0 eq) was added and it was stirred at the same temperature for 16 hours. Formaldehyde (117.9 mg, 1.45 mmol, 108 μL, 3.0 eq) was added followed by NaBH₃CN (60.9 mg, 968 μmol, 2.0 eq) and then stirred at 25° C. for 2 hours. The mixture was concentrated and the residue was purified by prep-HPLC (column: Xtimate C18 100*30 mm*3 um; mobile phase: [water (0.1% TFA)−ACN]; B %: 15%-50%, 10 min) to give 25a (0.4 g, 416.98 μmol, 86.11% yield) as colorless oil.

### Preparation of TAZ-25

To a solution of 25a (0.39 g, 406 μmol, 1.0 eq) in H₂O (20 mL) was added TFA (927 mg, 8.13 mmol, 602 μL, 20 eq) at 25° C. The mixture was stirred at 85° C. for 1 hour and then concentrated under reduced pressure at 50° C. The residue purified by prep-HPLC (column: Nano-micro Kromasil C18 100*30 mm 8 um; mobile phase: [water (0.1% TFA)−ACN]; B %: 10%-30%, 15 min) to afford TAZ-25 (0.18 g, 199.30 μmol, 49.02% yield) as light yellow oil. ¹H NMR (MeOD, 400 MHz) δ7.02 (s, 1H), 6.90 (s, 1H), 3.84-3.82 (m, 2H), 3.75-3.59 (m, 41H), 3.45-3.42 (m, 4H), 3.35 (s, 2H), 2.96-2.87 (m, 5H), 2.54 (t, J=6.4 Hz, 2H), 1.84-1.76 (m, 4H), 1.71-1.60 (m, 4H), 1.54-1.44 (m, 2H), 0.94-0.89 (m, 6H). LC/MS [M+H] 903.5 (calculated); LC/MS [M+H] 903.5 (observed).

### Example 26 Synthesis of 5-amino-2-benzyl-N,N-dipropyl-6H-thieno[3,2-b] azepine-7-carboxamide, TAZ-26

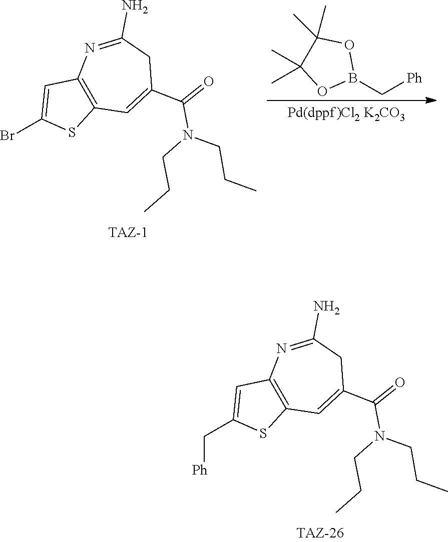 [SMILES: CCCN(CCC)C(=O)C1=Cc2sc(Br)cc2N=C(N)C1 ; CC1(C)OB(Cc2ccccc2)OC1(C)C ; CCCN(CCC)C(=O)C1=Cc2sc(Cc3ccccc3)cc2N=C(N)C1]

To a mixture of 5-amino-2-bromo-N,N-dipropyl-6H-thieno[3,2-b]azepine-7-carboxamide, TAZ-1 (0.11 g, 297 μmol, 1.0 eq), K₂CO₃ (82 mg, 594 μmol, 2 eq) and 2-benzyl-4,4,5,5-tetramethyl-1,3,2-dioxaborolane (324 mg, 1.49 mmol, 5.0 eq) in DMF (3 mL) and H₂O (0.2 mL) was added Pd(dppf)Cl₂ (11 mg, 14.8 μmol, 0.05 eq) at 25° C. under N₂. The mixture was stirred at 120° C. for 2 hours and then filtered and concentrated. The residue was purified by prep-HPLC (column: Xtimate C18 100*30 mm*3 um; mobile phase: [water(0.1% TFA)-ACN]; B %: 25%-45%, 10 min) to give TAZ-26 (16 mg, 41.9 μmol, 14.1% yield) as light yellow solid. ¹H NMR (MeOD, 400 MHz) δ7.40-7.21 (m, 5H), 6.98 (s, 1H), 6.88 (s, 1H), 4.17 (s, 2H), 3.41 (t, J=7.6 Hz, 4H), 3.34 (s, 2H), 1.66-1.62 (m, 4H), 0.96-0.86 (m, 6H). LC/MS [M+H] 382.2 (calculated); LC/MS [M+H] 382.1 (observed).

### Example 27 Synthesis of 5-amino-N7,N7-dipropyl-N2-pyrimidin-5-yl-6H-thieno[3,2-b]azepine-2,7-dicarboxamide, TAZ-27

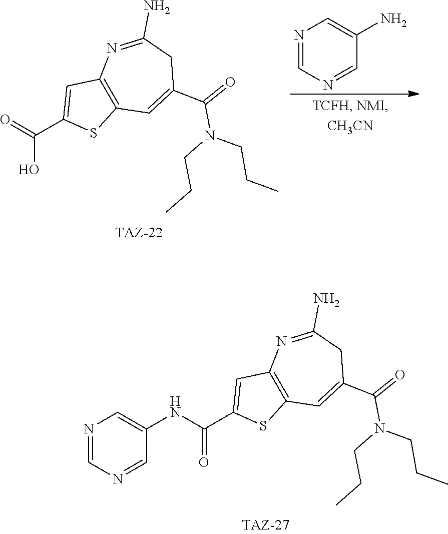 [SMILES: CCCN(CCC)C(=O)C1=Cc2sc(C(=O)O)cc2N=C(N)C1 ; Nc1cncnc1 ; CCCN(CCC)C(=O)C1=Cc2sc(C(=O)Nc3cncnc3)cc2N=C(N)C1]

To a mixture of 5-amino-7-(dipropylcarbamoyl)-6H-thieno[3,2-b]azepine-2-carboxylic acid, TAZ-22 (50 mg, 149 μmol, 1 eq), pyrimidin-5-amine (18.4 mg, 194 μmol, 1.3 eq) and 1-methylimidazole (42.8 mg, 522 μmol, 3.5 eq) in CH₃CN (2 mL) was added chloro-N,N,N′,N′-tetramethylformamidinium hexafluorophosphate, TCFH (50.19 mg, 179 μmol, 1.2 eq) at 25° C., and then stirred for 16 h at this temperature. The reaction mixture was quenched by addition of H₂O (10 mL) at 0° C., and then extracted with EtOAc (10 mL×3). The combined organic layers were washed with brine (5 mL), dried over Na₂SO₄, filtered and concentrated under reduced pressure. The residue was purified by prep-HPLC (TFA condition; column: Phenomenex Luna C18 100*30 mm*5 um; mobile phase: [water(0.1% TFA)−ACN]; B %: 10%-40%, 10 min) to give TAZ-27 (13 mg, 31.5 μmol, 21.14% yield) as off-white solid. ¹H NMR (MeOD, 400 MHz) δ9.17 (s, 2H), 8.94 (s, 1H), 7.90 (s, 1H), 7.18 (s, 1H), 3.54-3.40 (m, 6H), 1.76-1.59 (m, 4H), 0.99-0.90 (m, 6H). LC/MS [M+H] 413.2 (calculated); LC/MS [M+H] 413.1 (observed).

### Example 28 Synthesis of 2-amino-N,N-dipropyl-3H-benzo[4,5]thieno[3,2-b]azepine-4-carboxamide, TAZ-28

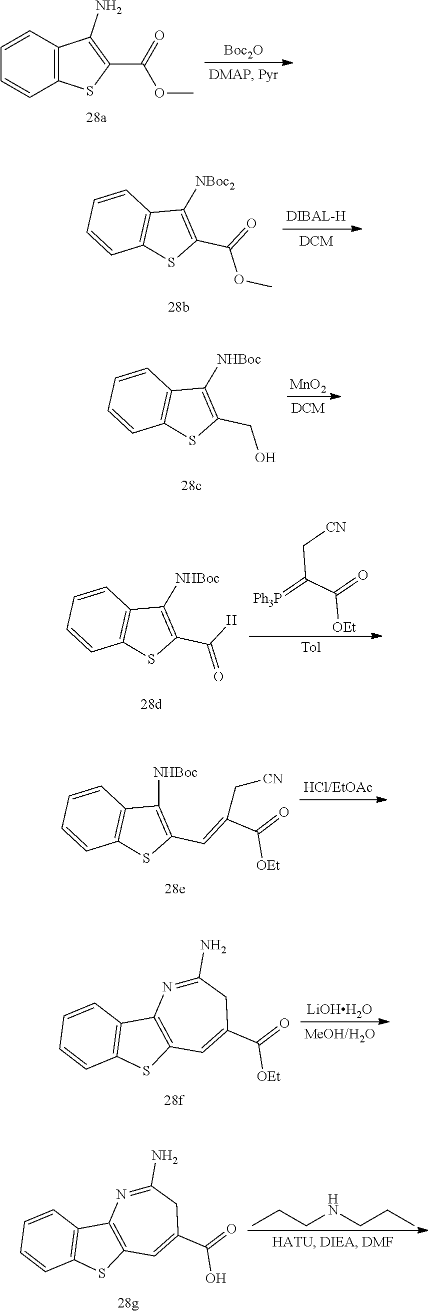

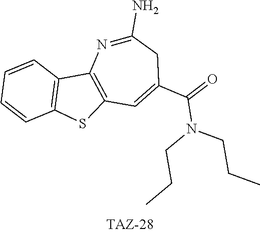 [SMILES: CCCN(CCC)C(=O)C1=Cc2sc3ccccc3c2N=C(N)C1]

### Preparation of methyl 3-[bis(tert-butoxycarbonyl)amino]benzothiophene-2-carboxylate, 28b

To a mixture of methyl 3-aminobenzothiophene-2-carboxylate, 28a (3 g, 14.5 mmol, 1.0 eq) in pyridine, Pyr (30 mL) was added DMAP (177 mg, 1.45 mmol, 0.1 eq). Then a solution of Boc₂O (6.32 g, 29.0 mmol, 6.65 mL, 2.0 eq) in pyridine (10 mL) was added to the mixture slowly at 0° C. and then stirred at 25° C. for 16 h. The mixture was concentrated in vacuum. The residue was dissolved in EtOAc(20 ml) and washed successively with aqueous sat. NaHCO₃ and brine. The mixture was dried over Na₂SO₄, filtered and concentrated. The residue was purified by silica gel chromatography (Petroleum ether/Ethyl acetate=1/0, 5/1) to afford methyl 3-[bis(tert-butoxycarbonyl)amino]benzothiophene-2-carboxylate (5.6 g, 13.7 mmol, 94.94% yield) as yellow solid. ¹H NMR (CDCl₃, 400 MHz) δ7.83 (d, J=7.6 Hz, 1H), 7.68 (d, J=7.6 Hz, 1H), 7.52-7.42 (m, 2H), 3.94 (s, 3H), 1.35 (s, 18H).

### Preparation of tert-butyl N-[2-(hydroxymethyl)benzothiophen-3-yl]carbamate, 28c

To a mixture of 28b (3.8 g, 9.33 mmol, 1.0 eq) in DCM (40 mL) was added DIBAL-H (1 M, 37.3 mL, 4.0 eq) slowly at 0° C. under N₂ and then stirred at the same temperature for 2h. The reaction was quenched with water (2 mL) and dried over Na₂SO₄, filtered and concentrated. The residue was purified by silica gel chromatography (Petroleum ether/Ethyl acetate=1/0, 3/1) to afford 28c (2.3 g, 8.23 mmol, 88.29% yield) as yellow solid. ¹H NMR (CDCl₃, 400 MHz) δ7.83-7.78 (m, 1H), 7.63 (dd, J=2.0, 6.8 Hz, 1H), 7.43-7.35 (m, 2H), 4.75 (d, J=6.4 Hz, 2H), 1.55 (s, 9H).

### Preparation of tert-butyl N-(2-formylbenzothiophen-3-yl)carbamate, 28d

To a solution of 28c (1.8 g, 6.44 mmol, 1.0 eq) in DCM (30 mL) was added MnO₂ (4.48 g, 51.6 mmol, 8.0 eq) in one portion at 25° C. and then stirred for 12 h. The reaction was filtered and concentrated. The residue was purified by column chromatography (SiO2, Petroleum ether/Ethyl acetate=1/0 to 5/1) to afford 28d (1.2 g, 4.33 mmol, 67.15% yield) as yellow solid. ¹H NMR (CDCl₃, 400 MHz) δ10.07 (s, 1H), 8.22 (s, 1H), 8.10 (d, J=8.0 Hz, 1H), 7.83 (d, J=8.0 Hz, 1H), 7.55-7.50 (m, 1H), 7.47-7.43 (m, 1H), 1.57 (s, 9H).

### Preparation of ethyl (E)-3-[3-(tert-butoxycarbonylamino)benzothiophen-2-yl]-2-(cyanomethyl)prop-2-enoate, 28e

To a solution of 28d (0.6 g, 2.16 mmol, 1.0 eq) and ethyl 3-cyano-2-(triphenyl-phosphanylidene)propanoate (1.09 g, 2.81 mmol, 1.3 eq) in toluene (15 mL) at 25° C. and it was stirred at 80° C. for 12h. Then the mixture was concentrated. The residue was diluted with water and extracted with EtOAc(30 mL×3). The organic layer was washed with brine, dried over Na₂SO₄, filtered and concentrated. The residue was purified by silica gel chromatography (Petroleum ether/Ethyl acetate=1/0, 5/1) to afford 28e (0.6 g, 1.55 mmol, 71.88% yield) as yellow solid. ¹H NMR (DMSO, 400 MHz) δ8.08 (d, J=7.2 Hz, 1H), 8.00 (s, 1H), 7.78 (d, J=7.2 Hz, 1H), 7.56-7.47 (m, 2H), 4.28 (q, J=7.2 Hz, 2H), 3.90 (s, 2H), 1.47 (s, 9H), 1.29 (t, J=7.2 Hz, 3H).

### Preparation of ethyl 2-amino-3H-benzothiopheno[3,2-b]azepine-4-carboxylate, 28f

To a solution of 28e (0.2 g, 518 μmol, 1.0 eq) in EtOAc (2 mL) was added HCl/EtOAc (10 mL) in one portion at 25° C. and it was stirred at 50° C. for 12h. The mixture was concentrated to give 28f (0.25 g, crude) as green solid.

### Preparation of 2-amino-3H-benzothiopheno[3,2-b]azepine-4-carboxylic acid, 28g

To a mixture of 28f (0.25 g, 774 μmol, 1.0 eq) in MeOH (6 mL) was added a solution of LiOH·H₂O (162 mg, 3.87 mmol, 5.0 eq) in H₂O (1 mL) at 25° C. The mixture was stirred at 50° C. for 12h. The mixture was quenched with aq. HCl (1M) until pH to 5, and then concentrated to remove MeOH. The desired solid precipitated from the mixture and then filtered to obtain 28g (0.15 g, crude) as yellow solid

### Preparation of 2-amino-N,N-dipropyl-3H-benzo[4,5]thieno[3,2-b]azepine-4-carboxamide, TAZ-28

To a mixture of 28 gm (0.05 g, 194 μmol, 1.0 eq) in DMF (2 mL) was added HATU (88.3 mg, 232 μmol, 1.2 eq) and DIEA (125 mg, 968 μmol, 169 μL, 5.0 eq) and it was stirred at 25° C. for 2 min. N-propylpropan-1-amine (25.5 mg, 252 μmol, 34.7 μL, 1.3 eq) was added to the mixture and then stirred for 1 h. The reaction mixture was filtered and purified by prep-HPLC (column: Nano-micro Kromasil C18 100*30 mm 8 um; mobile phase: [water (0.1% TFA)−ACN]; B %: 15%-45%, 10 min) to give TAZ-28 (19 mg, 55.64 μmol, 28.74% yield) as white solid. ¹H NMR (MeOD, 400 MHz) δ8.00-7.92 (m, 2H), 7.58-7.53 (m, 2H), 7.18 (s, 1H), 3.48 (s, 6H), 1.74-1.65 (m, 4H), 0.94 (s, 6H). LC/MS [M+H] 342.2 (calculated); LC/MS [M+H] 342.2 (observed).

### Example 29 Synthesis of tert-butyl (4-((5-amino-N-propyl-6H-thieno[3,2-b]azepine-7-carboxamido)methyl)benzyl)carbamate, TAZ-29

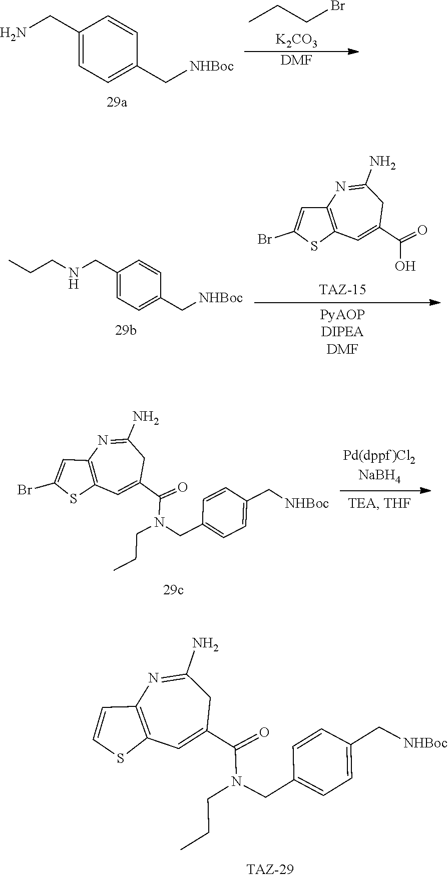

### Preparation of tert-butyl (4-((propylamino)methyl)benzyl)carbamate, 29b

tert-Butyl (4-(aminomethyl)benzyl)carbamate, 29a (0.98 g, 4.15 mmol, 1 eq.) was dissolved in 10 ml DMF. Potassium carbonate (2.9 g, 20.7 mmol, 5 eq.) was added, followed by propyl bromide (0.38 ml, 4.15 mmol, 1 eq.). The reaction mixture was stirred for 2 hours, then filtered, concentrated, and purified by reverse-phase chromatography to give 29b (0.36 g, 1.29 mmol, 31%). LC/MS [M+H] 279.21 (calculated); LC/MS [M+H] 279.24 (observed).

### Preparation of tert-butyl (4-((5-amino-2-bromo-N-propyl-6H-thieno[3,2-b]azepine-7-carboxamido)methyl)benzyl)carbamate, 29c

5-amino-2-bromo-6H-thieno[3,2-b]azepine-7-carboxylic acid, TAZ-15 (0.330 g, 1.15 mmol, 1 eq.) and 29b (0.32 g, 1.15 mmol, 1 eq.) were suspended in 5 ml DMF. DIPEA (1.2 ml, 6.9 mmol, 6 eq.) was added, followed by 7-aza-benzotriazol-1-yloxy-tripyrrolidino-phosphonium hexafluorophosphate, PyAOP (0.90 g, 1.72 mmol, 1.5 eq.). The reaction was monitored by LCMS. Upon consumption of starting material, the reaction mixture was added to 100 ml water, filtered, and the precipitate purified by flash chromatography (MeOH/DCM with 1% TEA) to give 29c (0.35 g, 0.64 mmol, 56%). LC/MS [M+H] 547.14/549.14 (calculated); LC/MS [M+H] 547.40/549.35 (observed).

### Preparation of TAZ-29

Intermediate 29c (0.35 g, 0.64 mmol, 1 eq.) was dissolved in 5 ml THF. Triethylamine (0.89 ml, 6.4 mmol, 10 eq.) and [1,1-bis(diphenylphosphino)ferrocene]palladium(II) dichloride, Pd(dppf)Cl₂ (0.023 g, 0.032 mmol, 0.05 eq.) were added, followed by sodium borohydride (0.12 g, 3.2 mmol, 5 eq.). After 2 hours, another portion of sodium borohydride was added (0.073 g, 1.9 mmol, 3 eq.) and the reaction stirred for 30 minutes. The reaction mixture was concentrated and purified by HPLC to give TAZ-29 (0.129 g, 0.28 mmol, 43%). LC/MS [M+H] 469.23 (calculated); LC/MS [M+H] 469.42 (observed).

### Example 30 Synthesis of 5-amino-N-(4-(aminomethyl)benzyl)-N-propyl-6H-thieno[3,2-b]azepine-7-carboxamide, TAZ-30

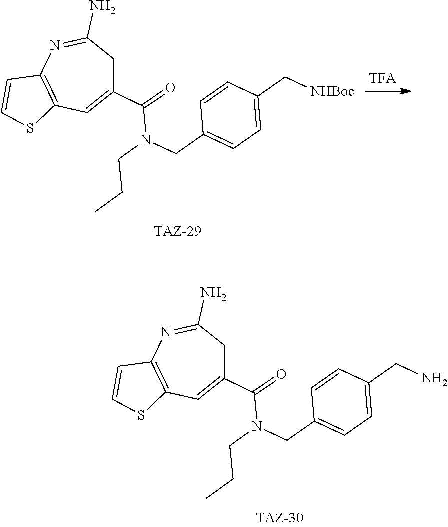 [SMILES: CCCN(Cc1ccc(CNC(=O)OC(C)(C)C)cc1)C(=O)C1=Cc2sccc2N=C(N)C1 ; CCCN(Cc1ccc(CN)cc1)C(=O)C1=Cc2sccc2N=C(N)C1]

tert-Butyl (4-((5-amino-N-propyl-6H-thieno[3,2-b]azepine-7-carboxamido)methyl)benzyl)carbamate, TAZ-29 (0.129 g, 0.28 mmol, 1 eq.) was dissolved in 100 μl TFA. After 15 minutes, the product was concentrated and purified by HPLC to give TAZ-30 (0.063 g, 0.17 mmol, 61%). LC/MS [M+H] 547.14/549.14 (calculated); LC/MS [M+H] 547.40/549.35 (observed).

### Example 52 Synthesis of 5-amino-N-[[4-(aminomethyl)-2-(trifluoromethyl)phenyl]methyl]-N-propyl-6H-thieno[3,2-b]azepine-7-carboxamide, TAZ-52

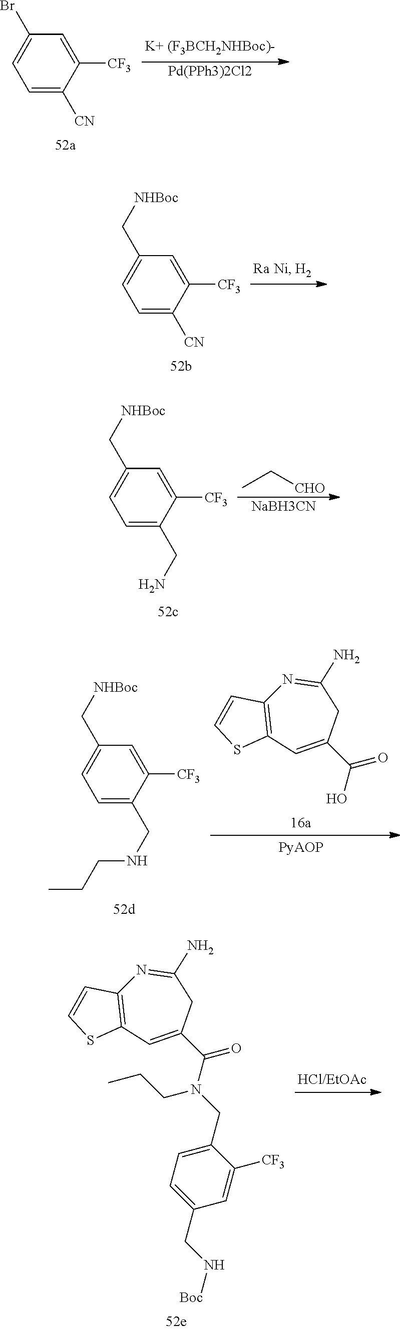

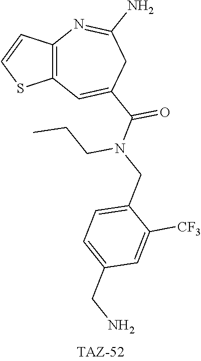 [SMILES: CCCN(Cc1ccc(CN)cc1C(F)(F)F)C(=O)C1=Cc2sccc2N=C(N)C1]

### Preparation of tert-butyl N-[[4-cyano-3-(trifluoromethyl)phenyl]methyl]carbamate, 52b

To a mixture of 4-bromo-2-(trifluoromethyl)benzonitrile, 52a (0.5 g, 2.00 mmol, 1.0 eq), potassium; (tert-butoxycarbonylamino)methyl-trifluoro-boranuide, also known as potassium (((tert-butoxycarbonyl)amino)methyl)trifluoroborane, CAS Reg. No. 1314538-55-0 (711 mg, 3.00 mmol, 1.5 eq) and Na₂CO₃ (678 mg, 6.40 mmol, 3.2 eq) in EtOH (20 mL) and H₂O (4 mL) was added Pd(PPh₃)₂Cl₂ (154 mg, 219 umol, 0.11 eq) under N₂. The suspension was degassed under vacuum and purged with N₂ several times and then stirred at 80° C. for 12 hours. The reaction was concentrated in vacuum to give a residue. The residue was poured into ice water (5 mL) and stirred for 5 min. The aqueous phase was extracted with ethyl acetate (5 mL×3). The combined organic phase was washed with brine (20 mL×2), dried with anhydrous Na₂SO₄, filtered and concentrated in vacuum. The residue was purified by silica gel chromatography (Petroleum ether/Ethyl acetate=10/1, 1/1) to afford 52b (0.5 g, 1.67 mmol, 83.2% yield) as yellow oil. ¹H NMR (MeOD, 400 MHz) δ7.95 (d, J=6.8 Hz, 1H), 7.81 (s, 1H), 7.71 (d, J=6.8 Hz, 1H), 4.36 (s, 2H), 1.45 (s, 9H).

### Preparation of tert-butyl N-[[4-(aminomethyl)-3-(trifluoromethyl)phenyl]methyl]carbamate, 52c

To a solution of 52b (0.5 g, 1.67 mmol, 1.0 eq) in MeOH (10 mL) was added NH₃·H₂O (17 mg, 166 umol, 33% purity, 0.1 eq) and Raney-Ni (1.43 g, 1.67 mmol, 10% purity, 1.0 eq) at 25° C. under N₂. The suspension was degassed under vacuum and purged with H₂ several times, and then stirred under H₂ (50 psi) at 25° C. for 10 hours. Then it was filtered and the filtrate was concentrated in vacuum. The residue was purified by silica gel chromatography (Petroleum ether/Ethyl acetate=10/1, 1/1) to afford 52c (200 mg, 657.23 umol, 39.47% yield) as yellow oil. ¹H NMR (MeOD, 400 MHz) δ 6.99-6.92 (m, 2H), 6.89-6.86 (m, 1H), 3.62 (s, 2H), 2.66 (s, 2H), 0.80 (s, 9H)

### Preparation of tert-butyl N-[[4-(propylaminomethyl)-3-(trifluoromethyl) phenyl]methyl]carbamate, 52d

To a mixture of 52c (190 mg, 624 umol, 1 eq) in MeOH (2 mL) and THE (2 mL) was added propanal (47 mg, 812 umol, 1.3 eq), after 30 min, NaBH₃CN (117 mg, 1.87 mmol, 3.0 eq) and AcOH (3 mg, 62 umol, 3 uL, 0.1 eq) was added at 25° C., and then stirred for 2 hours. The mixture was concentrated in vacuum. The residue was purified by prep-HPLC (column: Phenomenex Gemini-NX C18 75*30 mm*3 um; mobile phase: [water (10 mM NH₄HCO₃)−ACN]; B %: 30%-60%, 12 min) to afford 52d (100 mg, 277 umol, 44.39% yield, 96% purity) as white solid. ¹H NMR (MeOD, 400 MHz) δ7.65-7.57 (m, 2H), 7.53-7.50 (m, 1H), 4.27 (s, 2H), 3.91 (s, 2H), 2.58 (t, J=7.6 Hz, 2H), 1.63-1.59 (m, 2H), 1.45 (s, 9H), 0.93 (t, J=7.2 Hz, 3H)

### Preparation of tert-butyl N-[[4-[[(5-amino-6H-thieno[3,2-b]azepine-7-carbonyl)-propyl-amino]methyl]-3-(trifluoromethyl)phenyl]methyl]carbamate, 52e

To a solution of 5-amino-6H-thieno[3,2-b]azepine-7-carboxylic acid, 16a (24.0 mg, 115 umol, 1.0 eq) in DMF (1 mL) was added DIEA (74 mg, 577 umol, 100 uL, 5 eq) and PyAOP (66 mg, 127 umol, 1.1 eq) in one portion at 25° C., and it was stirred for 30 min, then 52d (60 mg, 173.22 umol, 1.5 eq) was added and then stirred for another 2 hours. After that, the reaction was concentrated and purified by prep-HPLC(column: Phenomenex Synergi C18 150*25*10 um; mobile phase: [water (0.1% TFA)−ACN]; B %: 25%-50%, 10 min) to afford 52e (15 mg, 27.95 umol, 24.21% yield) as white solid. ¹H NMR (MeOD, 400 MHz) δ7.75-7.68 (m, 1H), 7.65 (s, 1H), 7.57 (d, J=7.6 Hz, 1H), 7.44 (d, J=7.6 Hz, 1H), 7.30-7.10 (m, 2H), 4.92 (s, 2H), 4.29 (s, 2H), 3.48 (t, J=7.2 Hz, 2H), 3.32 (s, 2H), 1.66-1.64 (m, 2H), 1.45 (s, 9H), 0.94-0.86 (m, 3H). LC/MS [M+H] 537.2 (calculated); LC/MS [M+H] 537.1 (observed).

### Preparation of TAZ-52

To a solution of 52e (10 mg, 18.6 umol, 1.0 eq) in EtOAc (2 mL) was added HCl/EtOAc (4 M, 140 uL, 30 eq) in one portion at 25° C. The mixture was stirred at 25° C. for 3 hours. Followed, the mixture was concentrated to afford TAZ-52 (8 mg, 18.3 umol, 98.35% yield) as yellow solid. ¹H NMR (MeOD, 400 MHz) δ7.80 (s, 1H), 7.70-7.63 (m, 2H), 7.51 (d, J=7.6 Hz, 1H), 7.20-7.17 (m, 1H), 7.03 (d, J=4.0 Hz, 1H), 4.85 (s, 2H), 4.13 (s, 2H), 3.45-3.37 (m, 2H), 3.31 (s, 2H), 1.62-1.50 (m, 2H), 0.87-0.68 (m, 3H). LC/MS [M+H] 437.2 (calculated); LC/MS [M+H] 437.1 (observed).

### Example 54 Synthesis of 5-amino-N-[[4-(aminomethyl)-2-(trifluoromethyl)phenyl]methyl]-N-propyl-6H-thieno[3,2-b] azepine-7-carboxamide, TAZ-54

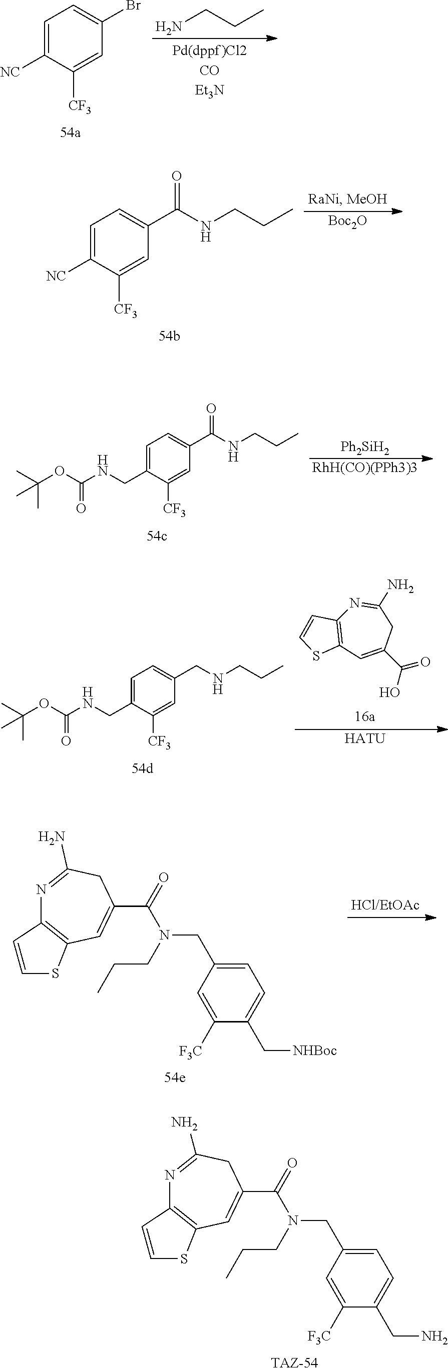

### Preparation of 4-cyano-N-propyl-2-(trifluoro methyl)benzamide, 54b

To a solution of 4-bromo-3-(trifluoromethyl)benzonitrile, 54a (4.00 g, 16.0 mmol, 1.0 eq) in DMF (20 mL) was added propan-1-amine (2.84 g, 48.0 mmol, 3.95 mL, 3.0 eq), Et₃N (4.86 g, 48.0 mmol, 6.68 mL, 3.0 eq) and Pd(dppf)Cl₂ (585 mg, 800 umol, 0.05 eq) under N₂. The suspension was degassed under vacuum and purged with CO several times, and the mixture was stirred under CO (50 psi) at 80° C. for 15 hours. Water (50 mL) was added to the mixture and the aqueous phase was extracted with ethyl acetate (30 mL*3), the combined organic phase was washed with brine (50 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1, 1/1) to afford 54b (4.00g, 15.6 mmol, 97.5% yield) as white solid. ¹H NMR (400 MHz, CDCl₃) δ 8.13 (s, 1H), 8.01 (d, J=8.0 Hz, 1H), 7.85 (d, J=8.0 Hz, 1H), 6.49 (br s, 1H), 3.37 (q, J=7.2 Hz, 2H), 1.66-1.54 (m, 2H), 0.92 (t, J=7.2 Hz, 3H)

### Preparation of tert-butyl N-[[4-(propyl carbamoyl)-3-(trifluoromethyl)phenyl]methyl]carbamate, 54c

To a solution of 54b (2.30 g, 8.98 mmol, 1.0 eq) in MeOH (30 mL) was added Raney Ni (0.5 g, 1.0 eq) and Boc₂O (9.80 g, 44.8 mmol, 10.3 mL, 5.0 eq) at 25° C. under N₂. The suspension was degassed under vacuum and purged with H₂ several times. The mixture was stirred under H₂ (50 psi) at 25° C. for 5 hours. The reaction mixture was filtered and the filtrate was concentrated in vacuum. The residue was purified by silica gel chromatography (column height: 250 mm, diameter: 100 mm, 100-200 mesh silica gel, Petroleum ether/Ethyl acetate=10/1, 1/1) to afford 54c (2.50 g, 6.94 mmol, 77.2% yield) as white solid. ¹H NMR (400 MHz, CDCl₃) δ 8.04 (s, 1H), 7.89 (d, J=8.0 Hz, 1H), 7.63 (d, J=8.0 Hz, 1H), 6.26 (s, 1H), 5.02 (s, 1H), 4.53 (d, J=6.0 Hz, 2H), 3.44 (q, J=6.8 Hz, 2H), 1.70-1.62 (m, 2H), 1.46 (s, 9H), 1.00 (t, J=7.6 Hz, 3H).

### Preparation of tert-butyl N-[[4-(propylaminomethyl)-3-(trifluoro methyl)phenyl]methyl]carbamate, 54d

To a mixture of 54c (1.03 g, 2.86 mmol, 1.0 eq) and RhH(CO)(PPh₃)₃(263 mg, 286 umol, 0.1 eq) in THE (30 mL) was added diphenylsilane (3.16 g, 17.1 mmol, 3.16 mL, 6.0 eq) in one portion at 20° C. under N₂, and then stirred at 20° C. for 8 hours. The reaction mixture was concentrated in vacuum, the residue was purified by silica gel chromatography (column height: 250 mm, diameter: 100 mm, 100-200 mesh silica gel, Petroleum ether/Ethyl acetate=10/1, 0/1 to Ethyl acetate/Methanol=5/1) to afford 54d (0.4 g, 1.15 mmol, 40.40% yield) as yellow oil. ¹H NMR (400 MHz, MeOD) δ7.89 (s, 1H), 7.77 (d, J=8.0 Hz, 1H), 7.66 (d, J=8.0 Hz, 1H), 4.48 (s, 2H), 4.29 (s, 2H), 3.08-3.01 (m, 2H), 1.82-1.71 (m, 2H), 1.48 (s, 9H), 1.05 (t, J=7.6 Hz, 3H)

### Preparation of tert-butyl N-[[4-[[(5-amino-6H-thieno[3,2-b]azepine-7-carbonyl)-propyl-amino] methyl]-3-(trifluoromethyl)phenyl]methyl]carbamate, 54e

To a solution of 5-amino-6H-thieno[3,2-b]azepine-7-carboxylic acid, 16a (12.0 mg, 57.7 umol, 1.0 eq) in DMF (1 mL) was added HATU (19.7 mg, 52.0 umol, 0.9 eq) and Et₃N (17.53 mg, 173 umol, 24.1 uL, 3.0 eq) at 20° C. under N₂, the mixture was stirred at 20° C. for 10 min, then 54d (20 mg, 57.7 umol, 1.0 eq) was added and then stirred at 20° C. for 2 hours. The reaction mixture was filtered, the filtrate was purified by prep-HPLC (column: Phenomenex Synergi C18 150*25*10 um; mobile phase: [water (0.1% TFA)−ACN]; B %: 30%-55%, 10 min) to afford 54e (5.00 mg, 7.47 umol, 12.9% yield, 97.1% purity, TFA) as white solid. ¹H NMR (400 MHz, MeOD) δ7.74 (d, J=5.6 Hz, 1H), 7.60-7.52 (m, 3H), 7.22 (s, 1H), 7.12 (br d, J=5.6 Hz, 1H), 4.82 (br s, 2H), 4.45 (s, 2H), 3.51 (br t, J=7.2 Hz, 2H), 3.42-3.35 (m, 2H), 1.75-1.64 (m, 2H), 1.49 (s, 9H), 0.96-0.90 (m, 3H). LC/MS [M+H] 537.2 (calculated); LC/MS [M+H] 537.1 (observed).

### Preparation of TAZ-54

To a solution of 54e (50.0 mg, 93.2 umol, 1.0 eq) in EtOAc (1 mL) was added HCl/EtOAc (4 M, 2.33 mL, 100 eq) at 20° C. and then stirred at 20° C. for 2 hours. The reaction mixture was concentrated in vacuum and freeze-drying to afford TAZ-54 (30.0 mg, 62.8 umol, 67.4% yield, 99.0% purity, HCl) as yellow solid. ¹H NMR (400 MHz, MeOD) δ7.67 (br s, 1H), 7.65-7.61 (m, 3H), 7.10 (s, 1H), 7.04 (d, J=5.2 Hz, 1H), 4.75 (s, 2H), 4.22 (s, 2H), 3.45-3.38 (m, 2H), 3.29 (br s, 2H), 1.63-1.52 (m, 2H), 0.79 (br t, J=7.2 Hz, 3H). LC/MS [M+H] 437.2 (calculated); LC/MS [M+H] 437.1 (observed).

### Example 65 Synthesis of 5-amino-2-[5-(dimethylamino)pentyl]-N-[[4-(methylaminomethyl)phenyl]methyl]-N-propyl-6H-thieno[3,2-b]azepine-7-carboxamide, TAZ-65

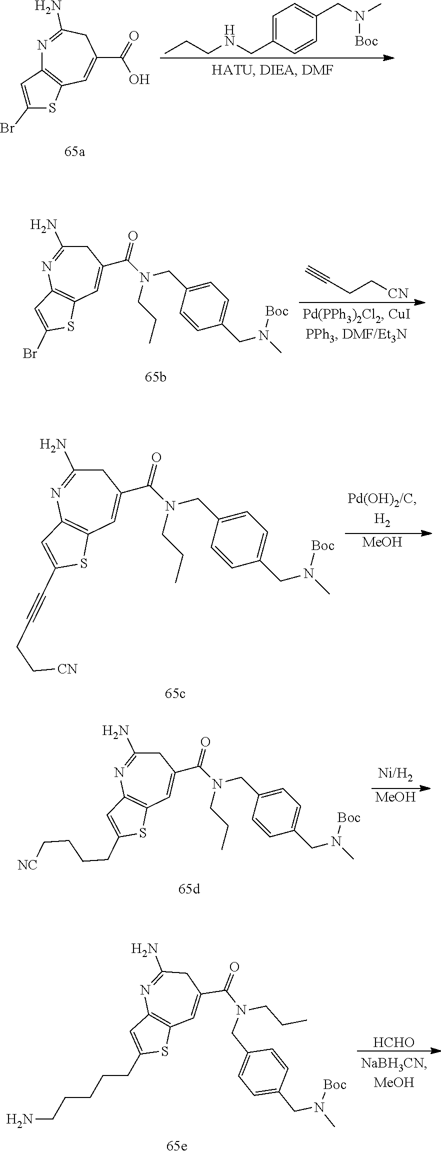

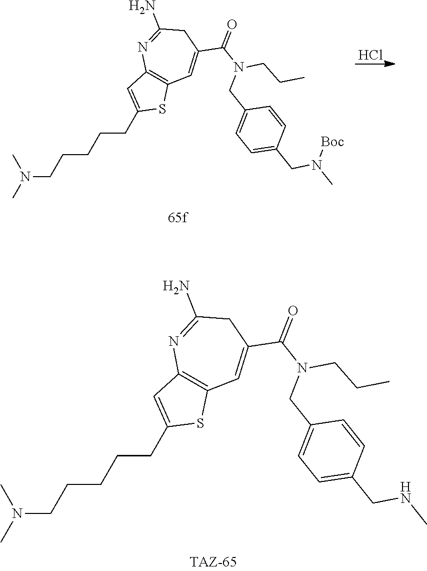

### Preparation of tert-butyl N-[[4-[[(5-amino-2-bromo-6H-thieno[3,2-b]azepine-7-carbonyl)-propyl-amino]methyl]phenyl]methyl]-N-methyl-carbamate, 65b

To a solution of 5-amino-2-bromo-6H-thieno[3,2-b]azepine-7-carboxylic acid, 65a (1.0 g, 3.48 mmol, 1.0 eq) in DMF (20 mL) was added HATU (1.46 g, 3.83 mmol, 1.1 eq), DIEA (1.35 g, 10.45 mmol, 1.82 mL, 3.0 eq) and tert-butyl N-methyl-N-[[4-(propylaminomethyl) phenyl]methyl]carbamate (1.07 g, 3.66 mmol, 1.05 eq), and then stirred for 1 hr at 25° C. The reaction mixture was quenched by addition H₂O (100 mL) and then extracted with EtOAc (100 mL×3). The combined organic layers were washed with brine (20 mL×2), dried over Na₂SO₄, filtered and concentrated. Finally, the residue was purified by column chromatography (SiO₂, Petroleum ether/Ethyl acetate=200/1 to 0/1) to give 65b (1.80 g, 3.21 mmol, 92.04% yield) as yellow oil. ¹H NMR (CDCl₃, 400 MHz) δ7.25-7.15 (m, 4H), 6.92 (s, 1H), 6.77 (s, 1H), 4.72 (s, 2H), 4.42 (s, 2H), 3.40 (t, J=7.2 Hz, 2H), 2.86 (s, 2H), 2.81 (s, 3H), 1.69-1.58 (m, 2H), 1.49 (s, 9H), 0.89 (t, J=7.2 Hz, 3H).

### Preparation of tert-butyl N-[[4-[[[5-amino-2-(4-cyanobut-1-ynyl)-6H-thieno[3,2-b]azepine-7-carbonyl]-propyl-amino]methyl]phenyl]methyl]-N-methyl-carbamate, 65c

To a mixture of 65b (2.25 g, 4.01 mmol, 1.0 eq) and pent-4-ynenitrile (951 mg, 12.0 mmol, 3.0 eq) in TEA (13.2 mL) and DMF (44 mL) was added Pd(PPh₃)₂Cl₂ (140.6 mg, 200.34 umol, 0.050 eq), CuI (152.6 mg, 801.38 umol, 0.20 eq) and PPh₃ (210 mg, 801 umol, 0.20 eq) at 15° C. and then stirred for 3 hrs at 140° C. under N₂ atmosphere. The reaction mixture was quenched by addition H₂O (220 mL) at 25° C., and then extracted with DCM/isopropanol=3/1 (200 mL×3). The combined organic layers were washed with H₂O (40 mL×4), dried over Na₂SO₄, filtered and concentrated. The residue was purified by column chromatography (SiO₂, Petroleum ether/Ethyl acetate=200/1 to 0/1 to Ethyl acetate/MeOH=50/1 to 5/1) to give 65c (1.62 g, 2.89 mmol, 72.23% yield) as yellow oil. ¹H NMR (CDCl₃, 400 MHz) δ 7.21-7.20 (m, 4H), 7.00 (s, 1H), 6.81 (s, 1H), 4.73 (s, 2H), 4.42 (s, 2H), 3.40 (t, J=6.4 Hz, 2H), 2.83-2.81 (m, 7H), 2.68-2.65 (m, 2H), 1.64-1.62 (m, 2H), 1.49 (s, 9H), 0.89 (t, J=7.2 Hz, 3H)

### Preparation of tert-butyl N-[[4-[[[5-amino-2-(4-cyanobutyl)-6H-thieno[3,2-b]azepine-7-carbonyl]-propyl-amino]methyl]phenyl]methyl]-N-methyl-carbamate, 65d

To a solution of 65c (1.47 g, 2.63 mmol, 1.0 eq) in MeOH (30 mL) was added Pd(OH)₂/C (369 mg, 262 umol, 10% purity, 0.10 eq) under N₂ atmosphere. The suspension was degassed and purged with H₂ for 3 times. The mixture was stirred under H₂ (50 Psi) at 25° C. for 12 hrs. The reaction mixture was filtered and the filtrate was concentrated to give 65d (1.15 g, 2.04 mmol, 77.67% yield) as yellow oil. ¹H NMR (CDCl3, 400 MHz) δ7.21-7.19 (m, 4H), 6.86 (s, 1H), 6.67 (s, 1H), 4.74 (s, 2H), 4.42 (s, 2H), 3.40 (t, J=6.4 Hz, 2H), 2.86-2.82 (m, 5H), 2.37 (t, J=6.8 Hz, 2H), 1.88-1.83 (m, 2H), 1.78-1.74 (m, 2H), 1.64-1.61 (m, 2H), 1.49 (m, 9H), 0.89 (t, J=7.6 Hz, 3H).

### Preparation of tert-butyl N-[[4-[[[5-amino-2-(5-aminopentyl)-6H-thieno[3,2-b]azepine-7-carbonyl]-propyl-amino]methyl]phenyl]methyl]-N-methyl-carbamate, 65e

To a solution of 65d (1.15 g, 2.04 mmol, 1.0 eq) in MeOH (23 mL) was added NH₃H₂O (2.86 g, 20.4 mmol, 3.14 mL, 25% purity, 10 eq) and Ni (100 mg) under N₂. The suspension was degassed under vacuum and purged with H₂ several times. The mixture was stirred under H₂ (50 psi) at 25° C. for 3 hrs. The reaction mixture was filtered and the filtrate was concentrated to give 65e (1.03 g, 1.81 mmol, 88.93% yield) as yellow oil.

### Preparation of tert-butyl N-[[4-[[[5-amino-2-[5-(dimethylamino)pentyl]-6H-thieno[3,2-b]azepine-7-carbonyl]-propyl-amino]methyl]phenyl]methyl]-N-methyl-carbamate, 65f

To a solution of 65e (300 mg, 528 umol, 1.0 eq) in MeOH (8 mL) was added AcOH (3.1 mg, 52.8 umol, 0.10 eq), HCHO (171.5 mg, 2.11 mmol, 157.3 uL, 37% purity, 4.0 eq) and NaBH₃CN (99.6 mg, 1.59 mmol, 3.0 eq), and then stirred for 1 hr at 25° C. The mixture was quenched by addition H₂O (10 mL) and concentrated to remove MeOH. The aqueous phase was extracted with DCM/isopropanol=3/1 (10 mL×3). The combined organic layers were dried over Na₂SO₄, filtered and concentrated to give 65f (314 mg, crude) as yellow oil.

### Preparation of TAZ-65

To a solution of 65f (50 mg, 83.9 umol, 1.0 eq) in H₂O (0.5 mL) was added HCl (12 M, 140 uL, 20.0 eq) and then stirred for 1 hr at 80° C. The mixture was concentrated in vacuum. The residue was purified by Prep-HPLC (column: Phenomenex Synergi C18 150*25*10 um; mobile phase: [water(0.1% TFA)−ACN]; B %: 5%-35%, 10 min) to give TAZ-65 (10 mg, 13.74 umol, 16.37% yield, 99.44% purity, 2TFA) as colorless oil. ¹H NMR (MeOD, 400 MHz,) δ 7.50-7.40 (m, 4H), 7.09 (s, 1H), 6.89 (s, 1H), 4.78 (s, 2H), 4.19 (s, 2H), 3.45-3.35 (m, 4H), 3.32 (s, 2H), 3.14-3.10 (m, 2H), 2.88 (s, 6H), 2.72 (s, 3H), 1.80-1.74 (m, 4H), 1.66-1.64 (m, 2H), 1.49-1.47 (m, 2H), 0.89-0.86 (m, 3H). LC/MS [M+H] 496.3 (calculated); LC/MS [M+H] 496.3 (observed).

### Example 109 Synthesis of 5-amino-2-(5-aminopentyl)-N-(3-(3,3-dimethylbutanamido)propyl)-N-propyl-6H-thieno[3,2-b]azepine-7-carboxamide, TAZ-109

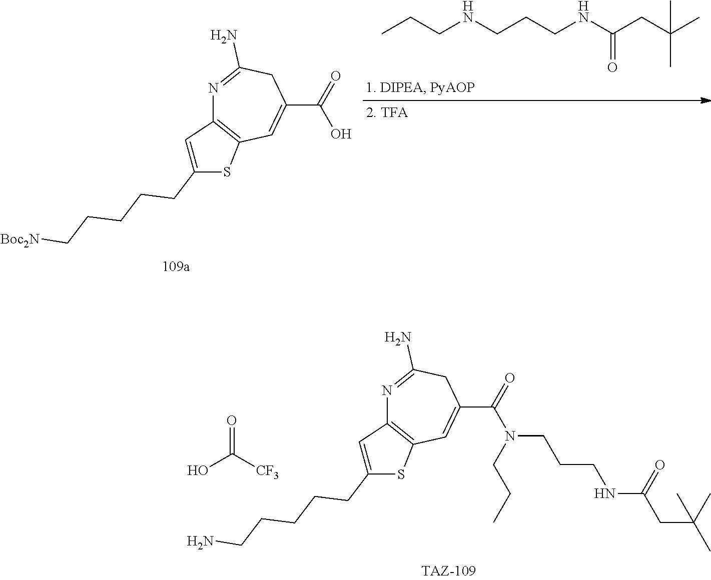

5-amino-2-(5-(bis(tert-butoxycarbonyl)amino)pentyl)-6H-thieno[3,2-b]azepine-7-carboxylic acid, 109a (115 mg, 0.23 mmol, 1 eq.) and 3,3-dimethyl-N-(3-(propylamino)propyl)butanamide (50 mg, 0.23, 1 eq.) were taken up in 8:3 ACN:DCM (2.75 ml). DIPEA (0.121 ml, 0.7 mmol, 3 eq.) was added, followed by 7-Aza-benzotriazol-1-yloxy-tripyrrolidino-phosphonium hexafluorophosphate, PyAOP (0.122 m, 0.23 mmol, 1 eq.). The solution was stirred at ambient temperature. Upon completion by LCMS, the reaction mixture was concentrated and purified by HPLC to afford [amide], which was subsequently dissolved in minimal TFA and allowed to stand for 15 minutes. The solution was concentrated and triturated with diethyl ether to afford TAZ-109 as the trifluoroacetate salt (61.4 mg, 0.102 mmol, 44%). LC/MS [M+H] 490.32 (calculated); LC/MS [M+H] 490.39 (observed).

### Example 133 Synthesis of tert-butyl (3-(5-amino-2-(5-aminopentyl)-N-propyl-6H-thieno[3,2-b]azepine-7-carboxamido)propyl)carbamate, TAZ-133

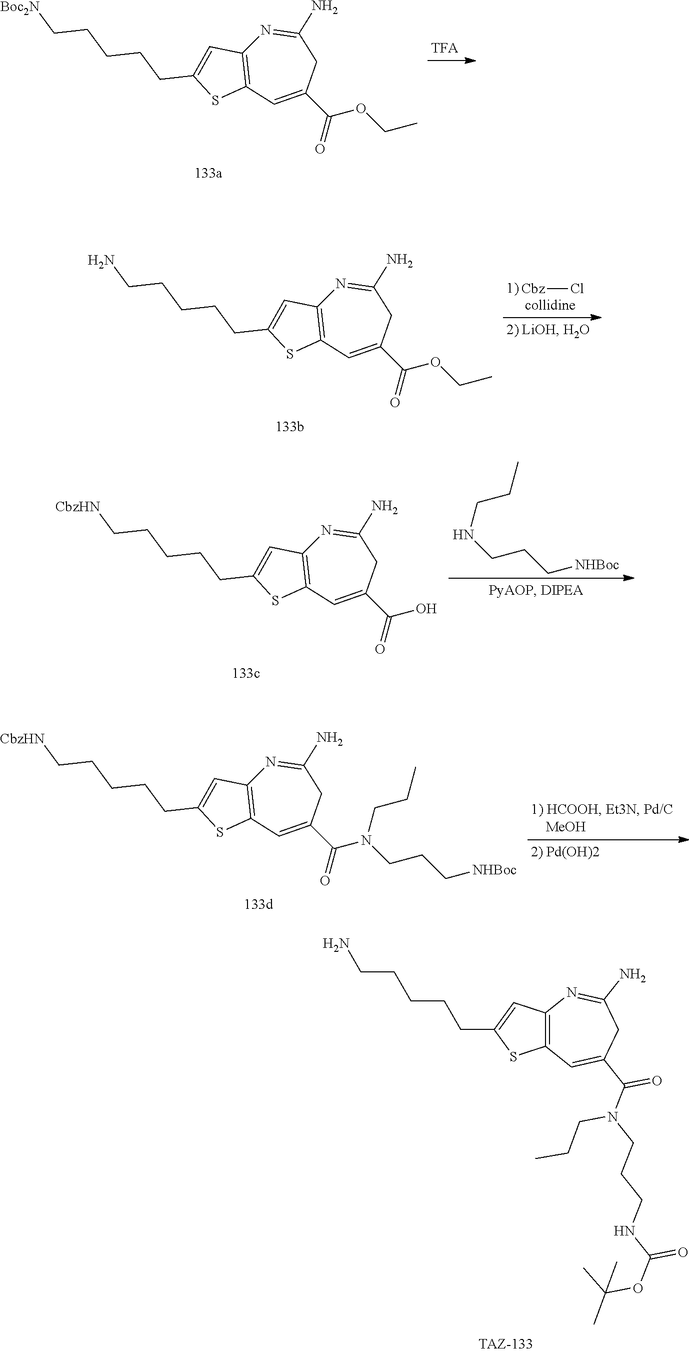

### Preparation of 5-(5-amino-7-(ethoxycarbonyl)-6H-thieno[3,2-b]azepin-2-yl)pentan-1-aminium trifluoroacetate, 133b

Ethyl 5-amino-2-(5-(bis(tert-butoxycarbonyl)amino)pentyl)-6H-thieno[3,2-b]azepine-7-carboxylate, 133a (0.188 g, 0.36 mmol, 1 equiv.) was dissolved in minimal TFA. Upon complete deprotection, the solution was concentrated and the product precipitated from diethyl ether to give 133b (0.082 g, 0.188 mmol, 52%) as a yellow powder. LC/MS [M+H] 322.16 (calculated); LC/MS [M+H] 322.25 (observed).

### Preparation of 5-amino-2-(5-(((benzyloxy)carbonyl)amino)pentyl)-6H-thieno[3,2-b]azepine-7-carboxylic acid, 133c

Intermediate 133b (0.296 g, 0.68 mmol, 1 equiv.) was suspended in 2 ml DMF. Collidine (0.27 ml, 2 mmol, 3 equiv.) was added, followed by benzyl chloroformate, Cbz-Cl, CAS Reg. No. 501-53-1 (0.1 ml, 0.68 mmol, 1 equiv.). The reaction was monitored by LCMS. Upon consumption of the amine starting material, the reaction was concentrated and redissolved in 7 ml 3:1:3 THF:MeOH:water. Lithium hydroxide (0.16 g, 6.8 mmol, 10 equiv.) was added and the reaction stirred at room temperature. Upon completion, the reaction mixture was concentrated and purified by reverse-phase HPLC to give 133c (0.246 g, 0.58 mmol, 85%). LC/MS [M+H] 428.16 (calculated); LC/MS [M+H] 428.32 (observed).

### Preparation of benzyl (5-(5-amino-7-((3-((tert-butoxycarbonyl)amino)propyl)(propyl)carbamoyl)-6H-thieno[3,2-b]azepin-2-yl)pentyl)carbamate, 133d

Intermediate 133c (0.29 g, 0.68 mol, 1 equiv.) and tert-butyl (3-(propylamino)propyl)carbamate (0.225 g, 1.0 mmol, 1.53 equiv.) were dissolved in 1 ml DMF. Diisopropylethylamine, DIPEA (0.59 ml, 1.0 mmol, 1.53 equiv.) was added, followed by PyAOP (0.541 g, 1.0 mmol, 1.53 equiv.). The reaction was stirred at room temperature, then concentrated and purified by reverse-phase flash chromatography to give 133d (0.201 g, 0.32 mmol, 47%). LC/MS [M+H] 626.34 (calculated); LC/MS [M+H] 626.51 (observed).

### Preparation of TAZ-133

Intermediate 133d (0.2 g, 0.32 mmol, 1 equiv.) was dissolved in 2 ml MeOH. Triethylamine (0.1 ml) and formic acid (0.049 ml, 1.29 mmol, 4 equiv.) were added, followed by 10% w/w Pd/C (0.04 g). The stirred reaction was heated to 60° C. After one hour, 20% w/w Pd(OH)₂ (0.02 g) was added. Upon completion, the reaction was filtered, concentrated, and purified by HPLC to give TAZ-133 0.139 g, 0.28 mmol, 88%). LC/MS [M+H] 492.30 (calculated); LC/MS [M+H] 492.45 (observed).

### Example 176 Synthesis of 5-amino-N-ethoxy-2-[2-(4-piperidyl)ethyl]-N-propyl-6H-thieno[3,2-b]azepine-7-carboxamide, TAZ-176

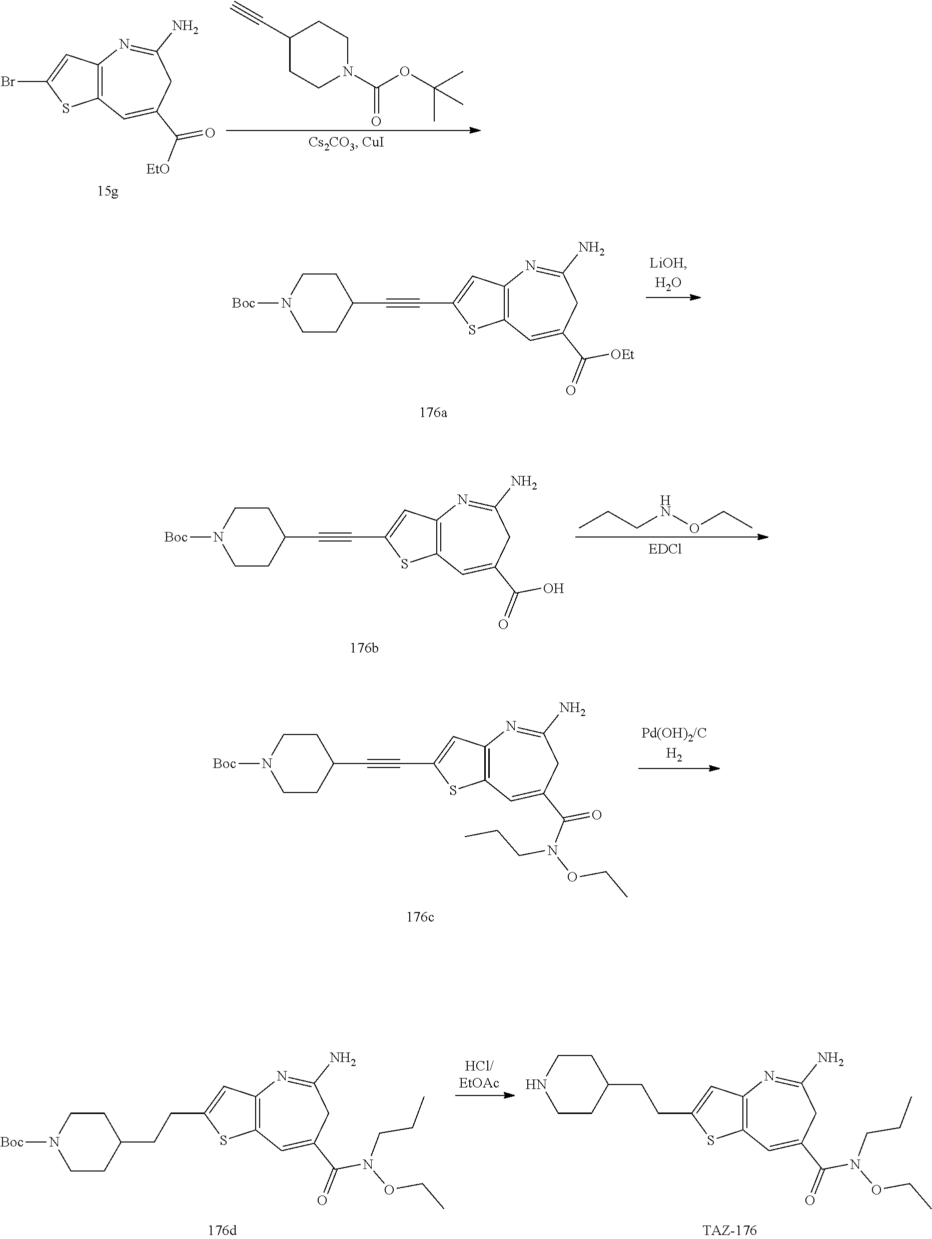

### Preparation of ethyl 5-amino-2-[2-(1-tert-butoxycarbonyl-4-piperidyl)ethynyl]-6H-thieno[3,2-b]azepine-7-carboxylate, 176a

To a mixture of ethyl 5-amino-2-bromo-6H-thieno[3,2-b]azepine-7-carboxylate, 15g (2 g, 6.35 mmol, 1.0 eq) in CH₃CN (60 mL) was added tert-butyl 4-ethynylpiperidine-1-carboxylate (1.73 g, 8.25 mmol, 1.3 eq), Cs₂CO₃ (6.20 g, 19.0 mmol, 3.0 eq), CuI (242 mg, 1.27 mmol, 0.2 eq) and Pd(PPh₃)₂Cl₂ (445 mg, 635 umol, 0.1 eq) in one portion at 25° C. under N₂ and it was stirred at 100° C. for 2 h. The mixture was concentrated to give a residue. The residue was purified by silica gel chromatography (column height: 250 mm, diameter: 100 mm, 100-200 mesh silica gel, Petroleum ether/Ethyl acetate=1/0,10/1) to afford 176a (3.5 g, crude) as yellow solid.

### Preparation of 5-amino-2-[2-(1-tert-butoxycarbonyl-4-piperidyl) ethynyl]-6H-thieno[3,2-b]azepine-7-carboxylic acid, 176b

To a mixture of 176a (3.5 g, 7.89 mmol, 1.0 eq) in EtOH (50 mL) and H₂O (8 mL) was added LiOH·H₂O (1.32 g, 31.6 mmol, 4.0 eq) in one portion at 25° C. and it was stirred at 30° C. for 2 h. The mixture was concentrated and the residue was diluted with water (30 mL). Then the mixture was filtered. The filter cake was triturated with CH₃CN at 25° C. for 0.5 h, then filtered to afford 176b (2.3 g, 5.54 mmol, 70.2% yield) as yellow solid.

### Preparation of tert-butyl 4-[2-[5-amino-7-[ethoxy(propyl)carbamoyl]-6H-thieno[3,2-b]azepin-2-yl]ethynyl]piperidine-1-carboxylate, 176c

To a mixture of 176b (1 g, 2.41 mmol, 1.0 eq) in DCM (10 mL) and DMA (10 mL) was added N-ethoxypropan-1-amine (353 mg, 2.53 mmol, 1.05 eq, HCl) and EDCI (1.85 g, 9.63 mmol, 4.0 eq) in one portion at 25° C. and it was stirred at 25° C. for 1 h. The mixture was concentrated to remove DCM, the residue was diluted with water (50 mL) and the pH of the mixture was adjusted to about 8 with sat, NaHCO₃, and then extracted with EtOAc (30 mL×3). The organic layer was washed with brine, dried over Na₂SO₄, filtered and concentrated. The residue was purified by silica gel chromatography (column height: 250 mm, diameter: 100 mm, 100-200 mesh silica gel, Ethyl acetate/MeOH=1/0, 10/1) to afford 176c (0.63 g, 1.26 mmol, 52.3% yield) as light yellow solid. ¹H NMR (MeOD, 400 MHz) δ7.27 (s, 1H), 6.88 (s, 1H), 3.89 (q, J=7.2 Hz, 2H), 3.74-3.70 (m, 2H), 3.69 (t, J=6.8 Hz, 2H), 3.25-3.19 (m, 3H), 2.99 (s, 2H), 1.89-1.85 (m, 2H), 1.79-1.67 (m, 2H), 1.65-1.59 (m, 2H), 1.47 (s, 9H), 1.15 (t, J=7.2 Hz, 3H), 0.95 (t, J=7.6 Hz, 3H). LC/MS [M+H] 501.2 (calculated); LC/MS [M+H] 501.1 (observed).

### Preparation of tert-butyl 4-[2-[5-amino-7-[ethoxy(propyl) carbamoyl]-6H-thieno[3,2-b]azepin-2-yl]ethyl]piperidine-1-carboxylate, 176d

To a solution of 176c (0.45 g, 899 umol, 1.0 eq) in MeOH (15 mL) was added Pd(OH)₂/C (0.2 g, 10% purity) under N₂. The suspension was degassed under vacuum and purged with H₂ several times. The mixture was stirred under H₂ (50 psi) at 25° C. for 12 hours. The mixture was filtered and concentrated. The residue was purified by silica gel chromatography (column height: 250 mm, diameter: 100 mm, 100-200 mesh silica gel, Petroleum ether/Ethyl acetate=1/0, 0/1) to afford 176d (0.3 g, 594 umol, 66.13% yield) as light yellow solid. ¹H NMR (MeOD, 400 MHz) δ7.32 (s, 1H), 6.65 (s, 1H), 4.06 (d, J=13.2 Hz, 2H), 3.89 (q, J=7.2 Hz, 2H), 3.69 (t, J=7.2 Hz, 2H), 2.98 (s, 2H), 2.85 (t, J=7.6 Hz, 2H), 2.73 (s, 2H), 1.77-1.73 (m, 4H), 1.69-1.60 (m, 2H), 1.58-1.50 (m, 1H), 1.45 (s, 9H), 1.16 (t, J=7.2 Hz, 3H), 1.13-1.05 (m, 2H), 0.95 (t, J=7.6 Hz, 3H). LC/MS [M+H] 501.2 (calculated); LC/MS [M+H] 505.3 (observed).

### Preparation of TAZ-176

To a mixture of 176d (0.3 g, 594 umol, 1.0 eq) in EtOAc (5 mL) was added HCl/EtOAc (4 M, 10 mL) in one portion at 25° C. and it was stirred at 25° C. for 0.5 h. The mixture was concentrated to give TAZ-176 (0.3 g, crude, HCl) as a yellow solid. ¹H NMR (MeOD, 400 MHz) δ7.45 (s, 1H), 6.93 (s, 1H), 3.93 (q, J=7.2 Hz, 2H), 3.72 (t, J=7.2 Hz, 2H), 3.45-3.37 (m, 4H), 3.05-2.91 (m, 4H), 2.02 (d, J=13.6 Hz, 2H), 1.80-1.65 (m, 5H), 1.52-1.35 (m, 2H), 1.18 (t, J=7.2 Hz, 3H), 0.97 (t, J=7.6 Hz, 3H). LC/MS [M+H] 405.2 (calculated); LC/MS [M+H] 405.1 (observed).

### Example 183 Synthesis of 5-amino-2-(azetidin-3-ylmethyl)-N-ethoxy-N-propyl-6H-thieno[3,2-b]azepine-7-carboxamide, TAZ-183

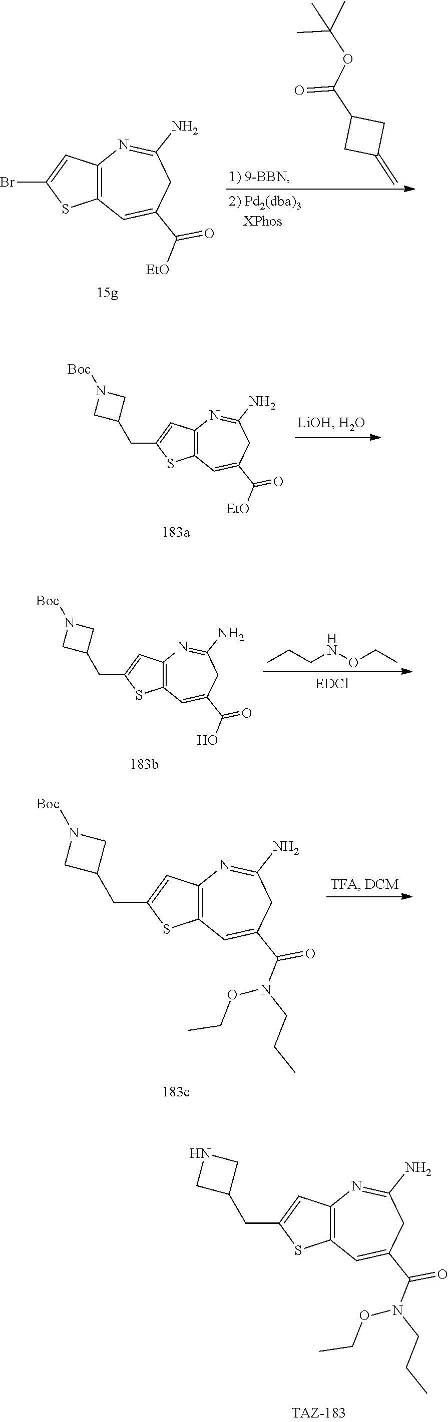

### Preparation of ethyl 5-amino-2-[(1-tert-butoxycarbonyl azetidin-3-yl)methyl]-6H-thieno[3,2-b]azepine-7-carboxylate, 183a

tert-Butyl 3-methyleneazetidine-1-carboxylate (2.25 g, 13.3 mmol, 2 eq) was treated with a 9-BBN (0.5 M, 53.3 mL, 4 eq) in THE (50 mL) and the mixture was heated at 70° C. for 4 hrs. The resulting mixture was transferred into a stirred mixture of ethyl 5-amino-2-bromo-6H-thieno[3,2-b]azepine-7-carboxylate, 15g (2.1 g, 6.66 mmol, 1 eq), Pd₂(dba)₃ (610 mg, 666 umol, 0.1 eq), XPhos (953 mg, 2.00 mmol, 0.3 eq) and Na₂CO₃ (2.12 g, 20.0 mmol, 3 eq) in dioxane (50 mL) and H₂O (5 mL). The resulting mixture was stirred at 100° C. for 12 hr under N₂, and then filtered, the filtrate was concentrated to remove THE and dioxane, EtOAc (100 mL) and water (100 mL) was poured into the mixture. The organic layer was washed with brine, dried over anhydrous Na₂SO₄, concentrated to give a residue. The residue was purified by column chromatography (SiO₂, Petroleum ether/Ethyl acetate=1:0 to 0:1, then EtOAc:MeOH=10:1) to give 183a (2 g, 2.47 mmol, 37.0% yield) as yellow solid.

### Preparation of 5-amino-2-[(1-tert-butoxycarbonylazetidin-3-yl)methyl]-6H-thieno[3,2-b]azepine-7-carboxylic acid, 183b

To a mixture of 183a (2 g, 4.93 mmol, 1 eq) in THE (10 mL) and H₂O (10 mL) was added LiOH·H₂O (621 mg, 14.8 mmol, 3 eq), and then stirred at 15° C. for 3 hr. The mixture was concentrated to remove THF, then the pH of the aqueous phase was adjusted to ˜7 with HCl(4M). The desired solid precipitated from the mixture, and filtered to give 183b (1.5 g, 3.97 mmol, 80.6% yield) as yellow solid. ¹H NMR (400 MHz, DMSO-d₆) δ7.65 (s, 1H), 6.94 (s, 2H), 6.66 (s, 1H), 4.05-3.93 (m, 2H), 3.70-3.55 (m, 2H), 3.07 (d, J=7.6 Hz, 2H), 2.88 (s, 2H), 2.80-2.65 (m, 1H), 1.43 (s, 9H).

### Preparation of tert-butyl 3-[[5-amino-7-[ethoxy(propyl)carbamoyl]-6H-thieno[3,2-b]azepin-2-yl]methyl]azetidine-1-carboxylate, 183c

To a mixture of 183b (0.2 g, 530 umol, 1 eq) and N-ethoxypropan-1-amine (96.2 mg, 689 umol, 1.3 eq, HCl) in DMA (3 mL) and DCM (3 mL) was added EDCI (406 mg, 2.12 mmol, 4 eq), and then stirred at 15° C. for 2 hr. The mixture was concentrated to give a residue.

The residue was purified by Prep-HPLC (column: Phenomenex Synergi C18 150*25*10 um; mobile phase: [water(0.1% TFA)−ACN]; B %: 25%-55%, 8 min) to give 183c (130 mg, 273 umol, 51.6% yield, 97.2% purity) as light-yellow solid. ¹H NMR (400 MHz, MeOD) δ7.47 (s, 1H), 6.93 (s, 1H), 4.07 (br t, J=8.4 Hz, 2H), 3.95 (q, J=7.2 Hz, 2H), 3.82-3.63 (m, 4H), 3.44 (s, 2H), 3.18 (d, J=7.6 Hz, 2H), 3.02-2.81 (m, 1H), 1.76 (sxt, J=7.2 Hz, 2H), 1.45 (s, 9H), 1.20 (t, J=7.2 Hz, 3H), 0.99 (t, J=7.2 Hz, 3H). LC/MS [M+H] 463.2 (calculated); LC/MS [M+H] 463.1 (observed).

### Preparation of TAZ-183

To a mixture of 183c (0.11 g, 238 umol, 1 eq) in DCM (10 mL) was added TFA (1.54 g, 13.5 mmol, 1 mL, 56.8 eq). The mixture was stirred at 15° C. for 1 hr. The pH of the mixture was adjusted to ˜7 with saturated aqueous solution of NaHCO₃, then extracted with DCM/i-PrOH(3:1, 10 mL*3). The organic layer was dried over Na₂SO₄, concentrated to give TAZ-183 (60 mg, 151 umol, 63.3% yield, 90.95% purity) as light yellow solid. ¹H NMR (400 MHz, MeOD) δ7.20 (s, 1H), 6.63 (s, 1H), 4.04 (br t, J=9.6 Hz, 2H), 3.83-3.75 (m, 4H), 3.59 (t, J=7.2 Hz, 2H), 3.36-3.30 (m, 1H), 3.05 (d, J=7.6 Hz, 2H), 2.88 (s, 2H), 1.62 (sxt, J=7.2 Hz, 2H), 1.08-1.00 (m, 3H), 0.85 (t, J=7.2 Hz, 3H). LC/MS [M+H] 363.2 (calculated); LC/MS [M+H] 363.1 (observed).

### Example 185 Synthesis of cyclobutyl N-[3-[[5-amino-2-(4-piperidylmethyl)-6H-thieno[3,2-b]azepine-7-carbonyl]-propyl-amino]propyl]carbamate, TAZ-185

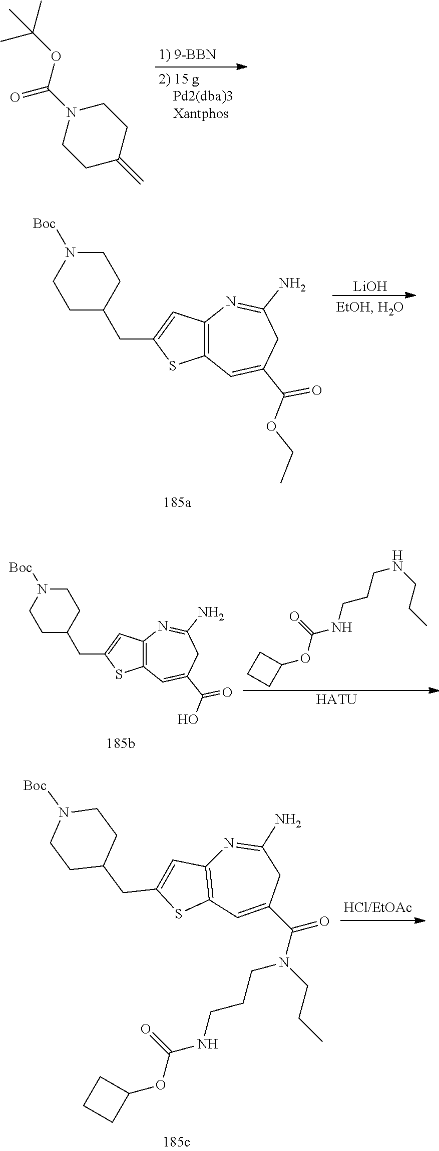

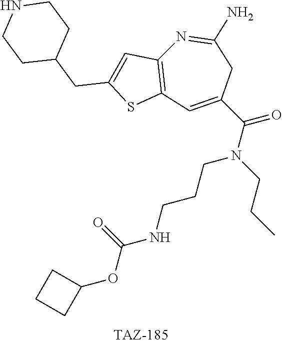 [SMILES: CCCN(CCCNC(=O)OC1CCC1)C(=O)C1=Cc2sc(CC3CCNCC3)cc2N=C(N)C1]

### Preparation of ethyl 5-amino-2-[(1-tert-butoxycarbonyl-4-piperidyl) methyl]-6H-thieno[3,2-b] azepine-7-carboxylate, 185a

A mixture of tert-butyl 4-methylenepiperidine-1-carboxylate (4.51 g, 22.8 mmol, 2.0 eq) and 9-BBN (1 M, 57.1 mL, 5.0 eq) was heated to 70° C. and stirred at 70° C. for 2 hours, then ethyl 5-amino-2-bromo-6H-thieno[3,2-b]azepine-7-carboxylate, 15g (3.60 g, 11.4 mmol, 1.0 eq), Xantphos (1.59 g, 2.74 mmol, 0.24 eq), Pd₂(dba)₃ (836 mg, 913 umol, 0.08 eq), K₂CO₃ (4.74 g, 34.2 mmol, 3.0 eq), H₂O (5 mL) and dioxane (50 mL) was added to this mixture after it was cooled to 20° C., then the mixture was stirred at 100° C. for 4 hours under N₂. Water (200 mL) was added and the aqueous phase was extracted with ethyl acetate (50 mL*4), the combined organic phase was washed with brine (100 mL*1), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1, 0/1) to afford 185a (1.8 g, 4.15 mmol, 36.35% yield) as brown oil.

### Preparation of 5-amino-2-[(1-tert-butoxycarbonyl-4-piperidyl)methyl]-6H-thieno[3,2-b]azepine-7-carboxylic acid, 185b

To a solution of L-185a (1.80 g, 4.15 mmol, 1.0 eq) in EtOH (3 mL) and H₂O (5 mL) was added LiOH·H₂O (696 mg, 16.6 mmol, 4.0 eq) in one portion at 20° C. under N₂, and then stirred at 20° C. for 4 hours. The reaction mixture was quenched with HCl (4 M) until pH=6, then EtOH was removed in vacuum. The precipitation was filtered and dried to afford 185b (1.20 g, 2.96 mmol, 71.2% yield) as yellow solid. ¹H NMR (400 MHz, DMSO-d₆) δ7.61 (s, 1H), 6.58 (s, 1H), 3.86 (d, 2.0 Hz, 2H), 2.92 (s, 2H), 2.65 (d, J=6.4 Hz, 2H), 1.65-1.60 (m, 3H), 1.35 (s, 9H), 1.07-0.96 (m, 2H)

### Preparation of tert-butyl 4-[[5-amino-7-[3-(cyclobutoxycarbonylamino) propyl-propyl-carbamoyl]-6H-thieno[3,2-b]azepin-2-yl]methyl]piperidine-1-carboxylate, 185c

To a mixture of L-185b (200 mg, 493 umol, 1.0 eq) and cyclobutyl N-[3-(propylamino) propyl]carbamate (148 mg, 591 umol, 1.2 eq, HCl) in DMF (2 mL) was added HATU (187 mg, 493 umol, 1.0 eq) and DIEA (191 mg, 1.48 mmol, 257 uL, 3.0 eq) in one portion at 20° C. under N₂, the mixture was stirred at 20° C. for 1 hours. Water (10 mL) was added and the aqueous phase was extracted with ethyl acetate (10 mL*3), the combined organic phase was washed with brine (15 mL*2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=5/1, 0/1 to Ethyl acetate/Methanol=10/1) to afford 185c (180 mg, 299 umol, 60.6% yield) as brown solid. ¹H NMR (400 MHz, MeOD) δ6.88 (s, 1H), 6.68 (s, 1H), 4.88-4.80 (m, 1H), 4.08 (d, J=12.4 Hz, 2H), 3.49 (t, J=7.2 Hz, 2H), 3.41 (t, J=7.6 Hz, 2H), 3.11 (s, 2H), 2.75 (d, J=6.8 Hz, 3H), 2.36-2.23 (m, 2H), 1.86-1.59 (m, 10H), 1.47 (s, 9H), 1.22-1.09 (m, 2H), 0.90 (t, J=4.0, 3H)

### Preparation of TAZ-185

To a solution of 185c (180 mg, 299 umol, 1.0 eq) in EtOAc (1 mL) was added HCl/EtOAc (4 M, 3.74 mL, 50 eq) in one portion at 20° C. under N₂, and then stirred at 20° C. for 1 hour. The reaction mixture was concentrated in vacuum to afford TAZ-185 (140 mg, 260 umol, 86.98% yield, HCl) as yellow oil.

### Example 198 Synthesis of 5-amino-N-ethoxy-2-(4-piperidylmethyl)-N-propyl-6H-thieno[3,2-b]azepine-7-carboxamide, TAZ-198

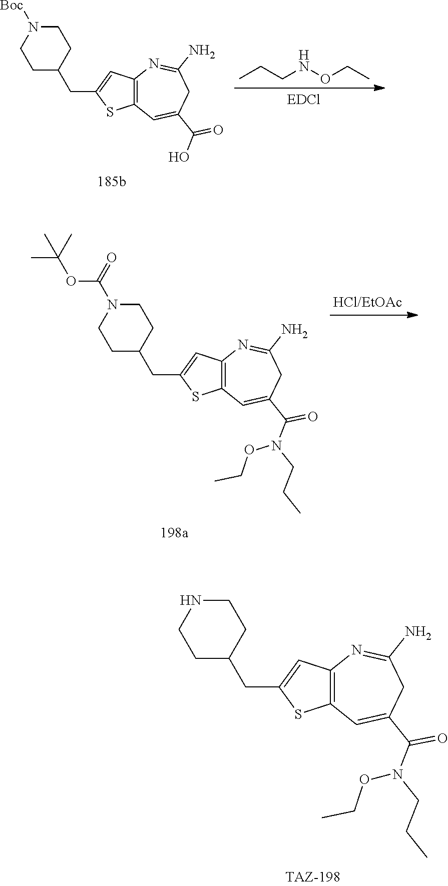

### Preparation of tert-butyl 4-[[5-amino-7-[ethoxy(propyl)carbamoyl]-6H-thieno[3,2-b]azepin-2-yl]methyl]piperidine-1-carboxylate, 198a

To a mixture of 5-amino-2-[(1-tert-butoxycarbonyl-4-piperidyl)methyl]-6H-thieno[3,2-b]azepine-7-carboxylic acid, 185b (200 mg, 493 umol, 1.0 eq) and N-ethoxypropan-1-amine (82.6 mg, 591 umol, 1.2 eq, HCl) in DMA (1 mL) and DCM (2 mL) was added EDCI (378 mg, 1.97 mmol, 4.0 eq) at 20° C. under N₂, and then stirred at 20° C. for 2 hours. DCM (2 mL) was removed in vacuum, then the aqueous phase was quenched with aq NaHCO₃ until pH=8, the water phase was extracted with EtOAc (15 mL*3), the combined organic phase was washed with brine (15 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=5/1, 0/1 to Ethyl acetate/Methanol=10/1) to afford 198a (150 mg, 305 umol, 61.9% yield) as brown solid. ¹H NMR (400 MHz, MeOD) δ7.35 (s, 1H), 6.68 (s, 1H), 4.08 (d, J=13.2 Hz, 2H), 3.90 (q, J=7.2 Hz, 2H), 3.71 (t, J=7.2 Hz, 2H), 3.33 (s, 2H), 3.08 (d, J=8.0 Hz 2H), 2.76 (d, J=6.8 Hz, 3H), 1.80-1.70 (m, 5H), 1.47 (s, 9H), 1.20-1.10 (m, 5H), 0.97 (t, J=7.2 Hz, 3H)

### Preparation of TAZ-198

To a solution of 198a (150 mg, 305 umol, 1.0 eq) in EtOAc (1 mL) was added HCl/EtOAc (4 M, 3.82 mL, 50 eq) at 20° C. under N₂, the mixture was stirred at 20° C. for 1 hour. The reaction mixture was concentrated in vacuum to afford TAZ-198 (100 mg, 234 umol, 76.6% yield, HCl) as yellow oil.

### Example 238 Synthesis of cyclobutyl N-[2-[[5-amino-2-(azetidin-3-ylmethyl)-6H-thieno[3,2-b] azepine-7-carbonyl]-propyl-amino] oxyethyl]carbamate, TAZ-238

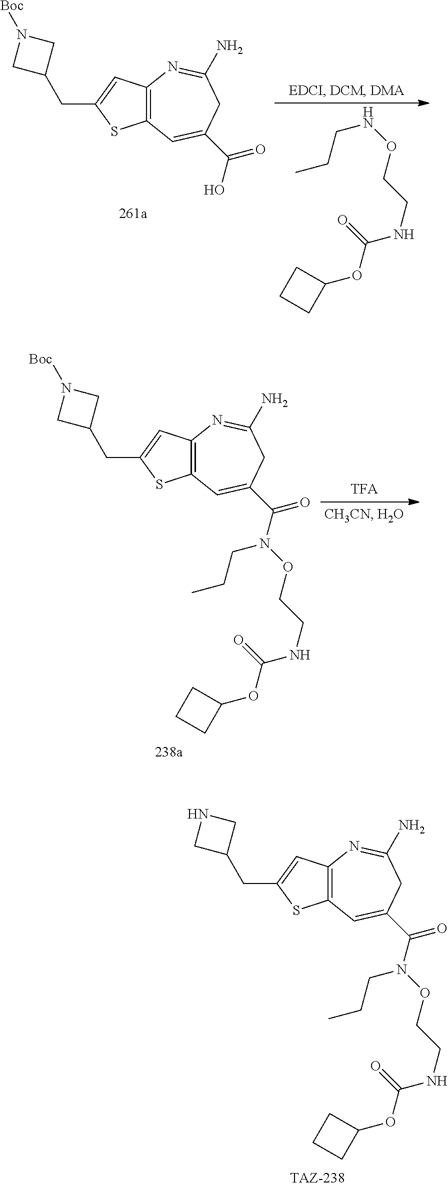

### Preparation of tert-butyl 3-[[5-amino-7-[2-(cyclobutoxycarbonylamino)ethoxy-propyl-carbamoyl]-6H-thieno[3,2-b]azepin-2-yl]methyl]azetidine-i-carboxylate, 238a

To a solution of 5-amino-2-[(1-tert-butoxycarbonylazetidin-3-yl)methyl]-6H-thieno[3,2-b]azepine-7-carboxylic acid, 261a (200 mg, 529.86 umol, 1 eq) and cyclobutyl N-[2-(propylaminooxy)ethyl]carbamate (174.09 mg, 688.82 umol, 1.3 eq, HCl) in DCM (2 mL) and DMA (2 mL) was added EDCI (304.72 mg, 1.59 mmol, 3 eq) at 0° C., and then stirred at 25° C. for 1 hr. The reaction mixture was concentrated under reduced pressure to give a residue. The residue was diluted with H₂O (10 mL) and the pH of the mixture was adjusted to ˜9 with aq. Na₂CO₃ at 0° C. and it was extracted with EtOAc (10 mL * 3). The combined organic layers were washed with brine (5 mL * 3), dried over Na₂SO₄, filtered and concentrated under reduced pressure to give a residue. The residue was purified by column chromatography (SiO2, Petroleum ether/Ethyl acetate=1/0 to 0/1) and then (SiO2, EtOAc:MeOH=1:0 to 3:1) to give 238a (0.2 g, 347.39 umol, 65.56% yield) as a yellow solid. ¹H NMR (MeOD, 400 MHz) δ7.32 (s, 1H), 6.69 (s, 1H), 4.81 (s, 1H), 4.08-3.99 (m, 2H), 3.89 (t, J=5.2 Hz, 2H), 3.72-3.62 (m, 4H), 3.29-3.25 (m, 2H), 3.09 (d, J=7.6 Hz, 2H), 3.02 (s, 2H), 2.95-2.83 (m, 1H), 2.32-2.21 (m, 2H), 2.05-1.94 (m, 2H), 1.77-1.54 (m, 4H), 1.45-1.40 (m, 9H), 0.94 (t, J=7.6 Hz, 3H). LC/MS [M+H] 576.3 (calculated); LC/MS [M+H] 576.3 (observed).

### Preparation of TAZ-238

To a mixture of 238a (0.31 g, 538 umol, 1.0 eq) in DCM (6 mL) was added TFA (1.23 g, 10.8 mmol, 797 uL, 20.0 eq) in one portion at 25° C. and then stirred at 25° C. for 2 h. The mixture was concentrated to give a residue, the residue was diluted with H₂O (15 mL), the mixture was extracted with MTBE(10 mL*2) to remove excess TFA, the aqueous phase was freeze-dried to afford TAZ-238 (0.38 g, 521 umol, 96.7% yield, 96.4% purity, TFA salt) as yellow solid. ¹H NMR (MeOD, 400 MHz) δ7.47 (s, 1H), 6.96 (s, 1H), 4.81-4.74 (m, 1H), 4.22-4.13 (m, 2H), 3.96-3.86 (m, 4H), 3.71 (t, J=7.2 Hz, 2H), 3.41 (s, 2H), 3.29-3.22 (m, 5H), 2.25 (d, J=7.2 Hz, 2H), 2.03-1.89 (m, 2H), 1.80-1.55 (m, 4H), 0.96 (t, J=7.6 Hz, 3H). LC/MS [M+H] 476.2 (calculated); LC/MS [M+H] 476.1 (observed).

### Example 253 Synthesis of 5-amino-2-(azetidin-3-ylmethyl)-N-isopropoxy-N-propyl-6H-thieno[3,2-b]azepine-7-carboxamide, TAZ-253

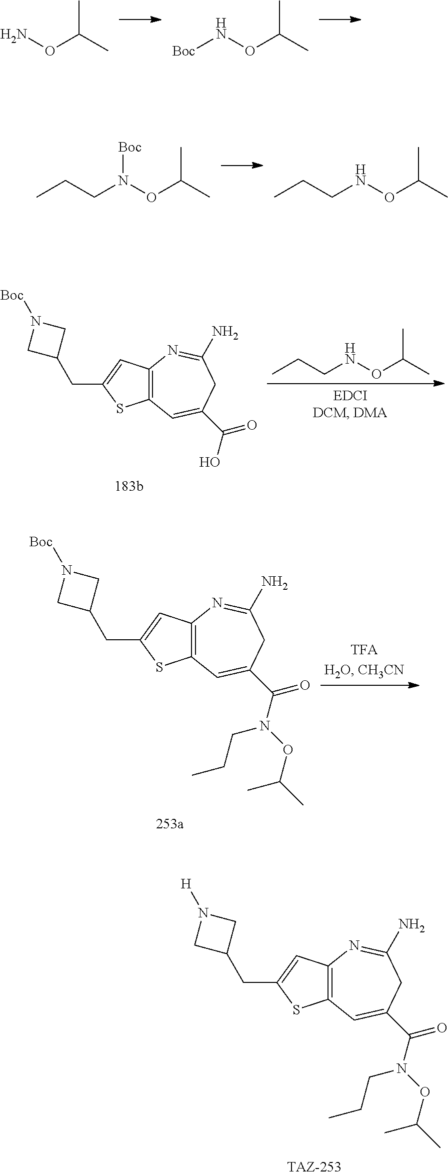

### Preparation of N-isopropoxypropan-1-amine

To a solution of 0-isopropylhydroxylamine (2 g, 17.9 mmol, 1 eq, HCl) in THE (15 mL) was added a solution of NaHCO₃ (3.01 g, 35.8 mmol, 1.39 mL, 2 eq) in H₂O (5 mL) and tert-butoxycarbonyl tert-butyl carbonate (5.87 g, 26.9 mmol, 6.18 mL, 1.5 eq), and then stirred at 20° C. for 2 h under N₂ atmosphere. H₂O (50 mL) was added to the mixture and then extracted with EtOAc (80 mL×3), the combined organic phase was washed with brine (50 mL×3), dried with anhydrous Na₂SO₄, filtered and concentrated in vacuum. The crude product was purified by silica gel chromatography eluted with (Petroleum ether:Ethyl acetate=1:0,1:1). tert-Butyl N-isopropoxycarbamate (2.81 g, 16.0 mmol, 89.46% yield) was obtained as light yellow oil. ¹H NMR (MeOD, 400 MHz) δ7.00 (br s, 1H), 4.10-1.00 (m, 1H), 1.49 (s, 9H), 1.22 (d, J=6.4 Hz, 6H).

To a solution of tert-butyl N-isopropoxycarbamate (2.80 g, 15.9 mmol, 1 eq) in DMF (10 mL) was added NaH (959 mg, 23.9 mmol, 60% purity, 1.5 eq) at 0° C. under N₂ and it was stirred for 0.5 h, and then 1-iodopropane (5.43 g, 32.0 mmol, 3.12 mL, 2 eq) was added. The mixture was stirred at 25° C. for 2 h under N₂ atmosphere. The reaction mixture was quenched at 0° C. by the addition of (50 mL) sat.NH₄Cl solution, then extracted with EtOAc (80 mL×3), the combined organic phase was washed with brine (30 mL×3), dried with anhydrous Na₂SO₄, filtered and concentrated in vacuum. The crude product was purified by silica gel chromatography eluted with Petroleum ether/Ethyl acetate=0:1-1:1. tert-butyl N-isopropoxy-N-propyl-carbamate (3.8 g, crude) was obtained as colorless oil. ¹H NMR (MeOD, 400 MHz) δ4.12-4.02 (m, 1H), 3.39 (t, J=7.2 Hz, 2H), 1.70-1.61 (m, 2H), 1.49 (s, 9H), 1.21 (d, J=6.0 Hz, 6H), 0.90 (t, J=7.2 Hz, 3H).

To a solution of tert-butyl N-isopropoxy-N-propyl-carbamate (3.2 g, 14.7 mmol, 1 eq) in EtOAc (5 mL) was added HCl/EtOAc (4 M, 55.2 mL, 15 eq), and then stirred at 20° C. for 2 h. The reaction mixture was concentrated under reduced pressure to give a residue. N-isopropoxypropan-1-amine (1.61 g, 10.48 mmol, 71.16% yield, HCl) was obtained as colorless oil. ¹H NMR (MeOD, 400 MHz) δ4.76-4.67 (m, 1H), 3.23-3.16 (m, 2H), 2.00-1.89 (m, 2H), 1.41 (d, J=6.2 Hz, 6H), 1.03 (t, J=7.6 Hz, 3H).

### Preparation of tert-butyl 3-[[5-amino-7-[isopropoxy(propyl)carbamoyl]-6H-thieno[3,2-b]azepin-2-yl]methyl]azetidine-1-carboxylate, 253a

To a mixture of 5-amino-2-[(1-tert-butoxycarbonylazetidin-3-yl)methyl]-6H-thieno [3,2-b]azepine-7-carboxylic acid, 183b (400 mg, 1.06 mmol, 1 eq) and N-isopropoxypropan-1-amine (244 mg, 1.59 mmol, 1.50 eq, HCl) in DMA (2 mL) and DCM (2 mL) was added EDCI (610 mg, 3.18 mmol, 3 eq), and then stirred at 20° C. for 1 h. The reaction mixture was added H₂O (20 mL) and then the pH of the mixture was adjusted to ˜8 with aq NaHCO₃, extracted with EtOAc (30 mL×3), the combined organic phase was washed with brine (10 mL×3), dried with anhydrous Na₂SO₄, filtered and concentrated in vacuum. The crude product was purified by silica gel chromatography eluted with (Ethyl acetate:Methanol=1:0,5:1) to give 253a (410 mg, 860 umol, 81.17% yield) as a light yellow solid. ¹H NMR (MeOD, 400 MHz) δ7.34 (s, 1H), 6.73 (s, 1H), 4.20-4.15 (m, 1H), 4.09-4.00 (m, 2H), 3.74-3.63 (m, 4H), 3.06 (d, J=8.0 Hz, 2H), 2.95 (s, 2H), 2.90-2.84 (m, 1H), 1.76-1.67 (m, 2H), 1.43 (s, 9H), 1.17 (d, J=6.2 Hz, 6H), 0.94 (t, J=7.6 Hz, 3H).

### Preparation of TAZ-253

To a solution of 253a (410 mg, 860 umol, 1 eq) in CH₃CN (2 mL) and H₂O (2 mL) was added TFA (785 mg, 6.88 mmol, 510 uL, 8 eq), and then stirred at 80° C. for 2 h under N₂ atmosphere. The reaction mixture was concentrated under reduced pressure to remove CH₃CN, the aqueous phase was extracted with and MTBE (15 mL×3) to remove excess TFA, the water phase was freeze-dried to give TAZ-253 (320 mg, 850 umol, 98.80% yield) as a light yellow solid. ¹H NMR (MeOD, 400 MHz) δ7.43 (s, 1H), 6.95 (s, 1H), 4.27-4.20 (m, 1H), 4.20-4.14 (m, 2H), 3.97-3.87 (m, 2H), 3.73 (br t, J=7.2 Hz, 2H), 3.40 (s, 2H), 3.30-3.25 (m, 3H), 1.81-1.69 (m, 2H), 1.18 (d, J=6.2 Hz, 6H), 0.96 (t, J=7.4 Hz, 3H). LC/MS [M+H] 377.2 (calculated); LC/MS [M+H] 377.1 (observed).

### Example 260 Synthesis of isopropyl N-[2-[[5-amino-2-(azetidin-3-ylmethyl)-6H-thieno [3,2-b]azepine-7-carbonyl]-propyl-amino] oxyethyl]carbamate, TAZ-260

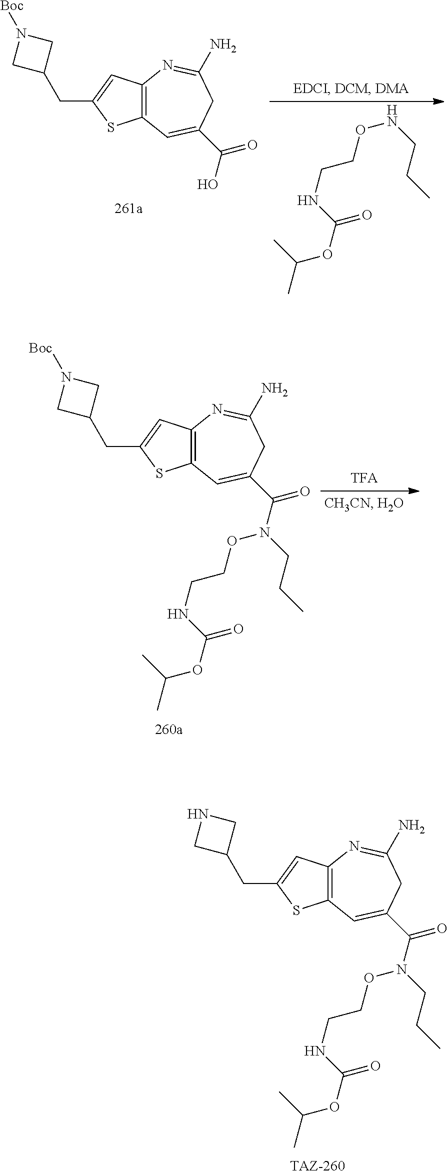 [SMILES: CC(C)(C)OC(=O)N1CC(Cc2cc3c(s2)C=C(C(=O)O)CC(N)=N3)C1 ; CCCNOCCNC(=O)OC(C)C ; CCCN(OCCNC(=O)OC(C)C)C(=O)C1=Cc2sc(CC3CN(C(=O)OC(C)(C)C)C3)cc2N=C(N)C1 ; CCCN(OCCNC(=O)OC(C)C)C(=O)C1=Cc2sc(CC3CNC3)cc2N=C(N)C1]

### Preparation of tert-butyl 3-[[5-amino-7-[2-(isopropoxycarbonylamino) ethoxy-propyl-carbamoyl]-6H-thieno[3,2-b] azepin-2-yl]methyl]azetidine-i-carboxylate, 260a

To a mixture of isopropyl N-[2-(propylaminooxy)ethyl]carbamate (158 mg, 654 umol, 1.3 eq, HCl) and 5-amino-2-[(1-tert-butoxycarbonylazetidin-3-yl)methyl]-6H-thieno [3,2-b]azepine-7-carboxylic acid, 261a (0.19 g, 503 umol, 1.0 eq) in DCM (4 mL) and DMA (0.5 mL) was added EDCI (289 mg, 1.51 mmol, 3.0 eq) in one portion at 25° C. and then stirred at 25° C. for 0.5 h. The mixture was concentrated to remove DCM. Then the mixture was diluted with water (20 mL), the pH of the aqueous phase was adjusted to ˜8 with sat.NaHCO₃ and then the mixture was extracted with EtOAc (20 mL×3). The organic layer was washed with brine, dried over Na₂SO₄, filtered and concentrated. The residue was purified by silica gel chromatography (column height: 250 mm, diameter: 100 mm, 100-200 mesh silica gel, Ethyl acetate/MeOH=1/0, 10/1) to afford 260a (0.18 g, 319 umol, 63.44% yield) as yellow oil. ¹H NMR (MeOH, 400 MHz) δ7.30 (s, 1H), 6.67 (s, 1H), 4.83-4.75 (m, 1H), 4.07-4.03 (m, 2H), 3.89 (t, J=5.4 Hz, 2H), 3.68 (t, J=7.0 Hz, 4H), 3.30-3.26 (m, 2H), 3.08 (d, J=7.6 Hz, 2H), 3.00 (s, 2H), 2.90-2.85 (m, 1H), 1.76-1.67 (m, 2H), 1.43 (s, 9H), 1.18 (d, J=6.0 Hz, 6H), 0.94 (t, J=7.2 Hz, 3H).

### Preparation of TAZ-260

To a mixture of 260a (0.18 g, 319 umol, 1.0 eq) in CH₃CN (2 mL) and H₂O (2 mL) was added TFA (291 mg, 2.55 mmol, 189 uL, 8.0 eq) at 25° C. and it was stirred at 80° C. for 0.5 h. The mixture was concentrated to remove CH₃CN. Then the mixture was extracted with MTBE (10 mL×3) to remove excess TFA. The water phase was freeze-dried to give TAZ-260 (0.25 g, 310.30 umol, 97.18% yield, TFA salt) as a yellow solid. ¹H NMR (MeOH, 400 MHz) δ7.47 (s, 1H), 6.96 (s, 1H), 4.81-4.76 (m, 1H), 4.23-4.09 (m, 2H), 3.98-3.86 (m, 4H), 3.71 (t, J=6.8 Hz, 2H), 3.41 (s, 2H), 3.29-3.18 (m, 5H), 1.83-1.64 (m, 2H), 1.25-1.12 (m, 6H), 0.96 (t, J=7.6 Hz, 3H). LC/MS [M+H] 464.2 (calculated); LC/MS [M+H] 464.1 (observed).

### Example 261 Synthesis of 5-amino-2-(azetidin-3-ylmethyl)-N-[2-(ethylcarbamoylamino)ethoxy]-N-propyl-6H-thieno[3,2-b]azepine-7-carboxamide, TAZ-261

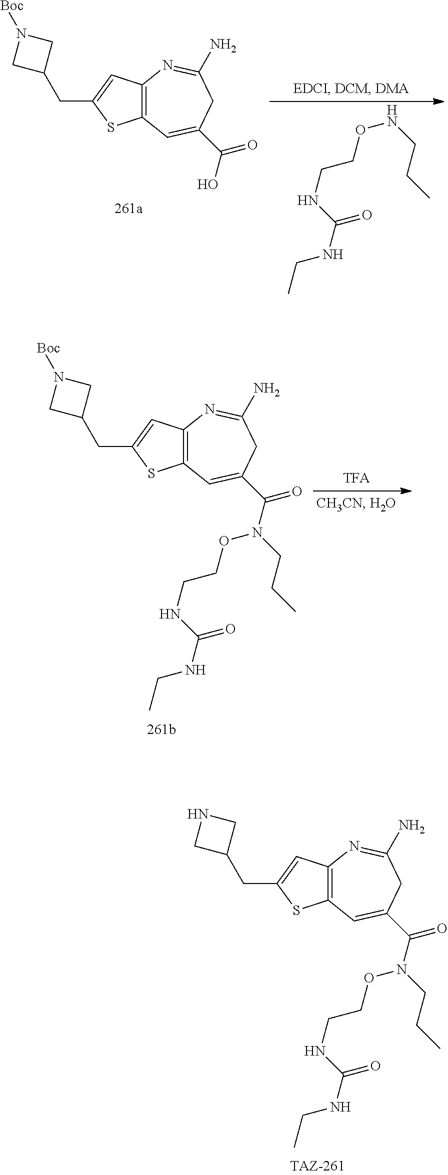 [SMILES: CC(C)(C)OC(=O)N1CC(Cc2cc3c(s2)C=C(C(=O)O)CC(N)=N3)C1 ; CCCNOCCNC(=O)NCC ; CCCN(OCCNC(=O)NCC)C(=O)C1=Cc2sc(CC3CN(C(=O)OC(C)(C)C)C3)cc2N=C(N)C1 ; CCCN(OCCNC(=O)NCC)C(=O)C1=Cc2sc(CC3CNC3)cc2N=C(N)C1]

### Preparation of tert-butyl 3-[[5-amino-7-[2-(ethylcarbamoylamino)ethoxy-propyl-carbamoyl]-6H-thieno[3,2-b]azepin-2-yl]methyl]azetidine-1-carboxylate, 261b

To a solution of 2-[(1-tert-butoxycarbonylazetidin-3-yl)methyl]-5-amino-6H-thieno[3,2-b]azepine-7-carboxylic acid, 261a (230 mg, 607.76 umol, 1 eq) and 1-ethyl-3-[2-(propylaminooxy)ethyl]urea (192 mg, 851 umol, 1.4 eq, HCl) in DCM (3.00 mL) and DMA (3.00 mL) was added EDCI (350 mg, 1.82 mmol, 3 eq), and then stirred at 25° C. for 1 h. The mixture was concentrated to remove DCM and diluted with water (20 mL) and the pH of the mixture was adjusted about 9 by sat, Na₂CO₃ and extracted with EtOAc (20 mL×3). The organic layer was washed with brine (20 mL×3), dried over Na₂SO₄, filtered and concentrated. The residue was purified by flash silica gel chromatography (ISCO®; 0.5 g SepaFlash® Silica Flash Column, Eluent of 0˜30% Ethyl acetate/MeOH @ 35 mL/min) to give 261b (270 mg, 492 umol, 80.97% yield) as light yellow solid. ¹H NMR (MeOD, 400 MHz) δ7.29 (s, 1H), 6.68 (s, 1H), 4.10-4.00 (m, 2H), 3.88 (t, J=5.2 Hz, 2H), 3.74-3.61 (m, 4H), 3.29 (br s, 2H), 3.12-3.05 (m, 4H), 3.01 (s, 2H), 2.95-2.83 (m, 1H), 1.72 (sxt, J=7.2 Hz, 2H), 1.43 (s, 9H), 1.06 (t, J=7.2 Hz, 3H), 0.94 (t, J=7.6 Hz, 3H).

### Preparation of TAZ-261

To a solution of 261b (270 mg, 492 umol, 1 eq) in CH₃CN (3.00 mL) and H₂O (3.00 mL) was added TFA (449 mg, 3.94 mmol, 291 uL, 8 eq), and then stirred at 80° C. for 1 h. The mixture was concentrated and diluted with water (20 mL) and extracted with MTBE (20 mL×2) to remove excess TFA and the aqueous phase was freeze-dried to give TAZ-261 (300 mg, 443.38 umol, 90.10% yield, 2TFA) as light yellow solid. ¹H NMR (MeOD, 400 MHz) δ7.45 (s, 1H), 6.96 (s, 1H), 4.21-4.13 (m, 2H), 3.97-3.86 (m, 4H), 3.71 (t, J=7.2 Hz, 2H), 3.42 (s, 2H), 3.30-3.20 (m, 3H), 3.06 (q, J=7.2 Hz, 2H), 1.80-1.68 (m, 2H), 1.05 (t, J=7.2 Hz, 3H), 0.96 (t, J=7.6 Hz, 3H). LC/MS [M+H] 449.2 (calculated); LC/MS [M+H] 449.1 (observed).

### Example L-1 Synthesis of 2,3,5,6-tetrafluorophenyl (E)-40-(5-amino-6H-thieno[3,2-b]azepine-7-carbonyl)-35-((3-cyanophenyl)imino)-4,7,10,13,16,19,22,25,28,31-decaoxa-34,36,40-triazatritetracontanoate, TAZ-L-1

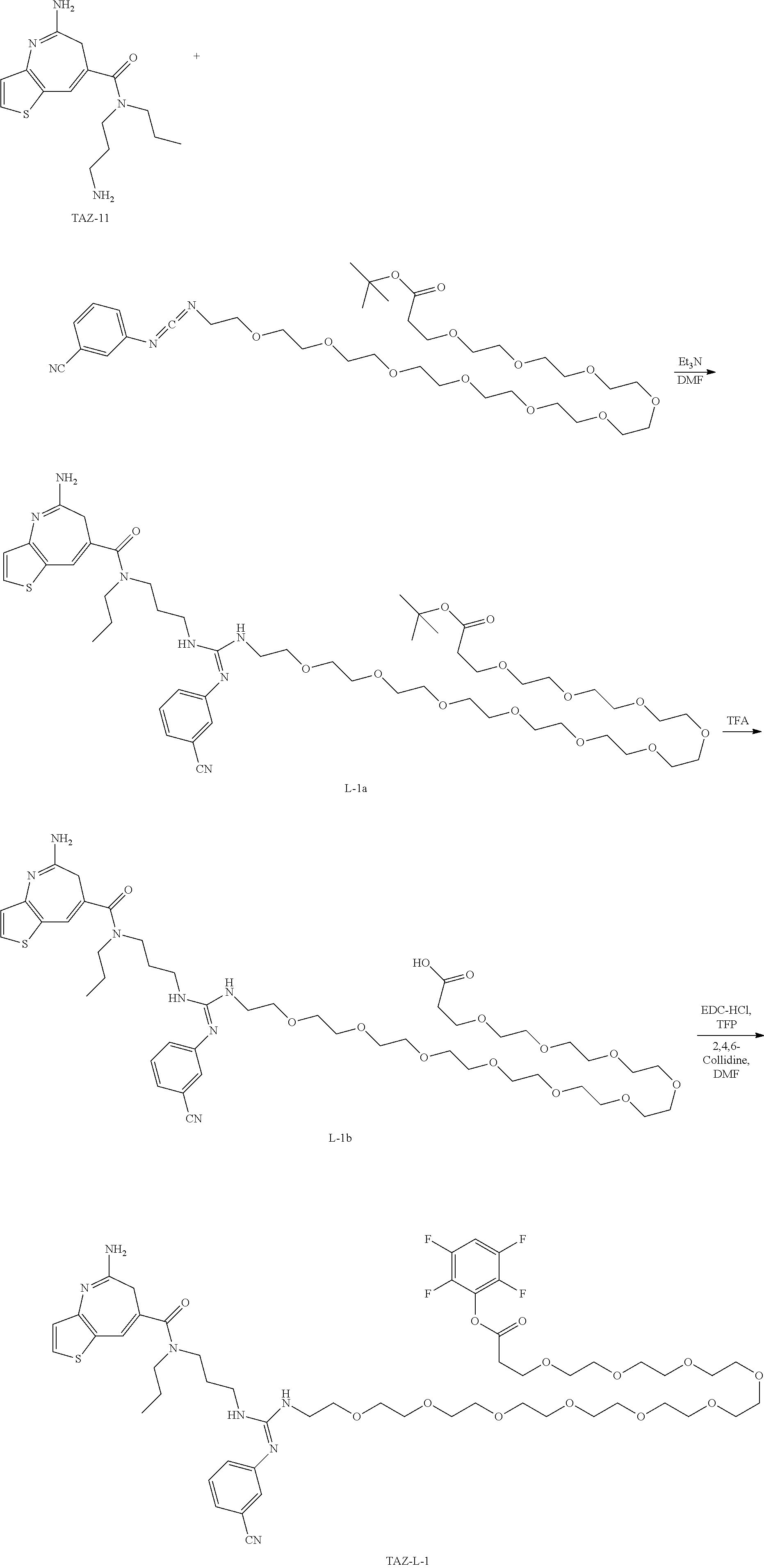

### Preparation of tert-butyl (E)-40-(5-amino-6H-thieno[3,2-b]azepine-7-carbonyl)-35-((3-cyanophenyl)imino)-4,7,10,13,16,19,22,25,28,31-decaoxa-34,36,40-triazatritetracontanoate, L-1a

TAZ-11 (0.05 g, 0.16 mmol, 1 eq.) and tert-butyl 1-((3-cyanophenyl)imino)-5,8,11,14,17,20,23,26,29,32-decaoxa-2-azapentatriacont-1-en-35-oate, PEG10-diimide (0.116 g, 0.16 mmol, 1 eq.) were dissolved in DMF. Triethylamine (0.068 ml, 0.49 mmol, 3 eq.) was added, and the reaction was stirred at ambient temperature. Upon consumption of amine starting material, the reaction was concentrated and purified by HPLC to give L-1a (0.102 g, 0.10 mmol, 62%). LC/MS [M+H] 1018.55 (calculated); LC/MS [M+H] 1018.91 (observed).

### Preparation of (E)-40-(5-amino-6H-thieno[3,2-b]azepine-7-carbonyl)-35-((3-cyanophenyl)imino)-4,7,10,13,16,19,22,25,28,31-decaoxa-34,36,40-triazatritetracontanoic acid, L-1b

L-1a (0.102 g, 0.100 mmol, 1 eq.) was dissolved in 100 μl TFA. After 15 minutes, the product was triturated with diethyl ether and then concentrated under vacuum to give L-1b (94.4 mg, 0.98 mmol, 98%). LC/MS [M+H] 962.49 (calculated); LC/MS [M+H] 962.85 (observed).

### Preparation of TAZ-L-1

L-1b (0.094 g, 0.098 mmol, 1 eq.) and 2,3,5,6-tetrafluorophenol, TFP (0.033 g, 0.20 mmol, 2 eq.) were dissolved in DMF. Collidine (0.064 ml, 0.49 mmol, 5 eq.) was added, followed by 1-ethyl-3-(3-dimethylaminopropyl)carbodiimide hydrochloride, EDC-HCl (0.038 g, 0.20 mmol, 2 eq.). The reaction was stirred at room temperature until complete, then purified by HPLC to give TAZ-L-1 (0.057 g, 0.051 mmol, 52%). LC/MS [M+H] 1110.48 (calculated); LC/MS [M+H] 1110.87 (observed).

### Example L-2 Synthesis of 2,3,5,6-tetrafluorophenyl (E)-41-(5-amino-6H-thieno[3,2-b]azepine-7-carbonyl)-35-((3-cyanophenyl)imino)-4,7,10,13,16,19,22,25,28,31-decaoxa-34,36,41-triazatetratetracont-38-ynoate, TAZ-L-2

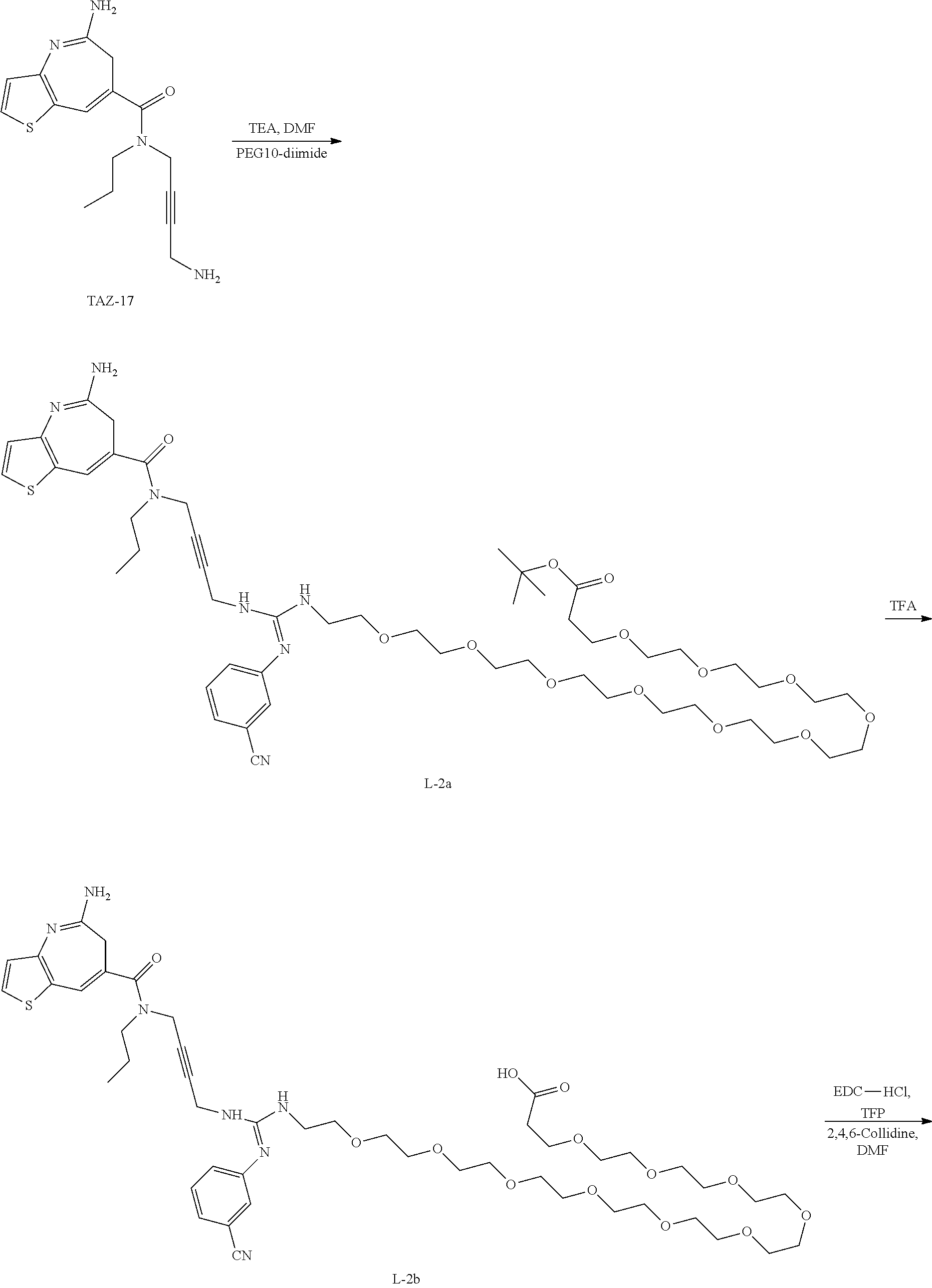

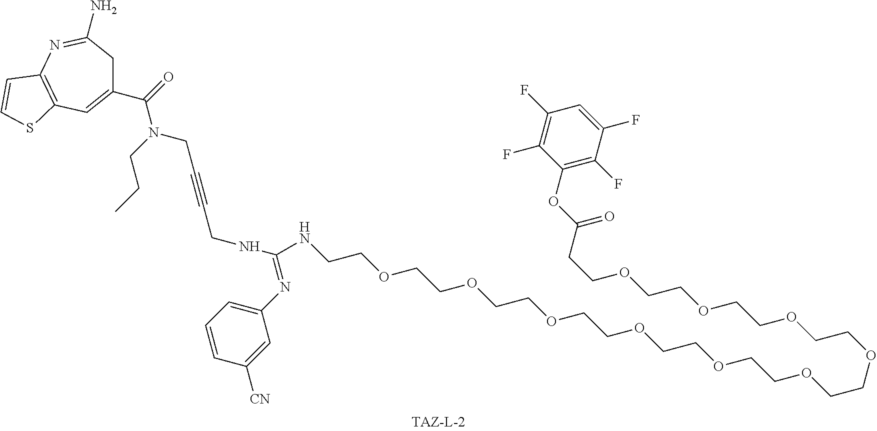 [SMILES: CCCN(CC#CCN/C(=N\c1cccc(C#N)c1)NCCOCCOCCOCCOCCOCCOCCOCCOCCOCCOCCC(=O)Oc1c(F)c(F)cc(F)c1F)C(=O)C1=Cc2sccc2N=C(N)C1]

### Preparation of tert-butyl (E)-41-(5-amino-6H-thieno[3,2-b]azepine-7-carbonyl)-35-((3-cyanophenyl)imino)-4,7,10,13,16,19,22,25,28,31-decaoxa-34,36,41-triazatetratetracont-38-ynoate, L-2a

TAZ-17 (0.05 g, 0.16 mmol, 1 eq.) and tert-butyl 1-((3-cyanophenyl)imino)-5,8,11,14,17,20,23,26,29,32-decaoxa-2-azapentatriacont-1-en-35-oate, PEG10-diimide (0.112 g, 0.16 mmol, 1 eq.) were dissolved in DMF. Triethylamine (0.066 ml, 0.47 mmol, 3 eq.) was added, and the reaction was stirred at ambient temperature. Upon consumption of amine starting material, the reaction was concentrated and purified by HPLC to give L-2a (0.120 g, 0.12 mmol, 74%). LC/MS [M+H] 1028.54 (calculated); LC/MS [M+H] 1028.92 (observed).

### Preparation of (E)-41-(5-amino-6H-thieno[3,2-b]azepine-7-carbonyl)-35-((3-cyanophenyl)imino)-4,7,10,13,16,19,22,25,28,31-decaoxa-34,36,41-triazatetratetracont-38-ynoic acid, L-2b

L-2a (0.120 g, 0.12 mmol, 1 eq.) was dissolved in 100 μl TFA. After 15 minutes, the product was concentrated and purified by HPLC to give L-2b (84.9 mg, 0.087 mmol, 75%). LC/MS [M+H] 972.47 (calculated); LC/MS [M+H] 972.83 (observed).

### Preparation of TAZ-L-2

L-2b (0.085 g, 0.087 mmol, 1 eq.) and TFP (0.029 g, 0.17 mmol, 2 eq.) were dissolved in DMF. Collidine (0.058 ml, 0.44 mmol, 5 eq.) was added, followed by 1-ethyl-3-(3-dimethylaminopropyl)carbodiimide hydrochloride, EDC-HCl (0.033 g, 0.17 mmol, 2 eq.). The reaction was stirred at room temperature until complete, then purified by HPLC to give TAZ-L-2 (0.057 g, 0.055 mmol, 62%). LC/MS [M+H] 1120.47 (calculated); LC/MS [M+H] 1120.85 (observed).

### Example L-3 Synthesis of 2,3,5,6-tetrafluorophenyl 39-(5-amino-7-(dipropylcarbamoyl)-6H-thieno[3,2-b]azepin-2-yl)-34-methyl-4,7,10,13,16,19,22,25,28,31-decaoxa-34-azanonatriacontanoate, TAZ-L-3

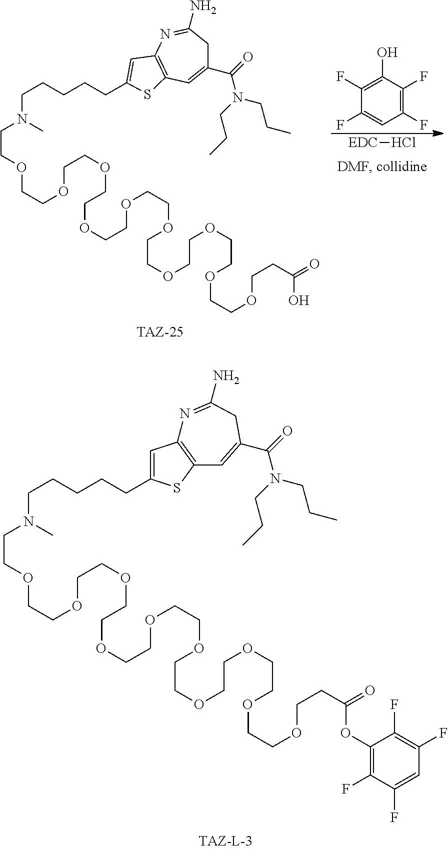

TAZ-25 (0.18 g, 0.20 mmol, 1 eq.) and TFP (0.066 g, 0.40 mmol, 2 eq.) were dissolved in 1 ml DMF. Collidine (0.13 ml, 1.0 mmol, 5 eq.) was added, followed by 1-ethyl-3-(3-dimethylaminopropyl)carbodiimide hydrochloride, EDC-HCl (0.115 g, 0.60 mmol, 3 eq.). The reaction was stirred at room temperature until complete, then purified by HPLC to give TAZ-L-3 (0.103 g, 0.098 mmol, 49%). LC/MS [M+H] 1051.53 (calculated); LC/MS [M+H] 1051.74 (observed).

### Example L-4 Synthesis of 2,3,5,6-tetrafluorophenyl 1-(4-((5-amino-N-propyl-6H-thieno[3,2-b]azepine-7-carboxamido)methyl)phenyl)-2-methyl-5,8,11,14,17,20,23,26,29,32-decaoxa-2-azapentatriacontan-35-oate, TAZ-L-4

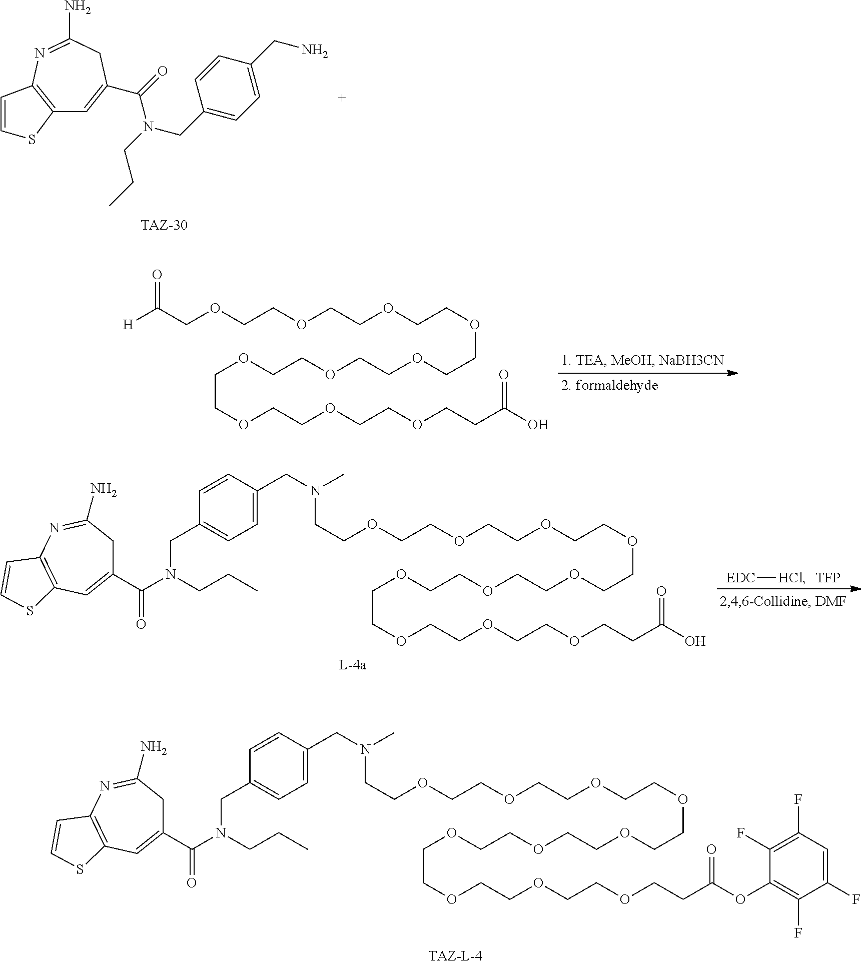

### Preparation of 1-(4-((5-amino-N-propyl-6H-thieno[3,2-b]azepine-7-carboxamido)methyl)phenyl)-2-methyl-5,8,11,14,17,20,23,26,29,32-decaoxa-2-azapentatriacontan-35-oic acid, L-4a

5-Amino-N-(4-(aminomethyl)benzyl)-N-propyl-6H-thieno[3,2-b]azepine-7-carboxamide, TAZ-30 (0.063 g, 0.17 mmol, 1 eq.) and 1-oxo-3,6,9,12,15,18,21,24,27,30-decaoxatritriacontan-33-oic acid (0.09 g, 0.17 mmol, 1 eq.) were dissolved in methanol. Triethylamine (0.14 ml, 1.0 mmol, 6 eq.) was added, followed by sodium cyanoborohydride (0.032 g, 0.51 mmol, 3 eq.). The reaction was monitored by LCMS. After 2 hours, formaldehyde (14 μl, 0.17 mmol, 37% w/w solution in water, 1 eq.) was added and the reaction stirred for an additional 30 minutes. Upon consumption of amine, the reaction was concentrated and purified by HPLC to give L-4a (0.045 g, 0.050 mmol, 29%). LC/MS [M+H] 895.47 (calculated); LC/MS [M+H] 895.80 (observed).

### Preparation of TAZ-L-4

Intermediate L-4a (0.045 g, 0.05 mmol, 1 eq.) and TFP (0.017 g, 0.10 mmol, 2 eq.) were dissolved in 1 ml DMF. Collidine (0.033 ml, 0.25 mmol, 5 eq.) was added, followed by 1-ethyl-3-(3-dimethylaminopropyl)carbodiimide hydrochloride, EDC-HCl (0.029 g, 0.15 mmol, 3 eq.). The reaction was stirred at room temperature until complete, then purified by HPLC to give TAZ-L-4 (0.031 g, 0.030 mmol, 59%). LC/MS [M+H] 1043.47 (calculated); LC/MS [M+H] 1043.79 (observed).

### Example L-10 Synthesis of (2,3,5,6-tetrafluorophenyl) 3-[2-[2-[2-[2-[2-[2-[2-[2-[2-[2-[[4-[[[5-amino-2-[5-(dimethylamino)pentyl]-6H-thieno[3,2-b]azepine-7-carbonyl]-propyl-amino]methyl]phenyl]methyl-methylamino]ethoxy]ethoxy]ethoxy]ethoxy]ethoxy]ethoxy]ethoxy]ethoxy]ethoxy]ethoxy]propanoate, TAZ-L-10

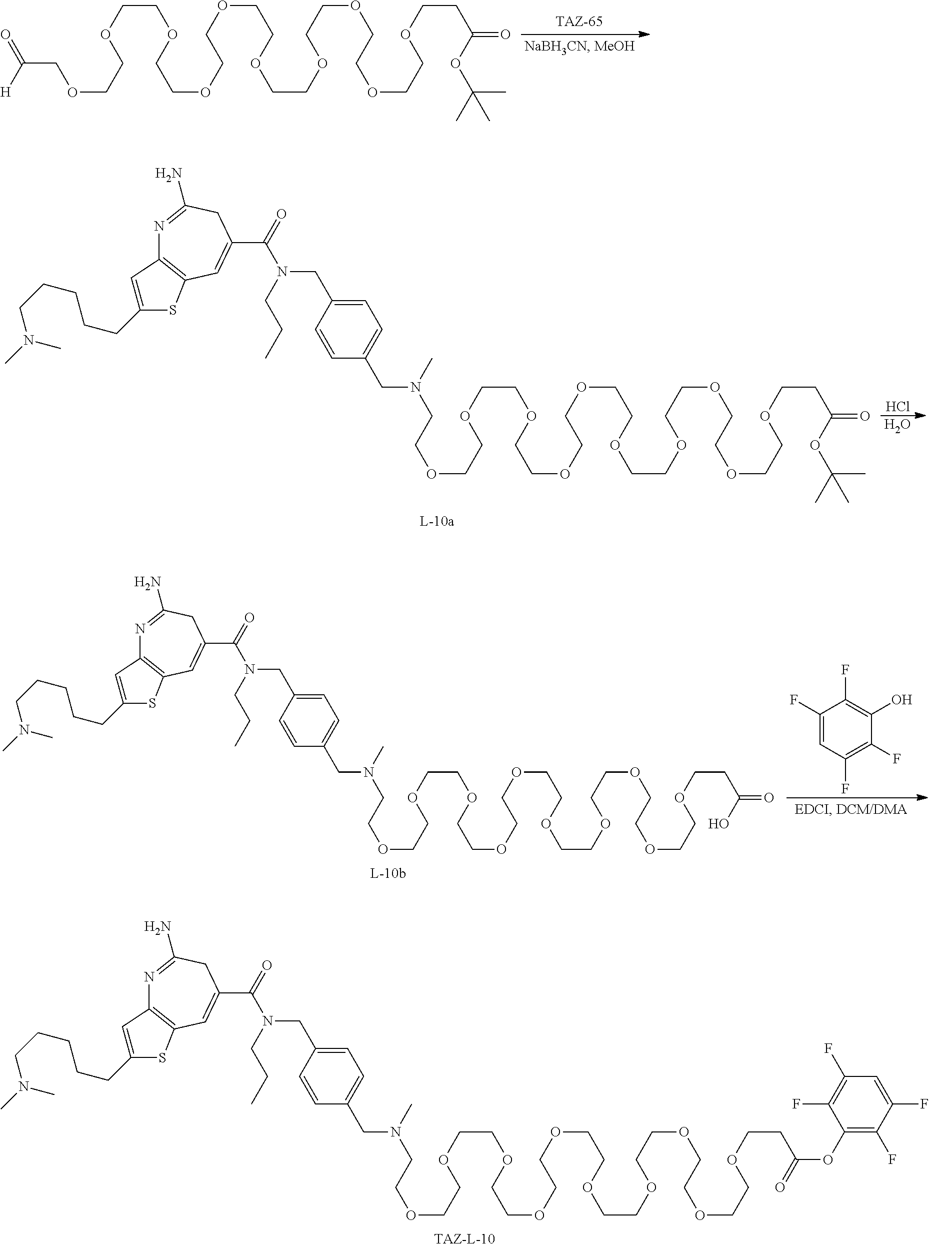

### Preparation of tert-butyl 3-[2-[2-[2-[2-[2-[2-[2-[2-[2-[2-[[4-[[[5-amino-2-[5-(dimethylamino)pentyl]-6H-thieno[3,2-b]azepine-7-carbonyl]-propyl-amino]methyl]phenyl]methyl-methyl-amino]ethoxy]ethoxy]ethoxy]ethoxy]ethoxy]ethoxy]ethoxy]ethoxy]ethoxy]ethoxy]propanoate, L-10a

To a solution of 5-amino-2-[5-(dimethylamino)pentyl]-N-[[4-(methylaminomethyl)phenyl] methyl]-N-propyl-6H-thieno[3,2-b]azepine-7-carboxamide, TAZ-65 (300 mg, 527 umol, 1.0 eq, 2 HCl) in MeOH (10 mL) was added tert-butyl 3-[2-[2-[2-[2-[2-[2-[2-[2-[2-(2-oxoethoxy) ethoxy]ethoxy]ethoxy]ethoxy]ethoxy]ethoxy]ethoxy]ethoxy]ethoxy]propanoate (617 mg, 1.06 mmol, 2.0 eq), AcOH (3.1 mg, 52.7 umol, 0.10 eq) and NaBH₃CN (66.3 mg, 1.06 mmol, 2.0 eq). The mixture was stirred for 12 hrs at 25° C. and then it was concentrated and purified by prep-HPLC (column: Nano-micro Kromasil C18 100*40 mm 10 um; mobile phase: [water (0.1% TFA)−ACN]; B %: 5%-45%, 8 min) to give L-10a (350 mg, 328 umol, 62.33% yield) as colorless oil.

### Preparation of 3-[2-[2-[2-[2-[2-[2-[2-[2-[2-[2-[[4-[[[5-amino-2-[5-(dimethylamino)pentyl]-6H-thieno[3,2-b]azepine-7-carbonyl]-propyl-amino]methyl]phenyl]methyl-methylamino]ethoxy]ethoxy]ethoxy]ethoxy]ethoxy]ethoxy]ethoxy]ethoxy]ethoxy]ethoxy]propanoic acid, L-10b

To a mixture of L-10a (100 mg, 84.8 umol, 1.0 eq, TFA) in H₂O (2 mL) was added HCl (12 M, 141 uL, 20.0 eq) at 25° C., and then stirred at 80° C. for 1 hr. The mixture was concentrated to afford L-10b (80.0 mg, 76.6 umol, 90.2% yield, HCl) as light yellow oil.

### Preparation of TAZ-L-10

To a mixture of L-10b (50.0 mg, 49.6 umol, 1.0 eq) in DCM (2 mL) and DMA (0.1 mL) was added 1-ethyl-3-(3-dimethylaminopropyl)carbodiimide hydrochloride, EDCI (95.0 mg, 495 umol, 10.0 eq) at 15° C. and then stirred for 0.5 hr. The mixture was concentrated in vacuum to give a residue. The residue was purified by prep-HPLC (column: Phenomenex Synergi C18 150*25*10 um; mobile phase: [water (0.1% TFA)−ACN]; B %: 25%-50%, 8 min) to obtain TAZ-L-10 (21.0 mg, 16.5 umol, 34.5% yield, TFA) as light yellow oil. ¹H NMR (MeOD, 400 MHz) δ 7.57-7.50 (m, 2H), 7.48-7.43 (m, 3H), 7.12 (s, 1H), 6.92 (s, 1H), 4.80 (s, 2H), 4.50-4.32 (m, 2H), 3.90-3.87 (m, 4H), 3.70-3.59 (m, 38H), 3.38-3.40 (m, 6H), 3.01-2.99 (m, 2H), 2.90-2.98 (m, 2H), 2.90 (s, 9H), 1.79-1.78 (m, 4H), 1.70-1.65 (m, 2H), 1.52-1.49 (m, 2H), 0.93-0.87 (m, 3H). LC/MS [M+H] 1156.6 (calculated); LC/MS [M+H] 1156.6 (observed).

### Example L-13 Synthesis of (2,3,5,6-tetrafluorophenyl) 3-[2-[2-[2-[2-[2-[2-[2-[2-[2-[2-[5-[5-amino-7-[[4-[(dimethylamino)methyl]phenyl]methyl-propyl-carbamoyl]-6H-thieno[3,2-b]azepin-2-yl]pentyl-methylamino]ethoxy]ethoxy]ethoxy]ethoxy]ethoxy]ethoxy]ethoxy]ethoxy]ethoxy]ethoxy]propanoate, TAZ-L-13

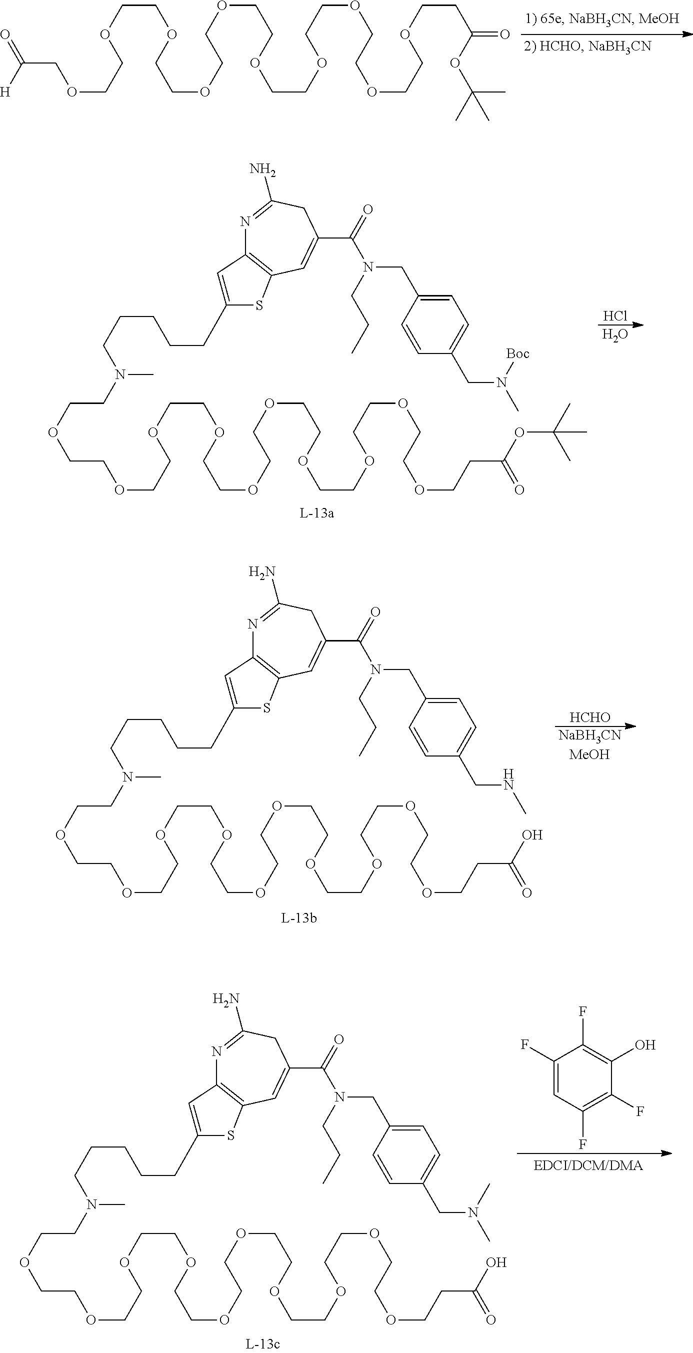

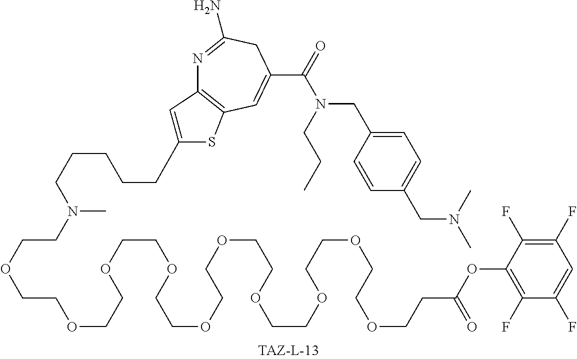 [SMILES: CCCN(Cc1ccc(CN(C)C)cc1)C(=O)C1=Cc2sc(CCCCCN(C)CCOCCOCCOCCOCCOCCOCCOCCOCCOCCOCCC(=O)Oc3c(F)c(F)cc(F)c3F)cc2N=C(N)C1]

### Preparation of tert-butyl 3-[2-[2-[2-[2-[2-[2-[2-[2-[2-[2-[5-[5-amino-7-[[4-[[tert- butoxycarbonyl(methyl)amino]methyl]phenyl]methyl-propyl-carbamoyl]-6H-thieno[3,2-b]azepin-2-yl]pentyl-methyl-amino]ethoxy]ethoxy]ethoxy]ethoxy]ethoxy]ethoxy]ethoxy]ethoxy]ethoxy]ethoxy]propanoate, L-13a

To a solution of tert-butyl N-[[4-[[[5-amino-2-(5-aminopentyl)-6H-thieno[3,2-b]azepine-7-carbonyl]-propyl-amino]methyl]phenyl]methyl]-N-methyl-carbamate, 65e (130 mg, 229 umol, 1.0 eq) in MeOH (50 mL) was added AcOH (13.7 mg, 228.96 umol, 13.0 uL, 1 eq) tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (200 mg, 343 umol, 1.50 eq) and NaBH₃CN (43.1 mg, 687 umol, 3.0 eq), and then stirred for 12 hrs at 25° C. Formaldehyde, HCHO (56 mg, 674 umol, 37% purity, 3.0 eq) and NaBH₃CN (22.0 mg, 343 umol, 1.5 eq) were added to the mixture and then stirred for another 2 hrs at 25° C. The reaction mixture was quenched by addition H₂O 2 mL and it was concentrated in vacuum to give a residue. The residue was purified by prep-HPLC (column: Phenomenex luna C18 250*50 mm*10 um; mobile phase: [water(0.1% TFA)-ACN]; B %: 30%-60%, 10 min) to give L-13a (100 mg, 86.92 umol, 38% yield) as colorless oil.

### Preparation of 3-[2-[2-[2-[2-[2-[2-[2-[2-[2-[2-[5-[5-amino-7-[[4-(methylaminomethyl)phenyl]methyl-propyl-carbamoyl]-6H-thieno[3,2-b]azepin-2-yl]pentyl-methyl-amino]ethoxy]ethoxy]ethoxy]ethoxy]ethoxy]ethoxy]ethoxy]ethoxy]ethoxy]ethoxy]propanoic acid, L-13b

To a solution of L-13a (100 mg, 86.9 umol, 1.0 eq) in H₂O (0.5 mL) was added HCl (12 M, 145 uL, 20.0 eq) and then stirred for 0.5 h at 80° C. The reaction mixture was concentrated under pressure to give L-13b (92 mg, crude) as colorless oil.

### Preparation of 3-[2-[2-[2-[2-[2-[2-[2-[2-[2-[2-[5-[5-amino-7-[[4-[(dimethylamino)methyl]phenyl]methyl-propyl-carbamoyl]-6H-thieno[3,2-b]azepin-2-yl]pentyl-methylamino]ethoxy]ethoxy]ethoxy]ethoxy]ethoxy]ethoxy]ethoxy]ethoxy]ethoxy]ethoxy]propanoic acid, L-13c

To a mixture of HCHO, formaldehyde (30.4 mg, 375 umol, 37% purity, 5.0 eq) and L-13b (80 mg, 74.9 umol, 1.0 eq, 2HCl) in MeOH (1 mL) was added NaBH₃CN (9.4 mg, 150 umol, 2.0 eq) at 15° C. and then stirred at 15° C. for 1 hr. The mixture was filtered and the filtrate was purified by prep-HPLC (column: Phenomenex luna C18 80*40 mm*3 um; mobile phase: [water(0.04% HCl)−ACN]; B %: 5%-35%, 7 min) to obtain L-13c (55 mg, 50.87 umol, 67.86% yield, 2HCl) as colorless oil. ¹H NMR (MeOD, 400 MHz) δ 7.56 (d, J=8.0 Hz, 2H), 7.48 (d, J=7.2 Hz, 2H), 7.13 (s, 1H), 6.95 (s, 1H), 4.83 (s, 2H), 4.35 (s, 2H), 3.76-3.70 (m, 2H), 3.70-3.62 (m, 40H), 3.50-3.47 (m, 6H), 3.35-3.28 (m, 2H), 2.94-2.91 (m, 5H), 2.88 (s, 6H), 2.56 (t, J=6.4 Hz, 2H), 1.82-1.81 (m, 3H), 1.69-1.65 (m, 2H), 1.53-1.49 (m, 2H), 0.91 (t, J=7.2 Hz, 3H)

### Preparation of TAZ-L-13

To a mixture of L-13c (50 mg, 49.6 umol, 1.0 eq) in DCM (2 mL) and DMA (0.1 mL) was added 1-ethyl-3-(3-dimethylaminopropyl)carbodiimide hydrochloride, EDCI (95.0 mg, 496 umol, 10.0 eq) at 15° C. and then stirred at 15° C. for 0.5 hr. The mixture was concentrated under reduced pressure to give a residue. The residue was purified by prep-HPLC (column: Phenomenex Synergi C18 150*25*10 um; mobile phase: [water(0.1% TFA)−ACN]; B %: 20%-40%, 10 min) to afford TAZ-L-13 (23 mg, 19.89 umol, 40.11% yield) as light yellow oil. ¹H NMR (MeOD, 400 MHz) δ 7.53-7.51 (m, 2H), 7.46-7.40 (m, 3H), 7.11 (s, 1H), 6.92 (s, 1H), 4.80 (s, 2H), 4.33 (s, 2H), 3.89 (t, J=6.4 Hz, 2H), 3.89-3.85 (m, 2H), 3.70-3.63 (m, 38H), 3.54-3.42 (m, 4H), 3.39 (s, 2H), 2.99 (t, J=6.0 Hz, 2H), 2.94-2.91 (m, 5H), 2.87 (s, 6H), 1.80 (d, J=6.4 Hz, 4H), 1.69-1.64 (m, 2H), 1.52-1.50 (m, 2H), 0.90 (t, J=6.8 Hz, 3H). LC/MS [M+H] 1156.6 (calculated); LC/MS [M+H] 1156.6 (observed).

### Example L-16 Synthesis of (2,3,5,6-tetrafluorophenyl) 3-[2-[2-[2-[2-[2-[2-[2-[2-[2-[2-[[4-[[(5-amino-6H-thieno [3,2-b]azepine-7-carbonyl)-propyl-amino]methyl]-2-(trifluoromethyl)phenyl]methyl-methyl-amino]ethoxy]ethoxy]ethoxy]ethoxy]ethoxy]ethoxy]ethoxy]ethoxy]ethoxy]ethoxy]propanoate, TAZ-L-16

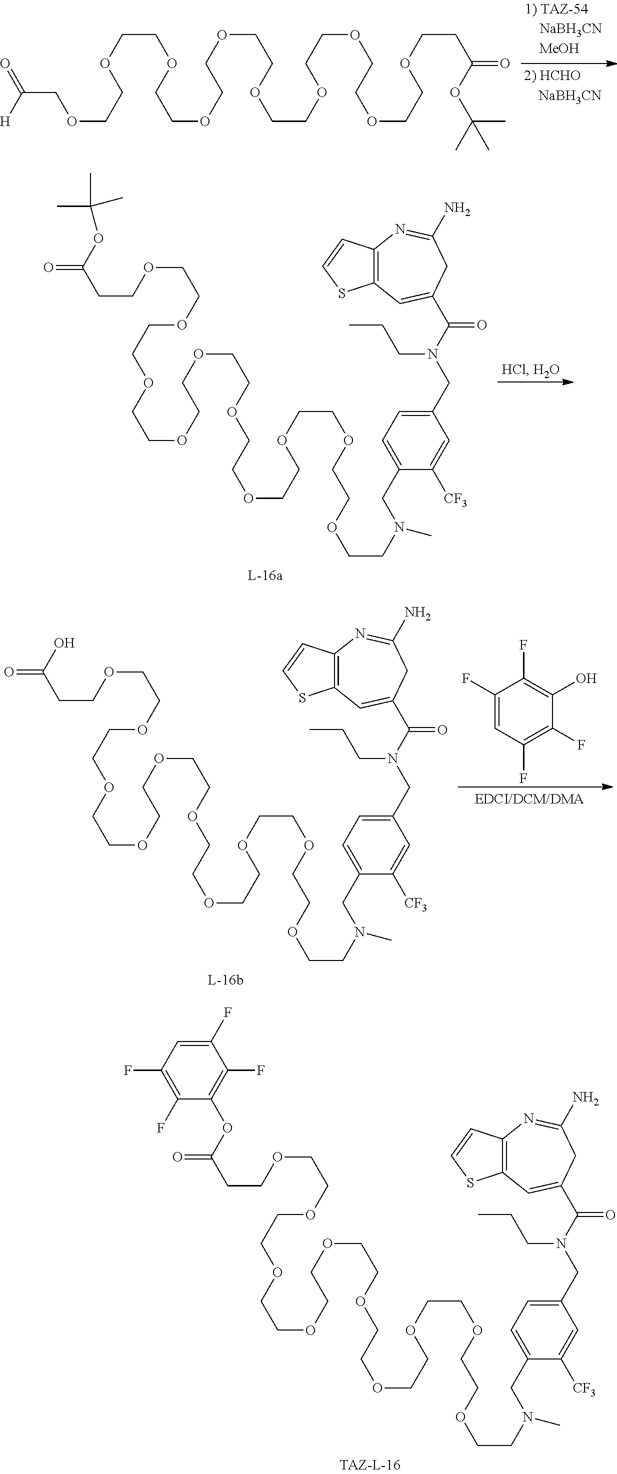

### Preparation of tert-butyl 3-[2-[2-[2-[2-[2-[2-[2-[2-[2-[2-[[4-[[(5-amino-6H-thieno[3,2-b]azepine-7-carbonyl)-propyl-amino]methyl]-2-(trifluoromethyl)phenyl]methyl-methyl-amino]ethoxy]ethoxy]ethoxy]ethoxy]ethoxy]ethoxy]ethoxy]ethoxy]ethoxy]ethoxy]propanoate, L-16a

To a mixture of 5-amino-N-[[4-(aminomethyl)-3-(trifluoromethyl)phenyl]methyl]-N-propyl-6H-thieno[3,2-b]azepine-7-carboxamide, TAZ-54 (80.0 mg, 145 umol, 1.0 eq, TFA) and tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (110 mg, 189 umol, 1.3 eq) in MeOH (20 mL) was added NaBH₃CN (22.8 mg, 363 umol, 2.5 eq) at 20° C. and then stirred 20 hrs at this temperature. HCHO (70.7 mg, 872 umol, 64.9 uL, 37% purity, 6.0 eq) and NaBH₃CN (22.8 mg, 363 umol, 2.5 eq) was added to the mixture, and it was stirred at 20° C. for another 1 hour. The reaction mixture was concentrated in vacuum and purified by prep-HPLC (column: Nano-micro Kromasil C18 100*40 mm 10 um; mobile phase: [water (0.1% TFA)−ACN]; B %: 3%-40%, 8 min) to afford L-16a (88.0 mg, 86.3 umol, 59.4% yield) as colorless oil. ¹H NMR (400 MHz, MeOD) δ7.90-7.83 (m, 2H), 7.83-7.78 (m, 1H), 7.76 (d, J=5.2 Hz, 1H), 7.22 (d, J=6.4 Hz, 1H), 7.16 (d, J=4.8 Hz, 1H), 4.73-4.55 (m, 2H), 3.93-3.84 (m, 2H), 3.71-3.70 (m, 4H), 3.68-3.57 (m, 38H), 3.49 (s, 2H), 3.41 (s, 2H), 2.95 (s, 3H), 2.48 (t, J=6.0 Hz, 2H), 1.75-1.67 (m, 2H), 1.47 (s, 9H), 0.93 (br t, J=7.6 Hz, 3H).

### Preparation of 3-[2-[2-[2-[2-[2-[2-[2-[2-[2-[2-[[4-[[(5-amino-6H-thieno[3,2-b]azepine-7-carbonyl)-propyl-amino]methyl]-2-(trifluoromethyl)phenyl]methyl-methyl-amino]ethoxy]ethoxy]ethoxy]ethoxy]ethoxy]ethoxy]ethoxy]ethoxy]ethoxy]ethoxy]propanoic acid, L-16b

To a solution of L-16a (78.0 mg, 76.5 umol, 1.0 eq) in H₂O (0.5 mL) and MeCN (0.1 mL) was added HCl (12 M, 191 uL, 30 eq) at 20° C. under N₂, the mixture was heated to 80° C. and then stirred for 1 hour. The reaction mixture was concentrated in vacuum to afford L-16b (70.0 mg, 72.6 umol, 94.9% yield) as colorless oil.

### Preparation of TAZ-L-16

To a mixture of L-16b (60 mg, 62.3 umol, 1 eq) and 2,3,5,6-tetrafluorophenol (51.7 mg, 311 umol, 5.0 eq) in DCM (1 mL) and DMA (0.1 mL) was added EDCI (59.7 mg, 311 umol, 5.0 eq) at 20° C. under N₂, and then stirred at 20° C. for 1 hour. The reaction mixture was concentrated in vacuum. The residue was purified by prep-HPLC (column: Phenomenex Synergi C18 150*25*10 um; mobile phase: [water (0.1% TFA)−ACN]; B %: 25%-50%, 7 min) to afford TAZ-L-16 (15 mg, 13.5 umol, 21.67% yield) as colorless oil. ¹H NMR (400 MHz, MeOD) δ7.88-7.83 (m, 2H), 7.82-7.78 (m, 1H), 7.76 (d, J=5.6 Hz, 1H), 7.48-7.41 (m, 1H), 7.22 (s, 1H), 7.16 (d, J=5.6 Hz, 1H), 4.96 (s, 2H), 3.92-3.87 (m, 4H), 3.71-3.58 (m, 38H), 3.52-3.48 (m, 2H), 3.41 (s, 2H), 3.34 (s, 2H), 2.99 (t, J=6.0 Hz, 2H), 2.95 (s, 3H), 1.75-1.66 (m, 2H), 0.92 (t, J=7.2 Hz, 3H). LC/MS [M+H] 1111.5 (calculated); LC/MS [M+H] 1111.5 (observed).

### Example L-22 Synthesis of (2,3,5,6-tetrafluorophenyl) 3-[2-[2-[2-[2-[2-[2-[2-[2-[2-[2-[[4-[[(5-amino-6H-thieno [3,2-b]azepine-7-carbonyl)-propyl-amino]methyl]-3-(trifluoromethyl)phenyl]methyl-methyl-amino]ethoxy]ethoxy]ethoxy]ethoxy]ethoxy]ethoxy]ethoxy]ethoxy]ethoxy]ethoxy]propanoate, TAZ-L-22

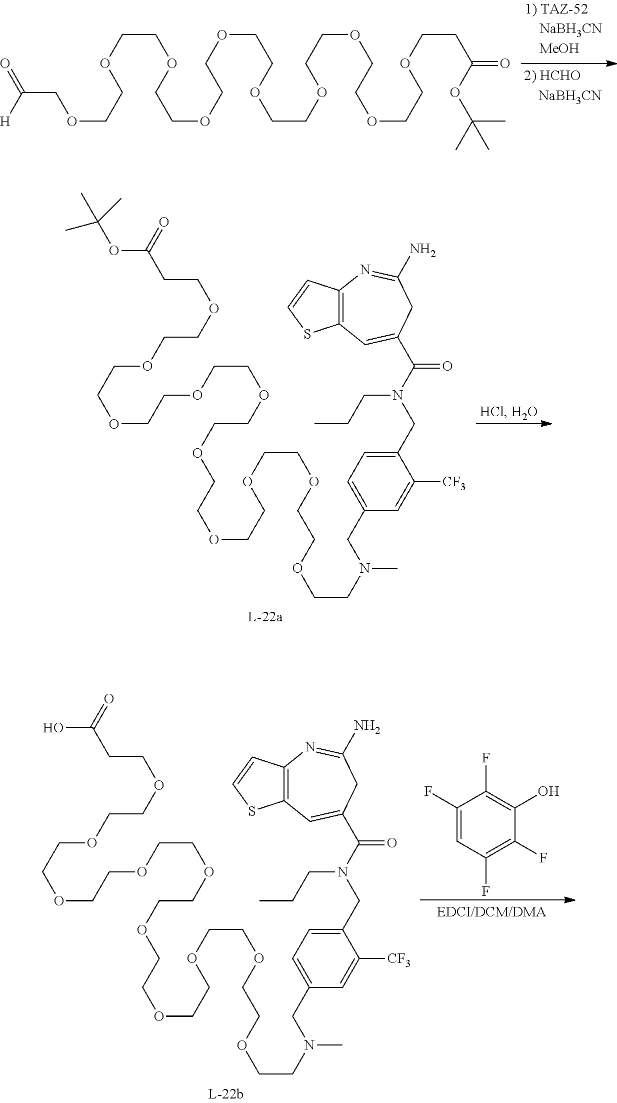

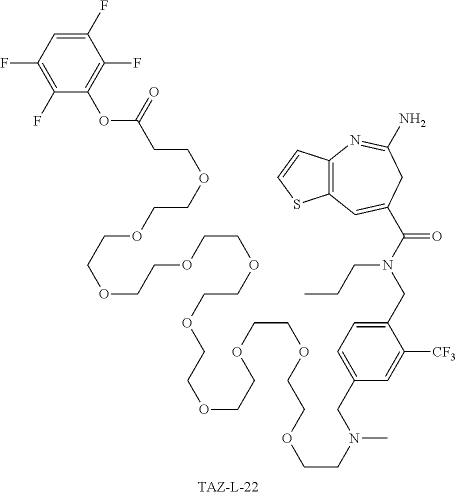 [SMILES: CCCN(Cc1ccc(CN(C)CCOCCOCCOCCOCCOCCOCCOCCOCCOCCOCCC(=O)Oc2c(F)c(F)cc(F)c2F)cc1C(F)(F)F)C(=O)C1=Cc2sccc2N=C(N)C1]

### Preparation of tert-butyl 3-[2-[2-[2-[2-[2-[2-[2-[2-[2-[2-[[4-[[(5-amino-6H-thieno[3,2-b]azepine-7-carbonyl)-propyl-amino]methyl]-3-(trifluoromethyl)phenyl]methyl-methyl-amino]ethoxy]ethoxy]ethoxy]ethoxy]ethoxy]ethoxy]ethoxy]ethoxy]ethoxy]ethoxy]propanoate, L-22a

To a mixture of tert-butyl 3-[2-[2-[2-[2-[2-[2-[2-[2-[2-(2-oxoethoxy)ethoxy]ethoxy]ethoxy] ethoxy]ethoxy]ethoxy]ethoxy]ethoxy]ethoxy]propanoate (51.9 mg, 89 umol, 1.4 eq) in MeOH (3 mL) was added 5-amino-N-[[4-(aminomethyl)-2-(trifluoromethyl)phenyl]methyl]-N-propyl-6H-thieno[3,2-b]azepine-7-carboxamide, TAZ-52 (30 mg, 63 umol, 1.0 eq, HCl) at 25° C. The mixture was stirred for 10 min, then NaBH₃CN (7.97 mg, 126.86 umol, 2 eq) was added and it was stirred at 25° C. for 23 hours, then formaldehyde (15.44 mg, 190.29 umol, 14.17 uL, 3 eq) and NaBH₃CN (7.97 mg, 126.86 umol, 2 eq) was added and the reaction was stirred for another 1 hour. Followed, the reaction mixture was concentrated and purified by prep-HPLC (column: Phenomenex Luna C18 150*30 mm*5 um; mobile phase: [water (0.1% TFA)−ACN]; B %: 25%-55%, 10 min) to give L-22a (60 mg, crude) as colorless oil.

### Preparation of 3-[2-[2-[2-[2-[2-[2-[2-[2-[2-[2-[[4-[[(5-amino-6H-thieno[3,2-b]azepine-7-carbonyl)-propyl-amino]methyl]-3-(trifluoromethyl)phenyl]methyl-methyl-amino]ethoxy]ethoxy]ethoxy]ethoxy]ethoxy]ethoxy]ethoxy]ethoxy]ethoxy]ethoxy]propanoic acid, L-22b

To a mixture of L-22a (60 mg, 58.87 umol, 1 eq) in H₂O (0.5 mL) was added HCl (12 M, 150 uL, 30 eq) in one portion at 15° C. and then stirred at 80° C. for 2 hours. The mixture was concentrated in vacuum to obtain L-22b (45 mg, 45.02 umol, 76.47% yield, HCl) as light yellow oil.

### Preparation of TAZ-L-22

To a mixture of L-22b (40 mg, 40 umol, 1.0 eq, HCl) in DCM (0.2 mL) and DMA (0.02 mL) was added 2,3,5,6-tetrafluorophenol (53.2 mg, 320 umol, 8.0 eq) and EDCI (76.7 mg, 400 umol, 10 eq) at 15° C., and then stirred for 30 min. The reaction was concentrated under reduced pressure at 30° C. and purified by prep-HPLC (column: Phenomenex Synergi C18 150*25*10 um; mobile phase: [water (0.1% TFA)−ACN]; B %: 20%-50%, 8 min) to afford TAZ-L-22 (24.7 mg, 22.23 umol, 55.55% yield) as light yellow oil. ¹H NMR (MeOD, 400 MHz) δ7.98 (s, 1H), 7.86-7.84 (m, 1H), 7.76-7.72 (m, 1H), 7.65 (d, J=8.0 Hz, 1H), 7.48-7.39 (m, 1H), 7.14 (d, J=5.2 Hz, 1H), 4.96 (s, 2H), 4.70-4.41 (m, 2H), 3.92-3.82 (m, 4H), 3.70-3.54 (m, 38H), 3.44-3.40 (m, 4H), 3.34 (s, 2H), 2.97 (t, J=6.0 Hz, 2H), 2.91 (s, 3H), 1.75-1.59 (m, 2H), 0.91 (t, J=6.8 Hz, 3H). LC/MS [M+H] 1111.5 (calculated); LC/MS [M+H] 1111.4 (observed).

### Example L-32 Synthesis of 2,3,5,6-tetrafluorophenyl 40-(5-amino-7-((3-(3,3-dimethylbutanamido)propyl)(propyl)carbamoyl)-6H-thieno[3,2-b]azepin-2-yl)-34-oxo-4,7,10,13,16,19,22,25,28,31-decaoxa-35-azatetracontanoate, TAZ-L-32

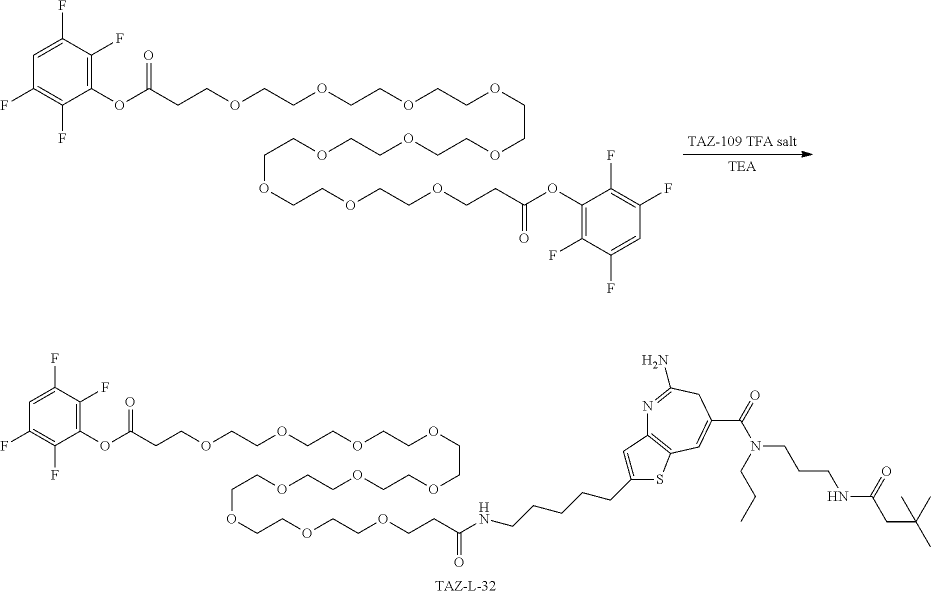 [SMILES: O=C(CCOCCOCCOCCOCCOCCOCCOCCOCCOCCOCCC(=O)Oc1c(F)c(F)cc(F)c1F)Oc1c(F)c(F)cc(F)c1F ; CCCN(CCCNC(=O)CC(C)(C)C)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1]

Bis(2,3,5,6-tetrafluorophenyl) 4,7,10,13,16,19,22,25,28,31-decaoxatetratriacontanedioate (10 mg, 0.12 mmol, 1 eq.) was dissolved in 0.5 ml acetonitrile, ACN. To this solution was added dropwise a solution of the trifluoroacetate salt of 5-amino-2-(5-aminopentyl)-N-(3-(3,3-dimethylbutanamido)propyl)-N-propyl-6H-thieno[3,2-b]azepine-7-carboxamide, TAZ-109 (7.4 mg, 0.012 mmol, 1 eq.) and triethylamine, TEA (0.01 ml, 0.073 mmol, 6 eq.) in 1 ml ACN. Upon completion, the reaction was purified by HPLC to TAZ-L-32 as a colorless glass (4 mg, 0.03 mmol, 28%). LC/MS [M+H] 1178.59 (calculated); LC/MS [M+H] 1178.83 (observed).

### Example L-34 Synthesis of 2,3,5,6-tetrafluorophenyl 39-(5-amino-7-((3-(3,3-dimethylbutanamido)propyl)(propyl)carbamoyl)-6H-thieno[3,2-b]azepin-2-yl)-34-methyl-4,7,10,13,16,19,22,25,28,31-decaoxa-34-azanonatriacontanoate, TAZ-L-34

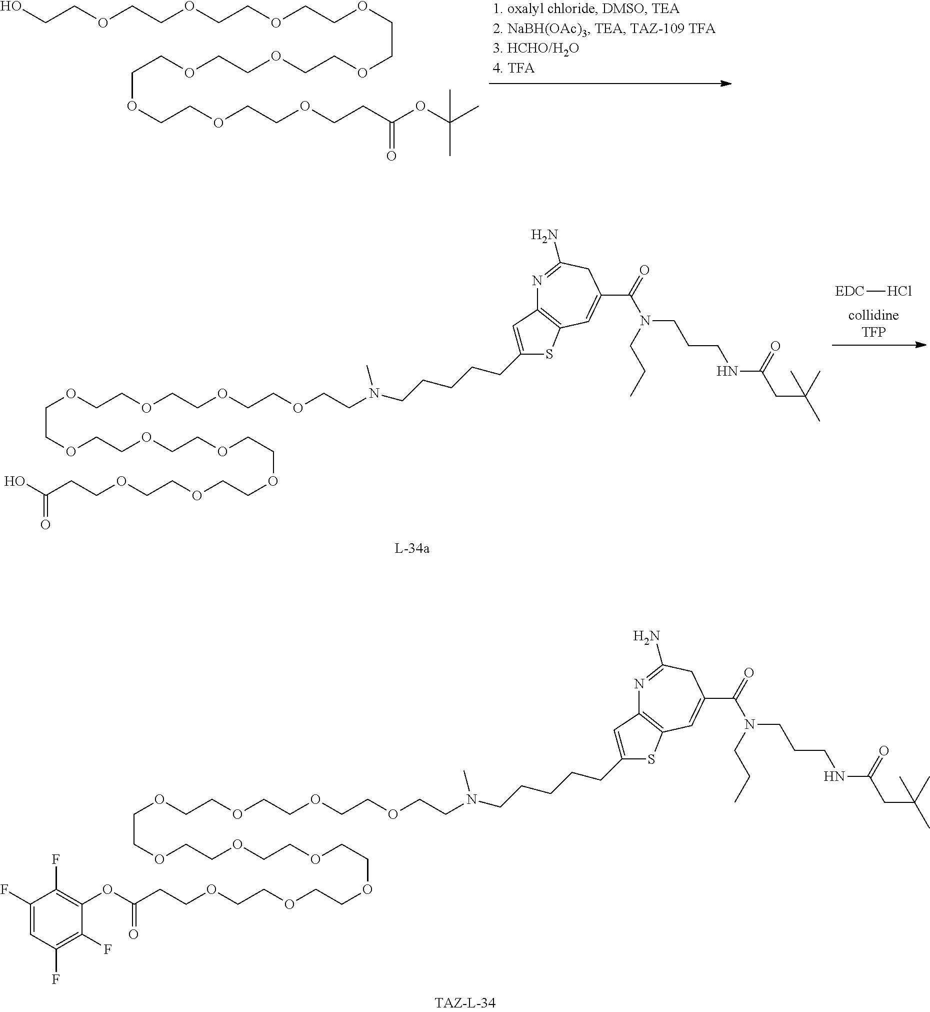

### Preparation of 39-(5-amino-7-((3-(3,3-dimethylbutanamido)propyl)(propyl)carbamoyl)-6H-thieno[3,2-b]azepin-2-yl)-34-methyl-4,7,10,13,16,19,22,25,28,31-decaoxa-34-azanonatriacontanoic acid, L-34a

Oxalyl chloride (0.023 ml, 0.27 mmol, 3 eq.) was dissolved in 2.5 ml DCM at −78° C. dimethylsulfoxide, DMSO (0.038 ml, 0.54 mmol, 6 eq.) was added dropwise. The reaction was stirred at −78° C. for 15 minutes, then tert-butyl 1-hydroxy-3,6,9,12,15,18,21,24,27,30-decaoxatritriacontan-33-oate (0.052 g, 0.089 mmol, 1 eq.) was added dropwise as a solution in 0.5 ml DCM. The reaction was stirred 30 minutes at −78° C., and then triethylamine, TEA (0.112 ml, 0.80 mmol, 9 eq.) was added dropwise. The reaction was stirred 30 more minutes at −78° C., then removed from cooling and allowed to warm to ambient temperature over 30 minutes to form the crude aldehyde intermediate. The trifluoroacetate salt of 5-amino-2-(5-aminopentyl)-N-(3-(3,3-dimethylbutanamido)propyl)-N-propyl-6H-thieno[3,2-b]azepine-7-carboxamide, TAZ-109 (0.054 g, 0.089 mmol, 1 eq.) and sodium triacetoxyborohydride, STAB (0.186 g, 0.88 mmol, 9.8 eq.) were suspended in 2 ml DCM with an additional 0.05 ml TEA. The crude aldehyde solution was added to the stirring solution. The reaction was stirred at room temperature for 3 hours, and then formaldehyde (0.0073 g, 0.089 mmol, 1 eq., 37 wt. % in H₂O) added. After 15 minutes, the reaction was concentrated and purified by HPLC to give as a colorless residue, which was dissolved in minimal TFA and allowed to stand for 15 minutes.

The solution was then concentrated and triturated with diethyl ether to give L-34a (0.042 g, 0.041 mmol, 46%). LC/MS [M+H] 1016.62 (calculated); LC/MS [M+H] 1016.95 (observed).

### Preparation of TAZ-L-34

Intermediate L-34a (0.042 g, 0.041 mmol, 1 eq.) and 2,3,5,6-tetrafluorophenol, TFP (0.014 g, 0.082 mmol, 2 eq.) were dissolved in 3 ml ACN. Collidine (0.054 ml, 0.406 mmol, 9.83 eq.) was added, followed by 1-ethyl-3-(3-dimethylaminopropyl)carbodiimide hydrochloride, EDC-HCl (0.017 g, 0.088 mmol, 2.13 eq.). The reaction was stirred at room temperature and monitored by LCMS, then diluted with 2 ml H₂O and purified by HPLC to give TAZ-L-34 (0.0198 g, 0.017 mmol, 41%). LC/MS [M+H] 1164.61 (calculated); LC/MS [M+H] 1164.81 (observed).

### Example L-52 Synthesis of 2,3,5,6-tetrafluorophenyl 40-(5-amino-7-(propyl(3-(2-(trifluoromethoxy)acetamido)propyl)carbamoyl)-6H-thieno[3,2-b]azepin-2-yl)-34-oxo-4,7,10,13,16,19,22,25,28,31-decaoxa-35-azatetracontanoate, TAZ-L-52

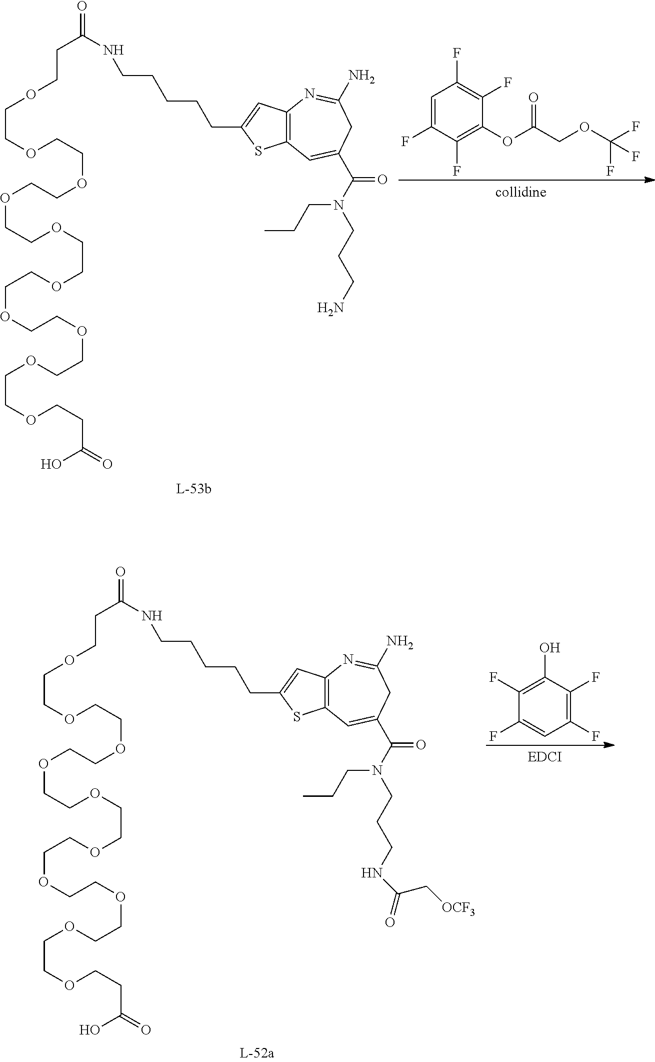 [SMILES: CCCN(CCCN)C(=O)C1=Cc2sc(CCCCCNC(=O)CCOCCOCCOCCOCCOCCOCCOCCOCCOCCOCCC(=O)O)cc2N=C(N)C1 ; O=C(COC(F)(F)F)Oc1c(F)c(F)cc(F)c1F ; CCCN(CCCNC(=O)COC(F)(F)F)C(=O)C1=Cc2sc(CCCCCNC(=O)CCOCCOCCOCCOCCOCCOCCOCCOCCOCCOCCC(=O)O)cc2N=C(N)C1 ; Oc1c(F)c(F)cc(F)c1F]

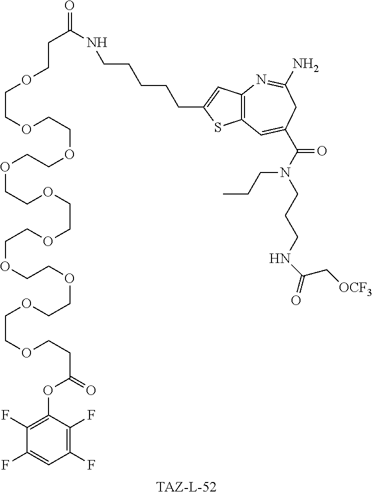 [SMILES: CCCN(CCCNC(=O)COC(F)(F)F)C(=O)C1=Cc2sc(CCCCCNC(=O)CCOCCOCCOCCOCCOCCOCCOCCOCCOCCOCCC(=O)Oc3c(F)c(F)cc(F)c3F)cc2N=C(N)C1]

### Preparation of 40-(5-amino-7-(propyl(3-(2-(trifluoromethoxy)acetamido)propyl)carbamoyl)-6H-thieno[3,2-b]azepin-2-yl)-34-oxo-4,7,10,13,16,19,22,25,28,31-decaoxa-35-azatetracontanoic acid, L-52a

2,3,5,6-Tetrafluorophenyl 2-(trifluoromethoxy)acetate (0.012 g, 0.041 mmol, 1 equiv.) and 3-(5-amino-2-(1-carboxy-33-oxo-3,6,9,12,15,18,21,24,27,30-decaoxa-34-azanonatriacontan-39-yl)-N-propyl-6H-thieno[3,2-b]azepine-7-carboxamido)propan-1-aminium trifluoroacetate, L-53b (0.038 g, 0.041 mmol, 1 equiv.) were combined in acetonitrile. Collidine (0.027 ml, 0.205 mmol, 5 equiv.) was added, and the reaction monitored by HPLC. Upon completion, the reaction was concentrated and purified by HPLC to give L-52a (0.07 g, 0.066 mmol, 160%) as a syrup containing a significant amount of residual collidine. The crude material was carried on to the next step without further purification. LC/MS [M+H] 1058.52 (calculated); LC/MS [M+H] 1058.84 (observed).

### Preparation of TAZ-L-52

Intermediate L-52a (0.07 g, 0.066 mmol, 1 equiv.) and 2,3,5,6-tetrafluorophenol (0.011 g, 0.66 mmol, 1 equiv.) were dissolved in 1 ml acetonitrile. Collidine (0.017 ml, 0.16 mmol, 2 equiv.) was added, followed by EDC (0.013 g, 0.66 mmol, 1 equiv.). The reaction was stirred at room temperature and monitored by LCMS, then diluted with water and purified by reverse-phase HPLC to give TAZ-L-52 (0.095 g, 0.075 mmol, 49%). LC/MS [M+H] 1206.51 (calculated); LC/MS [M+H] 1206.51 (observed).

### Example L-53 Synthesis of 2,3,5,6-tetrafluorophenyl 40-(5-amino-7-((3-((cyclobutoxycarbonyl)amino)propyl)(propyl)carbamoyl)-6H-thieno[3,2-b]azepin-2-yl)-34-oxo-4,7,10,13,16,19,22,25,28,31-decaoxa-35-azatetracontanoate, TAZ-L-53

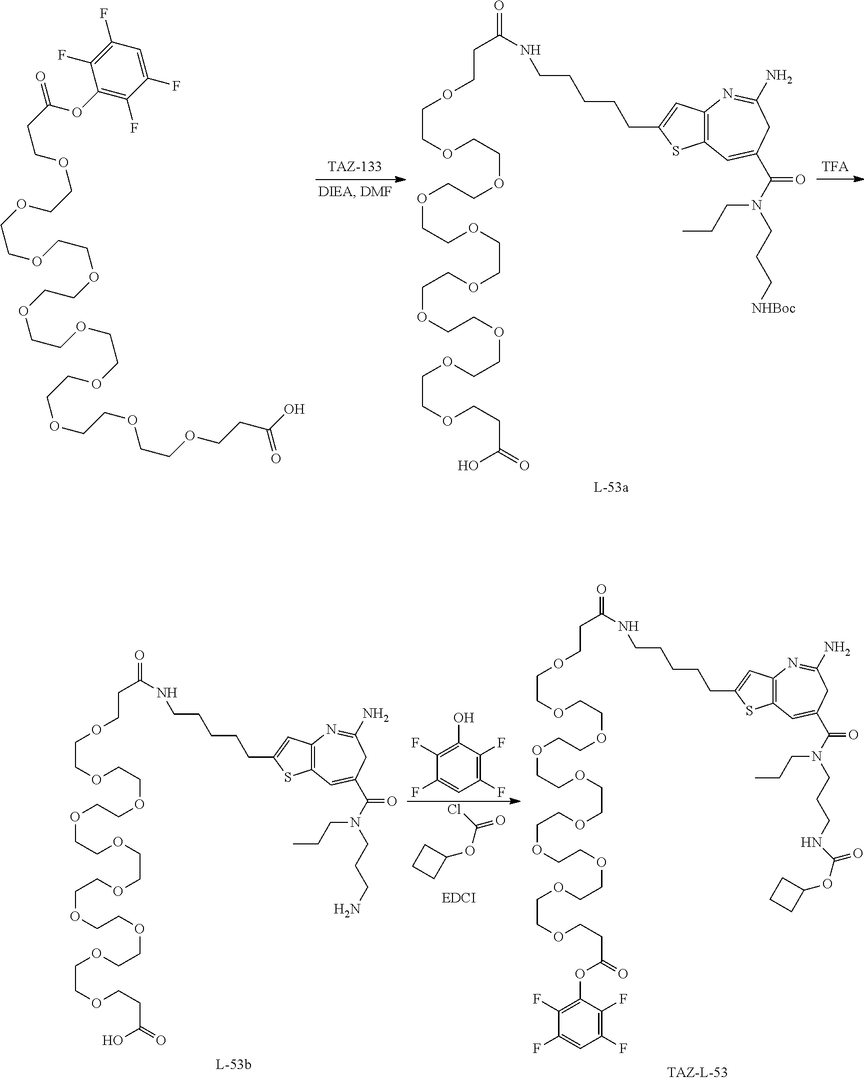

### Preparation of 40-(5-amino-7-((3-((tert-butoxycarbonyl)amino)propyl)(propyl)carbamoyl)-6H-thieno[3,2-b]azepin-2-yl)-34-oxo-4,7,10,13,16,19,22,25,28,31-decaoxa-35-azatetracontanoic acid, L-53a

Tert-butyl (3-(5-amino-2-(5-aminopentyl)-N-propyl-6H-thieno[3,2-b]azepine-7-carboxamido)propyl)carbamate, TAZ-133 (0.234 g, 0.48 mmol, 1 equiv.) and 34-oxo-34-(2,3,5,6-tetrafluorophenoxy)-4,7,10,13,16,19,22,25,28,31-decaoxatetratriacontanoic acid (0.34 g, 0.48 mmol, 1 equiv.) were dissolved in DMF. Triethylamine (0.33 g, 2.4 mmol, 5 equiv.) was added and the reaction stirred at room temperature. Upon consumption of amine starting material, the reaction was diluted with water and purified by reverse-phase HPLC to give L-53a (0.385 g, 0.40 mmol, 84%). LC/MS [M+H] 1032.58 (calculated); LC/MS [M+H] 1032.89 (observed).

### Preparation of 3-(5-amino-2-(1-carboxy-33-oxo-3,6,9,12,15,18,21,24,27,30-decaoxa-34-azanonatriacontan-39-yl)-N-propyl-6H-thieno[3,2-b]azepine-7-carboxamido)propan-1-aminium trifluoroacetate, L-53b

Intermediate L-53a (0.27 g, 0.26 mmol, 1 equiv.) was dissolved in minimal TFA and allowed to stand at room temperature. Upon complete consumption of starting material, the reaction was concentrated and triturated with diethyl ether to give L-53b (0.268 g, 0.256 mmol, 98%). LC/MS [M+H] 932.53 (calculated); LC/MS [M+H] 932.81 (observed).

### Preparation of TAZ-L-53

Cyclobutyl chloroformate (0.1 ml, 0.094 mmol, 1.24 equiv.), 2,3,5,6-tetrafluorophenol (0.065 g, 0.39 mmol, 5 equiv.), and collidine (0.103 ml, 0.78 mmol, 10 equiv.) were dissolved in 1 ml acetonitrile and allowed to stand for one hour. Intermediate L-53b (0.073 g, 0.078 mmol, 1 equiv.) was dissolved in this reaction mixture and the reaction monitored by LCMS. EDC (0.03 g, 0.157 mmol, 2 equiv.) was added to the solution upon consumption of the amine, and the reaction stirred at room temperature. Upon completion, the reaction was concentrated and purified by HPLC to give TAZ-L-53 (0.027 g, 0.023 mmol, 29%). LC/MS [M+H] 1178.56 (calculated); LC/MS [M+H] 1178.85 (observed).

### Example L-59 Synthesis of (R)-2-((5-(5-amino-7-((3-(3,3-dimethylbutanamido)propyl)(propyl)carbamoyl)-6H-thieno[3,2-b]azepin-2-yl)pentyl)carbamoyl)-4,37-dioxo-37-(2,3,5,6-tetrafluorophenoxy)-7,10,13,16,19,22,25,28,31,34-decaoxa-3-azaheptatriacontane-1-sulfonic acid, TAZ-L-59

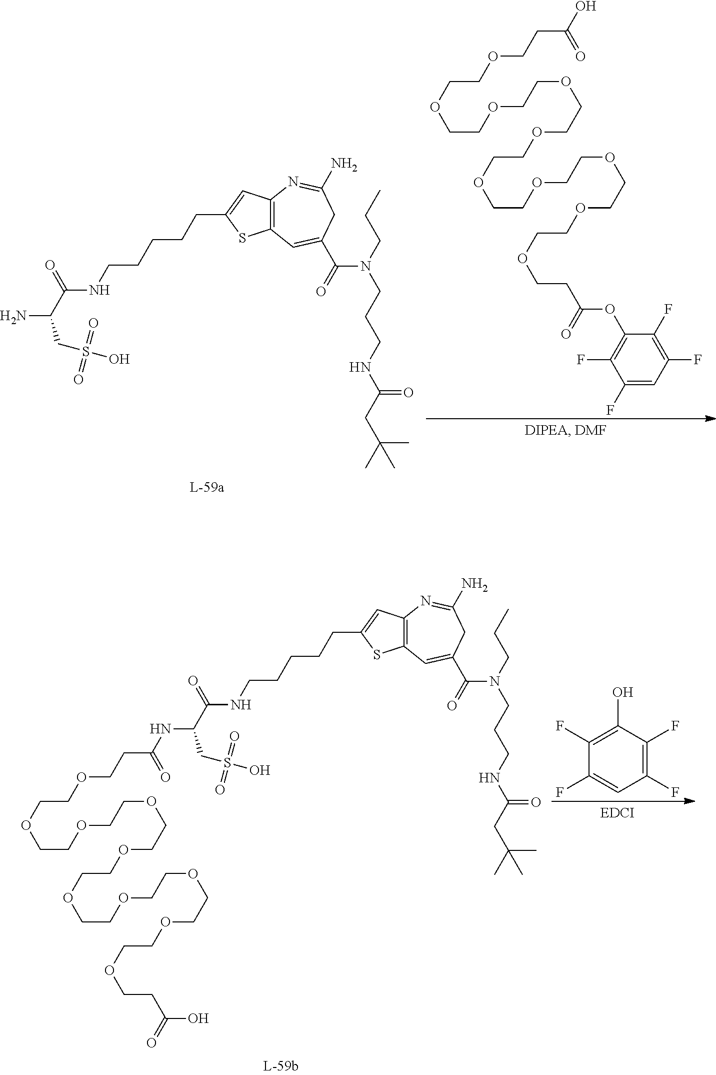

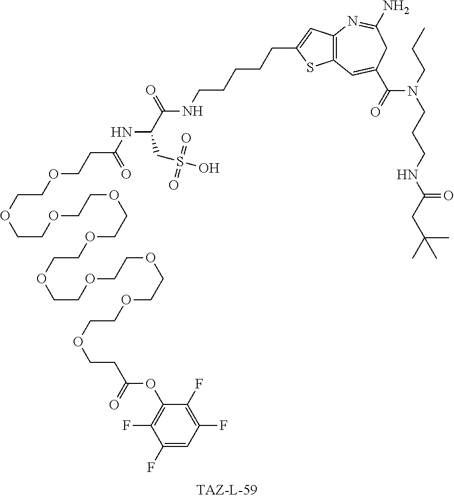 [SMILES: CCCN(CCCNC(=O)CC(C)(C)C)C(=O)C1=Cc2sc(CCCCCNC(=O)[C@H](CS(=O)(=O)O)NC(=O)CCOCCOCCOCCOCCOCCOCCOCCOCCOCCOCCC(=O)Oc3c(F)c(F)cc(F)c3F)cc2N=C(N)C1]

### Preparation of (R)-43-(5-amino-7-((3-(3,3-dimethylbutanamido)propyl)(propyl)carbamoyl)-6H-thieno[3,2-b]azepin-2-yl)-34,37-dioxo-36-(sulfomethyl)-4,7,10,13,16,19,22,25,28,31-decaoxa-35,38-diazatritetracontanoic acid, L-59b

(R)-2-amino-3-((5-(5-amino-7-((3-(3,3-dimethylbutanamido)propyl)(propyl)carbamoyl)-6H-thieno[3,2-b]azepin-2-yl)pentyl)amino)-3-oxopropane-1-sulfonic acid, L-59a (0.034 g, 0.053 mmol, 1 equiv.) was dissolved in DMF (1.5 ml). To this solution were added DIPEA (0.046 ml, 0.265 mmol, 5 equiv.), followed by 34-oxo-34-(2,3,5,6-tetrafluorophenoxy)-4,7,10,13,16,19,22,25,28,31-decaoxatetratriacontanoic acid (0.037 g, 0.053 mmol, 1 equiv.). The reaction was heated to 40° C. for 20 minutes, then cooled to room temperature and purified by reverse-phase HPLC to give L-59b (0.036 g, 0.30 mmol, 580%) as a yellow film. LC/MS [M+H] 1181.59 (calculated); LC/MS [M+H] 1181.87 (observed).

### Preparation of TAZ-L-59

Intermediate L-59b (0.036 g, 0.03 mmol, 1 equiv.) was dissolved in DMF. To this solution were added 2,3,5,6-tetrafluorophenol (0.02 g, 0.09 mmol, 3 equiv.), collidine (0.02 ml, 0.15 mmol, 5 equiv.), and EDCI (0.02 g, 0.09 mmol, 3 equiv.). The reaction was monitored by LCMS, and then concentrated and purified by HPLC to give TAZ-L-59 (0.034 g, 0.031 mmol, 84%). LC/MS [M+H] 1329.59 (calculated); LC/MS [M+H] 1329.88 (observed).

### Example L-83 Synthesis of (2,3,5,6-tetrafluorophenyl)3-[2-[2-[2-[2-[2-[2-[2-[2-[2-[2-[4-[[5-amino-7-[3-(cyclobutoxycarbonylamino)propyl-propyl-carbamoyl]-6H-thieno[3,2-b]azepin-2-yl]methyl]-1-piperidyl]ethoxy]ethoxy]ethoxy]ethoxy]ethoxy]ethoxy]ethoxy]ethoxy]ethoxy]ethoxy]propanoate, TAZ-L-83

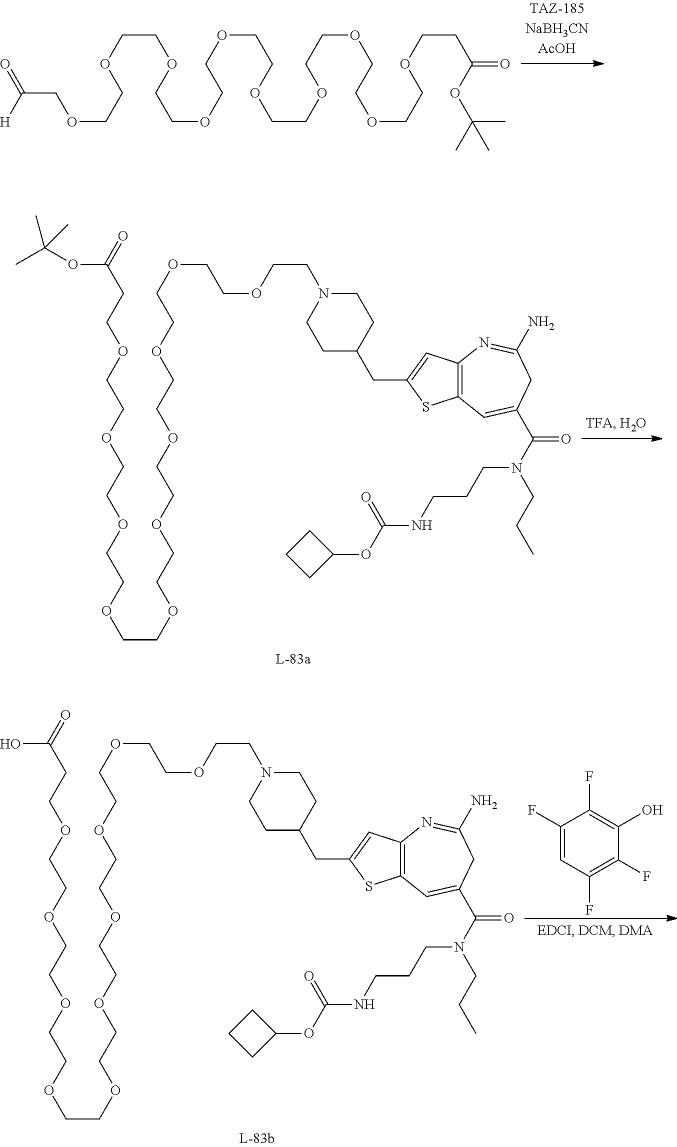

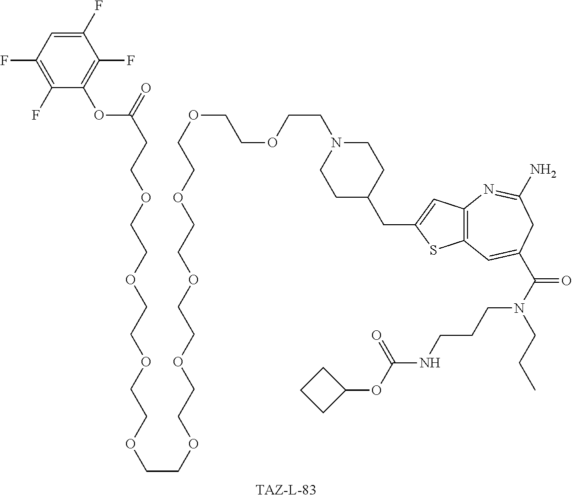 [SMILES: CCCN(CCCNC(=O)OC1CCC1)C(=O)C1=Cc2sc(CC3CCN(CCOCCOCCOCCOCCOCCOCCOCCOCCOCCOCCC(=O)Oc4c(F)c(F)cc(F)c4F)CC3)cc2N=C(N)C1]

### Preparation of tert-butyl 3-[2-[2-[2-[2-[2-[2-[2-[2-[2-[2-[4-[[5-amino-7-[3-(cyclobutoxycarbonylamino) propyl-propyl-carbamoyl]-6H-thieno[3,2-b]azepin-2-yl]methyl]-1-piperidyl]ethoxy]ethoxy]ethoxy]ethoxy]ethoxy]ethoxy]ethoxy]ethoxy]ethoxy]ethoxy]propanoate, L-83a

To a mixture of cyclobutyl N-[3-[[5-amino-2-(4-piperidylmethyl)-6H-thieno[3,2-b]azepine-7-carbonyl]-propyl-amino]propyl]carbamate, TAZ-185 (75.0 mg, 149 umol, 1.0 eq) and tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (437 mg, 747 umol, 5.0 eq) in MeOH (5 mL) was added NaBH₃CN (37.5 mg, 598 umol, 4.0 eq) in one portion at 20° C. under N₂, and then stirred at 20° C. for 40 hours. The reaction mixture was concentrated in vacuum and the residue was purified by prep-HPLC (column: Phenomenex Synergi C18 150*30 mm*4 um; mobile phase: [water(0.1% TFA)−ACN]; B %: 20%-50%, 8 min) to afford L-83a (100 mg, 93.4 umol, 62.5% yield) as colorless oil. ¹H NMR (400 MHz, MeOD) δ7.08 (s, 1H), 6.96 (s, 1H), 4.85-4.80 (m, 1H), 3.85 (d, J=4.8 Hz, 2H), 3.75-3.57 (m, 40H), 3.56-3.43 (m, 6H), 3.38 (s, 2H), 3.11 (dd, J=4.0, 5.4 Hz, 2H), 3.08-2.97 (m, 2H), 2.91 (d, J=6.4 Hz, 2H), 2.36-2.23 (m, 2H), 2.10-1.92 (m, 5H), 1.87-1.75 (m, 3H), 1.72-1.54 (m, 5H), 1.47 (s, 9H), 0.93 (s, 3H)

### Preparation of 3-[2-[2-[2-[2-[2-[2-[2-[2-[2-[2-[4-[[5-amino-7-[3-(cyclobutoxycarbonylamino)propyl-propyl-carbamoyl]-6H-thieno[3,2-b]azepin-2-yl]methyl]-1-piperidyl]ethoxy]ethoxy]ethoxy]ethoxy]ethoxy]ethoxy]ethoxy]ethoxy]ethoxy]ethoxy]propanoic acid, L-83b

To a solution of L-83a(100 mg, 93.4 umol, 1.0 eq) in MeCN (0.5 mL) and H₂O (2 mL) was added HCl (12 M, 233 uL, 30 eq) in one portion at 20° C. under N₂, and then stirred at 80° C. for 1 hour. The reaction mixture was concentrated in vacuum to afford L-83b (80.0 mg, 78.8 umol, 84.4% yield) as colorless oil.

### Preparation of TAZ-L-83

To a mixture of L-83b (80.0 mg, 78.8 umol, 1.0 eq) and 2,3,5,6-tetrafluorophenol (131 mg, 788 umol, 10 eq) in DCM (2 mL) and DMA (0.5 mL) was added EDCI (151 mg, 788 umol, eq) in one portion at 20° C. under N₂, the mixture was stirred at 20° C. for 1 hour. DCM (2 mL) was removed in vacuum and the mixture was filtered, the filtrate was purified by prep-HPLC (column: Phenomenex Synergi C18 150*30 mm*4 um; mobile phase: [water(0.1% TFA)-ACN]; B %: 20%-50%, 8 min) to afford TAZ-L-83 (43.0 mg, 36.3 umol, 46.0% yield, 98.20% purity) as colorless oil. ¹H NMR (400 MHz, MeOD) δ7.49-7.42 (m, 1H), 7.07 (s, 1H), 6.95 (s, 1H), 3.89 (t, J=6.0 Hz, 2H), 3.87-3.83 (m, 2H), 3.70-3.46 (m, 42H), 3.37 (s, 2H), 3.17-3.08 (m, 3H), 3.00 (t, J=6.0 Hz, 4H), 2.91 (d, J=6.8 Hz, 2H), 2.34-2.25 (m, 2H), 2.09-1.96 (m, 5H), 1.87-1.58 (m, 8H), 0.92 (t, J=4.0 Hz, 3H). LC/MS [M+H] 1162.6 (calculated); LC/MS [M+H] 1162.4 (observed).

### Example L-84 Synthesis of (2,3,5,6-tetrafluorophenyl)₃-[2-[2-[2-[2-[2-[2-[2-[2-[2-[2-[4-[[5-amino-7-[ethoxy(propyl)carbamoyl]-6H-thieno[3,2-b]azepin-2-yl]methyl]-1-piperidyl]ethoxy]ethoxy]ethoxy]ethoxy]ethoxy]ethoxy]ethoxy]ethoxy]ethoxy]ethoxy]propanoate, TAZ-L-84

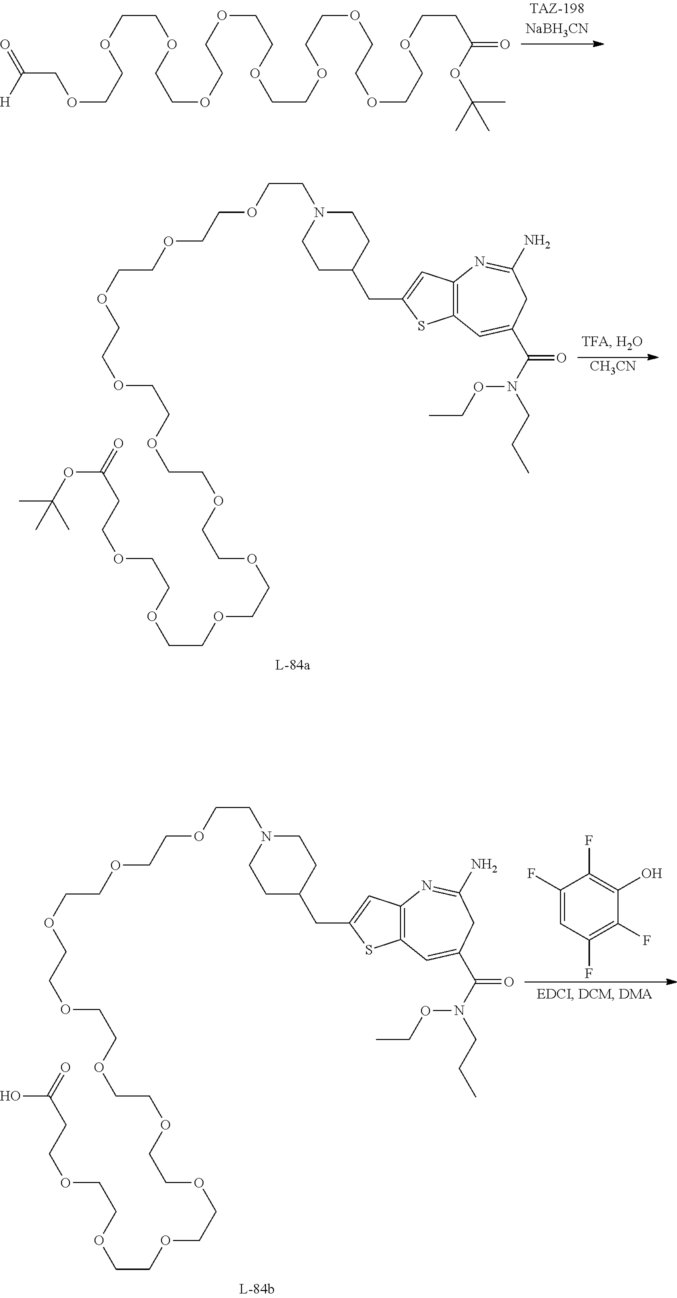

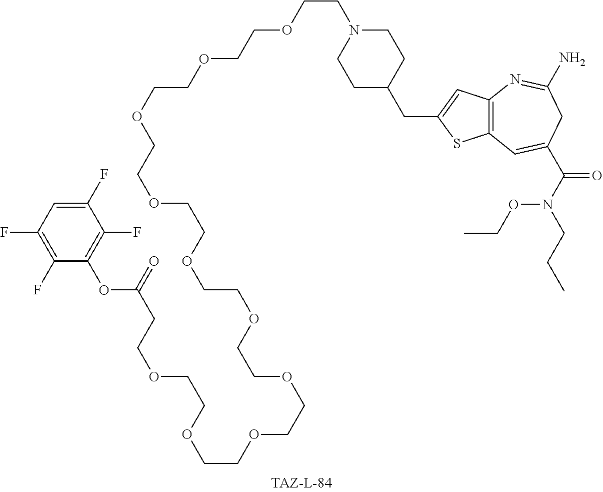 [SMILES: CCCN(OCC)C(=O)C1=Cc2sc(CC3CCN(CCOCCOCCOCCOCCOCCOCCOCCOCCOCCOCCC(=O)Oc4c(F)c(F)cc(F)c4F)CC3)cc2N=C(N)C1]

### Preparation of tert-butyl 3-[2-[2-[2-[2-[2-[2-[2-[2-[2-[2-[4-[[5-amino-7-[ethoxy(propyl)carbamoyl]-6H-thieno[3,2-b]azepin-2-yl]methyl]-1-piperidyl]ethoxy]ethoxy]ethoxy]ethoxy]ethoxy]ethoxy]ethoxy]ethoxy]ethoxy]ethoxy]propanoate, L-84a

To a mixture of 5-amino-N-ethoxy-2-(4-piperidylmethyl)-N-propyl-6H-thieno[3,2-b]azepine-7-carboxamide, TAZ-198 (60.0 mg, 153 umol, 1.0 eq) and tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (449 mg, 768 umol, 5.0 eq) in MeOH (5 mL) was added NaBH₃CN (28.9 mg, 460 umol, 3.0 eq) in one portion at 20° C. under N₂, and then stirred at 20° C. for 40 hours. The reaction mixture was concentrated in vacuum and the residue was purified by prep-HPLC (column: Phenomenex Synergi C18 150*30 mm*4 um; mobile phase: [water(0.1% TFA)−ACN]; B %: 20%-45%, 8 min) to afford L-84a (100 mg, 104 umol, 67.8% yield) as colorless oil. ¹H NMR (400 MHz, MeOD) δ7.48 (s, 1H), 6.97 (s, 1H), 3.95 (q, J=7.2 Hz, 2H), 3.85 (d, J=4.4 Hz, 2H), 3.77-3.65 (m, 40H), 3.45 (s, 2H), 3.01 (d, J=12.4 Hz, 2H), 2.92 (d, J=6.4 Hz, 2H), 2.49 (t, J=6.4 Hz, 2H), 2.05-2.00 (m, 3H), 1.82-1.72 (m, 2H), 1.66-1.54 (m, 2H), 1.47 (s, 9H), 1.20 (t, J=7.2 Hz, 3H), 1.00 (t, J=7.2 Hz, 3H).

### Preparation of 3-[2-[2-[2-[2-[2-[2-[2-[2-[2-[2-[4-[[5-amino-7-[ethoxy(propyl)carbamoyl]-6H-thieno [3,2-b]azepin-2-yl]methyl]-1-piperidyl]ethoxy]ethoxy]ethoxy]ethoxy]ethoxy]ethoxy]ethoxy]ethoxy]ethoxy]ethoxy]propanoic acid, L-84b

To a solution of L-84a (100 mg, 104 umol, 1.0 eq) in MeCN (0.5 mL) and H₂O (2 mL) was added HCl (12 M, 260.62 uL, 30 eq) in one portion at 20° C. under N₂, and then stirred at 80° C. for 1 hour. The reaction mixture was concentrated in vacuum. to afford L-84b (80 mg, 88.58 umol, 84.97% yield) as colorless oil.

### Preparation of TAZ-L-84

To a mixture of L-84b (80 mg, 88.5 umol, 1.0 eq) and 2,3,5,6-tetrafluorophenol (147 mg, 885 umol, 10 eq) in DCM (2 mL) and DMA (0.5 mL) was added EDCI (84.9 mg, 442 umol, 5.0 eq) in one portion at 20° C. under N₂, the mixture was stirred at 20° C. for 1 hour. DCM (2 mL) was removed in vacuum and the mixture was filtered, the residue was purified by prep-HPLC (column: Phenomenex Synergi C18 150*30 mm*4 um; mobile phase: [water(0.1% TFA)-ACN]; B %: 20%-50%, 8 min) to afford TAZ-L-84 (37 mg, 35.09 umol, 39.61% yield, 99.68% purity) as colorless oil. ¹H NMR (400 MHz, MeOD) δ7.48 (s, 1H), 7.46-7.41 (m, 1H), 6.96 (s, 1H), 3.95 (q, J=6.8 Hz, 3H), 3.89 (t, J=6.0 Hz, 2H), 3.87-3.83 (m, 2H), 3.71-3.61 (m, 40H), 3.44 (s, 2H), 3.37-3.34 (m, 3H), 3.05-2.95 (m, 5H), 2.91 (d, J=6.4 Hz, 2H), 2.05-1.95 (m, 2H), 1.82-1.72 (m, 2H), 1.66-1.54 (m, 2H), 1.20 (t, J=7.2 Hz, 3H), 0.99 (t, J=7.2 Hz, 3H). LC/MS [M+H] 1151.5 (calculated); LC/MS [M+H] 1151.3 (observed).

### Example L-87 Synthesis of (2,3,5,6-tetrafluorophenyl) 3-[2-[2-[2-[2-[2-[2-[2-[2-[2-[2-[4-[2-[5-amino-7-[ethoxy(propyl)carbamoyl]-6H-thieno[3,2-b]azepin-2-yl]ethyl]-1-piperidyl]ethoxy]ethoxy]ethoxy]ethoxy]ethoxy]ethoxy]ethoxy]ethoxy]ethoxy]ethoxy]propanoate, TAZ-L-87

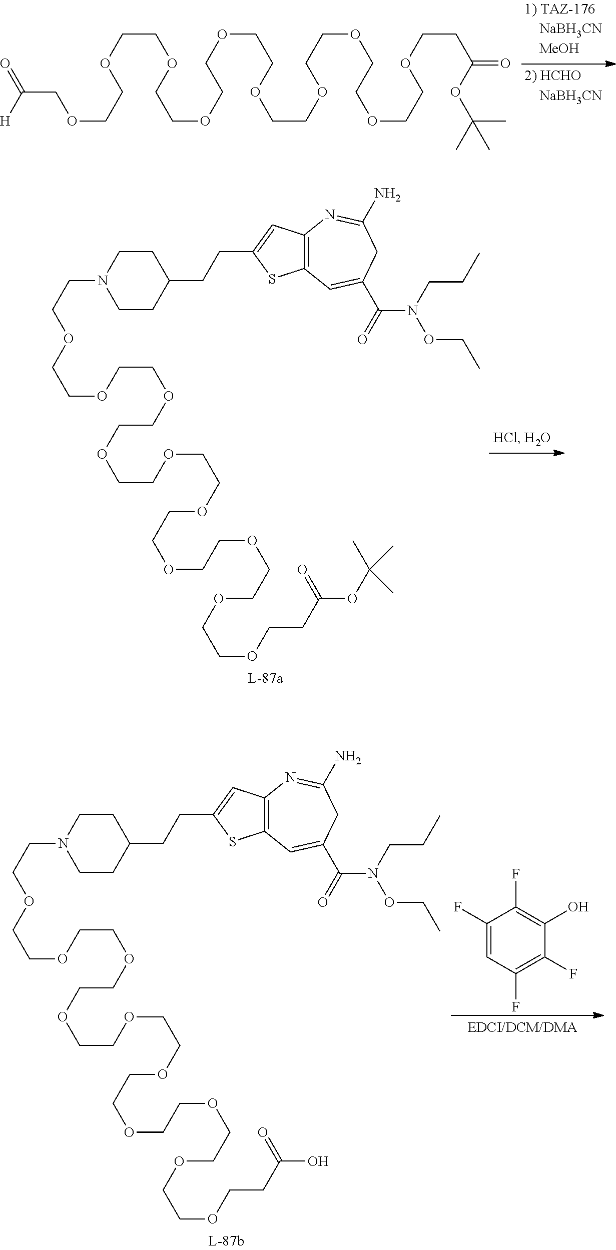

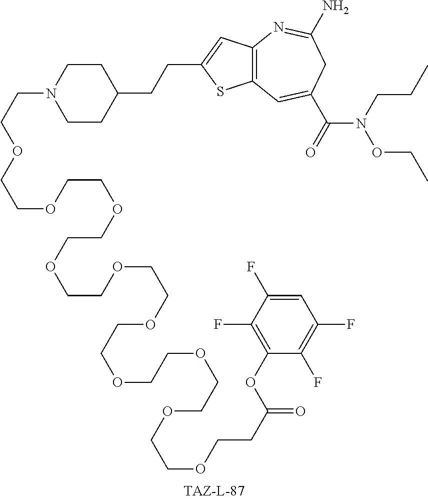 [SMILES: CCCN(OCC)C(=O)C1=Cc2sc(CCC3CCN(CCOCCOCCOCCOCCOCCOCCOCCOCCOCCOCCC(=O)Oc4c(F)c(F)cc(F)c4F)CC3)cc2N=C(N)C1]

### Preparation of tert-butyl 3-[2-[2-[2-[2-[2-[2-[2-[2-[2-[2-[4-[2-[5-amino-7-[ethoxy (propyl)carbamoyl]-6H-thieno[3,2-b]azepin-2-yl]ethyl]-1-piperidyl]ethoxy]ethoxy]ethoxy]ethoxy]ethoxy]ethoxy]ethoxy]ethoxy]ethoxy]ethoxy]propanoate, L-87a

To a mixture of 5-amino-N-ethoxy-2-[2-(4-piperidyl)ethyl]-N-propyl-6H-thieno[3,2-b]azepine-7-carboxamide, TAZ-176 (0.15 g, 340 umol, 1.0 eq, HCl) in MeOH (3 mL) was added tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (597 mg, 1.02 mmol, 3.0 eq) and NaBH₃CN (42.8 mg, 680 umol, 2.0 eq) in one portion at 25° C. and it was stirred at 25° C. for 12 h. The mixture was concentrated to give a residue. The residue was purified by prep-HPLC(column: Phenomenex luna C18 100*40 mm*5 um; mobile phase: [water(0.1% TFA)−ACN]; B %: 12%-42%, 8 min) to give L-87a (0.18 g, 165.55 umol, 48.67% yield, TFA) as yellow oil. ¹H NMR (MeOD, 400 MHz) δ7.45 (s, 1H), 6.94 (s, 1H), 3.96-3.90 (m, 2H), 3.86-3.82 (m, 2H), 3.75-3.71 (m, 2H), 3.70-3.67 (m, 6H), 3.66-3.59 (m, 36H), 3.42 (s, 2H), 3.06-2.90 (m, 4H), 2.47 (t, J=6.2 Hz, 2H), 2.13-2.00 (m, 2H), 1.87-1.60 (m, 6H), 1.60-1.48 (m, 2H), 1.45 (s, 10H), 1.18 (t, J=7.2 Hz, 3H), 0.97 (t, J=7.6 Hz, 3H).

### Preparation of 3-[2-[2-[2-[2-[2-[2-[2-[2-[2-[2-[4-[2-[5-amino-7-[ethoxy(propyl)carbamoyl]-6H-thieno[3,2-b]azepin-2-yl]ethyl]-1-piperidyl]ethoxy]ethoxy]ethoxy]ethoxy]ethoxy]ethoxy]ethoxy]ethoxy]ethoxy]ethoxy]propanoic acid, L-87b

To a mixture of L-87a (0.18 g, 166 umol, 1.0 eq, TFA) in H₂O (2.5 mL) and CH₃CN (0.3 mL) was added HCl (12 M, 345 uL, 25.0 eq) in one portion at 25° C. and it was stirred at 80° C. for 1 h. The mixture was concentrated to give L-87b (0.15 g, crude, HCl) as yellow oil.

### Preparation of TAZ-L-87

To a mixture of L-87b (0.05 g, 52.4 umol, 1.0 eq, HCl) in DCM (1 mL) and DMA (0.2 mL) was added 2,3,5,6-tetrafluorophenol (69.7 mg, 419 umol, 8.0 eq) and EDCI (101 mg, 524 umol, 10.0 eq) in one portion at 25° C. and it was stirred at 25° C. for 0.5 h. The mixture was concentrated to give a residue. The residue was purified by prep-HPLC(column: Phenomenex Synergi C18 150*30 mm*4 um; mobile phase: [water(0.1% TFA)−ACN]; B %: 20%-50%, 8 min) to give TAZ-L-87 (14.5 mg, 12.30 umol, 23.45% yield, TFA) as yellow oil. ¹H NMR (MeOD, 400 MHz) δ7.49-7.38 (m, 2H), 6.92 (s, 1H), 3.93 (q, J=8 Hz, 2H), 3.87 (t, J=6.0 Hz, 2H), 3.85-3.81 (m, 2H), 3.74-3.70 (m, 2H), 3.69-3.61 (m, 40H), 3.42 (s, 2H), 3.03-2.91 (m, 6H), 2.07 (d, J=13.6 Hz, 2H), 1.79-1.66 (m, 5H), 1.58-1.43 (m, 2H), 1.18 (t, J=7.2 Hz, 3H), 0.97 (t, J=7.6 Hz, 3H). LC/MS [M+H] 1065.5 (calculated); LC/MS [M+H] 1065.4 (observed).

### Example L-88 Synthesis of (2,3,5,6-tetrafluorophenyl) 3-[2-[2-[2-[2-[2-[2-[2-[2-[2-[3-[4-[2-[5-amino-7-[ethoxy(propyl)carbamoyl]-6H-thieno[3,2-b]azepin-2-yl]ethyl]-1-piperidyl]-3-oxo-propoxy]ethoxy]ethoxy]ethoxy]ethoxy]ethoxy]ethoxy]ethoxy]ethoxy]ethoxy]propanoate, TAZ-L-88

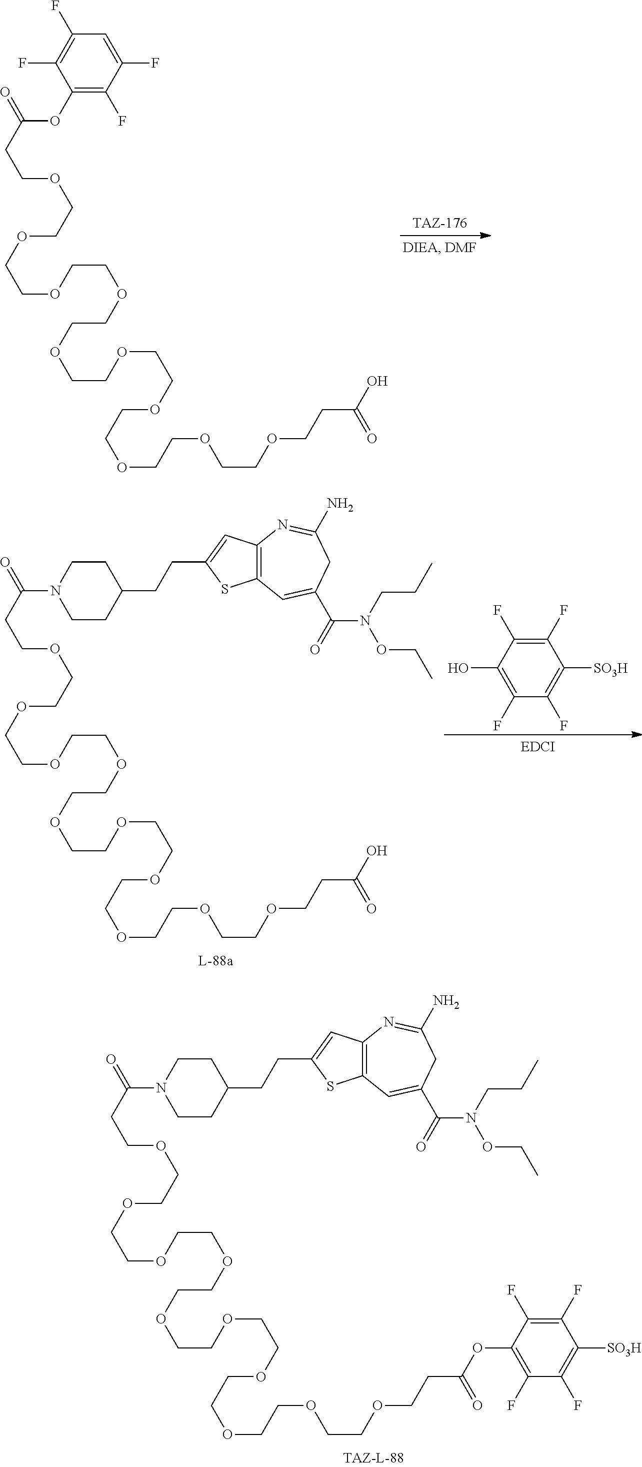

### Preparation of 3-[2-[2-[2-[2-[2-[2-[2-[2-[2-[3-[4-[2-[5-amino-7-[ethoxy(propyl) carbamoyl]-6H-thieno[3,2-b] azepin-2-yl]ethyl]-1-piperidyl]-3-oxo-propoxy]ethoxy]ethoxy]ethoxy]ethoxy]ethoxy]ethoxy]ethoxy]ethoxy]ethoxy]propanoic acid, L-88a

To a mixture of 5-amino-N-ethoxy-2-[2-(4-piperidyl)ethyl]-N-propyl-6H-thieno[3,2-b]azepine-7-carboxamide, TAZ-176 (60.0 mg, 136 umol, 1.0 eq, HCl) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (106 mg, 150 umol, 1.1 eq) in DMF (0.5 mL) was added DIEA (52.8 mg, 408 umol, 71.1 uL, 3.0 eq) in one portion at 25° C. and it was stirred at 25° C. for 0.5 h. Then the mixture was filtered and purified by prep-HPLC (column: Welch Xtimate C18 150*25 mm*5 um; mobile phase: [water(0.04% HCl)−ACN]; B %: 20%-35%, 8 min) to give L-88a (40 mg, 42.32 umol, 31.11% yield) as yellow oil.

### Preparation of TAZ-L-88

To a mixture of L-88a (40 mg, 40.8 umol, 1.0 eq) in DCM (1 mL) and DMA (0.2 mL) was added 2,3,5,6-tetrafluorophenol (54.1 mg, 326 umol, 8.0 eq) and EDCI (78.1 mg, 407 umol, 10.0 eq) in one portion at 25° C. and it was stirred at 25° C. for 0.5 h. The mixture was concentrated to give a residue, and the residue was purified by prep-HPLC (column: Phenomenex Synergi C18 150*30 mm*4 um; mobile phase: [water(0.1% TFA)−ACN]; B %: 30%-60%, 8 min) to give TAZ-L-88 (36.2 mg, 33.11 umol, 81.26% yield) as yellow oil. ¹H NMR (MeOD, 400 MHz) δ7.46 (s, 1H), 7.45-7.39 (m, 1H), 6.90 (s, 1H), 4.53 (d, J=13.2 Hz, 1H), 4.04 (d, J=13.6 Hz, 1H), 3.93 (q, J=7.2 Hz, 2H), 3.87 (t, J=6.0 Hz, 2H), 3.77-3.69 (m, 4H), 3.66-3.60 (m, 36H), 3.42 (s, 2H), 3.06 (t, J=12.0 Hz, 1H), 2.98 (t, J=6.0 Hz, 2H), 2.93 (t, J=7.6 Hz, 2H), 2.78-2.51 (m, 3H), 1.89-1.63 (m, 7H), 1.30-1.07 (m, 5H), 0.97 (t, J=7.6 Hz, 3H).

LC/MS [M+H] 1093.5 (calculated); LC/MS [M+H] 1093.4 (observed).

### Example L-92 Synthesis of (2,3,5,6-tetrafluorophenyl) 3-[2-[2-[2-[2-[2-[2-[2-[2-[2-[2-[3-[[5-amino-7-[ethoxy(propyl)carbamoyl]-6H-thieno[3,2-b]azepin-2-yl]methyl]azetidin-1-yl]ethoxy]ethoxy]ethoxy]ethoxy]ethoxy]ethoxy]ethoxy]ethoxy]ethoxy]ethoxy]propanoate, TAZ-L-92

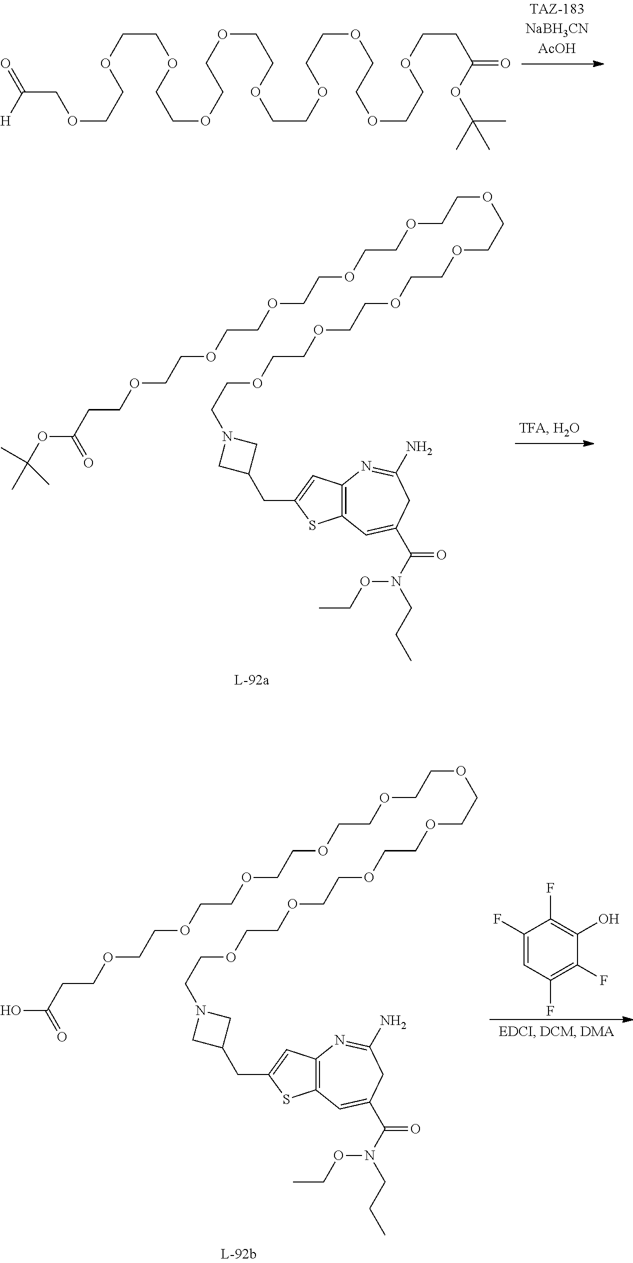

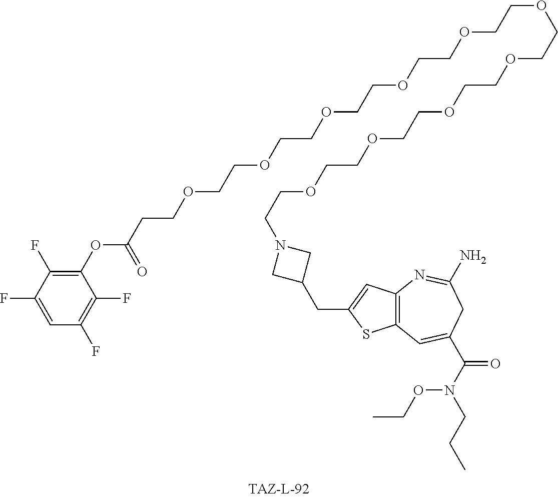 [SMILES: CCCN(OCC)C(=O)C1=Cc2sc(CC3CN(CCOCCOCCOCCOCCOCCOCCOCCOCCOCCOCCC(=O)Oc4c(F)c(F)cc(F)c4F)C3)cc2N=C(N)C1]

### Preparation of tert-butyl 3-[2-[2-[2-[2-[2-[2-[2-[2-[2-[2-[3-[[5-amino-7-[ethoxy(propyl)carbamoyl]-6H-thieno[3,2-b]azepin-2-yl]methyl]azetidin-1-yl]ethoxy]ethoxy]ethoxy]ethoxy]ethoxy]ethoxy]ethoxy]ethoxy]ethoxy]ethoxy]propanoate, L-92a

To a mixture of 5-amino-2-(azetidin-3-ylmethyl)-N-ethoxy-N-propyl-6H-thieno [3,2-b]azepine-7-carboxamide, TAZ-183 (0.06 g, 166 umol, 1 eq) and tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (290 mg, 497 umol, 3 eq) in MeOH (5 mL) was added NaBH₃CN (20.8 mg, 331 umol, 2 eq) and AcOH (9.94 mg, 166 umol, 1 eq), and then stirred at 15° C. for 10 hr. The mixture was concentrated to give a residue. The residue was purified prep-HPLC(TFA)(column: Phenomenex Synergi C18 150*30 mm*4 um; mobile phase: [water(0.1% TFA)−ACN]; B %: 20%-45%, 8 min) to give L-92a (20 mg, 19.1 umol, 11.6% yield, TFA) as yellow solid.

### Preparation of 3-[2-[2-[2-[2-[2-[2-[2-[2-[2-[2-[3-[[5-amino-7-[ethoxy(propyl)carbamoyl]-6H-thieno[3,2-b]azepin-2-yl]methyl]azetidin-1-yl]ethoxy]ethoxy]ethoxy]ethoxy]ethoxy]ethoxy]ethoxy]ethoxy]ethoxy]ethoxy]propanoic acid, L-92b

To a mixture of L-92a (20 mg, 19.1 umol, 1 eq, TFA) in H₂O (0.5 mL) was add TFA (10.9 mg, 95.7 umol, 5 eq) at 25° C., and then stirred at 80° C. for 4 hr. The mixture was concentrated to give a residue. The residue was purified by prep--HPLC(column: Phenomenex Gemini-NX C18 75*30 mm*3 um; mobile phase: [water(10 mM NH₄HCO3)−ACN]; B %: 25%-45%, 6 min) to give L-92b (13 mg, 14.9 umol, 77.6% yield) as colorless oil.

### Preparation of TAZ-L-92

To a mixture of L-92b (12 mg, 13.7 umol, 1 eq) and 2,3,5,6-tetrafluorophenol (18.2 mg, 110 umol, 8 eq) in DCM (1 mL) and DMA (0.1 mL) was added EDCI (26.3 mg, 137 umol, 10 eq), and then stirred at 15° C. for 0.5 hr. The mixture was concentrated to give a residue. The residue was purified by prep-HPLC(column: Phenomenex Synergi C18 150*30 mm*4 um; mobile phase: [water(0.1% TFA)−ACN]; B %: 20%-50%, 8 min) to give TAZ-L-92 (6.3 mg, 5.54 umol, 40.4% yield, 100% purity, TFA) as colorless oil. ¹H NMR (400 MHz, MeOD) δ7.57-7.32 (m, 2H), 6.98 (br s, 1H), 4.43-3.79 (m, 8H), 3.77-3.52 (m, 40H), 3.44 (s, 2H), 3.33-3.26 (m, 4H), 3.18-3.11 (m, 1H), 3.00 (t, J=6.0 Hz, 2H), 1.83-1.68 (m, 2H), 1.20 (t, J=7.2 Hz, 3H), 0.99 (t, J=7.6 Hz, 3H). LC/MS [M+H] 1023.5 (calculated); LC/MS [M+H] 1023.3 (observed).

### Example L-93 Synthesis of (2,3,5,6-tetrafluorophenyl) 3-[2-[2-[2-[2-[2-[2-[2-[2-[2-[3-[4-[[5-amino-7-[ethoxy(propyl)carbamoyl]-6H-thieno[3,2-b]azepin-2-yl]methyl]-1-piperidyl]-3-oxo-propoxy]ethoxy]ethoxy]ethoxy]ethoxy]ethoxy]ethoxy]ethoxy]ethoxy]ethoxy]propanoate, TAZ-L-93

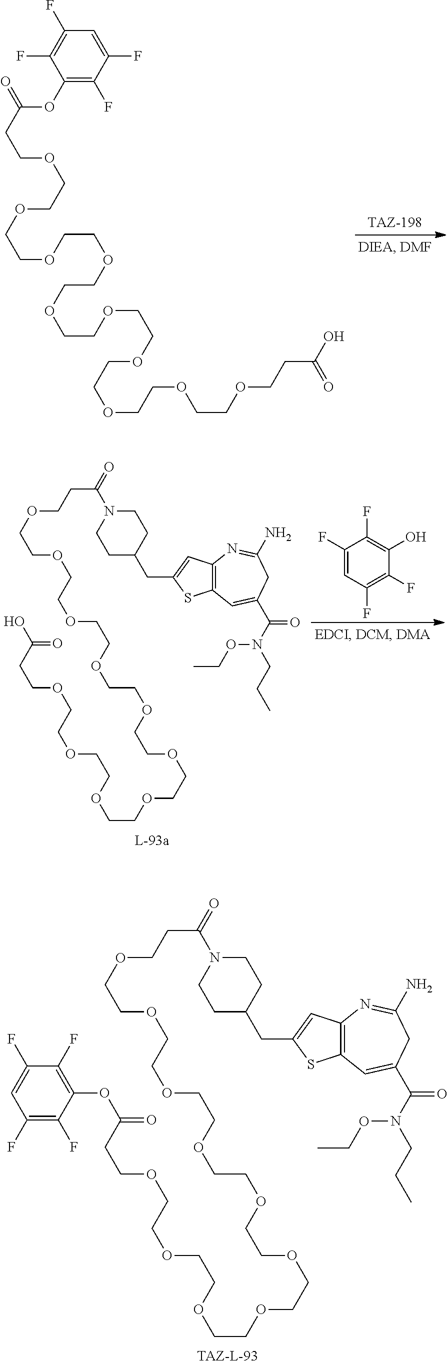 [SMILES: O=C(O)CCOCCOCCOCCOCCOCCOCCOCCOCCOCCOCCC(=O)Oc1c(F)c(F)cc(F)c1F ; CCCN(OCC)C(=O)C1=Cc2sc(CC3CCN(C(=O)CCOCCOCCOCCOCCOCCOCCOCCOCCOCCOCCC(=O)O)CC3)cc2N=C(N)C1 ; Oc1c(F)c(F)cc(F)c1F ; CCCN(OCC)C(=O)C1=Cc2sc(CC3CCN(C(=O)CCOCCOCCOCCOCCOCCOCCOCCOCCOCCOCCC(=O)Oc4c(F)c(F)cc(F)c4F)CC3)cc2N=C(N)C1]

### Preparation of 3-[2-[2-[2-[2-[2-[2-[2-[2-[2-[3-[4-[[5-amino-7-[ethoxy(propyl) carbamoyl]-6H-thieno[3,2-b]azepin-2-yl]methyl]-1-piperidyl]-3-oxo-propoxy]ethoxy]ethoxy]ethoxy]ethoxy]ethoxy]ethoxy]ethoxy]ethoxy]ethoxy]propanoic acid, L-93a

To a mixture of 5-amino-N-ethoxy-2-(4-piperidylmethyl)-N-propyl-6H-thieno[3,2-b]azepine-7-carboxamide, TAZ-198 (30.0 mg, 70.3 umol, 1.0 eq, HCl) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (54.6 mg, 77.3 umol, 1.1 eq) in DMF (0.5 mL) was added DIEA (27.2 mg, 210 umol, 36.7 uL, 3.0 eq) in one portion at 20° C. under N₂, and then stirred at 20° C. for 1 hour. The reaction mixture was filtered and the filtrate was purified by prep-HPLC (column: Xtimate C18 100*30 mm*3 um; mobile phase: [water (0.04% HCl)−ACN]; B %: 22%-45%, 8 min) to afford L-93a (60.0 mg, 64.4 umol, 91.7% yield) as colorless oil.

### Preparation of TAZ-L-93

To a mixture of L-93a (60.0 mg, 64.4 umol, 1.0 eq) and 2,3,5,6-tetrafluorophenol (107 mg, 644 umol, 10 eq) in DCM (2 mL) and DMA (0.5 mL) was added EDCI (123.53 mg, 644.37 umol, 10 eq) in one portion at 20° C. under N₂, and then stirred at 20° C. for 1 hour. The reaction mixture was filtered and the filtrate was purified by prep-HPLC (column: Phenomenex Synergi C18 150*30 mm*4 um; mobile phase: [water(0.1% TFA)−ACN]; B %: 30%-55%, 8 min) to afford TAZ-L-93 (44.2 mg, 40.2 umol, 62.4% yield, 98.2% purity) as colorless oil. ¹H NMR (400 MHz, MeOD) δ7.49 (s, 1H), 7.47-7.41 (m, 1H), 6.92 (s, 1H), 4.56 (d, J=12.8 Hz, 1H), 4.06 (d, J=13.6 Hz, 1H), 3.95 (q, J=7.2 Hz, 2H), 3.89 (t, J=6.0 Hz, 2H), 3.80-3.71 (m, 4H), 3.70-3.58 (m, 36H), 3.45 (s, 2H), 3.10 (t, J=12.0 Hz, 1H), 3.00 (t, J=6.0 Hz, 2H), 2.86 (d, J=7.0 Hz, 2H), 2.77-2.57 (m, 3H), 1.95-1.90 (m, 1H), 1.80-1.75 (m, 4H), 1.35-1.20 (m, 5H), 0.99 (t, J=7.2 Hz, 3H). LC/MS [M+H] 1079.5 (calculated); LC/MS [M+H] 1079.4 (observed).

### Example L-98 Synthesis of (2,3,5,6-tetrafluorophenyl)₃-[2-[2-[2-[2-[2-[2-[2-[2-[2-[3-[3-[[5-amino-7-[ethoxy(propyl)carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoate, TAZ-L-98

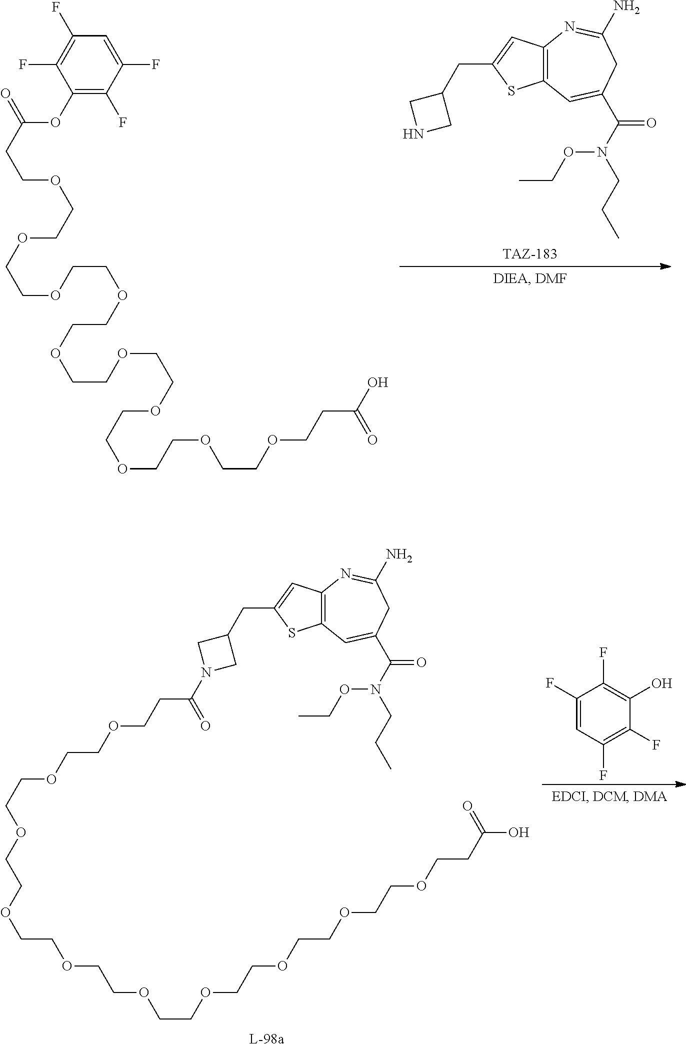 [SMILES: O=C(O)CCOCCOCCOCCOCCOCCOCCOCCOCCOCCOCCC(=O)Oc1c(F)c(F)cc(F)c1F ; CCCN(OCC)C(=O)C1=Cc2sc(CC3CNC3)cc2N=C(N)C1 ; CCCN(OCC)C(=O)C1=Cc2sc(CC3CN(C(=O)CCOCCOCCOCCOCCOCCOCCOCCOCCOCCOCCC(=O)O)C3)cc2N=C(N)C1 ; Oc1c(F)c(F)cc(F)c1F]

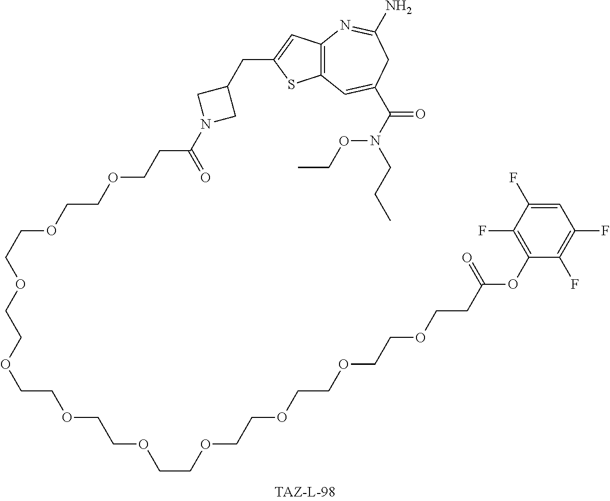 [SMILES: CCCN(OCC)C(=O)C1=Cc2sc(CC3CN(C(=O)CCOCCOCCOCCOCCOCCOCCOCCOCCOCCOCCC(=O)Oc4c(F)c(F)cc(F)c4F)C3)cc2N=C(N)C1]

### Preparation of 3-[2-[2-[2-[2-[2-[2-[2-[2-[2-[3-[3-[[5-amino-7-[ethoxy(propyl)carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoic acid, L-98a

To a mixture of 5-amino-2-(azetidin-3-ylmethyl)-N-ethoxy-N-propyl-6H-thieno[3,2-b]azepine-7-carboxamide, TAZ-183 (150 mg, 315 umol, 1 eq, TFA) and DIEA (102 mg, 787 umol, 137 uL, 2.5 eq) in DMF (2 mL) was added 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (223 mg, 315 umol, 1 eq), and then stirred at 20° C. for 0.5 hr. The mixture was concentrated to give a residue. The residue was purified by Prep-HPLC(column: Waters Xbridge Prep OBD C18 150*40 mm*10 um; mobile phase: [water(10 mM NH₄HCO₃)−ACN]; B %: 5%-40%, 8 min) to give L-98a (0.08 g, 88.6 umol, 28.1% yield) as yellow oil.

### Preparation of TAZ-L-98

To a mixture of L-98a (0.08 g, 88.6 umol, 1 eq) and 2,3,5,6-tetrafluorophenol (118 mg, 709 umol, 8 eq) in DCM (3 mL) and DMA (0.3 mL) was added EDCI (170 mg, 886 umol, 10 eq). The mixture was stirred at 15° C. for 0.5 hr. The mixture was concentrated to give a residue. The residue was purified by Prep-HPLC(column: Phenomenex Synergi C18 150*25*10 um; mobile phase: [water(0.1% TFA)−ACN]; B %: 25%-50%, 10 min and column: Phenomenex Synergi C18 150*25*10 um; mobile phase: [water(0.1% TFA)−ACN]; B %: 25%-50%, 8 min) to give TAZ-L-98 (34.3 mg, 31.9 umol, 36.0% yield, 97.7% purity) as colorless oil. ¹H NMR (400 MHz, MeOD) δ7.49-7.38 (m, 2H), 6.92 (s, 1H), 4.42-4.35 (m, 1H), 4.10 (t, J=9.2 Hz, 1H), 4.06-3.97 (m, 1H), 3.93 (q, J=7.2 Hz, 2H), 3.89-3.85 (m, 2H), 3.73-3.69 (m, 4H), 3.66-3.51 (m, 37H), 3.40 (s, 2H), 3.19 (br d, J=7.6 Hz, 2H), 2.99-2.96 (m, 3H), 2.48-2.24 (m, 2H), 1.74 (q, J=7.2 Hz, 2H), 1.18 (t, J=7.2 Hz, 3H), 0.97 (t, J=7.2 Hz, 3H). LC/MS [M+H] 1051.5 (calculated); LC/MS [M+H] 1051.3 (observed).

### Example L-128 Synthesis of 4-[3-[2-[2-[2-[2-[2-[2-[2-[2-[2-[3-[3-[[5-amino-7-[2-(cyclobutoxycarbonylamino)ethoxy-propyl-carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, TAZ-L-128

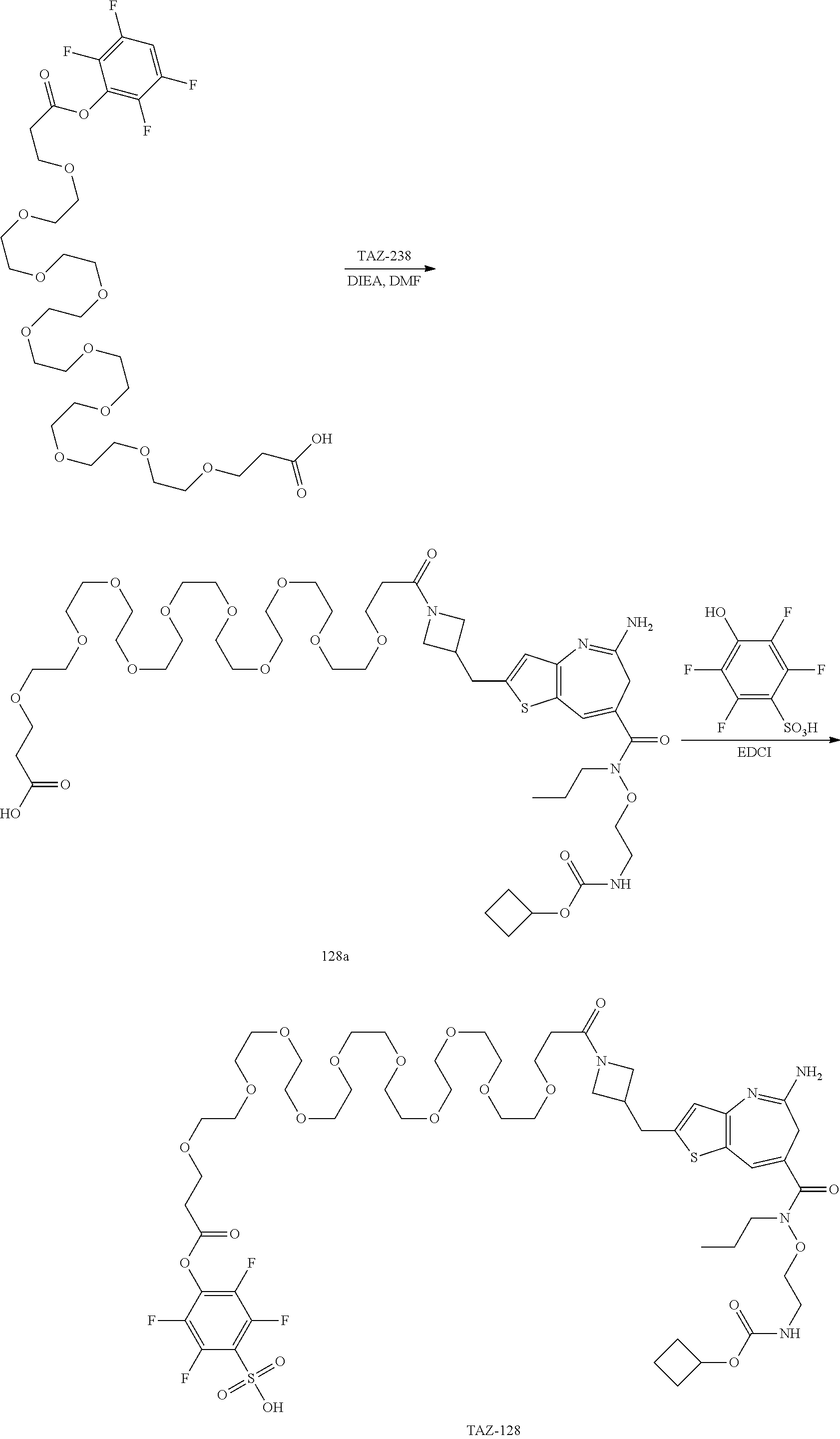 [SMILES: O=C(O)CCOCCOCCOCCOCCOCCOCCOCCOCCOCCOCCC(=O)Oc1c(F)c(F)cc(F)c1F ; CCCN(OCCNC(=O)OC1CCC1)C(=O)C1=Cc2sc(CC3CN(C(=O)CCOCCOCCOCCOCCOCCOCCOCCOCCOCCOCCC(=O)O)C3)cc2N=C(N)C1 ; O=S(=O)(O)c1c(F)c(F)c(O)c(F)c1F ; CCCN(OCCNC(=O)OC1CCC1)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1]

### Preparation of 3-[2-[2-[2-[2-[2-[2-[2-[2-[2-[3-[3-[[5-amino-7-[2-(cyclobutoxycarbonylamino)ethoxy-propyl-carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoic acid, 128a

To a mixture of cyclobutyl N-[2-[[5-amino-2-(azetidin-3-ylmethyl)-6H-thieno[3,2-b]azepine-7-carbonyl]-propyl-amino]oxyethyl]carbamate, TAZ-238 (0.12 g, 171 umol, 1.0 eq, TFA) in DMF (2 mL) was added DIEA (66.1 mg, 512 umol, 89.1 uL, 3.0 eq) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (121 mg, 171 umol, 1.0 eq) in one portion at 0° C. and then stirred at 0° C. for 0.5 h. The mixture was filtered and purified by prep-HPLC(column: Welch Xtimate C18 100*25 mm*3 um; mobile phase: [water(0.05% HCl)−ACN]; B %: 20%-40%, 8 min) to give 128a (45 mg, 42.8 umol, 25.1% yield, HCl) as yellow oil. ¹H NMR (MeOD, 400 MHz) δ7.51 (s, 1H), 6.96 (s, 1H), 4.81-4.76 (m, 1H), 4.40 (t, J=8.4 Hz, 1H), 4.11 (t, J=9.2 Hz, 1H), 4.03 (dd, J=5.6, 8.8 Hz, 1H), 3.92 (t, J=5.2 Hz, 2H), 3.77-3.68 (m, 7H), 3.66-3.55 (m, 36H), 3.42 (s, 2H), 3.30-2.25 (m, 2H), 3.20 (d, J=8.0 Hz, 2H), 3.06-2.89 (m, 1H), 2.54 (t, J=6.4 Hz, 2H), 2.44-2.31 (m, 2H), 2.30-2.20 (m, 2H), 2.05-1.90 (m, 2H), 1.80-1.68 (m, 3H), 1.66-1.52 (m, 1H), 0.96 (t, J=7.6 Hz, 3H)

### Preparation of TAZ-128

To a mixture of 128a (40 mg, 38.0 umol, 1.0 eq, HCl) and sodium 2,3,5,6-tetrafluoro-4-hydroxy-benzenesulfonate (50.9 mg, 190 umol, 5.0 eq) in DCM (1.5 mL) and DMA (0.2 mL) was added EDCI (51.0 mg, 266 umol, 7.0 eq) in one portion at 25° C. and then stirred at 25° C. for 0.5 h. Then the mixture was concentrated. The residue was purified by prep-HPLC(column: Phenomenex Synergi C18 150*25*10 um; mobile phase: [water(0.1% TFA)−ACN]; B %: 15%-45%, 8 min) to give TAZ-128 (35.2 mg, 28.3 umol, 74.5% yield) as yellow solid. ¹H NMR (MeOD, 400 MHz) δ7.49 (s, 1H), 6.96 (s, 1H), 4.79-4.73 (m, 1H), 4.43-4.35 (m, 1H), 4.16-3.98 (m, 2H), 3.97-3.83 (m, 4H), 3.77-3.67 (m, 5H), 3.66-3.57 (m, 34H), 3.42 (s, 2H), 3.30-3.25 (m, 4H), 3.19 (d, J=8.0 Hz, 2H), 2.98 (t, J=5.6 Hz, 3H), 2.44-2.19 (m, 4H), 2.03-1.89 (m, 2H), 1.80-1.54 (m, 4H), 0.96 (t, J=7.6 Hz, 3H). LC/MS [M+H] 1244.5 (calculated); LC/MS [M+H] 1244.2 (observed).

### Example L-131 Synthesis of 4-[3-[2-[2-[3-[4-[2-[5-amino-7-[ethoxy(propyl)carbamoyl]-6H-thieno [3,2-b]azepin-2-yl]ethyl]-1-piperidyl]-3-oxo-propoxy]ethoxy]ethoxy]propanoyloxy]-2,3,5,6-tetrafluoro-benzenesulfonic acid, TAZ-L-131

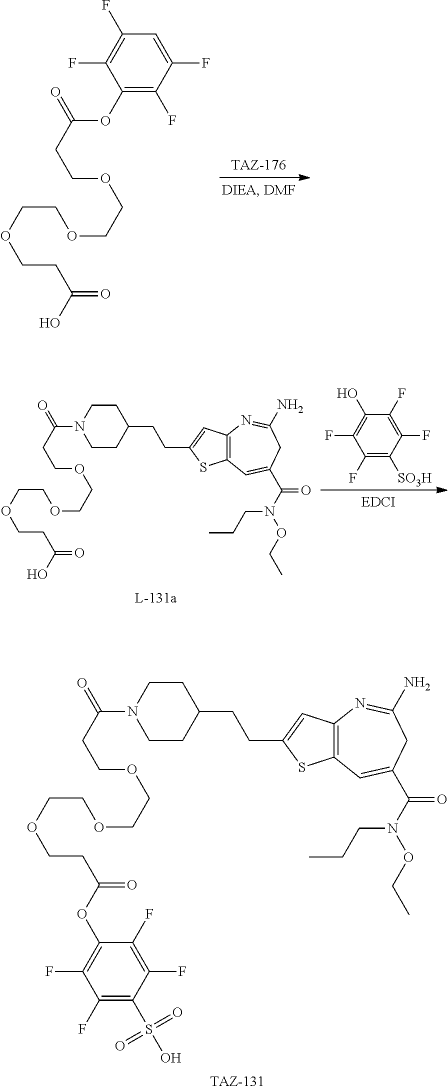 [SMILES: O=C(O)CCOCCOCCOCCC(=O)Oc1c(F)c(F)cc(F)c1F ; CCCN(OCC)C(=O)C1=Cc2sc(CCC3CCN(C(=O)CCOCCOCCOCCC(=O)O)CC3)cc2N=C(N)C1 ; O=S(=O)(O)c1c(F)c(F)c(O)c(F)c1F ; CCCN(OCC)C(=O)C1=Cc2sc(CCC3CCN(C(=O)CCOCCOCCOCCC(=O)Oc4c(F)c(F)c(S(=O)(=O)O)c(F)c4F)CC3)cc2N=C(N)C1]

### Preparation of 3-[2-[2-[3-[4-[2-[5-amino-7-[ethoxy(propyl)carbamoyl]-6H-thieno [3,2-b]azepin-2-yl]ethyl]-1-piperidyl]-3-oxo-propoxy]ethoxy]ethoxy]propanoic acid, L-131a

To a mixture of 5-amino-N-ethoxy-2-[2-(4-piperidyl)ethyl]-N-propyl-6H-thieno[3,2-b]azepine-7-carboxamide, TAZ-176 (0.1 g, 227 umol, 1.0 eq, HCl) in DMF (2 mL) was added DIEA (87.9 mg, 680 umol, 118 uL, 3.0 eq) and 3-[2-[2-[3-oxo-3-(2,3,5,6-tetrafluorophenoxy)propoxy] ethoxy]ethoxy]propanoic acid (90.3 mg, 227 umol, 1.0 eq) in one portion at 0° C. and it was stirred at 0° C. for 0.5 h. The mixture was purified by prep-HPLC(column: Welch Xtimate C18 100*25 mm*3 um; mobile phase: [water(0.05% HCl)-ACN]; B %: 15%-35%, 8 min) to give L-131a (0.06 g, 94.22 umol, 41.55% yield) as yellow oil. ¹H NMR (MeOD, 400 MHz) δ7.46 (s, 1H), 6.89 (s, 1H), 4.54 (d, J=13.6 Hz, 1H), 4.04 (d, J=13.6 Hz, 1H), 3.93 (q, J=7.2 Hz, 2H), 3.77-3.69 (m, 6H), 3.63-3.58 (m, 8H), 3.42 (s, 2H), 3.15-3.01 (m, 1H), 2.94 (t, J=7.6 Hz, 2H), 2.75-2.51 (m, 5H), 1.88-1.78 (m, 2H), 1.78-1.71 (m, 2H), 1.70-1.65 (m, 2H), 1.29-1.07 (m, 5H), 0.97 (t, J=7.6 Hz, 3H).

### Preparation of TAZ-L-131

To a mixture of L-131a (0.06 g, 89.1 umol, 1.0 eq, HCl) and sodium 2,3,5,6-tetrafluoro-4-hydroxy-benzenesulfonate (95.6 mg, 356umol, 4.0 eq) in DCM (1.5 mL) and DMA (0.3 mL) was added EDCI (103 mg, 535 umol, 6.0 eq) in one portion at 25° C. and it was stirred at 25° C. for 0.5 h. The mixture was concentrated to give a residue, and the residue was purified by prep-HPLC(column: Phenomenex Synergi C18 150*25*10 um; mobile phase: [water(0.2% FA)-ACN]; B %: 20%-45%, 8 min) to give TAZ-L-131 (31.7 mg, 36.65 umol, 41.13% yield) as white solid. ¹H NMR (MeOD, 400 MHz) δ7.43 (s, 1H), 6.88 (s, 1H), 4.51 (d, J=13.2 Hz, 1H), 4.01 (d, J=13.6 Hz, 1H), 3.96-3.84 (m, 4H), 3.78-3.69 (m, 4H), 3.68-3.54 (m, 8H), 3.42 (s, 2H), 3.07-3.01 (m, 1H), 2.98 (t, J=5.6 Hz, 2H), 2.88 (t, J=7.2 Hz, 2H), 2.74-2.53 (m, 3H), 1.84-1.68 (m, 4H), 1.68-1.55 (m, 3H), 1.25-1.03 (m, 5H), 0.97 (t, J=7.6 Hz, 3H). LC/MS [M+H] 865.3 (calculated); LC/MS [M+H] 865.3 (observed).

### Example L-133 Synthesis of 4-[3-[2-[2-[2-[2-[2-[2-[2-[2-[2-[2-[3-[[5-amino-7-[2-(cyclobutoxycarbonylamino)ethoxy-propyl-carbamoyl]-6H-thieno[3,2-b]azepin-2-yl]methyl]azetidin-1-yl]ethoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, TAZ-L-133

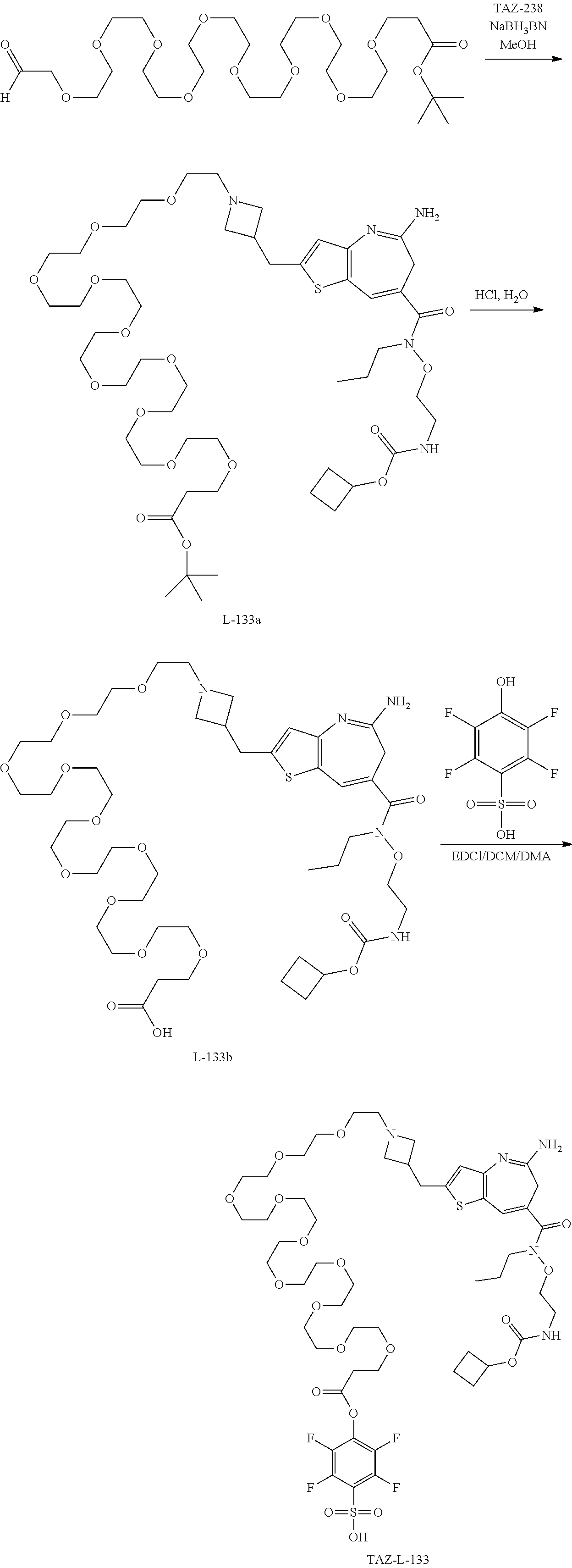

### Preparation of tert-butyl 3-[2-[2-[2-[2-[2-[2-[2-[2-[2-[2-[3-[[5-amino-7-[2-(cyclobutoxycarbonylamino)ethoxy-propyl-carbamoyl]-6H-thieno[3,2-b]azepin-2-yl]methyl]azetidin-1-yl]ethoxy]ethoxy]ethoxy]ethoxy]ethoxy]ethoxy]ethoxy]ethoxy]ethoxy]ethoxy]propanoate, L-133a

To a mixture of cyclobutyl N-[2-[[5-amino-2-(azetidin-3-ylmethyl)-6H-thieno[3,2-b]azepine-7-carbonyl]-propyl-amino]oxyethyl]carbamate, TAZ-238 (0.14 g, 199 umol, 1.0 eq, TFA) and tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (151 mg, 259 umol, 1.3 eq) in MeOH (3 mL) was added NaBH₃CN (25.0 mg, 398 umol, 2.0 eq) in one portion at 25° C. and then stirred at 25° C. for 12 h. The mixture was concentrated. The residue was purified by prep-HPLC(column: Phenomenex Synergi C18 150*25*10 um; mobile phase: [water(0.1% TFA)−ACN]; B %: 20%-50%, 8 min) to give L-133a (0.2 g, 173 umol, 86.8% yield, TFA) as yellow oil. ¹H NMR (MeOD, 400 MHz) δ7.49 (s, 1H), 6.98 (s, 1H), 4.85-4.80 (m, 1H), 4.44-4.25 (m, 2H), 4.18-3.97 (m, 2H), 3.92 (t, J=5.2 Hz, 2H), 3.74-3.67 (m, 6H), 3.66-3.61 (m, 40H), 3.42 (s, 2H), 3.28-3.23 (m, 3H), 2.47 (td, J=1.6, 6.4 Hz, 2H), 2.31-2.19 (m, 2H), 2.02-1.91 (m, 2H), 1.76-1.71 (m, 3H), 1.67-1.54 (m, 1H), 1.45 (s, 9H), 0.96 (t, J=7.6 Hz, 3H).

### Preparation of 3-[2-[2-[2-[2-[2-[2-[2-[2-[2-[2-[3-[[5-amino-7-[2-(cyclobutoxycarbonylamino)ethoxy-propyl-carbamoyl]-6H-thieno[3,2-b]azepin-2-yl]methyl]azetidin-1-yl]ethoxy]ethoxy]ethoxy]ethoxy]ethoxy]ethoxy]ethoxy]ethoxy]ethoxy]ethoxy]propanoic acid, L-133b

To a mixture of L-133a (0.2 g, 173 umol, 1.0 eq, TFA) in H₂O (3 mL) and CH₃CN (0.5 mL) was added HCl (12 M, 216 uL, 15.0 eq) in one portion at 25° C. and then stirred at 80° C. for 0.5 h. The mixture was concentrated. The residue was purified by prep-HPLC(column: Welch Xtimate C18 100*25 mm*3 um; mobile phase: [water(0.05% HCl)−ACN]; B %: 10%-30%, 8 min) to give L-133b (40 mg, 39.0 umol, 22.6% yield, HCl) as yellow oil.

### Preparation of TAZ-L-133

To a mixture of L-133b (40 mg, 39.0 umol, 1.0 eq, HCl) and sodium 2,3,5,6-tetrafluoro-4-hydroxy-benzenesulfonate (41.9 mg, 156 umol, 4.0 eq) in DCM (1 mL) and DMA (0.1 mL) was added EDCI (52.4 mg, 273 umol, 7.0 eq) in one portion at 25° C. and then stirred at 25° C. for 0.5 h. The mixture was filtered and concentrated. The residue was purified by prep-HPLC(column: Phenomenex Synergi C18 150*25*10 um; mobile phase: [water(0.1% TFA)-ACN]; B %: 10%-30%, 8 min) to give TAZ-L-133 (14.7 mg, 12.1 umol, 30.9% yield) as yellow oil. ¹H NMR (MeOD, 400 MHz) δ7.48 (s, 1H), 7.02-6.96 (m, 1H), 4.84-4.84 (m, 1H), 4.41-4.25 (m, 2H), 4.15-4.14 (m, 1H), 4.19-3.97 (m, 2H), 3.95-3.84 (m, 4H), 3.76-3.68 (m, 4H), 3.68-3.58 (m, 38H), 3.52-3.44 (m, 2H), 3.42 (s, 2H), 3.28-3.21 (m, 3H), 2.99 (t, J=5.6 Hz, 2H), 2.25 (q, J=8.4 Hz, 2H), 2.04-1.90 (m, 2H), 1.80-1.54 (m, 4H), 0.96 (t, J=7.6 Hz, 3H). LC/MS [M+H] 1216.5 (calculated); LC/MS [M+H] 1216.6 (observed).

### Example L-139 Synthesis of 4-[3-[2-[2-[2-[2-[2-[2-[2-[2-[2-[3-[3-[[5-amino-7-[isopropoxy(propyl)carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, TAZ-L-139

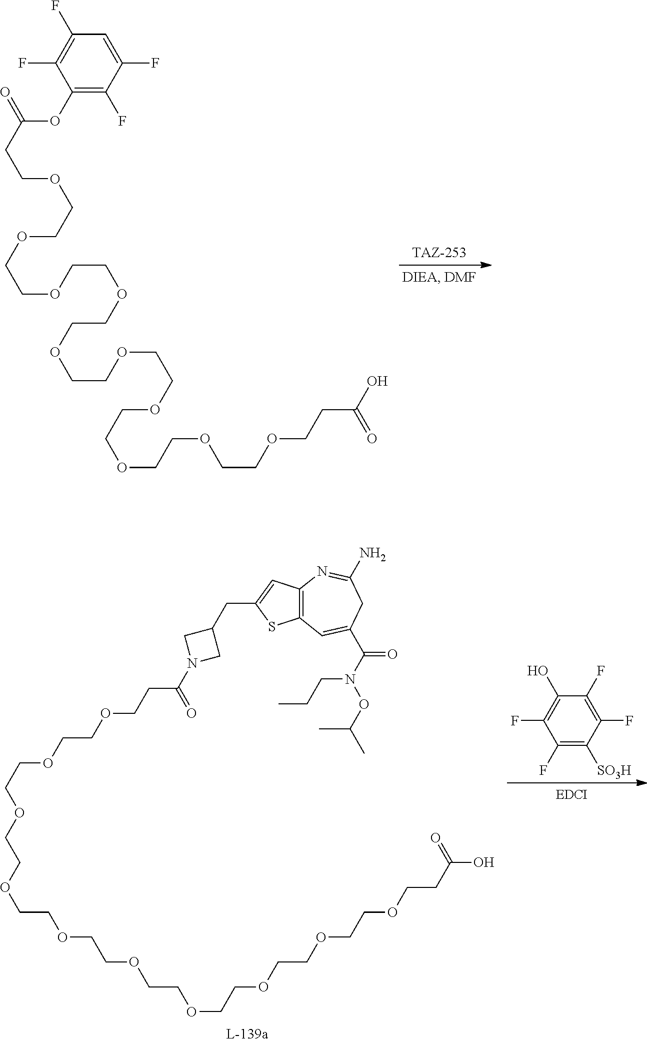 [SMILES: O=C(O)CCOCCOCCOCCOCCOCCOCCOCCOCCOCCOCCC(=O)Oc1c(F)c(F)cc(F)c1F ; CCCN(OC(C)C)C(=O)C1=Cc2sc(CC3CN(C(=O)CCOCCOCCOCCOCCOCCOCCOCCOCCOCCOCCC(=O)O)C3)cc2N=C(N)C1 ; O=S(=O)(O)c1c(F)c(F)c(O)c(F)c1F]

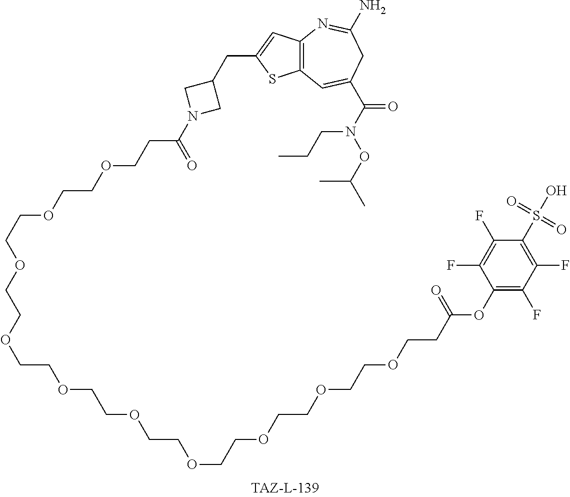 [SMILES: CCCN(OC(C)C)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1]

### Preparation of 3-[2-[2-[2-[2-[2-[2-[2-[2-[2-[3-[3-[[5-amino-7-[isopropoxy(propyl) carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoic acid, L-139a

To a mixture of 5-amino-2-(azetidin-3-ylmethyl)-N-isopropoxy-N-propyl-6H-thieno[3,2-b]azepine-7-carboxamide, TAZ-253 (100 mg, 204 umol, 1 eq, TFA) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (158 mg, 224 umol, 1.1 eq) in THE (3 mL) was added Et₃N (61.9 mg, 612 umol, 85.1 uL, 3 eq), and then stirred at 20° C. for 1 h. The residue was poured into water (5 mL) and the pH of the mixture was adjusted to about 6 with 1M HCl. The aqueous phase was extracted with ethyl acetate (8 mL×1)-discarded, the aqueous phase was further extracted with dichloromethane/isopropyl alcohol=3:1 (8 mL×3), the combined organic phase was dried with anhydrous Na₂SO₄, filtered and concentrated in vacuum to give L-139a (150 mg, 164 umol, 80.23% yield) as a light yellow oil.

### Preparation of TAZ-L-139

To a solution of L-139a (100 mg, 109 umol, 1 eq) in DCM (1.5 mL) and DMA (0.5 mL) was added sodium 2,3,5,6-tetrafluoro-4-hydroxy-benzenesulfonate (117 mg, 436 umol, 4 eq) and EDCI (83.6 mg, 436 umol, 4 eq), and then stirred at 20° C. for 2 h. The reaction mixture was filtered and concentrated under residue pressure. The residue was purified by prep-HPLC (column: Phenomenex Synergi C18 150*25*10 um; mobile phase: [water(0.1% TFA)−ACN]; B %: 15%-35%, 8 min) to give TAZ-L-139 (69.7 mg, 55.3 umol, 50.76% yield, TFA) as a light yellow solid. ¹H NMR (MeOD, 400 MHz) δ7.45 (s, 1H), 6.96 (s, 1H), 4.85-4.80 (m, 1H), 4.39 (t, J=8.6 Hz, 1H), 4.26-4.20 (m, 1H), 4.11 (t, J=9.2 Hz, 1H), 4.02 (dd, J=5.2, 8.5 Hz, 1H), 3.87 (t, J=5.8 Hz, 2H), 3.82-3.67 (m, 6H), 3.66-3.58 (m, 34H), 3.42 (s, 2H), 3.18 (d, J=7.8 Hz, 2H), 3.05-2.95 (m, 3H), 2.42-2.31 (m, 2H), 1.79-1.69 (m, 2H), 1.18 (d, J=6.2 Hz, 6H), 0.95 (t, J=7.4 Hz, 3H). LC/MS [M+H] 1145.4 (calculated); LC/MS [M+H] 1145.4 (observed).

### Example L-144 Synthesis of 4-[3-[2-[2-[2-[2-[2-[2-[2-[2-[2-[3-[3-[[5-amino-7-[2-(isopropoxycarbonylamino)ethoxy-propyl-carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, TAZ-L-144

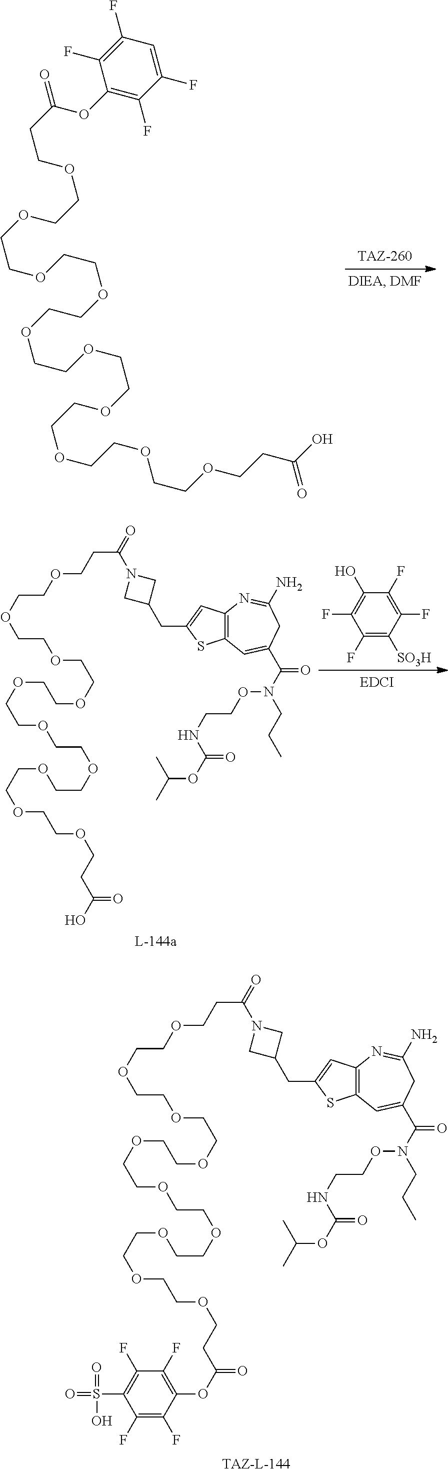 [SMILES: O=C(O)CCOCCOCCOCCOCCOCCOCCOCCOCCOCCOCCC(=O)Oc1c(F)c(F)cc(F)c1F ; CCCN(OCCNC(=O)OC(C)C)C(=O)C1=Cc2sc(CC3CN(C(=O)CCOCCOCCOCCOCCOCCOCCOCCOCCOCCOCCC(=O)O)C3)cc2N=C(N)C1 ; O=S(=O)(O)c1c(F)c(F)c(O)c(F)c1F ; CCCN(OCCNC(=O)OC(C)C)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1]

### Preparation of 3-[2-[2-[2-[2-[2-[2-[2-[2-[2-[3-[3-[[5-amino-7-[2-(isopropoxycarbonylamino)ethoxy-propyl-carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoic acid, L-144a

To a mixture of isopropyl N-[2-[[5-amino-2-(azetidin-3-ylmethyl)-6H-thieno[3,2-b]azepine-7-carbonyl]-propyl-amino]oxyethyl]carbamate, TAZ-260 (0.13 g, 161 umol, 1.0 eq, TFA salt) in THE (4 mL) was added Et₃N (49.0 mg, 484 umol, 67.4 uL, 3.0 eq) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (114 mg, 161 umol, 1.0 eq) in one portion at 0° C. and then stirred at 0° C. for 0.5 h. The mixture was diluted with water (5 mL) and the pH of the mixture was adjusted to ˜6 with TFA. Then it was extracted with EtOAc (10 mL)-discarded. The water phase was further extracted with DCM:i-PrOH=3:1(10 mL×3). The organic layer was dried over Na₂SO₄, filtered and concentrated to give L-144a (0.21 g, crude, 3TFA) as yellow oil.

### Preparation of TAZ-L-144

To a mixture of L-144a (0.2 g, 199 umol, 1.0 eq) in DCM (3 mL) and DMA (0.3 mL) was added sodium; 2,3,5,6-tetrafluoro-4-hydroxy-benzenesulfonate (267 mg, 996 umol, 5.0 eq) and EDCI (267 mg, 1.39 mmol, 7.0 eq) in one portion at 25° C. and then stirred at 25° C. for 0.5 h. The mixture was concentrated to give a residue. The residue was purified by prep-HPLC(column: Phenomenex Synergi C18 150*25*10 um; mobile phase: [water(0.1% TFA)-ACN]; B %: 15%-40%, 8 min) to give TAZ-L-144 (98.2 mg, 79.69 umol, 40.01% yield) as light yellow oil. ¹H NMR (MeOH, 400 MHz) δ7.49 (s, 1H), 6.96 (s, 1H), 4.82-4.74 (m, 1H), 4.39 (t, J=8.8 Hz, 1H), 4.11 (t, J=9.2 Hz, 1H), 4.03 (dd, J=5.6, 9.2 Hz, 1H), 3.93 (t, J=5.2 Hz, 2H), 3.87 (t, J=6.0 Hz, 2H), 3.76-3.69 (m, 5H), 3.68-3.52 (m, 38H), 3.43 (s, 2H), 3.18 (d, J=7.6 Hz, 2H), 2.98 (t, J=6.0 Hz, 3H), 2.43-2.30 (m, 2H), 1.79-1.68 (m, 2H), 1.17 (d, J=6.4 Hz, 6H), 0.95 (t, J=7.6 Hz, 3H). LC/MS [M+H] 1232.5 (calculated); LC/MS [M+H] 1232.7 (observed).

### Example L-145 Synthesis of 4-[3-[2-[2-[2-[2-[2-[2-[2-[2-[2-[3-[3-[[5-amino-7-[2-(ethylcarbamoylamino)ethoxy-propyl-carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic acid, TAZ-L-145

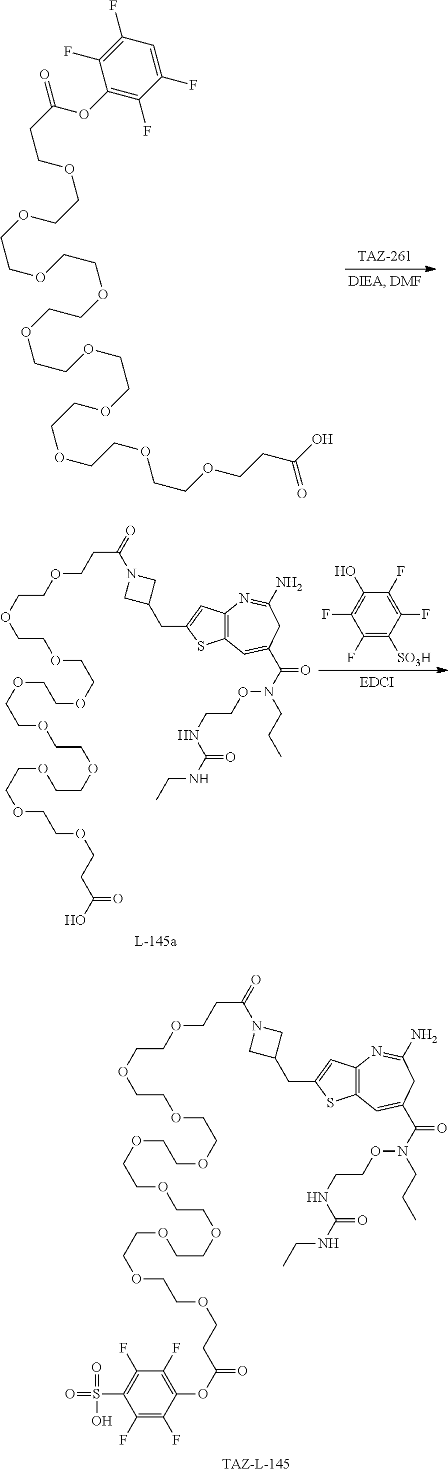 [SMILES: O=C(O)CCOCCOCCOCCOCCOCCOCCOCCOCCOCCOCCC(=O)Oc1c(F)c(F)cc(F)c1F ; CCCN(OCCNC(=O)NCC)C(=O)C1=Cc2sc(CC3CN(C(=O)CCOCCOCCOCCOCCOCCOCCOCCOCCOCCOCCC(=O)O)C3)cc2N=C(N)C1 ; O=S(=O)(O)c1c(F)c(F)c(O)c(F)c1F ; CCCN(OCCNC(=O)NCC)C(=O)C1=Cc2sc(CC3CN(C(=O)CCOCCOCCOCCOCCOCCOCCOCCOCCOCCOCCC(=O)Oc4c(F)c(F)c(S(=O)(=O)O)c(F)c4F)C3)cc2N=C(N)C1]

### Preparation of 3-[2-[2-[2-[2-[2-[2-[2-[2-[2-[3-[3-[[5-amino-7-[2-(ethylcarbamoylamino)ethoxy-propyl-carbamoyl]-6H-thieno[3,2-b]azepin-2-yl]methyl]azetidin-1-yl]-3-oxo-propoxy]ethoxy]ethoxy]ethoxy]ethoxy]ethoxy]ethoxy]ethoxy]ethoxy]ethoxy]propanoic acid, L-145a

To a solution of 5-amino-2-(azetidin-3-ylmethyl)-N-[2-(ethylcarbamoylamino) ethoxy]-N-propyl-6H-thieno[3,2-b]azepine-7-carboxamide, TAZ-261 (120 mg, 177 umol, 1 eq, TFA) in THE (3.00 mL) was added Et₃N (54.0 mg, 532 umol, 3 eq) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethoxy]propanoic acid (125 mg, 177 umol, 1 eq), and then stirred at 0° C. for 1 h. The mixture was diluted with water (10 mL) and the pH of the mixture was adjusted to about pH 6 by TFA and extracted with MTBE (10 mL)-discarded and the water phase was further extracted with DCM:i-PrOH=3:1 (20 mL×3). The organic layer was washed with brine (30 mL×3), dried over Na₂SO₄, filtered and concentrated to give L-145a (170 mg, 172 umol, 96.90% yield) as light yellow oil.

### Preparation of TAZ-L-145

To a solution of L-145a (170 mg, 172 umol, 1 eq) and sodium 2,3,5,6-tetrafluoro-4-hydroxybenzenesulfonate (184 mg, 687 umol, 4 eq) in DCM (3.00 mL) and DMA (0.15 mL) was added EDCI (132 mg, 687 umol, 4 eq), and then stirred at 25° C. for 1 h. The mixture was concentrated to give a residue. The residue was purified by prep-HPLC (column: Phenomenex Synergi C18 150*25*10 um; mobile phase: [water(0.1% TFA)−ACN]; B %: 20%-40%, 8 min) to give TAZ-L-145 (103 mg, 77.37 umol, 45.02% yield, TFA) as light yellow oil. ¹H NMR (MeOD, 400 MHz) δ7.47 (s, 1H), 6.95 (s, 1H), 4.39 (t, J=8.5 Hz, 1H), 4.11 (t, J=9.0 Hz, 1H), 4.02 (dd, J=5.3, 8.8 Hz, 1H), 3.89 (td, J=5.6, 13.9 Hz, 4H), 3.76-3.68 (m, 6H), 3.67-3.56 (m, 36H), 3.44 (s, 2H), 3.18 (d, J=7.5 Hz, 2H), 3.07 (q, J=7.3 Hz, 3H), 2.98 (t, J=5.9 Hz, 2H), 2.44-2.30 (m, 2H), 1.79-1.69 (m, 2H), 1.05 (t, J=7.2 Hz, 3H), 0.96 (t, J=7.4 Hz, 3H). LC/MS [M+H] 1217.5 (calculated); LC/MS [M+H] 1217.6 (observed).

### Example L-147 Synthesis of 4-((40-(5-amino-7-((2-((cyclobutoxycarbonyl)amino)ethoxy)(propyl)carbamoyl)-6H-thieno[3,2-b]azepin-2-yl)-34-oxo-4,7,10,13,16,19,22,25,28,31-decaoxa-35-azatetracontanoyl)oxy)-2,3,5,6-tetrafluorobenzenesulfonic acid, TAZ-L-147

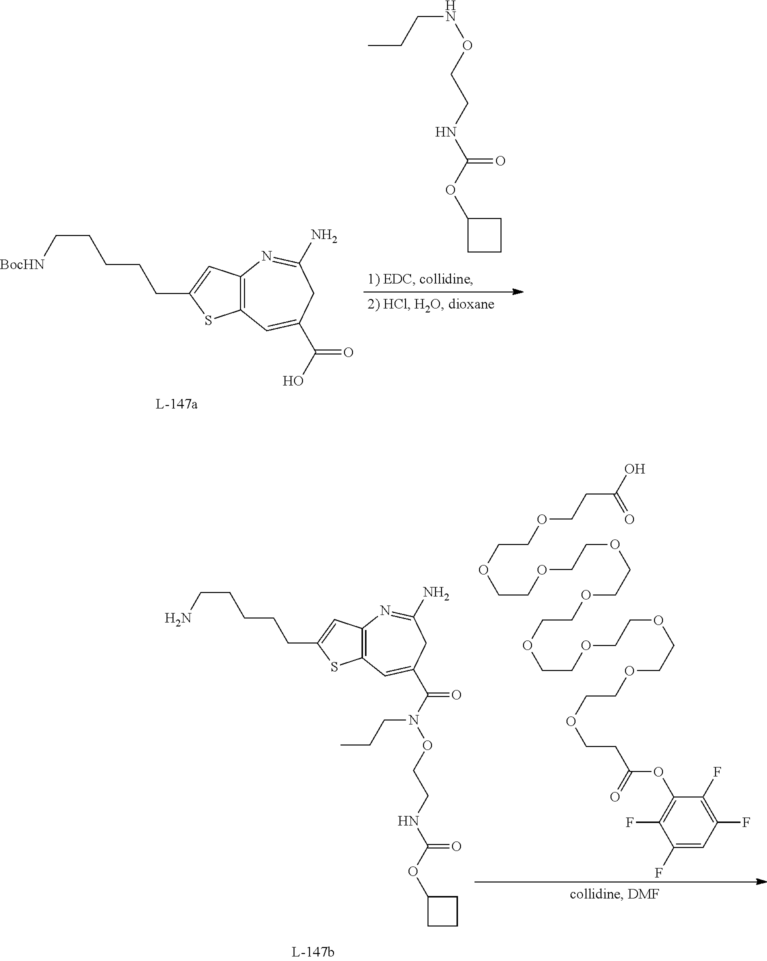 [SMILES: CC(C)(C)OC(=O)NCCCCCc1cc2c(s1)C=C(C(=O)O)CC(N)=N2 ; CCCNOCCNC(=O)OC1CCC1 ; CCCN(OCCNC(=O)OC1CCC1)C(=O)C1=Cc2sc(CCCCCN)cc2N=C(N)C1 ; O=C(O)CCOCCOCCOCCOCCOCCOCCOCCOCCOCCOCCC(=O)Oc1c(F)c(F)cc(F)c1F]

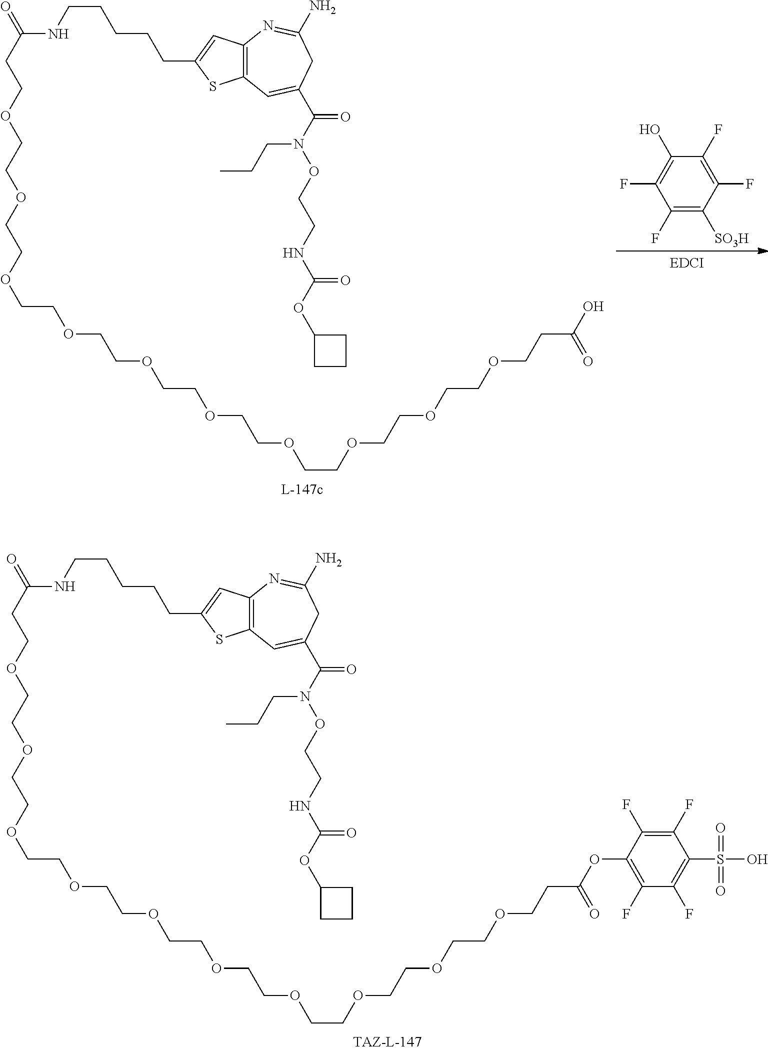 [SMILES: CCCN(OCCNC(=O)OC1CCC1)C(=O)C1=Cc2sc(CCCCCNC(=O)CCOCCOCCOCCOCCOCCOCCOCCOCCOCCOCCC(=O)O)cc2N=C(N)C1 ; O=S(=O)(O)c1c(F)c(F)c(O)c(F)c1F ; CCCN(OCCNC(=O)OC1CCC1)C(=O)C1=Cc2sc(CCCCCNC(=O)CCOCCOCCOCCOCCOCCOCCOCCOCCOCCOCCC(=O)Oc3c(F)c(F)c(S(=O)(=O)O)c(F)c3F)cc2N=C(N)C1]

### Preparation of 5-(5-amino-7-((2-((cyclobutoxycarbonyl)amino)ethoxy)(propyl)carbamoyl)-6H-thieno[3,2-b]azepin-2-yl)pentan-1-aminium chloride, L-147b

5-Amino-2-(5-((tert-butoxycarbonyl)amino)pentyl)-6H-thieno[3,2-b]azepine-7-carboxylic acid, L-147a (0.78 g, 1.98 mmol, 1 equiv.) and cyclobutyl (2-((propylamino)oxy)ethyl)carbamate (0.5 g, 1.98 mmol, 1 equiv.) were combined in DMF. Collidine (0.52 ml, 3.9 mmol, 1.98 equiv.) was added, followed by EDCI, also known as EDC, 1-ethyl-3-(3-dimethylaminopropyl)carbodiimide, CAS Reg. No. 1892-57-5 (0.38 g, 1.98 mmol, 1 equiv.). The reaction monitored by LCMS, then concentrated and purified by reverse-phase flash chromatography. Combined fractions were lyophilized, then taken up in 4 N HCl/dioxane. The deprotected product was purified by reverse-phase flash chromatography to give L-147b (0.8 g, 1.51 mmol, 82%). LC/MS [M+H] 492.26 (calculated); LC/MS [M+H] 492.45 (observed).

### Preparation of 40-(5-amino-7-((2-((cyclobutoxycarbonyl)amino)ethoxy)(propyl)carbamoyl)-6H-thieno[3,2-b]azepin-2-yl)-34-oxo-4,7,10,13,16,19,22,25,28,31-decaoxa-35-azatetracontanoic acid, L-147c

Intermediate L-147b (0.148 g, 0.3 mmol, 1 equiv.) and 34-oxo-34-(2,3,5,6-tetrafluorophenoxy)-4,7,10,13,16,19,22,25,28,31-decaoxatetratriacontanoic acid (0.23 g, 0.32 mmol, 1.07 equiv.) were dissolved in 3 ml DMF. Collidine (0.2 ml, 1.5 mmol, 5 equiv.) was added, and the reaction stirred at ambient temperature. The reaction was purified by reverse-phase HPLC to give L-147c (0.16 g, 0.16 mmol, 52%). LC/MS [M+H] 1032.54 (calculated); LC/MS [M+H] 1032.81 (observed).

### Preparation of TAZ-L-147

Intermediate L-147c (0.16 g, 0.155 mmol, 1 equiv.) and 2,3,5,6-tetrafluoro-4-hydroxybenzenesulfonic acid (0.083 g, 0.31 mmol, 2 equiv.) were dissolved in 2 ml DMF. Collidine (0.1 ml, 0.78 mmol, 5 equiv.) was added, followed by EDC (0.045 g, 0.23 mmol, 1.5 equiv.). The reaction was stirred at room temperature and monitored by LCMS, then diluted with water and purified by reverse-phase HPLC to give TAZ-L-147 (0.095 g, 0.075 mmol, 49%). LC/MS [M+H] 1260.49 (calculated); LC/MS [M+H] 1260.70 (observed).

### Example 201 Preparation of Macromolecule-Supported Compounds (MSC)

In an exemplary procedure, a macromolecule is buffer-exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, Mo.). The eluates are then each adjusted to a concentration of about 1-10 mg/ml using the buffer and then sterile filtered. The macromolecule is pre-warmed to 20-30° C. and rapidly mixed with 2-20 (e.g., 7-40) molar equivalents of thienoazepine-linker (TAZ-L) compound of Formula II. The reaction is allowed to proceed for about 16 hours at 30° C. and the MSC is separated from reactants by running over two successive G-25 desalting columns equilibrated in phosphate buffered saline (PBS) at pH 7.2 to provide the MSC of Table 3. Adjuvant-macromolecule ratio (DAR) is determined by liquid chromatography mass spectrometry analysis using a C4 reverse phase column on an ACQUITY™ UPLC H-class (Waters Corporation, Milford, Mass.) connected to a XEVO™ G-2-XS TOF mass spectrometer (Waters Corporation).

For conjugation, the macromolecule may be dissolved in a aqueous buffer system known in the art that will not adversely impact the stability or antigen-binding specificity of the macromolecule. Phosphate buffered saline may be used. The thienoazepine-linker (TAZ-L) intermediate compound is dissolved in a solvent system comprising at least one polar aprotic solvent as described elsewhere herein. In some such aspects, thienoazepine-linker (TAZ-L) intermediate is dissolved to a concentration of about 5 mM, about 10 mM, about 20 mM, about 30 mM, about 40 mM or about 50 mM, and ranges thereof such as from about 5 mM to about 50 mM or from about 10 mM to about 30 mM in pH 8 Tris buffer (e.g., 50 mM Tris). In some aspects, the thienoazepine-linker intermediate is dissolved in DMSO (dimethylsulfoxide), DMA (dimethylacetamide) or acetonitrile, or another suitable dipolar aprotic solvent.

Alternatively in the conjugation reaction, an equivalent excess of thienoazepine-linker (TAZ-L) intermediate solution may be diluted and combined with macromolecule solution. The thienoazepine-linker intermediate solution may suitably be diluted with at least one polar aprotic solvent and at least one polar protic solvent, examples of which include water, methanol, ethanol, n-propanol, and acetic acid. The molar equivalents of thienoazepine-linker intermediate to antibody may be about 1.5:1, about 3:1, about 5:1, about 10:1, about 15:1, or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS. The conjugation reaction is typically complete in a range from about 1 hour to about 16 hours. After the reaction is complete, a reagent may be added to the reaction mixture to quench the reaction. If macromolecule thiol groups are reacting with a thiol-reactive group such as maleimide of the thienoazepine-linker intermediate, unreacted macromolecule thiol groups may be reacted with a capping reagent. An example of a suitable capping reagent is ethylmaleimide.

Following conjugation, the MSC may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, tangential flow filtration, and combinations thereof. For instance, purification may be preceded by diluting the MSC, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 202 HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4×10⁴ cells/well with substrate containing HEK detection medium and immunostimulatory molecules. Activity was measured using a plate reader at 620-655 nm wavelength.

### Example 203 Assessment of Macromolecule-Supported Compound (MSC) Activity In Vitro

This example shows that Macromolecule-supported compounds (MSC) may be effective at eliciting myeloid activation and useful for the treatment of cancer.

Isolation of Human Antigen Presenting Cells: Human myeloid antigen presenting cells (APCs) were negatively selected from human peripheral blood obtained from healthy blood donors (Stanford Blood Center, Palo Alto, Calif.) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >90% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.

Myeloid APC Activation Assay: 2×10⁵ APCs were incubated in 96-well plates (Corning, Corning, N.Y.) containing iscove's modified dulbecco's medium, IMDM (Lonza) supplemented with 10% FBS, 100 U/mL penicillin, 100 μg/mL (micrograms per milliliter) streptomycin, 2 mM L-glutamine, sodium pyruvate, non-essential amino acids, and where indicated, various concentrations of unconjugated (naked) PD-L1 or HER2 antibodies and MSC (as prepared according to the Example above). Trastuzumab and avelumab were used as the antibody constructs. Cell-free supernatants were analyzed after 18 hours via ELISA to measure TNFα secretion as a readout of a proinflammatory response.

All references, including publications, patent applications, and patents, cited herein are hereby incorporated by reference to the same extent as if each reference were individually and specifically indicated to be incorporated by reference and were set forth in its entirety herein.

1. A macromolecule-supported compound comprising a macromolecular support covalently attached to one or more 5-amino-thienoazepine moieties by a linker, and having Formula I:
Ms-[L-TAZ]ₚ I
or a pharmaceutically acceptable salt thereof,
wherein:
Ms is the macromolecular support, selected from the group consisting of a peptide, a nucleotide, a carbohydrate, a lipid, an antibody construct, a biopolymer, a nanoparticle, and an immune checkpoint inhibitor;
p is an integer from 1 to 50;
TAZ is the 5-amino-thienoazepine moiety having the formula: 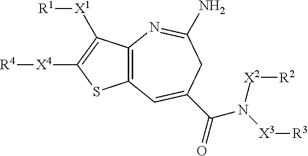
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-C(═O)—(C₁-C₁₂ alkyldiyl)-N(R⁵)—*;
—(C₂-C₉ heterocyclyl)-NR⁵—C(═NR^{5a})NR⁵—*;
—(C₂-C₉ heterocyclyl)-NR⁵—(C₆-C₂₀ aryldiyl)-(C₁-C₁₂ alkyldiyl)-N(R⁵)—*;
—(C₂-C₉ heterocyclyl)-(C₆-C₂₀ aryldiyl)-*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—(C₁-C₂₀ heteroaryl)-N(R⁵)C(═O)—(C₁-C₁₂ alkyldiyl)-N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)C(═O)—(C₁-C₁₂ alkyldiyl)-N(R⁵)—*;
—N(R⁵)—(C₂-C₅ heteroaryl);
—N(R⁵)—S(═O)₂—(C₁-C₁₂ alkyl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
X¹, X², X³, and X⁴ are independently selected from the group consisting of a bond, C(═O), C(═O)N(R⁵), O, N(R⁵), S, S(O)₂, and S(O)₂N(R⁵);
R⁵ is selected from the group consisting of H, C₆-C₂₀ aryl, C₃-C₁₂ carbocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
—C(═O)-(PEG)-;
—C(═O)-(PEG)-C(═O)—;
—C(═O)-(PEG)-O—;
—C(═O)-(PEG)-C(═O)-(PEP)-;
—C(═O)-(PEG)-C(═O)N(R⁵)—(C₁-C₁₂ alkyldiyl)-;
—C(═O)-(PEG)-C(═O)N(R⁵)—(C₁-C₁₂ alkyldiyl)-N(R⁵)C(═O)—(C₂-C₅ monoheterocyclyldiyl)-;
—C(═O)-(PEG)-C(═O)N(R⁵)—(C₁-C₁₂ alkyldiyl)-(MCgluc)-;
—C(═O)-(PEG)-C(═O)-(MCgluc)-;
—C(═O)-(PEG)-C(═O)-(PEP)-N(R⁵)—(C₁-C₁₂ alkyldiyl)-;
—C(═O)-(PEG)-C(═O)-(PEP)-N(R⁵)—(C₁-C₁₂ alkyldiyl)-N(R⁵)C(═O)—(C₂-C₅ monoheterocyclyldiyl)-;
—C(═O)-(PEG)-N(R⁵)—;
—C(═O)-(PEG)-N(R⁵)C(═O)—;
—C(═O)-(PEG)-N(R⁵)-(PEG)-C(═O)-(PEP)-;
—C(═O)-(PEG)-N⁺(R⁵)₂-(PEG)-C(═O)-(PEP)-;
—C(═O)-(PEG)-C(═O)—N(R⁵)CH(AA₁)C(═O)-(PEG)-C(═O)-(PEP)-;
—C(═O)-(PEG)-C(═O)—N(R⁵)CH(AA₁)C(═O)—N(R⁵)—(C₁-C₁₂ alkyldiyl)-;
—C(═O)-(PEG)-SS—(C₁-C₁₂ alkyldiyl)-OC(═O)—;
—C(═O)-(PEG)-SS—(C₁-C₁₂ alkyldiyl)-C(═O)—;
—C(═O)—(C₁-C₁₂ alkyldiyl)-C(═O)-(PEP)-;
—C(═O)—(C₁-C₁₂ alkyldiyl)-C(═O)-(PEP)-N(R⁵)—(C₁-C₁₂ alkyldiyl)-;
—C(═O)—(C₁-C₁₂ alkyldiyl)-C(═O)-(PEP)-N(R⁵)—(C₁-C₁₂ alkyldiyl)-N(R⁵)—C(═O);
—C(═O)—(C₁-C₁₂ alkyldiyl)-C(═O)-(PEP)-N(R⁵)—(C₁-C₁₂ alkyldiyl)-N(R⁵)C(═O)—(C₂-C₅ monoheterocyclyldiyl)-;
—C(═O)—CH₂CH₂OCH₂CH₂—(C₁-C₂₀ heteroaryldiyl)-CH₂O-(PEG)-C(═O)-(MCgluc)-;
—C(═O)—CH₂CH₂OCH₂CH₂—(C₁-C₂₀ heteroaryldiyl)-CH₂O-(PEG)-C(═O)-(MCgluc)-N(R⁵)—(C₁-C₁₂ alkyldiyl)-N(R⁵)C(═O)—(C₂-C₅ monoheterocyclyldiyl)-; and
-(succinimidyl)-(CH₂)m-C(═O)-(PEP)-N(R⁵)—(C₁-C₁₂ alkyldiyl)-N(R⁵)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
PEP has the formula: 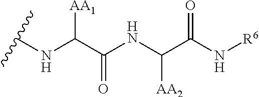
where AA₁ and AA₂ are independently selected from an amino acid side chain, or AA₁ or AA₂ and an adjacent nitrogen atom form a 5-membered ring proline amino acid, and the wavy line indicates a point of attachment;
R⁶ is selected from the group consisting of C₆-C₂₀ aryldiyl and C₁-C₂₀ heteroaryldiyl, substituted with —CH₂O—C(═O)— and optionally with: 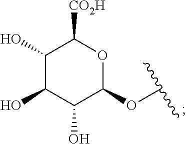 [SMILES: *O[C@@H]1O[C@H](C(=O)O)[C@@H](O)[C@H](O)[C@H]1O]
and
MCgluc is selected from the groups: 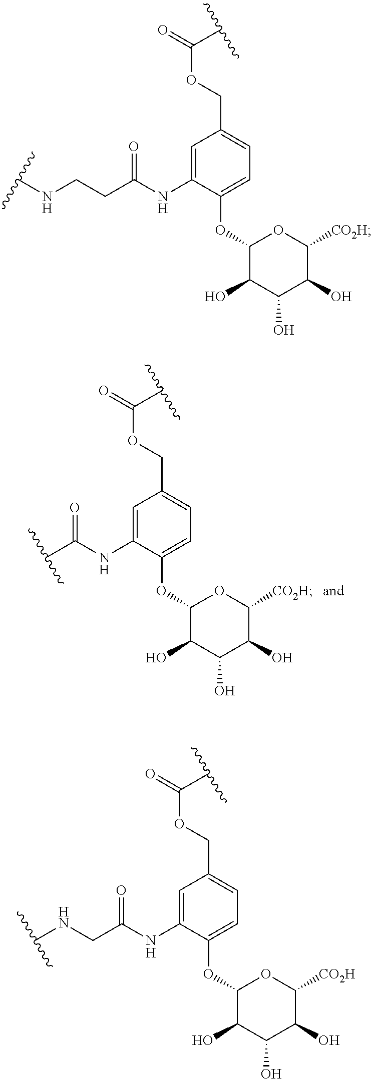
where q is 1 to 8, and AA is an amino acid side chain; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂₀H, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

2-11. (canceled)

12. The macromolecule-supported compound of claim 1 wherein PEP has the formula: 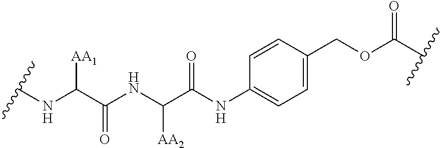
wherein AA₁ and AA₂ are independently selected from a side chain of a naturally-occurring amino acid, or AA₁ or AA₂ with an adjacent nitrogen atom form a 5-membered ring proline amino acid.

13. (canceled)

14. The macromolecule-supported compound of claim 1 wherein PEP has the formula:

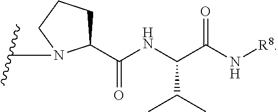

15. The macromolecule-supported compound of claim 1 wherein MCgluc has the formula:

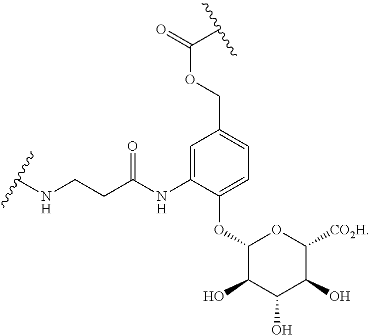 [SMILES: *NCCC(=O)Nc1cc(COC(*)=O)ccc1O[C@@H]1O[C@H](C(=O)O)[C@@H](O)[C@H](O)[C@H]1O]

16. The macromolecule-supported compound of claim 1 wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

17. The macromolecule-supported compound of claim 16 wherein AA₁ is —CH(CH₃)₂, and AA₂ is —CH₂CH₂CH₂NHC(O)NH₂.

18. The macromolecule-supported compound of claim 1 wherein AA₁ and AA₂ are independently selected from GlcNAc aspartic acid, —CH₂SO₃H, and —CH₂OPO₃H.

19. The macromolecule-supported compound of claim 1 wherein X¹ is a bond, and R¹ is H.

20. The macromolecule-supported compound of claim 1 wherein X² is a bond, and R² is C₁-C₈ alkyl.

21. The macromolecule-supported compound of claim 1 wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

22. The macromolecule-supported compound of claim 21 wherein R² and R³ are each independently selected from —CH₂CH₂CH₃, —OCH₂CH₃, —CH₂CH₂CF₃, and —CH₂CH₂CH₂OH.

23. The macromolecule-supported compound of claim 21 wherein R² is C₁-C₈ alkyl and R³ is —(C₁-C₈ alkyldiyl)-N(R⁵)CO₂R⁴.

24. The macromolecule-supported compound of claim 23 wherein R² is —CH₂CH₂CH₃ and R³ is —CH₂CH₂CH₂NHCO₂(t-Bu).

25. The macromolecule-supported compound of claim 21 wherein R² and R³ are each —CH₂CH₂CH₃.

26. The macromolecule-supported compound of claim 1 wherein X³—R³ is selected from the group consisting of:

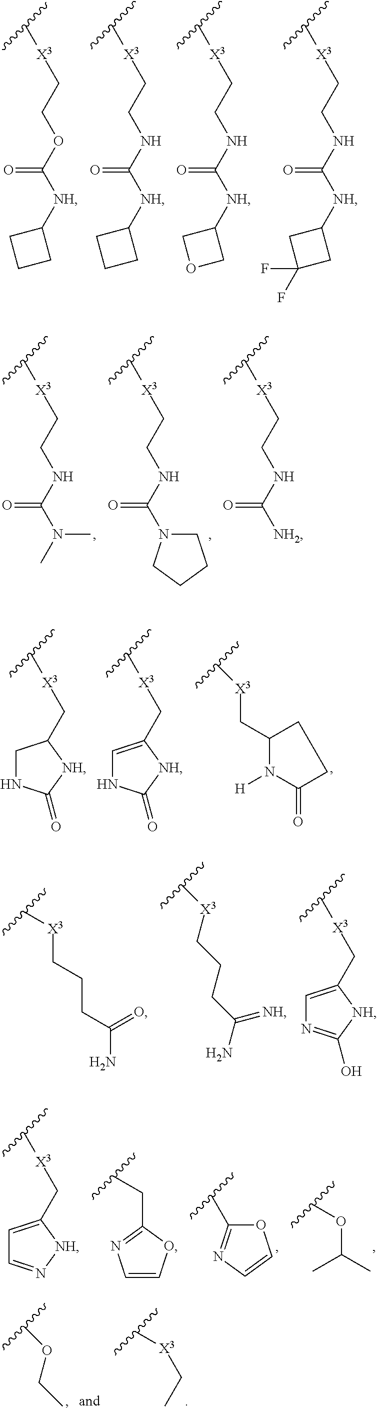

27. The macromolecule-supported compound of claim 1 wherein one of R² and R³ is selected from:
—(C₁-C₁₂ alkyldiyl)-N(R⁵)—*;
—(C₁-C₁₂ alkyldiyl)-O—(C₁-C₁₂ alkyldiyl)-N(R⁵)—*;
—(C₁-C₁₂ alkyldiyl)-N(R⁵)C(═NR⁵)—N(R⁵)—*;
—(C₁-C₁₂ alkyldiyl)-(C₆-C₂₀ aryldiyl)-(C₁-C₁₂ alkyldiyl)-N(R⁵)—*;
—(C₁-C₁₂ alkyldiyl)-(C₆-C₂₀ aryldiyl)-(C₁-C₁₂ alkyldiyl)-N(R⁵)—C(═NR⁵)N(R⁵)—*;
—(C₂-C₆ alkynyldiyl)-N(R⁵)—*; and
—(C₂-C₆ alkynyldiyl)-N(R⁵)C(═NR⁵)N(R⁵)—*;
X² and X³ are a bond, and where the asterisk * indicates the attachment site of L.

28. The macromolecule-supported compound of claim 1 wherein L is selected from the group consisting of:
—C(═O)-(PEG)-;
—C(═O)-(PEG)-C(═O)—;
—C(═O)-(PEG)-O—;
—C(═O)-(PEG)-N(R⁵)—; and
—C(═O)-(PEG)-N(R⁵)C(═O)—.

29. The macromolecule-supported compound of claim 1 selected from Formulae Ia-Ic:

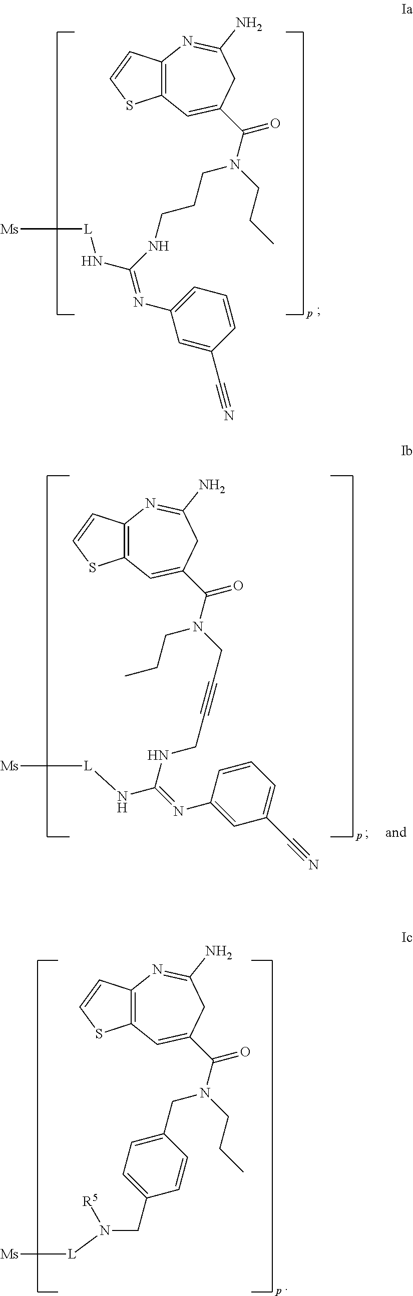

30. The macromolecule-supported compound of claim 1 selected from Formulae Id-Ih:

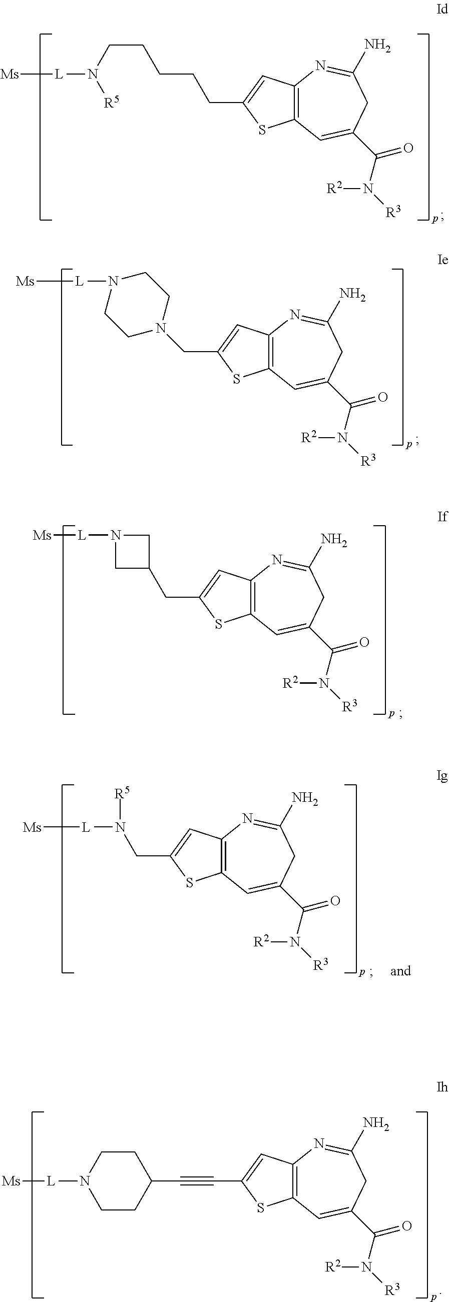

31-55. (canceled)

56. A method of preparing a macromolecule-supported compound of Formula I of claim 1 wherein a 5-amino-thienoazepine-linker compound selected from Tables 2a-c is conjugated with the macromolecular support.

57. (canceled)

58. A method for treating cancer comprising administering a therapeutically effective amount of a macromolecule-supported compound according to claim 1, to a patient in need thereof, wherein the cancer is selected from bladder cancer, urinary tract cancer, urothelial carcinoma, lung cancer, non-small cell lung cancer, Merkel cell carcinoma, colon cancer, colorectal cancer, gastric cancer, and breast cancer.

59. The method of claim 58, wherein the cancer is susceptible to a pro-inflammatory response induced by TLR7 and/or TLR8 agonism.

60. The method of claim 58, wherein the cancer is selected from a PD-L1-expressing cancer, a HER2-expressing cancer, and a CEA-expressing cancer.

61-63. (canceled)

64. The method of claim 58, wherein the cancer is selected from triple-negative breast cancer, metastatic Merkel cell carcinoma, HER2 overexpressing gastric cancer, and gastroesophageal junction adenocarcinoma.

65-67. (canceled)
